(19) United States
(12) Patent Application Publication
(10) Pub. No.: US 2025/0179483 A1
(43) Pub. Date: Jun. 5, 2025

(54) GENOME EDITING COMPOSITIONS AND METHODS FOR TREATMENT OF GLYCOGEN STORAGE DISEASE TYPE 1B

(71) Applicant: Prime Medicine, Inc., Cambridge, MA (US)

(72) Inventors: Christopher Austin Hart, Cambridge, MA (US); Jonathan M. Levy, Cambridge, MA (US)

(21) Appl. No.: 18/712,191

(22) PCT Filed: Nov. 23, 2022
(86) PCT No.: PCT/US22/50895

§ 371 Date: May 21, 2024

Related U.S. Application Data
(60) Provisional application No. 63/282,515, filed on Nov. 23, 2021.
(60) Provisional application No. 63/282,516, filed on Nov. 23, 2021.

(51) Int. Cl. C12N 15/11; C07K 14/47; C12N 9/12; C12N 9/22; C12N 15/88

(52) U.S. Cl. CPC C12N 15/111; C07K 14/4702; C12N 9/1276; C12N 9/22; C12N 15/88; C07K 2319/80; C12N 2310/20; C12Y 207/07049

(57) ABSTRACT

Provided herein are compositions and methods of using prime editing systems comprising prime editors and prime editing guide RNAs for treatment of genetic disorders.

### CROSS REFERENCE TO RELATED APPLICATIONS

This application is a § 371 national-stage application based on PCT/US2022/050895, filed Nov. 23, 2022, which claims the benefit of priority to U.S. Provisional Application No. 63/282,515, filed Nov. 23, 2021, and U.S. Provisional Application No. 63/282,516, filed Nov. 23, 2021, the entire contents of each are hereby incorporated by reference.

### REFERENCE TO SEQUENCE LISTING

The instant application contains a Sequence Listing which has been submitted electronically in XML format via EFS-Web, and is hereby incorporated by reference in its entirety. Said XML copy, created on Feb. 20, 2023, is named PMB-00725.xml and is 1,753,195 bytes in size is hereby incorporated by reference in its entirety.

### BACKGROUND

Diseases, such as Glycogen Storage Disease Type 1B, may be caused in humans by disruption to the SLC37A4 gene (OMIM #602671), and manifest with widespread systemic effects secondary to the liver's inability to break down glycogen. SLC37A4 encodes the glucose-6-phosphate transporter (or translocase) (G6PT1) protein, which contributes to glucose homeostasis by regulating glucose-6-phosphate transport from the cytoplasm to the lumen of the endoplasmic reticulum. SLC37A4 is located in the human genome at 11q23.3 (chr11:119,024,111-119,030,876 (GRCh38/hg38)). The predominant isoform of G6PT1 is a 429-amino acid protein (GenBank accession CAA75608.1, SEQ ID NO: 1; cDNA sequence NM_001164277.2, SEQ ID NO:2). A frequent disease-causing mutation of SLC37A4 is G339C, in which a G-to-T transversion at position 1015 of the coding sequence in exon 8 (Chr11:119025299, GRCh38) causes a missense mutation in codon 339 from glycine (GGT) to cysteine (TGT). Another frequent disease-causing mutation of SLC37A4 is c.1042-1043delCT (L348fs), in which the deletion of two nucleotides (CT) at positions 1042-1043 of the SLC37A4 coding sequence causes a frameshift mutation that persists for 61 incorrect amino acid residues and terminates with a stop codon after amino acid 400.

### SUMMARY

Glycogen storage disease type 1B can be treated by gene editing because the G339C mutation or L348fs mutation in the SLC37A4 gene is amenable to prime editing, methods and compositions for which are described herein. The G339C mutation in SLC37A4 may be corrected, for example, by a T->G edit at position 1015 of the coding sequence, thus restoring the missense mutation to wild-type. The L348fs mutation in SLC37A4 may be corrected, for example, by insertion of CT at position 1042 of the coding sequence, thus restoring the frameshift mutation to wild-type.

Provided herein, in some embodiments, are methods and compositions for prime editing of alterations in a target sequence in a target gene, for example, the SLC37A4 gene. The target SLC37A4 gene may comprise double stranded DNA. As exemplified in FIG. 1, in an embodiment, the target gene is edited by prime editing.

Without wishing to be bound by any particular theory, the prime editing process may search specific targets and edit endogenous sequences in a target gene, e.g., the SLC37A4 gene. As exemplified in FIG. 1, the spacer sequence of a PEgRNA recognizes and anneals with a search target sequence in a target strand of the target gene. A prime editing complex may generate a nick in the target gene on the edit strand which is the complementary strand of the target strand. The prime editing complex may then use a free 3′ end formed at the nick site of the edit strand to initiate DNA synthesis, where a primer binding site (PBS) of the PEgRNA complexes with the free 3′ end, and a single stranded DNA is synthesized using an editing template of the PEgRNA as a template. The editing template may comprise one or more nucleotide edits compared to the endogenous target SLC37A4 gene sequence. Accordingly, the newly-synthesized single stranded DNA also comprises the nucleotide edit(s) encoded by the editing template. Through removal of an editing target sequence on the edit strand of the target gene and DNA repair, the intended nucleotide edit(s) included in the newly synthesized single stranded DNA are incorporated into the target SLC37A4 gene.

Therefore, in some aspects, provided herein are prime editing guide RNAs (PEgRNAs) comprising a spacer (e.g., an spacer disclosed herein) that comprises a region of complementarity to a search target sequence on a target strand of an SLC37A4 gene, an editing template (e.g., an editing template disclosed herein) that comprises a region of complementarity to an editing target sequence on a non-target strand of the SLC37A4 gene, and a gRNA core that associates with a prime editor comprising a DNA binding domain and a DNA polymerase domain, wherein the target strand and the non-target strand are complementary to each other, and wherein the editing target sequence is in exon 8 of the SLC37A4 gene.

In some aspects, provided herein are PEgRNAs comprising a spacer (e.g., an spacer disclosed herein) that comprises a region of complementarity to a search target sequence on a target strand of an SLC37A4 gene, an editing template (e.g., an editing template disclosed herein) that comprises a region of complementarity to an editing target sequence on a non-target strand of the SLC37A4 gene, and a gRNA core that associates with a prime editor comprising a DNA binding domain and a DNA polymerase domain, wherein target strand and the non-target strand are complementary to each other. In some embodiments, the editing target sequence comprises a codon encoding cysteine at position 339. In some embodiments, the editing target sequence comprises a 2-nucleotide deletion corresponding to positions 1042-1043 of a coding sequence of an SLC37A4 wild-type gene. In some embodiments, the editing target sequence comprises a codon encoding cysteine corresponding to position 339 of a SLC37A4 wild-type peptide.

In some aspects, provided herein are PEgRNAs comprising a spacer (e.g., an spacer disclosed herein) that comprises a region of complementarity to a search target sequence on a target strand of an SLC37A4 gene, an editing template (e.g., an editing template disclosed herein) that comprises a region of complementarity to an editing target sequence on a non-target strand of the SLC37A4 gene, and a gRNA core that associates with a prime editor comprising a DNA binding domain and a DNA polymerase domain, wherein the target strand and the non-target strand are complementary to each other, and wherein the editing target sequence i) comprises position 1015 of the SLC37A4 gene coding sequence; and/or ii) position 1042 of a SLC37A4 gene coding sequence.

In some embodiments, the editing target sequence comprises position 1015 of a SLC37A4 gene coding sequence. In some embodiments, the editing target sequence comprises position 1042 of a SLC37A4 gene coding sequence.

In some aspects, provided herein are PEgRNAs comprising a spacer that comprises a region of complementarity to a search target sequence on a target strand of a SLC37A4 gene, an editing template (e.g., an editing template disclosed herein) that comprises a region of complementarity to an editing target sequence on a non-target strand of the SLC37A4 gene, and a gRNA core that associates with a prime editor comprising a DNA binding domain and a DNA polymerase domain, wherein the target strand and the non-target strand are complementary to each other, wherein the editing target sequence comprises i) a T at position 1015 of the SLC37A4 gene coding sequence, and/or ii) a deletion at positions 1042-1043 of a SLC37A4 gene coding sequence.

In some embodiments, the editing target sequence comprises a T at position 1015 of a SLC37A4 gene coding sequence. In some embodiments, the editing target sequence comprises a deletion at positions 1042-1043 of a SLC37A4 gene coding sequence.

In some embodiments, the editing template is about 3 to 40 nucleotides in length. In some embodiments, the editing template is about 10 to 30 nucleotides in length. In some embodiments, the editing template comprises a region of complementarity to a region downstream of a nick site in the non-target strand.

In some embodiments, the gRNA core is between the spacer and the editing template.

In some embodiments, the PEgRNA comprises a primer binding site (PBS) that comprises a region of complementarity to a region upstream of a nick site in the non-target strand. In some embodiments, the PBS comprises a region of complementarity to a region immediately upstream of a nick site in the non-target strand.

In some embodiments, the PBS and the editing template are directly adjacent to each other. In some embodiments, the PBS is comprises partial complementary, or full complementary, to the spacer. In some embodiments, the PBS is about 2 to 20 nucleotides in length (e.g., such as 8-16 nucleotides in length). In some embodiments, the editing template comprises an intended nucleotide edit compared to the SLC37A4 gene. In some embodiments, the PEgRNA guides the prime editor to incorporate the intended nucleotide edit into the SLC37A4 gene when contacted with the SLC37A4 gene.

In some embodiments, the prime editor synthesizes a single stranded DNA encoded by the editing template, wherein the single stranded DNA replaces the editing target sequence and results in incorporation of the intended nucleotide edit into a region corresponding to the editing target in the SLC37A4 gene. In some embodiments, the search target sequence is complementary to a protospacer sequence in the SLC37A4 gene, and the protospacer sequence is adjacent to a search target adjacent motif (PAM) in the SLC37A4 gene.

In some embodiments, the PEgRNA guides the prime editor to incorporate the intended nucleotide edit in the PAM when contacted with the SLC37A4 gene.

In some embodiments, the PEgRNA guides the prime editor to incorporate the intended nucleotide edit about 0 to 27 base pairs downstream of the 5′ end of the PAM when contacted with the SLC37A4 gene.

In some embodiments, the intended nucleotide edit comprises a single nucleotide substitution compared to the region corresponding to the editing target sequence in the SLC37A4 gene. In some embodiments, the editing target sequence comprises a mutation that encodes a cysteine amino acid substitution as compared to a wild type SLC37A4 protein as set forth in SEQ ID NO: 1. In some embodiments, the intended nucleotide edit comprises a T>G nucleotide substitution at position 1015 in the coding sequence of the SLC37A4 gene.

In some embodiments, the intended nucleotide edit comprises an insertion of two nucleotides compared to the region corresponding to the editing target in the SLC37A4 gene. In some embodiments, the editing target sequence comprises a frameshift mutation beginning at amino acid position L348 compared to a wild type SLC37A4 protein as set forth in SEQ ID NO: 1. In some embodiments, the intended nucleotide edit comprises a CT insertion at position 1042 in a coding sequence of the SLC37A4 gene.

In some embodiments, wherein the editing target sequence comprises a mutation associated with Glycogen storage disease type 1B. In some embodiments, wherein the editing template comprises a wild type SLC37A4 gene sequence. In some embodiments, the PEgRNA results in correction of the mutation when contacted with the SLC37A4 gene.

In some embodiments, the spacer comprises a sequence listed in Table 8.

In some embodiments, the editing template comprises a sequence listed in Table 9a, Table 10a, Table 11a, Table 12a, Table 13a, Table 14a, Table 15a, Table 16a, Table 17a, Table 18a, Table 19a, Table 20a, Table 21a, Table 22a, Table 23a, and Table 24a.

In some embodiments, the PBS comprises a sequence listed in Table 9b, Table 10b, Table 11b, Table 12b, Table 13b, Table 14b, Table 15b, Table 16b, Table 17b, Table 18b, Table 19b, Table 20b, Table 21b, Table 22b, Table 23b, and Table 24b.

In some embodiments, the spacer comprises the sequence of S01, the editing template comprises a sequence listed in Table 9a, and the PBS comprises a sequence listed in Table 9b. In some embodiments, the spacer comprises the sequence of S02, the editing template comprises a sequence listed in Table 10a, and the PBS comprises a sequence listed in Table 10b. In some embodiments, the spacer comprises the sequence of S03, the editing template comprises a sequence listed in Table 11a, and the PBS comprises a sequence listed in Table 11b. In some embodiments, the spacer comprises the sequence of S04, the editing template comprises a sequence listed in Table 12a, and the PBS comprises a sequence listed in Table 12b. In some embodiments, the spacer comprises the sequence of S05, the editing template comprises a sequence listed in Table 13a, and the PBS comprises a sequence listed in Table 13b. In some embodiments, the spacer comprises the sequence of S06, the editing template comprises a sequence listed in Table 14a, and the PBS comprises a sequence listed in Table 14b. In some embodiments, the spacer comprises the sequence of S07, the editing template comprises a sequence listed in Table 15a, and the PBS comprises a sequence listed in Table 15b. In some embodiments, the spacer comprises the sequence of S08, the editing template comprises a sequence listed in Table 16a, and the PBS comprises a sequence listed in Table 16b. In some embodiments, the spacer comprises the sequence of S09, the editing template comprises a sequence listed in Table 17a, and the PBS comprises a sequence listed in Table 17b. In some embodiments, the spacer comprises the sequence of S10, the editing template comprises a sequence listed in Table 18a, and the PBS comprises a sequence listed in Table 18b. In some embodiments, the spacer comprises the sequence of S11, the editing template comprises a sequence selected from the editing template sequences listed in Table 19a, and the PBS comprises a sequence selected from the PBS sequences listed in Table 19b. In some embodiments, the spacer comprises the sequence of S12, the editing template comprises a sequence selected from the editing template sequences listed in Table 20a, and the PBS comprises a sequence selected from the PBS sequences listed in Table 20b. In some embodiments, the spacer comprises the sequence of S13, the editing template comprises a sequence selected from the editing template sequences listed in Table 21a, and the PBS comprises a sequence selected from the PBS sequences listed in Table 21b. In some embodiments, the spacer comprises the sequence of S14, the editing template comprises a sequence selected from the editing template sequences listed in Table 22a, and the PBS comprises a sequence selected from the PBS sequences listed in Table 22b. In some embodiments, the spacer comprises the sequence of S15, the editing template comprises a sequence selected from the editing template sequences listed in Table 23a, and the PBS comprises a sequence selected from the PBS sequences listed in Table 23b. In some embodiments, the spacer comprises the sequence of S16, the editing template comprises a sequence selected from the editing template sequences listed in Table 24a, and the PBS comprises a sequence selected from the PBS sequences listed in Table 24b.

One embodiment of this disclosure provides a PEgRNA comprising an RTT and a PBS sequence listed in Table 9c, Table 10c, Table 11c, Table 12c, Table 13c, Table 14c, Table 15c, Table 16c, Table 17c, Table 18c, Table 19c, Table 20c, Table 21c, Table 22c, Table 23c, or Table 24c.

In some embodiments, the intended nucleotide edit comprises an insertion of CT at position 1042 in the coding sequence of the SLC37A4 gene. In some embodiments, the spacer comprises the sequence of S07 and the editing template comprises a sequence selected from the editing template sequences listed in Table 15a. In some embodiments, the spacer comprises the sequence of S08 and the editing template comprises a sequence selected from the editing template sequences listed in Table 16a. In some embodiments, the spacer comprises the sequence of S09 and the editing template comprises a sequence selected from the editing template sequences listed in Table 17a. In some embodiments, the spacer comprises the sequence of S10 and the editing template comprises a sequence selected from the editing template sequences listed in Table 18a. In some embodiments, the intended nucleotide edit comprises a T>G nucleotide substitution at position 1015 in the coding sequence of the SLC37A4 gene. In some embodiments, the spacer comprises the sequence of S12 and the editing template comprises a sequence selected from the editing template sequences listed in Table 20a. In some embodiments, the spacer comprises the sequence of S13 and the editing template comprises a sequence selected from the editing template sequences listed in Table 21a.

One embodiment of this disclosure provides a PEgRNA system comprising the PEgRNA according to an embodiment disclosed herein and further comprising a nick guide RNA (ngRNA), wherein the ngRNA comprises an ng spacer that comprises a region of complementarity to a second search target sequence in the SLC37A4 gene.

In some embodiments, the second search target sequence is on the non-target strand of the SLC37A4 gene. In some embodiments, the ng spacer comprises a sequence listed in Table 8a, Table 8b, Table 8c, or Table 8d.

One embodiment of this disclosure provides a PEgRNA system comprising a PEgRNA comprising an RTT and a PBS sequence listed in Tables 9c, 10c, 11c, 12c, 13c, or 14c and an ngRNA comprising a sequence listed in Table 8a. In some embodiments, the PEgRNA comprises RTT and PBS sequences listed in Tables 15c, 16c, 17c, or 18c and the ngRNA comprises a sequence listed in Table 8b.

One embodiment of this disclosure provides a PEgRNA system comprising a PEgRNA comprising a combination of an RTT and a PBS sequence as listed in Tables 19c, 20c, 21c, 22c, 23c, or 24c and an ngRNA comprising a sequence selected from the ngRNA sequences listed in Table 8c or Table 8d.

In some embodiments, a PEgRNA that corrects the G339C mutation, if present, can also correct a nearby mutation, L348fs, in which the deletion of two nucleotides (CT) at positions 1042-1043 of the SLC37A4 coding sequence causes a frameshift mutation that persists for 61 incorrect amino acid residues and terminates with a stop codon after amino acid 400. PEgRNAs having the sequences of spacers S07, S08, S09, or S10, and RTT sequences selected from SEQ ID NO: 180 through SEQ ID NO: 241 (i.e., Tables 15a, 16a, 17a, and 18a, for the respective spacers) are capable of editing one or both G339C and L348fs mutations. Examples include PEG-0781 through PEG-1203 (comprising the RTT and PBS combinations as provided in Tables 15c, 16c, 17c, and 18c).

One embodiment of this disclosure provides a prime editing complex comprising: (i) a PEgRNA disclosed herein and (ii) a prime editor comprising a DNA binding domain and a DNA polymerase domain.

In some embodiments, the DNA binding domain is a CRISPR associated (Cas) protein domain. In some embodiments, the Cas protein domain has nickase activity. In some embodiments, the Cas protein domain is a Cas9. In some embodiments, the Cas9 comprises a mutation in an HNH domain. In some embodiments, the Cas9 comprises a H840A mutation in the HNH domain. In some embodiments, the Cas protein domain is a Cas12b. In some embodiments, the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ.

In some embodiments, the DNA polymerase domain is a reverse transcriptase. In some embodiments, the reverse transcriptase is a retrovirus reverse transcriptase. In some embodiments, the reverse transcriptase is a Moloney murine leukemia virus (M-MLV) reverse transcriptase. In some embodiments, the DNA polymerase and the DNA binding domain are fused or linked to form a fusion protein. In some embodiments, the fusion protein comprises the sequence of SEQ ID NO: 53.

One embodiment of this disclosure provides a lipid nanoparticle (LNP) or ribonucleoprotein (RNP) comprising the prime editing complex of an embodiment disclosed herein or a component thereof.

One embodiment of this disclosure provides a polynucleotide encoding the PEgRNA of an embodiment disclosed herein, the PEgRNA system of an embodiment disclosed herein, or the fusion protein of an embodiment disclosed herein.

In some embodiments, the polynucleotide is an mRNA. In some embodiments, the polynucleotide is operably linked to a regulatory element. In some embodiments, the regulatory element is an inducible regulatory element. One embodiment of this disclosure provides a vector comprising the polynucleotide of an embodiment disclosed herein. In some embodiments, the vector is an AAV vector.

One embodiment of this disclosure provides an isolated cell comprising the PEgRNA of an embodiment disclosed herein, the PEgRNA system of an embodiment disclosed herein, the prime editing complex of an embodiment disclosed herein, the LNP or RNP of an embodiment disclosed herein, the polynucleotide of an embodiment disclosed herein, or the vector of an embodiment disclosed herein.

One embodiment of this disclosure provides a pharmaceutical composition comprising (i) the PEgRNA of an embodiment disclosed herein, the PEgRNA system of an embodiment disclosed herein, the prime editing complex of an embodiment disclosed herein, the LNP or RNP of an embodiment disclosed herein, the polynucleotide of an embodiment disclosed herein, the vector of an embodiment disclosed herein, or the cell of an embodiment disclosed herein; and (ii) a pharmaceutically acceptable carrier.

One embodiment of this disclosure provides a method for editing an SLC37A4 gene, the method comprising contacting the SLC37A4 gene with (i) a PEgRNA disclosed herein or the PEgRNA system disclosed herein and (ii) a prime editor comprising a DNA binding domain and a DNA polymerase domain, wherein the PEgRNA directs the prime editor to incorporate the intended nucleotide edit in the SLC37A4 gene, thereby editing the SLC37A4 gene.

One embodiment of this disclosure provides a method for editing an SLC37A4 gene, the method comprising contacting the SLC37A4 gene with the prime editing complex of an embodiment disclosed herein, wherein the PEgRNA directs the prime editor to incorporate the intended nucleotide edit in the SLC37A4 gene, thereby editing the SLC37A4 gene.

In some embodiments, the prime editor synthesizes a single stranded DNA encoded by the editing template, wherein the single stranded DNA replaces the editing target sequence and results in incorporation of the intended nucleotide edit into a region corresponding to the editing target in the SLC37A4 gene.

In some embodiments, the SLC37A4 gene is in a cell. In some embodiments, the cell is a mammalian cell. In some embodiments, the cell is a human cell. In some embodiments, the cell is a primary cell. In some embodiments, the cell is a liver cell. In some embodiments, the cell is a hepatocyte. In some embodiments, the cell is a cholangiocyte. In some embodiments, the cell is a kidney cell. In some embodiments, the cell is a proximal tubule cell. In some embodiments, the cell is a Müller cell. In some embodiments, the cell is in a subject. In some embodiments, the subject is a human.

In some embodiments, the cell is from a subject having Glycogen storage disease type 1B. In some embodiments, the method further comprises administering the cell to the subject after incorporation of the intended nucleotide edit.

One embodiment of this disclosure provides a cell generated by the method of an embodiment disclosed herein.

One embodiment of this disclosure provides a population of cells generated by the method of an embodiment disclosed herein.

One embodiment of this disclosure provides a method for treating Glycogen storage disease type 1B in a subject in need thereof, the method comprising administering to the subject (i) a PEgRNA disclosed herein or a PEgRNA system disclosed herein and (ii) a prime editor comprising a DNA binding domain and a DNA polymerase domain, wherein the PEgRNA directs the prime editor to incorporate the intended nucleotide edit in the SLC37A4 gene in the subject, thereby treating Glycogen storage disease type 1B in the subject.

One embodiment of this disclosure provides a method for treating Glycogen storage disease type 1B in a subject in need thereof, the method comprising administering to the subject the prime editing complex of an embodiment disclosed herein, the LNP or RNP of an embodiment disclosed herein, or the pharmaceutical composition of an embodiment disclosed herein, wherein the PEgRNA directs the prime editor to incorporate the intended nucleotide edit in the SLC37A4 gene in the subject, thereby treating Glycogen storage disease type 1B in the subject.

In some embodiments, the subject is a human. In some embodiments, the SLC37A4 gene in the subject comprises a mutation that encodes a G339C amino acid substitution as compared to a wild type SLC37A4 protein as set forth in SEQ ID NO: 1. In some embodiments, the SLC37A4 gene comprises a mutation that encodes an G339C amino acid substitution as compared to a wild type SLC37A4 protein as set forth in SEQ ID NO: 1.

In some embodiments, the SLC37A4 gene (e.g., a gene in the subject) comprises a mutation that encodes a L348fs amino acid frameshift mutation as compared to a wild type SLC37A4 protein as set forth in SEQ ID NO: 1. In some embodiments, the SLC37A4 gene comprises a mutation that has a 2-nucleotide CT deletion at coding sequence position 1042 as compared to a wild type SLC37A4 gene.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 depicts a schematic of a prime editing guide RNA (PEgRNA) binding to a double stranded target DNA sequence.

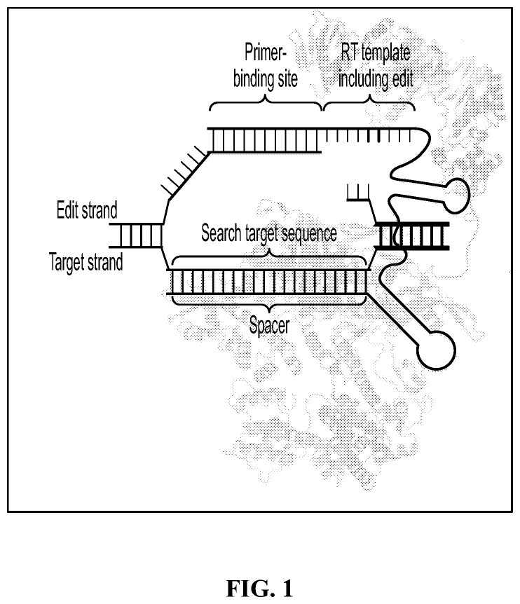

FIG. 2 depicts a PEgRNA architectural overview in an exemplary schematic of PEgRNA designed for a prime editor.

FIG. 3 is a schematic showing the spacer and gRNA core part of an exemplary guide RNA, in two separate molecules. The rest of the PEgRNA structure is not shown.

### DETAILED DESCRIPTION

Provided herein, in some embodiments, are compositions and methods to edit the target gene SLC37A4 with prime editing. In certain embodiments, provided herein are compositions and methods for correction of mutations in the SLC37A4 gene associated with Glycogen storage disease type 1B. Compositions provided herein can comprise prime editors (PEs) that may use engineered guide polynucleotides, e.g., prime editing guide RNAs (PEgRNAs), that can direct PEs to specific DNA targets and can encode DNA edits on the target gene SLC37A4 that serve a variety of functions, including direct correction of disease-causing mutations.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains,” “contain,” and variants thereof are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within an acceptable error range for the particular value as determined by one of ordinary skill in the art, which will depend in part on how the value is measured or determined, i.e, the limitations of the measurement system. For example, “about” can mean within 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include retinal cells (e.g., photoreceptors, retinal pigment epithelium cells, Müller cells), epithelial cells (e.g., mammary epithelial cells, intestinal epithelial cells, hepatocytes), endothelial cells, glial cells, neural cells, hair cells, formed elements of the blood (e.g., lymphocytes, bone marrow cells), precursors of any of these somatic cell types, and stem cells. In some embodiments, the cell is a fibroblast. In some embodiments, the cell is a stem cell. In some embodiments, the cell is a pluripotent stem cell. In some embodiments, the cell is an induced pluripotent stem cell (iPSC). In some embodiments, the cell is a retinal progenitor cell. In some embodiments, the cell is a retinal precursor cell. In some embodiments, the cell is an embryonic stem cell (ESC). In some embodiments, the cell is a human stem cell. In some embodiments, the cell is a human pluripotent stem cell. In some embodiments, the cell is a human fibroblast. In some embodiments, the cell is an induced human pluripotent stem cell. In some embodiments, the cell is a human stem cell. In some embodiments, the cell is a human embryonic stem cell. In some embodiments, the cell is a human retinal progenitor cell. In some embodiments, the cell is a human retinal precursor cell.

In some embodiments, a cell is not isolated from an organism but forms part of a tissue or organ of an organism, e.g., a mammal, such as a human. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a liver cell. In some embodiments, the cell is a hepatocyte. In some embodiments, the cell is a cholangiocyte. In some embodiments, the cell is a kidney cell. In some embodiments, the cell is a proximal tubule cell. In some embodiments, the cell is a Müller cell. In some embodiments, the cell is a human stem cell.

In some embodiments, the cell is a differentiated cell. In some embodiments, cell is a fibroblast. In some embodiments, the cell is differentiated from an induced pluripotent stem cell. In some embodiments, the cell is any of a liver cell, a hepatocyte, or a cholangiocyte differentiated from an iPSC, ESC or a liver progenitor cell.

In some embodiments, the cell is a differentiated human cell. In some embodiments, cell is a human fibroblast. In some embodiments, the cell is differentiated from an induced human pluripotent stem cell. In some embodiments, the cell is any of a liver cell, a hepatocyte, or a cholangiocyte differentiated from a human iPSC, a human ESC or a human retinal progenitor cell.

In some embodiments, the cell comprises a prime editor or a prime editing composition. In some embodiments, the cell is from a human subject. In some embodiments, the human subject has a disease or condition associated with a mutation to be corrected by prime editing, for example, Glycogen storage disease type 1B. In some embodiments, the cell is from a human subject, and comprises a prime editor or a prime editing composition for correction of the mutation. In some embodiments, the cell is from the human subject and the mutation has been edited or corrected by prime editing. In some embodiments, the cell is in a human subject, and comprises a prime editor or a prime editing composition for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide includes both naturally occurring amino acids and non-naturally occurring amino acids. In some embodiments, a protein or polypeptide is modified.

In some embodiments, a protein or polypeptide is an isolated protein or an isolated polypeptide. The term “isolated” means free or substantially free from components which normally accompany it as found in the natural state or environment. For example, a polypeptide naturally present in a living animal is not isolated, when present in that living animal in its natural state, and the same polypeptide substantially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, primer binding site or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program https://fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

Amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G).

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to base pair with each other. Complementary polynucleotides may base pair via hydrogen bonding, which may be Watson Crick, Hoogsteen or reversed Hoogsteen hydrogen bonding. For example, an adenine on one polynucleotide molecule will base pair to a guanine on a second polynucleotide molecule and a cytosine on one polynucleotide molecule will base pair to a thymine or uracil on a second polynucleotide molecule. Two polynucleotide molecules are complementary to each other when a first polynucleotide molecule comprising a first nucleotide sequence can base pair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can base pair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will base pair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., a mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be up to about 100 years of age. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” may refer to a quantity of a composition, for example a composition comprising a construct, that can be sufficient to result in a desired activity upon introduction into a subject as disclosed herein.

An effective amount of the prime editing compositions can be provided to the target gene or cell, whether the cell is ex vivo or in vivo.

An effective amount can be the amount to induce, for example, at least about a 2-fold change (increase or decrease) or more in the amount of target nucleic acid modulation (e.g., expression of SLC37A4 gene to produce functional SLC37A4 G6PT1 protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target SLC37A4 gene to produce functional G6PT1).

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

An effective amount can be the amount to induce, when administered to a population of cells, a certain percentage of the population of cells to have a correction of the G339C mutation or L348fs mutation. For example, in some embodiments, an effective amount can be the amount to induce, when administered to or introduced to a population of cells, installation of one or more intended nucleotide edits that correct a c. 1015 G->T (encoding G339C amino acid substitution) mutation in the SLC37A4 gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells. For example, in some embodiments, an effective amount can be the amount to induce, when administered to or introduced to a population of cells, installation of one or more intended nucleotide edits that correct a c. 1042delCT (L348fs) mutation in the SLC37A4 gene, in at least about 1%, 2%, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of the population of cells.

### Prime Editing

The term “prime editing” refers to programmable editing of a target DNA using a prime editor complexed with a PEgRNA to incorporate an intended nucleotide edit (also referred to herein as a nucleotide change) into the target DNA through target-primed DNA synthesis. A target polynucleotide, e.g., a target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease In some embodiments, a specific PAM is characteristic of a specific programmable DNA binding protein, e.g., a Cas nickase or a Cas nuclease A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two base pairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 base pairs upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 base pairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

A “primer binding site” (PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other.

In some embodiments, the single stranded portion of the PEgRNA comprising both the PBS and the editing template is complementary or substantially complementary to an endogenous sequence on the PAM strand (i.e. the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits. In some embodiments, the editing template is about 3 to 40 nucleotides in length. In some embodiments, the editing template is about 10 to 30 nucleotides in length. For example, the editing template may be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 nucleotides in length. In some embodiments, the editing template is no more than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides in length.

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. In some embodiments, the editing template of a PEgRNA is complementary to a sequence in the edit strand except for one or more mismatches at the intended nucleotide edit positions in the editing template partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

The DNA polymerases can be wild type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol IV family DNA polymerase. In some embodiments, the DNA polymerase is a E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA Polymerase is a Pol III family DNA polymerase. In some embodiments, the DNA Polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UITma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT. An exemplary sequence of a wild type M-MLV RT is provided in SEQ ID NO: 50. In some embodiments, the prime editor comprises a M-MLV RT comprising a H8Y amino acid substitution. Collectively, the wild type M-MLV RT and the H8Y M-MLV RT are referred to as reference M-MLV RTs.

In some embodiments, the prime editor comprises a M-MMLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the reference M-MMLV RT as set forth in SEQ ID NO: 50, where X is any amino acid other than the amino acid in the corresponding reference M-MLV. In some embodiments, the prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the reference M-MMLV RT as set forth in SEQ ID NO: 50. In some embodiments, the prime editor comprises a M-MLV RT comprising one or more amino acid substitutions D200N, T330P, L603W, T306K, and W313F as compared to the reference-MMLV RT as set forth in SEQ ID NO: 50. In some embodiments, the prime editor comprises a M-MLV RT comprising amino acid substitutions D200N, T330P, L603W, T306K, and W313F as compared to the reference M-MMLV RT as set forth in SEQ ID NO: 50. In some embodiments, a prime editor comprising the D200N, T330P, L603W, T306K, and W313F as compared to the reference M-MMLV RT maybe referred to as a “PE2” prime editor, and the corresponding prime editing system a PE2 prime editing system.

In some embodiments, an RT variant may be a functional fragment of a reference RT that have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a reference RT. In some embodiments, the RT variant comprises a fragment of a reference RT, e.g., a reference RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding reference RT (M-MLV reverse transcriptase) (e.g., SEQ ID NO: 50).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a wild type RT. In some embodiments, the reference RT is a wild type M-MLV RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a wild type RT. In some embodiments, the reference RT is a wild type M-MLV RT. In still other embodiments, the RT truncated variant has a truncation at the N-terminal and the C-terminal end compared to a reference RT, e.g., a wild type RT. In some embodiments, the N-terminal truncation and the C-terminal truncation are of the same length. In some embodiments, the N-terminal truncation and the C-terminal truncation are of different lengths.

For example, the prime editors disclosed herein may include a functional variant of a wild type M-MLV reverse transcriptase. In some embodiments, the prime editor comprises a functional variant of a wild type M-MLV RT, wherein the functional variant of M-MLV RT is truncated after amino acid position 502 compared to a wild type M-MLV RT as set forth in SEQ ID NO: 50. In some embodiments, the functional variant of M-MLV RT further comprises a D200X, T306X, W313X, and/or T330X amino acid substitution compared to compared to a wild type M-MLV RT as set forth in SEQ ID NO: 50, wherein X is any amino acid other than the original amino acid. In some embodiments, the functional variant of M-MLV RT further comprises a D200N, T306K, W313F, and/or T330P amino acid substitution compared to a wild type M-MLV RT as set forth in SEQ ID NO: 50, wherein X is any amino acid other than the original amino acid. A DNA sequence encoding a prime editor comprising this truncated RT is 522 bp smaller than PE2, and therefore makes its potentially useful for applications where delivery of the DNA sequence is challenging due to its size (i.e., adeno-associated virus and lentivirus delivery). In some embodiments, a prime editor comprises a M-MLV RT variant, wherein the M-MLV RT consists of the following amino acid sequence:

(SEQ ID NO: 48)
TLNIEDEYRLHETSKEPDVSLGSTWLSDFPQAWAETGGMGLAVRQAPLI
IPLKATSTPVSIKQYPMSQEARLGIKPHIQRLLDQGILVPCQSPWNTPL
LPVKKPGTNDYRPVQDLREVNKRVEDIHPTVPNPYNLLSGLPPSHQWYT
VLDLKDAFFCLRLHPTSQPLFAFEWRDPEMGISGQLTWTRLPQGFKNSP
TLFNEALHRDLADFRIQHPDLILLQYVDDLLLAATSELDCQQGTRALLQ
TLGNLGYRASAKKAQICQKQVKYLGYLLKEGQRWLTEARKETVMGQPTP
KTPRQLREFLGKAGFCRLFIPGFAEMAAPLYPLTKPGTLFNWGPDQQKA
YQEIKQALLTAPALGLPDLTKPFELFVDEKQGYAKGVLTQKLGPWRRPV
AYLSKKLDPVAAGWPPCLRMVAAIAVLTKDAGKLTMGQPLVILAPHAVE
ALVKQPPDRWLSNARMTHYQALLLDTDRVQFGPVVALNPATLLPLPEEG
LQHNCLDNSRLIN.

In some embodiments, the functional variant of M-MLV RT comprises a D200N, T306K, W313F, T330P, and L603W amino acid substitution compared to a reference M-MLV RT.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.I2) RT. In some embodiments, the prime editor comprises a retron RT.

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas Φ, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

An exemplary Streptococcus pyogenes Cas9 (SpCas9) amino acid sequence is provided in SEQ ID NO: 51.

In some embodiments, a prime editor comprises a Cas9 protein from Staphylococcus lugdunensis (Slu Cas9). An exemplary amino acid sequence of a Slu Cas9 is provided in SEQ ID NO: 52.

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO: 51, or a corresponding mutation thereof. In some embodiments, the Cas9 comprise a D10A mutation as compared to a wild type SpCas9 as set forth in SEQ ID NO: 51, or a corresponding mutation thereof. In some embodiments, the Cas9 polypeptide comprise a mutation at amino acid D10, G12, and/or G17 as compared to a wild type SpCas9 as set forth in SEQ ID NO: 51, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 51, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 51, or a corresponding mutation thereof. In some embodiments, the Cas9 polypeptide comprise a H840A mutation as compared to a wild type SpCas9 as set forth in SEQ ID NO: 51, or a corresponding mutation thereof. In some embodiments, the Cas9 polypeptide comprise a mutation at amino acid E762, D839, H840, N854, N856, N863, H982, H983, A984, D986, and/or a A987 as compared to a wild type SpCas9 as set forth in SEQ ID NO: 51, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 51, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a DIOX mutation compared to a wild type SpCas9 as set forth in SEQ ID NO: 51 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 51, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 1 below. It should be appreciated that for each of the variants provided, the Cas protein comprises one or more of the amino acid substitutions as indicated compared to a wild type Cas protein sequence, for example, the Cas9 as set forth in SEQ ID NO: 51. The PAM motifs as shown in Table 1 below are in the order of 5′ to 3′.

The nucleotides listed in Table 1 are represented by the base codes as provided in the Handbook on Industrial Property Information and Documentation, World Intellectual Property Organization (WIPO) Standard ST.26, Version 1.4. For example, an “R” in Table 1 represents the nucleotide A or G, and “W” in Table 1 represents A or T.

TABLE 1
Cas protein variants and corresponding PAM sequences

| Variant | PAM |
|---|---|
| spCas9 (wild type) | NGG, NGA, |
| | NAG, NGNGA |
| spCas9- VRVRFRR R1335V, L1111R, D1135V, | NG |
| G1218R, E1219F, A1322R, T1337R | |
| spCas9-VQR (D1135V, R1335Q, T1337R) | NGA |
| spCas9-EQR (D1135E, R1335Q, T1337R) | NGA |
| spCas9-VRER (D1135V, G1218R, R1335E, T1337R) | NGCG |
| spCas9-VRQR (D1135V, G1218R, R1335Q, T1337R) | NGA |
| Cas9-NG (L1111R, D1135V, G1218R, E1219F, | NGN |
| A1322R, T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| sRGN1, sRGN2, sRGN4, sRGN3.1, sRGN3.3 | NNGG |
| saCas9 | NNGRRT, |
| | NNGRRN |
| saCas9-KKH (E782K, N968K, R1015H) | NNNRRT |
| spCas9-MQKSER (D1135M, S1136Q, G1218K, | NGCG/NGCN |
| E1219S, R1335E, T1337R) | |
| spCas9-LRKIQK (D1135L, S1136R, G1218K, | NGTN |
| E1219I, R1335Q, T1337K) | |
| spCas9-LRVSQK (D1135L, S1136R, G1218V, | NGTN |
| E1219S, R1335Q, T1337K) | |
| spCas9-LRVSQL(D1135L, S1136R, G1218V, | NGTN |
| E1219S, R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135V, R221K, A262T, R324L, N394K, S409I, S409I, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, I1322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 51.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations 1367K, G368D, I369K, H371L, T375S, T376G, and T1227K as compared to the wild type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus—[original C-terminus]—[original N-terminus]—C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus—[original C-terminus]—[optional linker]—[original N-terminus]—C-terminus. In some embodiments, a circular permutant Cas9 comprises any one of the following structures (amino acid positions as set forth in SEQ ID NO: 51):
N-terminus—[1268-1368]—[optional linker]—[1-1267]—C-terminus;
N-terminus—[1168-1368]—[optional linker]—[1-1167]—C-terminus;
N-terminus—[1068-1368]—[optional linker]—[1-1067]—C-terminus;
N-terminus—[968-1368]—[optional linker]—[1-967]—C-terminus;
N-terminus—[868-1368]—[optional linker]—[1-867]—C-terminus;
N-terminus—[768-1368]—[optional linker]—[1-767]—C-terminus;
N-terminus—[668-1368]—[optional linker]—[1-667]—C-terminus;
N-terminus—[568-1368]—[optional linker]—[1-567]—C-terminus;
N-terminus—[468-1368]—[optional linker]—[1-467]—C-terminus;
N-terminus—[368-1368]—[optional linker]—[1-367]—C-terminus;
N-terminus—[268-1368]—[optional linker]—[1-267]—C-terminus;
N-terminus—[168-1368]—[optional linker]—[1-167]—C-terminus;
N-terminus—[68-1368]—[optional linker]—[1-67]—C-terminus;
N-terminus—[10-1368]—[optional linker]—[1-9]—C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 51-1368 amino acids of UniProtKB-Q99ZW2:
N-terminus—[102-1368]—[optional linker]—[1-101]—C-terminus;
N-terminus—[1028-1368]—[optional linker]—[1-1027]—C-terminus;
N-terminus—[1041-1368]—[optional linker]—[1-1043]—C-terminus;
N-terminus—[1249-1368]—[optional linker]—[1-1248]—C-terminus; or
N-terminus—[1300-1368]—[optional linker]—[1-1299]—C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 51-1368 amino acids of UniProtKB-Q99ZW2 N-terminus—[103-1368]—[optional linker]—[1-102]—C-terminus:
N-terminus—[1029-1368]—[optional linker]—[1-1028]—C-terminus;
N-terminus—[1042-1368]—[optional linker]—[1-1041]—C-terminus;
N-terminus—[1250-1368]—[optional linker]—[1-1249]—C-terminus; or
N-terminus—[1301-1368]—[optional linker]—[1-1300]—C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, thee C-terminal fragment may correspond to the 95% or more of the C-terminal amino acids of a Cas9 (e.g., amino acids about 1300-1368 as set forth in SEQ ID NO: 51 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID NO: 51 or a ortholog or a variant thereof). The N-terminal portion may correspond to 95% or more of the N-terminal amino acids of a Cas9 (e.g., amino acids about 1-1300 as set forth in SEQ ID NO: 51 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID NO: 51 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID NO: 51 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID NO: 51 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID NO: 51 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e/g/as set forth in SEQ ID NO: 51 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID NO: 51 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 51: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID NO: 51 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP¹⁸¹, Cas9-CP¹⁹⁹, Cas9-CP²³⁰, Cas9-CP²⁷⁰, Cas9-CP³¹⁰, Cas9-CP¹⁰¹⁰, Cas9-CP¹⁰¹⁶, Cas9-CP¹⁰²³, Cas9-CP¹⁰²⁹, Cas9-CP¹⁰⁴¹, Cas9-CP¹²⁴⁷, Cas9-CP¹²⁴⁹, and Cas9-CP¹²⁸², respectively. This description is not meant to be limited to making CP variants from SEQ ID NO: 51, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 51). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

Exemplary Cas proteins and nomenclature are shown in Table 2 below:

TABLE 2
Exemplary Cas proteins and nomenclature

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a Cas Φ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas Φ protein. In some embodiments, the Cas protein is a Cas12e, Cas12d, Cas13, or Cas Φ nickase.

### Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase, that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 7), KRTADGSEFESPKKKRKV (SEQ ID NO: 8), KRTADGSEFEPKKKRKV (SEQ ID NO: 9), NLSKRPAAIKKAGQAKKKK (SEQ ID NO: 10), RQRRNELKRSF (SEQ ID NO: 11), or NQSSNFGPMKGGNFGGRSSGPYGGGGQYFAKPRNQGGY (SEQ ID NO: 12).

In some embodiments, a NLS is a monopartite NLS. For example, in some embodiments, a NLS is a SV40 large T antigen NLS PKKKRKV (SEQ ID NO: 13). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the sequence KRXXX XXXXXXXKKKL (Xenopus nucleoplasmin NLS) (SEQ ID NO: 14), wherein X is any amino acid. In some embodiments, the NLS comprises a nucleoplasmin NLS sequence KRPAATKKAGQAKKKK (SEQ ID NO: 15). In some embodiments, a NLS is a noncanonical sequences such as M9 of the hnRNP A1 protein, the influenza virus nucleoprotein NLS, and the yeast Gal4 protein NLS. In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table 3 below.

TABLE 3
Exemplary nuclear localization sequences

| | | SEQ |
|---|---|---|
| | | ID |
| Description | Sequence | NO: |
| NLS of SV40 | PKKKRKV | 16 |
| Large T-AG | | |
| NLS | MKRTADGSEFESPKKKRKV | 17 |
| NLS | MDSLLMNRRKFLYQFKNVRWAKGRRETYLC | 7 |
| NLS of | AVKRPAATKKAGQAKKKKLD | 18 |
| Nucleoplasmin | | |
| NLS of EGL- | MSRRRKANPTKLSENAKKLAKEVEN | 19 |
| 13 | | |
| NLS of C-Myc | PAAKRVKLD | 20 |
| NLS of Tus- | KLKIKRPVK | 21 |
| protein | | |
| NLS of | VSRKRPRP | 22 |
| polyoma large | | |
| T-AG | | |
| NLS of | EGAPPAKRAR | 23 |
| Hepatitis D | | |
| virus antigen | | |
| NLS of murine | PPQPKKKPLDGE | 24 |
| p53 | | |
| NLS of PE1 | SGGSKRTADGSEFEPKKKRKV | 25 |
| and PE2 | | |
| NLS | KRTADGSEFESPKKKRKV | 6021 |
| NLS | KRTADGSEFEPKKKRKV | 6022 |
| NLS | NLSKRPAAIKKAGQAKKKK | 6023 |
| NLS | RQRRNELKRSF | 6024 |
| NLS | NQSSNFGPMKGGNFGGRSSGPYGGGGQYFAKPRNQGGY | 6025 |
| NLS of | KRXXXXXXXXXXKKKL | 6026 |
| Xenopus | | |
| nucleoplasmin | | |
| NLS of | KRPAATKKAGQAKKKK | 6027 |
| Xenopus | | |
| nucleoplasmin | | |
| NLS | SGGSSGGSKRTADGSEFESPKKKRKVSGGSSGGS | 6028 |
| NLS | SGGSKRTADGSEFESPKKKRKV | 6029 |
| NLS | GSGPAAKRVKLD | 6030 |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C. essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last β-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

In some embodiments, a prime editing complex comprises a fusion protein comprising a DNA binding domain (e.g., Cas9 (H840A)) and a reverse transcriptase (e.g., a variant MMLV RT) having the following structure: [NLS]—[Cas9 (H840A)]—[linker]—[MMLV_RT(D200N)(T330P)(L603W)(T306K)(W313F)], and a desired PEgRNA. In some embodiments, the prime editing complex comprises a prime editor fusion protein that has the amino acid sequence of SEQ ID NO: 53.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 26). In some embodiments, the amino acid sequence of the MCP is:

(SEQ ID NO: 27)
GSASNFTQFVLVDNGGTGDVTVAPSNFANGVAEWISSNSRSQAYKVTCS
VRQSSAQNRKYTIKVEVPKVATQTVGGEELPVAGWRSYLNMELTIPIFA
TNSDCELIVKAMQGLLKDGNPIPSAIAANSGIY.

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 28), (G)n (SEQ ID NO: 29), (EAAAK)n (SEQ ID NO: 30), (GGS)n (SEQ ID NO: 31), (SGGS)n (SEQ ID NO: 32), (XP)n (SEQ ID NO: 33), or any combination thereof, wherein n is independently an integer between 1 and 30, and wherein X is any amino acid. In some embodiments, the linker comprises the amino acid sequence (GGS)n (SEQ ID NO: 34), wherein n is 1, 3, or 7. In some embodiments, the linker comprises the amino acid sequence SGSETPGTSESATPES (SEQ ID NO: 35). In some embodiments, the linker comprises the amino acid sequence SGGSSGGSSGS ETPGTSESATPESSGGSSGGS (SEQ ID NO: 36). In some embodiments, the linker comprises the amino acid sequence SGGSGGSGGS (SEQ ID NO: 37). In some embodiments, the linker comprises the amino acid sequence SGGS (SEQ ID NO: 38). In other embodiments, the linker comprises the amino acid sequence

(SEQ ID NO: 39)
SGGSSGGSSGSETPGTSESATPESAGSYPYDVPDYAGSAAPAAKKKKLD
GSGSGGSSGGS.

In some embodiments, a linker comprises 1-100 amino acids. In some embodiments, the linker comprises the amino acid sequence SGSETPGTSESATPES (SEQ ID NO: 40). In some embodiments, the linker comprises the amino acid sequence SGGSSGGSSGSETPGTSESATPESSGGSSGGS (SEQ ID NO: 41). In some embodiments, the linker comprises the amino acid sequence SGGSGGSGGS (SEQ ID NO: 42). In some embodiments, the linker comprises the amino acid sequence SGGS (SEQ ID NO: 43). In some embodiments, the linker comprises the amino acid sequence GGSGGS (SEQ ID NO: 44), GGSGGSGGS (SEQ ID NO: 45), SGGSSGGSSGSETPGTSESATPESAGSYPYDVPDYAGSAAPAAKKKKLDGSGSGGSSG GS (SEQ ID NO: 46), or SGGSSGGSSGSETPGTSESATPESSGGSSGGSS (SEQ ID NO: 47).

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring.

The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2—[DNA binding domain]—[polymerase]—COOH; or NH2—[polymerase]—[DNA binding domain]—COOH, wherein each instance of “]—[” indicates the presence of an optional linker sequence. In some embodiments, a prime editor comprises a fusion protein and a DNA polymerase domain provided in trans, wherein the fusion protein comprises the structure NH2—[DNA binding domain]—[RNA-protein recruitment polypeptide]—COOH. In some embodiments, a prime editor comprises a fusion protein and a DNA binding domain provided in trans, wherein the fusion protein comprises the structure NH2—[DNA polymerase domain]—[RNA-protein recruitment polypeptide]—COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

In some embodiments, a prime editor fusion protein comprises a Cas9 (H840A) nickase and a wild type M-MLV RT (referred to as “PE1”, and a prime editing system or composition referred to as PE1 system or PE1 composition). In some embodiments, a prime editor fusion protein comprises one or more individual components of PE1. In some embodiments, a prime editor fusion protein comprises a Cas9 (H840A) nickase and a M-MLV RT that has amino acid substitutions D200N, T330P, T306K, W313F, and L603W compared to a wild type M-MLV RT (the fusion protein referred to as “PE2”, and a prime editing system or composition referred to as PE2 system or PE2 composition). The amino acid sequence of an exemplary PE2 and its individual components in shown in Table 4. In some embodiments, a prime editor fusion protein is PE2. In some embodiments, a prime editor fusion protein comprises one or more individual components of PE2. In some embodiments, a prime editor fusion protein comprises a Cas9 (R221K, N349K, H840A) nickase and a M-MLV RT that has amino acid substitutions D200N, T330P, T306K, W313F, and L603W compared to a wild type M-MLV RT (the fusion protein referred to as “PEmax”, and a prime editing system or composition referred to as PEmax system or PEmax composition). The amino acid sequence of an exemplary PEmax and its individual components in shown in Table 4B. In some embodiments, a prime editor fusion protein is PEmax. In some embodiments, a prime editor fusion protein comprises one or more individual components of PEmax.

In various embodiments, a prime editor fusion proteins comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to PE1, PE2, or any of the prime editor fusion sequences described herein or known in the art.

TABLE 4
Amino acid sequence of PE2 and its individual components

| DESCRIPTION | SEQUENCE |
|---|---|
| PE2 fusion | MKRTADGSEFESPKKKRKVDKKYSIGLDIGTNSVGWAVITDEYKVPSKKFKVLGNTDRHSIKKNL |
| protein | IGALLFDSGETAEATRLKRTARRRYTRRKNRICYLQEIFSNEMAKVDDSFFHRLEESFLVEEDKK |
| CAS9(H840A)- | HERHPIFGNIVDEVAYHEKYPTIYHLRKKLVDSTDKADLRLIYLALAHMIKFRGHFLIEGDLNPDN |
| MMLV_RT | SDVDKLFIQLVQTYNQLFEENPINASGVDAKAILSARLSKSRRLENLIAQLPGEKKNGLFGNLIAL |
| D200N T330P | SLGLTPNFKSNFDLAEDAKLQLSKDTYDDDLDNLLAQIGDQYADLFLAAKNLSDAILLSDILRVN |
| L603W T306K | TEITKAPLSASMIKRYDEHHQDLTLLKALVRQQLPEKYKEIFFDQSKNGYAGYIDGGASQEEFYK |
| W313F (SEQ ID | FIKPILEKMDGTEELLVKLNREDLLRKQRTFDNGSIPHQIHLGELHAILRRQEDFYPFLKDNREKI |
| NO: 53) | EKILTFRIPYYVGPLARGNSRFAWMTRKSEETITPWNFEEVVDKGASAQSFIERMTNFDKNLPNE |
| | KVLPKHSLLYEYFTVYNELTKVKYVTEGMRKPAFLSGEQKKAIVDLLFKTNRKVTVKQLKEDYF |
| | KKIECFDSVEISGVEDRFNASLGTYHDLLKIIKDKDFLDNEENEDILEDIVLTLTLFEDREMIEERLK |
| | TYAHLFDDKVMKQLKRRRYTGWGRLSRKLINGIRDKQSGKTILDFLKSDGFANRNFMQLIHDDS |
| | LTFKEDIQKAQVSGQGDSLHEHIANLAGSPAIKKGILQTVKVVDELVKVMGRHKPENIVIEMARE |
| | NQTTQKGQKNSRERMKRIEEGIKELGSQILKEHPVENTQLQNEKLYLYYLQNGRDMYVDQELDI |
| | NRLSDYDVDAIVPQSFLKDDSIDNKVLTRSDKNRGKSDNVPSEEVVKKMKNYWRQLLNAKLITQ |
| | RKFDNLTKAERGGLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVYGDYKVYDVRKMIA |
| | KSEQEIGKATAKYFFYSNIMNFFKTEITLANGEIRKRPLIETNGETGEIVWDKGRDFATVRKVLSM |
| | PQVNIVKKTEVQTGGFSKESILPKRNSDKLIARKKDWDPKKYGGFDSPTVAYSVLVVAKVEKGK |
| | SKKLKSVKELLGITIMERSSFEKNPIDFLEAKGYKEVKKDLIIKLPKYSLFELENGRKRMLASAGE |
| | LQKGNELALPSKYVNFLYLASHYEKLKGSPEDNEQKQLFVEQHKHYLDEIIEQISEFSKRVILAD |
| | ANLDKVLSAYNKHRDKPIREQAENIIHLFTLTNLGAPAAFKYFDTTIDRKRYTSTKEVLDATLIHQ |
| | SITGLYETRIDLSQLGGDSGGSSGGSSGSETPGTSESATPESSGGSSGGSSTLNIEDEYRLHETS |
| | KEPDVSLGSTWLSDFPQAWAETGGMGLAVRQAPLIIPLKATSTPVSIKQYPMSQEARLGIKPHIQR |
| | LLDQGILVPCQSPWNTPLLPVKKPGTNDYRPVQDLREVNKRVEDIHPTVPNPYNLLSGLPPSHQW |
| | YTVLDLKDAFFCLRLHPTSQPLFAFEWRDPEMGISGQLTWTRLPQGFKNSPTLFNEALHRDLADF |
| | RIQHPDLILLQYVDDLLLAATSELDCQQGTRALLQTLGNLGYRASAKKAQICQKQVKYLGYLLKEG |
| | QRWLTEARKETVMGQPTPKTPRQLREFLGKAGFCRLFIPGFAEMAAPLYPLTKPGTLFNWGPDQ |
| | QKAYQEIKQALLTAPALGLPDLTKPFELFVDEKQGYAKGVLTQKLGPWRRPVAYLSKKLDPVAAG |
| | WPPCLRMVAAIAVLTKDAGKLTMGQPLVILAPHAVEALVKQPPDRWLSNARMTHYQALLLDTDRV |
| | QFGPVVALNPATLLPLPEEGLQHNCLDILAEAHGTRPDLTDQPLPDADHTWYTDGSSLLQEGQRK |
| | AGAAVTTETEVIWAKALPAGTSAQRAELIALTQALKMAEGKKLNVYTDSRYAFATAHIHGEIYRRR |
| | GWLTSEGKEIKNKDEILALLKALFLPKRLSIIHCPGHQKGHSAEARGNRMADQAARKAAITETPDT |
| | STLLIENSSPSGGSKRTADGSEFEPKKKRKV |
| | KEY: |
| | NUCLEAR LOCALIZATION SEQUENCE (NLS) |
| | CAS9(H840A) |
| | 33-AMINO ACID LINKER |
| | M-MLV REVERSE TRANSCRIPTASE |
| PE2-N-terminal | MKRTADGSEFESPKKKRKV |
| NLS (SEQ ID | |
| NO: 17) | |
| PE2-CAS9 | DKKYSIGLDIGTNSVGWAVITDEYKVPSKKFKVLGNTDRHSIKKNLIGALLFDSGETAEATRLKRTA |
| (H840A) (MET | RRRYTRRKNRICYLQEIFSNEMAKVDDSFFHRLEESFLVEEDKKHERHPIFGNIVDEVAYHEKYPTI |
| MINUS) (SEQ ID | YHLRKKLVDSTDKADLRLIYLALAHMIKFRGHFLIEGDLNPDNSDVDKLFIQLVQTYNQLFEENPINA |
| NO: 58) | SGVDAKAILSARLSKSRRLENLIAQLPGEKKNGLFGNLIALSLGLTPNFKSNFDLAEDAKLQLSKDT |
| | YDDDLDNLLAQIGDQYADLFLAAKNLSDAILLSDILRVNTEITKAPLSASMIKRYDEHHQDLTLLKAL |
| | VRQQLPEKYKEIFFDQSKNGYAGYIDGGASQEEFYKFIKPILEKMDGTEELLVKLNREDLLRKQRT |
| | FDNGSIPHQIHLGELHAILRRQEDFYPFLKDNREKIEKILTFRIPYYVGPLARGNSRFAWMTRKSEE |
| | TITPWNFEEVVDKGASAQSFIERMTNFDKNLPNEKVLPKHSLLYEYFTVYNELTKVKYVTEGMRK |
| | PAFLSGEQKKAIVDLLFKTNRKVTVKQLKEDYFKKIECFDSVEISGVEDRFNASLGTYHDLLKIIKDK |
| | DFLDNEENEDILEDIVLTLTLFEDREMIEERLKTYAHLFDDKVMKQLKRRRYTGWGRLSRKLINGIR |
| | DKQSGKTILDFLKSDGFANRNFMQLIHDDSLTFKEDIQKAQVSGQGDSLHEHIANLAGSPAIKKGIL |
| | QTVKVVDELVKVMGRHKPENIVIEMARENQTTQKGQKNSRERMKRIEEGIKELGSQILKEHPVEN |
| | TQLQNEKLYLYYLQNGRDMYVDQELDINRLSDYDVDAIVPQSFLKDDSIDNKVLTRSDKNRGKSD |
| | NVPSEEVVKKMKNYWRQLLNAKLITQRKFDNLTKAERGGLSELDKAGFIKRQLVETRQITKHVAQI |
| | LDSRMNTKYDENDKLIREVKVITLKSKLVSDFRKDFQFYKVREINNYHHAHDAYLNAVVGTALIKKY |
| | PKLESEFVYGDYKVYDVRKMIAKSEQEIGKATAKYFFYSNIMNFFKTEITLANGEIRKRPLIETNGET |
| | GEIVWDKGRDFATVRKVLSMPQVNIVKKTEVQTGGFSKESILPKRNSDKLIARKKDWDPKKYGGF |
| | DSPTVAYSVLWVAKVEKGKSKKLKSVKELLGITIMERSSFEKNPIDFLEAKGYKEVKKDLIIKLPKYS |
| | LFELENGRKRMLASAGELQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYFDTTIDRKR |
| | YTSTKEVLDATLIHQSITGLYETRIDLSQLGGD |
| PE2-linker | SGGSSGGSSGSETPGTSESATPESSGGSSGGSS |
| between CAS9 | |
| domain and RT | |
| domain (33 | |
| amino acids) | |
| (SEQ ID NO: 59) | |
| PE2-MMLV_RT | TLNIEDEYRLHETSKEPDVSLGSTWLSDFPQAWAETGGMGLAVRQAPLIIPLKATSTPVSIKQYPM |
| D200N T330P | SQEARLGIKPHIQRLLDQGILVPCQSPWNTPLLPVKKPGTNDYRPVQDLREVNKRVEDIHPTVPNP |
| L603W T306K | YNLLSGLPPSHQWYTVLDLKDAFFCLRLHPTSQPLFAFEWRDPEMGISGQLTWTRLPQGFKNSP |
| W313F (SEQ ID | TLFNEALHRDLADFRIQHPDLILLQYVDDLLLAATSELDCQQGTRALLQTLGNLGYRASAKKAQIC |
| NO: 49) | QKQVKYLGYLLKEGQRWLTEARKETVMGQPTPKTPRQLREFLGKAGFCRLFIPGFAEMAAPLYP |
| | LTKPGTLFNWGPDQQKAYQEIKQALLTAPALGLPDLTKPFELFVDEKQGYAKGVLTQKLGPWRR |
| | PVAYLSKKLDPVAAGWPPCLRMVAAIAVLTKDAGKLTMGQPLVILAPHAVEALVKQPPDRWLSNA |
| | RMTHYQALLLDTDRVQFGPVVALNPATLLPLPEEGLQHNCLDILAEAHGTRPDLTDQPLPDADHT |
| | WYTDGSSLLQEGQRKAGAAVTTETEVIWAKALPAGTSAQRAELIALTQALKMAEGKKLNVYTDSR |
| | YAFATAHIHGEIYRRRGWLTSEGKEIKNKDEILALLKALFLPKRLSIIHCPGHQKGHSAEARGNRMA |
| | DQAARKAAITETPDTSTLLIENSSP |
| PE2-C-terminal | SGGSKRTADGSEFEPKKKRKV |
| NLS (SEQ ID | |
| NO: 25) | |

TABLE 4B
Amino acid sequence of PEmax and its individual components

| DESCRIPTION | SEQUENCE |
|---|---|
| PEmax fusion | MKRTADGSEFESPKKKRKVDKKYSIGLDIGTNSVGWAVITDEYKVPSKKFKVLG |
| protein (SEQ | NTDRHSIKKNLIGALLFDSGETAEATRLKRTARRRYTRRKNRICYLQEIFSNEMA |
| ID NO: 6031) | KVDDSFFHRLEESFLVEEDKKHERHPIFGNIVDEVAYHEKYPTIYHLRKKLVDST |
| | DKADLRLIYLALAHMIKFRGHFLIEGDLNPDNSDVDKLFIQLVQTYNQLFEENPI |
| | NASGVDAKAILSARLSKSRKLENLIAQLPGEKKNGLFGNLIALSLGLTPNFKSNF |
| | DLAEDAKLQLSKDTYDDDLDNLLAQIGDQYADLFLAAKNLSDAILLSDILRVNT |
| | EITKAPLSASMIKRYDEHHQDLTLLKALVRQQLPEKYKEIFFDQSKNGYAGYIDG |
| | GASQEEFYKFIKPILEKMDGTEELLVKLKREDLLRKQRTFDNGSIPHQIHLGELH |
| | AILRRQEDFYPFLKDNREKIEKILTFRIPYYVGPLARGNSRFAWMTRKSEETITPW |
| | NFEEVVDKGASAQSFIERMTNFDKNLPNEKVLPKHSLLYEYFTVYNELTKVKYV |
| | TEGMRKPAFLSGEQKKAIVDLLFKTNRKVTVKQLKEDYFKKIECFDSVEISGVE |
| | DRFNASLGTYHDLLKIIKDKDFLDNEENEDILEDIVLTLTLFEDREMIEERLKTYA |
| | HLFDDKVMKQLKRRRYTGWGRLSRKLINGIRDKQSGKTILDFLKSDGFANRNF |
| | MQLIHDDSLTFKEDIQKAQVSGQGDSLHEHIANLAGSPAIKKGILQTVKVVDELV |
| | KVMGRHKPENIVIEMARENQTTQKGQKNSRERMKRIEEGIKELGSQILKEHPVE |
| | NTQLQNEKLYLYYLQNGRDMYVDQELDINRLSDYDVDAIVPQSFLKDDSIDNK |
| | VLTRSDKNRGKSDNVPSEEVVKKMKNYWRQLLNAKLITQRKFDNLTKAERGG |
| | LSELDKAGFIKRQLVETRQITKHVAQILDSRMNTKYDENDKLIREVKVITLKSKL |
| | VSDFRKDFQFYKVREINNYHHAHDAYLNAVVGTALIKKYPKLESEFVYGDYKV |
| | YDVRKMIAKSEQEIGKATAKYFFYSNIMNFFKTEITLANGEIRKRPLIETNGETGE |
| | IVWDKGRDFATVRKVLSMPQVNIVKKTEVQTGGFSKESILPKRNSDKLIARKKD |
| | WDPKKYGGFDSPTVAYSVLVVAKVEKGKSKKLKSVKELLGITIMERSSFEKNPI |
| | DFLEAKGYKEVKKDLIIKLPKYSLFELENGRKRMLASAGELQKGNELALPSKYV |
| | NFLYLASHYEKLKGSPEDNEQKQLFVEQHKHYLDEIIEQISEFSKRVILADANLD |
| | KVLSAYNKHRDKPIREQAENIIHLFTLTNLGAPAAFKYFDTTIDRKRYTSTKEVL |
| | DATLIHQSITGLYETRIDLSQLGGDSGGSSGGSKRTADGSEFESPKKKRKVSGGSS |
| | GGSTLNIEDEYRLHETSKEPDVSLGSTWLSDFPQAWAETGGMGLAVRQAPLIIPL |
| | KATSTPVSIKQYPMSQEARLGIKPHIQRLLDQGILVPCQSPWNTPLLPVKKPGTN |
| | DYRPVQDLREVNKRVEDIHPTVPNPYNLLSGLPPSHQWYTVLDLKDAFFCLRLH |
| | PTSQPLFAFEWRDPEMGISGQLTWTRLPQGFKNSPTLFNEALHRDLADFRIQHPD |
| | LILLQYVDDLLLAATSELDCQQGTRALLQTLGNLGYRASAKKAQICQKQVKYL |
| | GYLLKEGQRWLTEARKETVMGQPTPKTPRQLREFLGKAGFCRLFIPGFAEMAAP |
| | LYPLTKPGTLFNWGPDQQKAYQEIKQALLTAPALGLPDLTKPFELFVDEKQGYA |
| | KGVLTQKLGPWRRPVAYLSKKLDPVAAGWPPCLRMVAAIAVLTKDAGKLTMG |
| | QPLVILAPHAVEALVKQPPDRWLSNARMTHYQALLLDTDRVQFGPVVALNPAT |
| | LLPLPEEGLQHNCLDILAEAHGTRPDLTDQPLPDADHTWYTDGSSLLQEGQRKA |
| | GAAVTTETEVIWAKALPAGTSAQRAELIALTQALKMAEGKKLNVYTDSRYAFA |
| | TAHIHGEIYRRRGWLTSEGKEIKNKDEILALLKALFLPKRLSIIHCPGHQKGHSAE |
| | ARGNRMADQAARKAAITETPDTSTLLIENSSPSGGSKRTADGSEFESPKKKRKV |
| | GSGPAAKRVKLD |
| PEmax-N- | MKRTADGSEFESPKKKRKV |
| terminal NLS | |
| (SEQ ID NO: | |
| 6032) | |
| PEmax-CAS9 | DKKYSIGLDIGTNSVGWAVITDEYKVPSKKFKVLGNTDRHSIKKNLIGALLFDSG |
| (R221K N394K | ETAEATRLKRTARRRYTRRKNRICYLQEIFSNEMAKVDDSFFHRLEESFLVEEDK |
| H840A) (not | KHERHPIFGNIVDEVAYHEKYPTIYHLRKKLVDSTDKADLRLIYLALAHMIKFRG |
| including N- | HFLIEGDLNPDNSDVDKLFIQLVQTYNQLFEENPINASGVDAKAILSARLSKSRK |
| terminal Met in | LENLIAQLPGEKKNGLFGNLIALSLGLTPNFKSNFDLAEDAKLQLSKDTYDDDLD |
| Cas9) (SEQ ID | NLLAQIGDQYADLFLAAKNLSDAILLSDILRVNTEITKAPLSASMIKRYDEHHQD |
| NO: 6033) | LTLLKALVRQQLPEKYKEIFFDQSKNGYAGYIDGGASQEEFYKFIKPILEKMDGT |
| | EELLVKLKREDLLRKQRTFDNGSIPHQIHLGELHAILRRQEDFYPFLKDNREKIEK |
| | ILTFRIPYYVGPLARGNSRFAWMTRKSEETITPWNFEEVVDKGASAQSFIERMTN |
| | FDKNLPNEKVLPKHSLLYEYFTVYNELTKVKYVTEGMRKPAFLSGEQKKAIVDL |
| | LFKTNRKVTVKQLKEDYFKKIECFDSVEISGVEDRFNASLGTYHDLLKIIKDKDF |
| | LDNEENEDILEDIVLTLTLFEDREMIEERLKTYAHLFDDKVMKQLKRRRYTGWG |
| | RLSRKLINGIRDKQSGKTILDFLKSDGFANRNFMQLIHDDSLTFKEDIQKAQVSG |
| | QGDSLHEHIANLAGSPAIKKGILQTVKVVDELVKVMGRHKPENIVIEMARENQT |
| | TQKGQKNSRERMKRIEEGIKELGSQILKEHPVENTQLQNEKLYLYYLQNGRDM |
| | YVDQELDINRLSDYDVDAIVPQSFLKDDSIDNKVLTRSDKNRGKSDNVPSEEVV |
| | KKMKNYWRQLLNAKLITQRKFDNLTKAERGGLSELDKAGFIKRQLVETRQITK |
| | HVAQILDSRMNTKYDENDKLIREVKVITLKSKLVSDFRKDFQFYKVREINNYHH |
| | AHDAYLNAVVGTALIKKYPKLESEFVYGDYKVYDVRKMIAKSEQEIGKATAKY |
| | FFYSNIMNFFKTEITLANGEIRKRPLIETNGETGEIVWDKGRDFATVRKVLSMPQ |
| | VNIVKKTEVQTGGFSKESILPKRNSDKLIARKKDWDPKKYGGFDSPTVAYSVLV |
| | VAKVEKGKSKKLKSVKELLGITIMERSSFEKNPIDFLEAKGYKEVKKDLIIKLPK |
| | YSLFELENGRKRMLASAGELQKGNELALPSKYVNFLYLASHYEKLKGSPEDNE |
| | QKQLFVEQHKHYLDEIIEQISEFSKRVILADANLDKVLSAYNKHRDKPIREQAENI |
| | IHLFTLTNLGAPAAFKYFDTTIDRKRYTSTKEVLDATLIHQSITGLYETRIDLSQL |
| | GGD |
| PEmax- | SGGSSGGSKRTADGSEFESPKKKRKVSGGSSGGS |
| SGGSx2- | |
| bpSV40NLS- | |
| SGGSx2 linker | |
| (SEQ ID NO: | |
| 6028) | |
| PEmax- | TLNIEDEYRLHETSKEPDVSLGSTWLSDFPQAWAETGGMGLAVRQAPLIIPLKAT |
| MMLV_RT | STPVSIKQYPMSQEARLGIKPHIQRLLDQGILVPCQSPWNTPLLPVKKPGTNDYR |
| D200N T330P | PVQDLREVNKRVEDIHPTVPNPYNLLSGLPPSHQWYTVLDLKDAFFCLRLHPTS |
| L603W T306K | QPLFAFEWRDPEMGISGQLTWTRLPQGFKNSPTLFNEALHRDLADFRIQHPDLIL |
| W313F (SEQ | LQYVDDLLLAATSELDCQQGTRALLQTLGNLGYRASAKKAQICQKQVKYLGYL |
| ID NO: 6034) | LKEGQRWLTEARKETVMGQPTPKTPRQLREFLGKAGFCRLFIPGFAEMAAPLYP |
| | LTKPGTLFNWGPDQQKAYQEIKQALLTAPALGLPDLTKPFELFVDEKQGYAKG |
| | VLTQKLGPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVALNPATLL |
| | PLPEEGLQHNCLDILAEAHGTRPDLTDQPLPDADHTWYTDGSSLLQEGQRKAGA |
| | AVTTETEVIWAKALPAGTSAQRAELIALTQALKMAEGKKLNVYTDSRYAFATA |
| | HIHGEIYRRRGWLTSEGKEIKNKDEILALLKALFLPKRLSIIHCPGHQKGHSAEAR |
| | GNRMADQAARKAAITETPDTSTLLIENSSP |
| PEmax-C- | SGGSKRTADGSEFESPKKKRKV |
| terminal linker- | |
| NLS (SEQ ID | |
| NO: 6029) | |
| PEmax-C- | GSGPAAKRVKLD |
| terminal linker- | |
| NLS2 (SEQ ID | |
| NO: 6030) | |

### PEgRNA for Editing of SLC37A4 Gene

The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm.

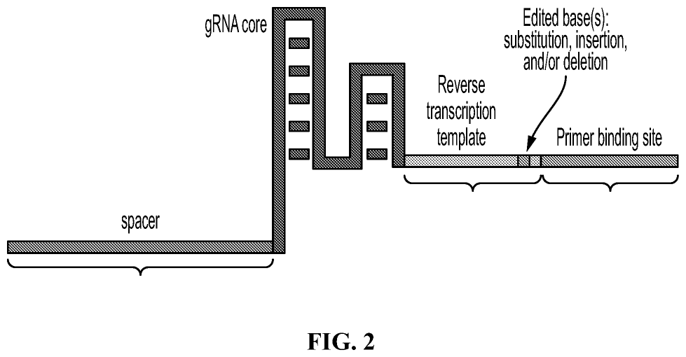

In certain embodiments, the extension arm comprises a primer binding site sequence (PBS) that can initiate target-primed DNA synthesis. In some embodiments, the PBS is complementary or substantially complementary to a free 3′ end on the edit strand of the target gene at a nick site generated by the prime editor. In some embodiments, the extension arm further comprises an editing template that comprises one or more intended nucleotide edits to be incorporated in the target gene by prime editing. In some embodiments, the editing template is a template for an RNA-dependent DNA polymerase domain or polypeptide of the prime editor, for example, a reverse transcriptase domain. The reverse transcriptase editing template may also be referred to herein as an RT template, or RTT. In some embodiments, the editing template comprises partial complementarity to an editing target sequence in the target gene, e.g., an SLC37A4 gene. In some embodiments, the editing template comprises substantial or partial complementarity to the editing target sequence except at the position of the intended nucleotide edits to be incorporated into the target gene. An exemplary architecture of a PEgRNA including its components is as demonstrated in FIG. 2.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase.

Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. In some embodiments, a PEgRNA comprises a PBS and an editing template sequence in 5′ to 3′ order. In some embodiments, the gRNA core of a PEgRNA of this disclosure may be located in between a spacer and an extension arm of the PEgRNA. In some embodiments, the gRNA core of a PEgRNA may be located at the 3′ end of a spacer. In some embodiments, the gRNA core of a PEgRNA may be located at the 5′ end of a spacer. In some embodiments, the gRNA core of a PEgRNA may be located at the 3′ end of an extension arm. In some embodiments, the gRNA core of a PEgRNA may be located at the 5′ end of an extension arm. In some embodiments, the PEgRNA comprises, from 5′ to 3′: a spacer, a gRNA core, and an extension arm. In some embodiments, the PEgRNA comprises, from 5′ to 3′: a spacer, a gRNA core, an editing template, and a PBS. In some embodiments, the PEgRNA comprises, from 5′ to 3′: an extension arm, a spacer, and a gRNA core. In some embodiments, the PEgRNA comprises, from 5′ to 3′: an editing target, a PBS, a spacer, and a gRNA core.

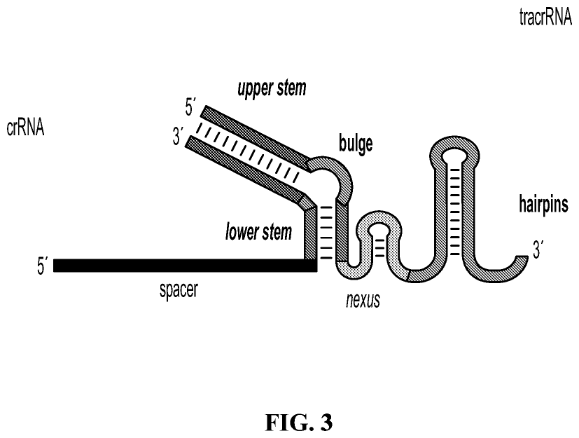

In some embodiments, a PEgRNA comprises a single polynucleotide molecule that comprises the spacer sequence, the gRNA core, and the extension arm. In some embodiments, a PEgRNA comprises multiple polynucleotide molecules, for example, two polynucleotide molecules. In some embodiments, a PEgRNA comprise a first polynucleotide molecule that comprises the spacer and a portion of the gRNA core, and a second polynucleotide molecule that comprises the rest of the gRNA core and the extension arm. In some embodiments, the gRNA core portion in the first polynucleotide molecule and the gRNA core portion in the second polynucleotide molecule are at least partly complementary to each other. In some embodiments, the PEgRNA may comprise a first polynucleotide comprising the spacer and a first portion of a gRNA core comprising, which may be also be referred to as a crRNA. In some embodiments, the PEgRNA comprise a second polynucleotide comprising a second portion of the gRNA core and the extension arm, wherein the second portion of the gRNA core may also be referred to as a trans-activating crRNA, or tracr RNA. In some embodiments, the crRNA portion and the tracr RNA portion of the gRNA core are at least partially complementary to each other. In some embodiments, the partially complementary portions of the crRNA and the tracr RNA form a lower stem, a bulge, and an upper stem, as exemplified in FIG. 3.

In some embodiments, a spacer sequence comprises a region that has substantial complementarity to a search target sequence on the target strand of a double stranded target DNA, e.g., a SLC37A4 gene. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

Exemplary sequences for PEgRNA spacers are provided in Table 8.

The extension arm of a PEgRNA may comprise a primer binding site (PBS) and an editing template (e.g., an RTT). The extension arm may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that hybridizes with a free 3′ end of a single stranded DNA in the target gene (e.g., the SLC37A4 gene) generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene (e.g., the SLC37A4 gene). In some embodiments, the PBS is perfectly complementary, or has 100% complementary, to a region of the edit strand of the target gene (e.g., the SLC37A4 gene).

Exemplary sequences for PBS are provided in Tables 9b, 10b, 11b, 12b, 13b, 14b, 15b, 16b, 17b, 18b, 19b, 20b, 21b, 22b, 23b, and 24b for spacers S01, S02, S03, S04, S05, S06, S07, S08, S09, S10, S11, S12, S13, S14, S15, and S16, respectively.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

Exemplary sequences for RTT are provided in Tables 9a, 10a, 11a, 12a, 13a, 14a, 15a, 16a, 17a, 18a, 19a, 20a, 21a, 22a, 23a, and 24a for spacers S01, S02, S03, S04, S05, S06, S07, S08, S09, S10, S11, S12, S13, S14, S15, and S16, respectively.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated in the target gene. In some embodiments, the editing template comprises a nucleotide sequence comprising about 85% to about 95% complementarity to an editing target sequence in the edit strand in the target gene (e.g., the SLC37A4 gene). In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene (e.g., the SLC37A4 gene).

An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-to-A substitution. In some embodiments, a nucleotide substitution comprises a T-to-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the SLC37A4 gene to be edited. Position of the intended nucleotide edit(s) relevant to other components of the PEgRNA, or to particular nucleotides (e.g., mutations) in the SLC37A4 target gene may vary. In some embodiments, the nucleotide edit is in a region of the PEgRNA corresponding to or homologous to the protospacer sequence. In some embodiments, the nucleotide edit is in a region of the PEgRNA corresponding to a region of the SLC37A4 gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream of the 5′ most nucleotide of the PAM sequence in the edit strand of the target gene. By 0 base pair upstream or downstream of a reference position, it is meant that the intended nucleotide is immediately upstream or downstream of the reference position. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream of the 5′ most nucleotide of the PAM sequence. In some embodiments, the nucleotide edit is incorporated at a position corresponding to 3 base pairs upstream of the 5′ most nucleotide of the PAM sequence. In some embodiments, the nucleotide edit in is incorporated at a position corresponding to 4 base pairs upstream of the 5′ most nucleotide of the PAM sequence. In some embodiments, the nucleotide edit is incorporated at a position corresponding to 5 base pairs upstream of the 5′ most nucleotide of the PAM sequence. In some embodiments, the nucleotide edit in the editing template is at a position corresponding to 6 base pairs upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs downstream of the 5′ most nucleotide of the PAM sequence in the edit strand of the target gene. In some embodiments, a nucleotide edit is incorporated at a position corresponding to about 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs downstream of the 5′ most nucleotide of the PAM sequence. In some embodiments, a nucleotide edit is incorporated at a position corresponding to 3 base pairs downstream of the 5′ most nucleotide of the PAM sequence. In some embodiments, a nucleotide edit is incorporated at a position corresponding to 4 base pairs downstream of the 5′ most nucleotide of the PAM sequence. In some embodiments, a nucleotide edit is incorporated at a position corresponding to 5 base pairs downstream of the 5′ most nucleotide of the PAM sequence. In some embodiments, a nucleotide edit is incorporated at a position corresponding to 6 base pairs downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

In some embodiments, the position of a nucleotide edit incorporation in the target gene may be determined based on position of the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, position of an intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides downstream of the nick site on the PAM strand (or the non-target strand, or the edit strand) of the double stranded target DNA. In some embodiments, position of the intended nucleotide edit in the editing template may be referred to by aligning the editing template with the partially complementary editing target sequence on the edit strand, and referring to nucleotide positions on the editing strand where the intended nucleotide edit is incorporated. Accordingly, in some embodiments, a nucleotide edit in an editing template is at a position corresponding to a position about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, or 150 nucleotides apart from the nick site. In some embodiments, a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides apart from the nick site. In some embodiments, when referred to in the context of the PAM strand (or the non-target strand, or the edit strand), a nucleotide edit in an editing template is at a position corresponding to a position about 0 to 2 nucleotides, 0 to 4 nucleotides, 0 to 6 nucleotides, 0 to 8 nucleotides, 0 to 10 nucleotides, 2 to 4 nucleotides, 2 to 6 nucleotides, 2 to 8 nucleotides, 2 to 10 nucleotides, 2 to 12 nucleotides, 4 to 6 nucleotides, 4 to 8 nucleotides, 4 to 10 nucleotides, 4 to 12 nucleotides, 4 to 14 nucleotides, 6 to 8 nucleotides, 6 to 10 nucleotides, 6 to 12 nucleotides, 6 to 14 nucleotides, 6 to 16 nucleotides, 8 to 10 nucleotides, 8 to 12 nucleotides, 8 to 14 nucleotides, 8 to 16 nucleotides, 8 to 18 nucleotides, 10 to 12 nucleotides, 10 to 14 nucleotides, 10 to 16 nucleotides, 10 to 18 nucleotides, 10 to 20 nucleotides, 12 to 14 nucleotides, 12 to 16 nucleotides, 12 to 18 nucleotides, 12 to 20 nucleotides, 12 to 22 nucleotides, 14 to 16 nucleotides, 14 to 18 nucleotides, 14 to 20 nucleotides, 14 to 22 nucleotides, 14 to 24 nucleotides, 16 to 18 nucleotides, 16 to 20 nucleotides, 16 to 22 nucleotides, 16 to 24 nucleotides, 16 to 26 nucleotides, 18 to 20 nucleotides, 18 to 22 nucleotides, 18 to 24 nucleotides, 18 to 26 nucleotides, 18 to 28 nucleotides, 20 to 22 nucleotides, 20 to 24 nucleotides, 20 to 26 nucleotides, 20 to 28 nucleotides, 20 to 30 nucleotides, 30 to 40 nucleotides, 40 to 50 nucleotides, 50 to 60 nucleotides, 60 to 70 nucleotides, 70 to 80 nucleotides, 80 to 90 nucleotides, 90 to 100 nucleotides, 100 to 110 nucleotides, 110 to 120 nucleotides, 120 to 130 nucleotides, 130 to 140 nucleotides, or 140 to 150 nucleotides downstream from the nick site. The relative positions of the intended nucleotide edit(s) and nick site may be referred to by numbers. For example, in some embodiments, the nucleotide immediately downstream of the nick site on a PAM strand (or the non-target strand, or the edit strand) may be referred to as at position 0. The nucleotide immediately upstream of the nick site on the PAM strand (or the non-target strand, or the edit strand) may be referred to as at position −1. The nucleotides downstream of position 0 on the PAM strand may be referred to as at positions +1, +2, +3, +4, . . . +n, and the nucleotides upstream of position −1 on the PAM strand may be referred to as at positions −2, −3, −4, . . . , −n. Accordingly, in some embodiments, the nucleotide in the editing template that corresponds to position 0 when the editing template is aligned with the partially complementary editing target sequence by complementarity may also be referred to as position 0 in the editing template, the nucleotides in the editing template corresponding to the nucleotides at positions +1, +2, +3, +4, . . . , +n on the PAM strand of the double stranded target DNA may also be referred to as at positions +1, +2, +3, +4, . . . , +n in the editing template, and the nucleotides in the editing template corresponding to the nucleotides at positions −1, −2, −3, −4, . . . , −n on the PAM strand on the double stranded target DNA may also be referred to as at positions −1, −2, −3, −4, . . . , −n on the editing template, even though when the PEgRNA is viewed as a standalone nucleic acid, positions +1, +2, +3, +4, . . . , +n are 5′ of position 0 and positions −1, −2, −3, −4, . . . , −n are 3′ of position 0 in the editing template. In some embodiments, an intended nucleotide edit is at position +n of the editing template relative to position 0. Accordingly, the intended nucleotide edit may be incorporated at position +n of the PAM strand of the double stranded target DNA (and subsequently, the target strand of the double stranded target DNA) by prime editing. The number n may be referred to as the nick to edit distance. When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs upstream to the 5′ most nucleotide of the PBS. In some embodiments, the intended nucleotide edit is 0 to 2 base pairs, 0 to 4 base pairs, 0 to 6 base pairs, 0 to 8 base pairs, 0 to 10 base pairs, 2 to 4 base pairs, 2 to 6 base pairs, 2 to 8 base pairs, 2 to 10 base pairs, 2 to 12 base pairs, 4 to 6 base pairs, 4 to 8 base pairs, 4 to 10 base pairs, 4 to 12 base pairs, 4 to 14 base pairs, 6 to 8 base pairs, 6 to 10 base pairs, 6 to 12 base pairs, 6 to 14 base pairs, 6 to 16 base pairs, 8 to 10 base pairs, 8 to 12 base pairs, 8 to 14 base pairs, 8 to 16 base pairs, 8 to 18 base pairs, 10 to 12 base pairs, 10 to 14 base pairs, 10 to 16 base pairs, 10 to 18 base pairs, 10 to 20 base pairs, 12 to 14 base pairs, 12 to 16 base pairs, 12 to 18 base pairs, 12 to 20 base pairs, 12 to 22 base pairs, 14 to 16 base pairs, 14 to 18 base pairs, 14 to 20 base pairs, 14 to 22 base pairs, 14 to 24 base pairs, 16 to 18 base pairs, 16 to 20 base pairs, 16 to 22 base pairs, 16 to 24 base pairs, 16 to 26 base pairs, 18 to 20 base pairs, 18 to 22 base pairs, 18 to 24 base pairs, 18 to 26 base pairs, 18 to 28 base pairs, 20 to 22 base pairs, 20 to 24 base pairs, 20 to 26 base pairs, 20 to 28 base pairs, or 20 to 30 base pairs upstream to the 5′ most nucleotide of the PBS.

The corresponding positions of the intended nucleotide edit incorporated in the target gene may also be referred to based on the nicking position generated by a prime editor based on sequence homology and complementarity. For example, in embodiments, the distance between the nucleotide edit to be incorporated into the target SLC37A4 gene and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 base pairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

In some embodiments, the editing template extends beyond a nucleotide edit to be incorporated to the target SLC37A4 gene sequence. For example, in some embodiments, the editing template comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 base pairs 3′ to the nucleotide edit to be incorporated to the target SLC37A4 gene sequence. In some embodiments, the editing template comprises at least 4 to 30 base pairs 3′ to the nucleotide edit to be incorporated to the target SLC37A4 gene sequence. In some embodiments, the editing template comprises at least 4 to 25 base pairs 3′ to the nucleotide edit to be incorporated to the target SLC37A4 gene sequence. In some embodiments, the editing template comprises at least 4 to 20 base pairs 3′ to the nucleotide edit to be incorporated to the target SLC37A4 gene sequence. In some embodiments, the editing template comprises at least 4 to 30 base pairs 5′ to the nucleotide edit to be incorporated to the target SLC37A4 gene sequence. In some embodiments, the editing template comprises at least 4 to 25 base pairs 5′ to the nucleotide edit to be incorporated to the target SLC37A4 gene sequence. In some embodiments, the editing template comprises at least 4 to 20 base pairs 5′ to the nucleotide edit to be incorporated to the target SLC37A4 gene sequence.

In some embodiments, the editing template comprises an adenine at the first nucleobase position (e.g., for a PEgRNA following 5′-spacer-gRNA core-RTT-PBS-3′ orientation, the 5′ most nucleobase is the “first base”). In some embodiments, the editing template comprises a guanine at the first nucleobase position (e.g., for a PEgRNA following 5′-spacer-gRNA core-RTT-PBS-3′ orientation, the 5′ most nucleobase is the “first base”). In some embodiments, the editing template comprises an uracil at the first nucleobase position (e.g., for a PEgRNA following 5′-spacer-gRNA core-RTT-PBS-3′ orientation, the 5′ most nucleobase is the “first base”). In some embodiments, the editing template comprises a cytosine at the first nucleobase position (e.g., for a PEgRNA following 5′-spacer-gRNA core-RTT-PBS-3′ orientation, the 5′ most nucleobase is the “first base”). In some embodiments, the editing template does not comprise a cytosine at the first nucleobase position (e.g., for a PEgRNA following 5′-spacer-gRNA core-RTT-PBS-3′ orientation, the 5′ most nucleobase is the “first base”).

The editing template of a PEgRNA may encode a new single stranded DNA (e.g., by reverse transcription) to replace a target sequence in the target gene. In some embodiments, the editing target sequence in the edit strand of the target gene is replaced by the newly synthesized strand, and the nucleotide edit(s) are incorporated in the region of the target gene. In some embodiments, the target gene is an SLC37A4 gene. In some embodiments, the editing template of the PEgRNA encodes a newly synthesized single stranded DNA that comprises a wild type SLC37A4 gene sequence. In some embodiments, the newly synthesized DNA strand replaces the editing target sequence in the target SLC37A4 gene, wherein the editing target sequence (or the endogenous sequence complementary to the editing target sequence on the target strand of the SLC37A4 gene) comprises a mutation compared to a wild type SLC37A4 gene. In some embodiments, the mutation is associated with retinal degenerative disease, such as Glycogen storage disease type 1B.

In some embodiments, the editing target sequence comprises a mutation in exon 8 of the SLC37A4 gene as compared to a wild type SLC37A4 gene. In some embodiments, the editing target sequence comprises a mutation that is located at position 1015 of the coding sequence of the G6PT1/SLC37A4 protein. In some embodiments, the editing target sequence comprises a c. 1015G->T mutation (on the sense strand) or a C->A mutation (on the antisense strand) at position 1015 of the coding sequence of the G6PT1 protein. In some embodiments, the editing target sequence comprises a mutation that is located at position 1042 of the coding sequence of the G6PT1/SLC37A4 protein. In some embodiments, the editing target sequence comprises a c. 1042delCT mutation (on the sense strand) or a delAG mutation (on the antisense strand) at position 1042 of the coding sequence of the G6PT1/SLC37A4 protein.

In some embodiments, the editing template comprises one or more intended nucleotide edits compared to the sequence on the target strand of the SLC37A4 gene that is complementary to the editing target sequence. In some embodiments, the editing template encodes a single stranded DNA that comprises one or more intended nucleotide edits compared to the editing target sequence. In some embodiments, the single stranded DNA replaces the editing target sequence by prime editing, thereby incorporating the one or more intended nucleotide edits. In some embodiments, the one or more intended nucleotide edits comprises a G-T substitution at a position corresponding to position 1015 of the coding sequence of the G6PT1 protein compared to the editing target sequence. In some embodiments, the one or more intended nucleotide edits comprises an A-C substitution in the anti-sense strand at a position corresponding to position 1015 of the coding sequence of the G6PT1 protein compared to the editing target sequence. In some embodiments, the one or more intended nucleotide edits comprises a CT insertion at a position corresponding to position 1042 of the coding sequence of the G6PT1 protein compared to the editing target sequence. In some embodiments, the one or more intended nucleotide edits comprises an AG insertion in the anti-sense strand at a position corresponding to position 1042 of the coding sequence of the G6PT1 protein compared to the editing target sequence. In some embodiments, incorporation of the one or more intended nucleotide edits corrects the mutation in the editing target sequence to wild type nucleotides at corresponding positions in the SLC37A4 gene. As used herein, “correcting” a mutation means restoring a wild type sequence at the place of the mutation in the double stranded target DNA, e.g. target gene, by prime editing. In some embodiments, the editing template comprises and/or encodes a wild type SLC37A4 gene sequence.

In some embodiments, incorporation of the one or more intended nucleotide edits does not correct the mutation in the editing target sequence to wild type sequence, but allows for expression of a functional G6PT1 protein encoded by the SLC37A4 gene. For example, in some embodiments, incorporation of the one or more intended nucleotide edits results in one or more codons that are different from a wild type codon but encode one or more amino acids same as the wild type G6PT1 protein. In some embodiments, incorporation of the one or more intended nucleotide edits results in one or more codons that encode one or more amino acids different from the wild type G6PT1 protein, but allows for expression of a functional G6PT1 protein.

A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core comprises regions and secondary structures involved in binding with specific CRISPR Cas proteins. For example, in a Cas9 based prime editing system, the gRNA core of a PEgRNA may comprise one or more regions of a base paired “lower stem” adjacent to the spacer sequence and a base paired “upper stem” following the lower stem, where the lower stem and upper stem may be connected by a “bulge” comprising unpaired RNAs. The gRNA core may further comprise a “nexus” distal from the spacer sequence, followed by a hairpin structure, e.g., at the 3′ end, as exemplified in FIG. 3. In some embodiments, the gRNA core comprises modified nucleotides as compared to a wild type gRNA core in the lower stem, upper stem, and/or the hairpin. For example, nucleotides in the lower stem, upper stem, an/or the hairpin regions may be modified, deleted, or replaced. In some embodiments, RNA nucleotides in the lower stem, upper stem, an/or the hairpin regions may be replaced with one or more DNA sequences. In some embodiments, the gRNA core comprises unmodified or wild type RNA sequences in the nexus and/or the bulge regions. In some embodiments, the gRNA core does not include long stretches of A-T pairs, for example, a GUUUU-AAAAC pairing element.

In some embodiments, the gRNA core comprises the sequences (as with all RNA sequences provided herein, the T residues in the below sequences may be replaced with U residues): GTTTTAGAGCTAGAAATAGCAAGTTAAAATAAGGCTAGTCCGTTATCAACTTGAA AAAGTGGCACCGAGTCGGTGC (SEQ ID NO: 6035); GITTGAGAGCTAGAAATAGCAAGTTTAAATAAGGCTAGTCCGTTATCAACTTGAA AAAGTGGGACCGAGTCGGTCC (SEQ ID NO: 6036), or GTTTAAGAGCTATGCTGGAAACAGCATAGCAAGTTTAAATAAGGCTAGTCCGTTA TCAACTTGAAAAAGTGGCACCGAGTCGGTGC (SEQ ID NO: 6037). In some embodiments, the gRNA core comprises the sequence GTTTTAGAGCTAGAAATAGCAAGTTAAAATAAGGCTAGTCCGTTATCAACTTGAA AAAGTGGCACCGAGTCGGTGC (SEQ ID NO: 6035). Any gRNA core sequences known in the art are also contemplated in the prime editing compositions described herein.

In some embodiments, the gRNA core comprises the sequence: GUUUUAGAGCUAGAAAUAGCAAGUUAAAAUAAGGCUAGUCCGUUAUCAACUU GAAAAAGUGGCACCGAGUCGGUGC (SEQ ID NO: 54); GUUUGAGAGCUAGAAAUAGCAAGUUUAAAUAAGGCUAGUCCGUUAUCAACUU GAAAAAGUGGGACCGAGUCGGUCC (SEQ ID NO: 55), or GUUUAAGAGCUAUGCUGGAAACAGCAUAGCAAGUUUAAAUAAGGCUAGUCCG UUAUCAACUUGAAAAAGUGGCACCGAGUCGGUGC (SEQ ID NO: 56). In some embodiments, the gRNA core comprises the sequence GUUUUAGAGCUAGAAAUAGCAAGUUAAAAUAAGGCUAGUCCGUUAUCAACUU GAAAAAGUGGCACCGAGUCGGUGC (SEQ ID NO: 54). Any gRNA core sequences known in the art are also contemplated in the prime editing compositions described herein.

A PEgRNA may also comprise optional modifiers, e.g., 3′ end modifier region and/or an 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, the secondary structure comprises a pseudoknot. In some embodiments, the secondary structure comprises a pseudoknot derived from a virus. In some embodiments, the secondary structure comprises a pseudoknot of a Moloney murine leukemia virus (M-MLV) genome (a mpknot). In some embodiments, the secondary structure comprises a nucleotide sequence selected from the group consisting of GGGUCAGGAGCCCCCCCCCUGAACCCAGGAUAACCCUCAAAGUCGGGGGGCAAC C (SEQ ID NO: 6038), GUCAGGGUCAGGAGCCCCCCCCCUGAACCCAGGAUAACCCUCAAAGUCGGGGGG CAACCC (SEQ ID NO: 371), GGGUCAGGAGCCCCCCCCCUGAACCCAGGAAAACCCUCAAAGUCGGGGGGCAAC CC (SEQ ID NO: 6039), GGGUCAGGAGCCCCCCCCCUGCACCCAGGAAAACCCUCAAAGUCGGGGGGCAACC C (SEQ ID NO: 6040), GGGUCAGGAGCCCCCCCCCUGCACCCAGGAUAACCCUCAAAGUCGGGGGGCAACC C (SEQ ID NO: 6041), GUCAGGGUCAGGAGCCCCCCCCCUGAACCCAGGAAAACCCUCAAAGUCGGGGGG CAACCC (SEQ ID NO: 372), GUCAGGGUCAGGAGCCCCCCCCCUGCACCCAGGAAAACCCUCAAAGUCGGGGGG CAACCC (SEQ ID NO: 373), and GGGUCAGGAGCCCCCCCCCUGAACCCAGGAUAACCCUCAAAGUCGGGGGGC (SEQ ID NO: 374), or a nucleotide sequence having at least 80%, 85%, 90%, 95%, 96%, 97%, 98%, or 99% sequence identity therewith. In some embodiments, the secondary structure comprises a nucleotide sequence of GGGUCAGGAGCCCCCCCCCUGAACCCAGGAUAACCCUCAAAGUCGGGGGGC (SEQ ID NO: 375), or a nucleotide sequence having at least 80%, 85%, 90%, 95%, 96%, 97%, 98%, or 99% sequence identity therewith.

In some embodiments, the secondary structure comprises a quadruplex. In some embodiments, the secondary structure comprises a G-quadruplex. In some embodiments, the secondary structure comprises a nucleotide sequence selected from the group consisting of gq2 (UGGUGGUGGUGGU) (SEQ ID NO: 376), stk40 (GGGACAGGGCAGGGACAGGG) (SEQ ID NO: 377), apc2 (GGGUCCGGGUCUGGGUCUGGG) (SEQ ID NO: 378), stard3 (GGGCAGGGUCUGGGCUGGG) (SEQ ID NO: 379), Uns1 (GGGCUGGGAUGGGAAAGGG) (SEQ ID NO: 380), ceacam4 (GGGCUCUGGGUGGGCCGGG) (SEQ ID NO: 381), erc1 (GGGCUGGGCUGGGCAGGG) (SEQ ID NO: 382), pitpnm3 (GGGUGGGCUGGGAAGGG) (SEQ ID NO: 383), rlf (GGGAGGGAGGGCUAGGG) (SEQ ID NO: 384), ube3c (GGGCAGGGCUGGGAGGG) (SEQ ID NO: 385), taf15 (GGGUGGGAGGGCUGGG) (SEQ ID NO: 386), and xml (GCGUAACCUCCAUCCGAGUUGCAAGAGAGGGAAACGCAGUCUC) (SEQ ID NO: 387), or a nucleotide sequence having at least 80%, 85%, 90%, 95%, 96%, 97%, 98%, or 99% sequence identity therewith.

In some embodiments, the secondary structure comprises aP4-P6 domain of a Group I intron. In some embodiments, the secondary structure comprises the nucleotide sequence of GGAAUUGCGGGAAAGGGGUCAACAGCCGUUCAGUACCAAGUCUCAGGGGAAA CUUUGAGAUGGCCUUGCAAAGGGUAUGGUAAUAAGCUGACGGACAUGGUCCU AACCACGCAGCCAAGUCCUAAGUCAACAGAUCUUCUGUUGAUAUGGAUGCAGU UCA (SEQ ID NO: 388), or a nucleotide sequence having at least 80%, 85%, 90%, 95%, 96%, 97%, 98%, or 99% sequence identity therewith.

In some embodiments, the secondary structure comprises a riboswitch aptamer. In some embodiments, the secondary structure comprises a riboswitch aptamer derived from a prequeosine-1 riboswitch aptamer. In some embodiments, the secondary structure comprises a modified prequeosine-1 riboswitch aptamer. In some embodiments, the secondary structure comprises a nucleotide sequence selected from the group consisting of UUGACGCGGUUCUAUCUAGUUACGCGUUAAACCAACUAGAAA (SEQ ID NO: 389), UUGACGCGGUUCUAUCUACUUACGCGUUAAACCAACUAGAAA (SEQ ID NO: 390), CGCGAGUCUAGGGGAUAACGCGUUAAACUUCCUAGAAGGCGGUU (SEQ ID NO: 391), CGCGGAUCUAGAUUGUAACGCGUUAAACCAUCUAGAAGGCGGUU (SEQ ID NO: 392), CGCGUCGCUACCGCCCGGCGCGUUAAACACACUAGAAGGCGGUU (SEQ ID NO: 393), and CGCGGUUCUAUCUAGUUACGCGUUAAACCAACUAGAA (SEQ ID NO: 394), or a nucleotide sequence having at least 80%, 85%, 90%, 95%, 96%, 97%, 98%, or 99% sequence identity therewith. In some embodiments, the secondary structure comprises a nucleotide sequence selected from the group consisting of UUGACGCGGUUCUAUCUAGUUACGCGUUAAACCAACUAGAAA (SEQ ID NO: 389), CGCGAGUCUAGGGGAUAACGCGUUAAACUUCCUAGAAGGCGGUU (SEQ ID NO: 391), CGCGGAUCUAGAUUGUAACGCGUUAAACCAUCUAGAAGGCGGUU (SEQ ID NO: 392), CGCGUCGCUACCGCCCGGCGCGUUAAACACACUAGAAGGCGGUU (SEQ ID NO: 393), and CGCGGUUCUAUCUAGUUACGCGUUAAACCAACUAGAA (SEQ ID NO: 394), or a nucleotide sequence having at least 80%, 85%, 90%, 95%, 96%, 97%, 98%, or 99% sequence identity therewith. In some embodiments, the secondary structure comprises a nucleotide sequence of and CGCGGUUCUAUCUAGUUACGCGUUAAACCAACUAGAA (SEQ ID NO: 394), or a nucleotide sequence having at least 80%, 85%, 90%, 95%, 96%, 97%, 98%, or 99% sequence identity therewith.

In some embodiments, the secondary structure is linked to one or more other component of a PEgRNA via a linker. For example, in some embodiments, the secondary structure is at the 3′ end of the PEgRNA and is linked to the 3′ end of a PBS via a linker. In some embodiments, the secondary structure is at the 5′ end of the PEgRNA and is linked to the 5′ end of a spacer via a linker. In some embodiments, the linker is a nucleotide linker that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the linker is 5 to 10 nucleotides in length. In some embodiments, the linker is 10 to 20 nucleotides in length. In some embodiments, the linker is 15 to 25 nucleotides in length. In some embodiments, the linker is 8 nucleotides in length.

In some embodiments, the linker is designed to minimize base pairing between the linker and another component of the PEgRNA. In some embodiments, the linker is designed to minimize base pairing between the linker and the spacer. In some embodiments, the linker is designed to minimize base pairing between the linker and the PBS. In some embodiments, the linker is designed to minimize base pairing between the linker and the editing template. In some embodiments, the linker is designed to minimize base pairing between the linker and the sequence of the RNA secondary structure. In some embodiments, the linker is optimized to minimize base pairing between the linker and another component of the PEgRNA, in order of the following priority: spacer, PBS, editing template and then scaffold. In some embodiments, base paring probability is calculated using ViennaRNA 2.0 under standard parameters (37° C., 1 M NaCl, 0.05 M MgCl2).

In some embodiments, the PEgRNA comprises a RNA secondary structure and/or a linker disclosed in Nelson et al. Engineered pegRNAs improve prime editing efficiency. Nat Biotechnol. (2021), the entirety of which is incorporated herein by reference.

In some embodiments, a PEgRNA is transcribed from a nucleotide encoding the PEgRNA, for example, a DNA plasmid encoding the PEgRNA. In some embodiments, the PEgRNA comprises a self-cleaving element. In some embodiments, the self-cleaving element improves transcription and/or processing of the PEgRNA when transcribed form the nucleotide encoding the PEgRNA. In some embodiments, the PEgRNA comprises a hairpin or a RNA quadruplex. In some embodiments, the PEgRNA comprises a self-cleaving ribozyme element, for example, a hammerhead, a pistol, a hatchet, a hairpin, a VS, a twister, or a twister sister ribozyme. In some embodiments, the PEgRNA comprises a HDV ribozyme. In some embodiments, the PEgRNA comprises a hairpin recognized by Csy4. In some embodiments, the PEgRNA comprises an ENE motif. In some embodiments, the PEgRNA comprises an element for nuclear expression (ENE) from MALAT1 Inc RNA. In some embodiments, the PEgRNA comprises an ENE element from Kaposi's sarcoma-associated herpesvirus (KSHV). In some embodiments, the PEgRNA comprises a 3′ box of a U1 snRNA. In some embodiments, the PEgRNA forms a circular RNA.

In some embodiments, the PEgRNA comprises a RNA secondary structure or a motif that improves binding to the DNA-RNA duple or enhances PEgRNA activity. In some embodiments, the PEgRNA comprises a sequence derived from a native nucleotide element involved in reverse transcription, e.g., initiation of retroviral transcription. In some embodiments, the PEgRNA comprises a sequence of, or derived from, a primer binding site of a substrate of a reverse transcriptase, a polypurine tract (PPT), or a kissing loop. In some embodiments, the PEgRNA comprises a dimerization motif, a kissing loop, or a GNRA tetraloop-tetraloop receptor pair that results in circularization of the PEgRNA. In some embodiments, the PEgRNA comprises a RNA secondary structure of a motif that results in physical separation of the spacer and the PBS of the PEgRNA, thereby prevents occlusion of the spacer and improves PEgRNA activity. In some embodiments, the PEgRNA comprises a secondary structure or motif, e.g., a 5′ or 3′ extension in the spacer region that form a toehold or hairpin, wherein the secondary structure or motif competes favorably against annealing between the spacer and the PBS of the PEgRNA, thereby prevents occlusion of the spacer and improves PEgRNA activity.

In some embodiments, a PEgRNA comprises the sequence GGCCGGCAUGGUCCCAGCCUCCUCGCUGGCGCCGGCUGGGCAACAUGCUUCGG CAUGGCGAAUGGGAC (SEQ ID NO: 395) at the 3′ end. In some embodiments, a PEgRNA comprises the structure [spacer]—[gRNA core]—[editing template]—[PBS]—GGCCGGCAUGGUCCCAGCCUCCUCGCUGGCGCCGGCUGGGCAACAUGCUUCGG CAUGGCGAAUGGGAC (SEQ ID NO: 395), or [spacer]—[gRNA core]—[editing template]—[PBS]—GGCCGGCAUGGUCCCAGCCUCCUCGCUGGCGCCGGCUGGGCAACAUGCUUCGG CAUGGCGAAUGGGAC-(U)n, wherein n is an integer between 3 and 7 (SEQ ID NO: 6120). The structure derived from hepatitis D virus (HDV) is italicized.

In some embodiments, the PEgRNA comprises the sequence GGUGGGAGACGUCCCACC (SEQ ID NO: 396) at the 5′ end and/or the sequence UGGGAGACGUCCCACC (SEQ ID NO: 397) at the 3′ end. In some embodiments, the PEgRNA comprises the following structure (M-MLV kissing loop): GGUGGGAGACGUCCCACC (SEQ ID NO: 396)—[spacer]—[gRNA core]—[editing template]—[PBS]—UGGGAGACGUCCCACC (SEQ ID NO: 397), or GGUGGGAGACGUCCCACC (SEQ ID NO: 396)—[spacer]—[gRNA core]—[editing template]—[PBS]—UGGGAGACGUCCCACC-(U)n, wherein n is an integer between 3 and 7 (SEQ ID NO: 6069). The kissing loop structure is italicized.

In some embodiments, the PEgRNA comprises the sequence GAGCAGCAUGGCGUCGCUGCUCAC (SEQ ID NO: 398) at the 5′ end and/or the sequence CCAUCAGUUGACACCCUGAGG (SEQ ID NO: 399) at the 3′ end. In some embodiments, the PEgRNA comprises the following structure (VS ribozyme kissing loop): GAGCAGCAUGGCGUCGCUGCUCAC (SEQ ID NO: 398)—[spacer]—[gRNA core]—[editing template]—[PBS]—CCAUCAGUUGACACCCUGAGG (SEQ ID NO: 399), or GAGCAGCAUGGCGUCGCUGCUCAC (SEQ ID NO: 398)—[spacer]—[gRNA core]—[editing template]—[PBS]—CCAUCAGUUGACACCCUGAGG-(U)n, wherein n is an integer between 3 and 7 (SEQ ID NO: 6121).

In some embodiments, the PEgRNA comprises the sequence GCAGACCUAAGUGGUGACAUAUGGUCUG (SEQ ID NO: 400) at the 5′ end and/or the sequence CAUGCGAUUAGAAAUAAUCGCAUG (SEQ ID NO: 401) at the 3′ end. In some embodiments, the PEgRNA comprises the following structure (tetraloop and receptor): GCAGACCUAAGUGGUGACAUAUGGUCUG (SEQ ID NO: 400)—[spacer]—[gRNA core]—[editing template]—[PBS]—CAUGCGAUUAGAAAUAAUCGCAUG (SEQ ID NO: 401), or GCAGACCUAAGUGGUGACAUAUGGUCUG (SEQ ID NO: 400)—[spacer]—[gRNA core]—[editing template]—[PBS]—CAUGCGAUUAGAAAUAAUCGCAUG-(U)n (SEQ ID NO: 6122), wherein n is an integer between 3 and 7. The tetraloop/tetraloop recepter structure is italicized.

In some embodiments, the PEgRNA comprises the sequence GGCCGGCAUGGUCCCAGCCUCCUCGCUGGCGCCGGCUGGGCAACAUGCUUCGG CAUGGCGAAUGGGAC (SEQ ID NO: 395) or UCUGCCAUCAAAGCUGCGACCGUGCUCAGUCUGGUGGGAGACGUCCCACCGGC CGGCAUGGUCCCAGCCUCCUCGCUGGCGCCGGCUGGGCAACAUGCUUCGGCAU GGCGAAUGGGAC (SEQ ID NO: 6074) at the 3′ end.

In some embodiments, a PEgRNA comprises a gRNA core that comprises a modified direct repeat compared to the sequence of a naturally occurring CRISPR-Cas guide RNA scaffold, for example, a Cas9 gRNA scaffold. In some embodiments, the PEgRNA comprises a “flip and extension (F+E)” gRNA core, wherein one or more base pairs in a direct repeat is modified. In some embodiments, the PEgRNA comprises a first direct repeat (the first paring element or the lower stem), wherein a Uracil is changed to a Adenine (such that in the stem region, a U-A base pair is changed to a A-U base pair). In some embodiments, the PEgRNA comprises a first direct repeat wherein the fourth U-A base pair in the stem is changed to a A-U base pair. In some embodiments, the PEgRNA comprises a first direct repeat wherein one or more U-A base pair is changed to a G-C or C-G base pair. For example, in some embodiments, the PEgRNA comprises a first direct repeat comprising a modification to a GUUUU-AAAAC pairing element, wherein one or more of the U-A base pairs is changed to a A-U base pair, a G-C base pair, or a C-G base pair. In some embodiments, the PEgRNA comprises an extended first direct repeat.

In some embodiments, a PEgRNA comprises a gRNA core comprises the sequence

(SEQ ID NO: 403)
GUUUUAGAGCUAUACGUAGCAAGUUAAAAUAAGGCUAGUCCGUUAUCAA
CUUUACGAAGUGGCACCGAGUCGGUGC
or
(SEQ ID NO: 6075)
GUUUUAGAGCUAUACGUAGCAAGUUAAAAUAAGGCUAGUCCGUUAUCAA
CUUUACGAAGUGGGACCGAGUCGGUCC.

In some embodiments, a PEgRNA comprises a gRNA core comprising the sequence

(SEQ ID NO: 404)
GUUUUAGAGCUAGCUCAUGAAAAUGAGCUAGCAAGUUAAAAUAAGGCUA
GUCCGUUAUCAACUUGAAAAAGUGGGACCGAGUCGGUCC.

In some embodiments, a PEgRNA comprises a gRNA core comprising the sequence

(SEQ ID NO: 6036)
GUUUGAGAGCUAGAAAUAGCAAGUUUAAAUAAGGCUAGUCCGUUAUCAA
CUUGAAAAAGUGGGACCGAGUCGGUCC.

In some embodiments, a PEgRNA comprises a gRNA core comprising the sequence

(SEQ ID NO: 405)
GUUUAAGAGCUAGAAAUAGCAAGUUUAAAUAAGGCUAGUCCGUUAUCAA
CUUGAAAAAGUGGCACCGAGUCGGUGC.

In some embodiments, a PEgRNA comprises a gRNA core comprising the sequence

(SEQ ID NO: 406)
GUUUUAGAGCUAUGCUGGAAACAGCAUAGCAAGUUAAAAUAAGGCUAGU
CCGUUAUCAACUUGAAAAAGUGGCACCGAGUCGGUGC.

In some embodiments, a PEgRNA comprises a gRNA core comprising the sequence

(SEQ ID NO: 56)
GUUUAAGAGCUAUGCUGGAAACAGCAUAGCAAGUUUAAAUAAGGCUAGU
CCGUUAUCAACUUGAAAAAGUGGCACCGAGUCGGUGC.

In some embodiments, a PEgRNA or a nick guide RNA (ngRNA) may be chemically synthesized, or may be assembled or cloned and transcribed from a DNA sequence, e.g., a plasmid DNA sequence, or by any RNA oligonucleotide synthesis method known in the art. In some embodiments, DNA sequence that encodes a PEgRNA (or ngRNA) may be designed to append one or more nucleotides at the 5′ end or the 3′ end of the PEgRNA (or nick guide RNA) encoding sequence to enhance PEgRNA transcription. For example, in some embodiments, a DNA sequence that encodes a PEgRNA (or nick guide RNA) (or an ngRNA) may be designed to append a nucleotide G at the 5′ end. Accordingly, in some embodiments, the PEgRNA (or nick guide RNA) may comprise an appended nucleotide G at the 5′ end. In some embodiments, a DNA sequence that encodes a PEgRNA (or nick guide RNA) may be designed to append a sequence that enhances transcription, e.g., a Kozak sequence, at the 5′ end. In some embodiments, a DNA sequence that encodes a PEgRNA (or nick guide RNA) may be designed to append the sequence CACC or CCACC at the 5′ end. Accordingly, in some embodiments, the PEgRNA (or nick guide RNA) may comprise an appended sequence CACC or CCACC at the 5′ end. In some embodiments, a DNA sequence that encodes a PEgRNA (or nick guide RNA) may be designed to append the sequence TTT, TTTT, TTTTT, TTTTTT, TTTTTTT at the 3′ end. Accordingly, in some embodiments, the PEgRNA (or nick guide RNA) may comprise an appended sequence UUU, UUUU, UUUUU, UUUUUU, or UUUUUUU at the 3′ end.

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, a PEgRNA or ngRNA may include a modifying sequence at the 3′end having the sequence AACAUUGACGCGUCUCUACGUGGGGGCGCG (SEQ ID NO: 57).

In some embodiments, the ngRNA is a guide RNA which contains a variable spacer sequence and a guide RNA scaffold or core region that interacts with the DNA binding domain, e.g., Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene, e.g., the SLC37A4 gene. A prime editing system, composition, or complex comprising a ngRNA may be referred to as a “PE3” prime editing systemPE3 prime editing composition, or PE3 prime editing complex.

In some embodiments, the ng search target sequence is located on the non-target strand, within 10 base pairs to 100 base pairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system maybe referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 4, the gRNA core of a PEgRNA may comprise one or more regions of a base paired lower stem, a base paired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a 2′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA may be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

Unless otherwise indicated, references to nucleotide positions in human chromosomes are as set forth in human genome assembly consortium Human build 38 (GRCh38), GenBank accession GCF_000001405.38.

Provided herein in some embodiments are example sequences for PEgRNAs, including PEgRNA spacers, PBS, RTT, and ngRNA spacers for a prime editing system comprising a nuclease that recognizes the PAM sequence “NG.” In some embodiments, a PAM motif on the edit strand comprises an “NG” motif, wherein N is any nucleotide.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, e.g., a SLC37A4 gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene, e.g., a SLC37A4 gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g., the target SLC37A4 gene, upon the contacting of the PE composition with the target gene. In some embodiments, the DNA binding domain of the PE associates with the PEgRNA. In some embodiments, the PE binds the target gene, e.g., a SLC37A4 gene, directed by the PEgRNA. Accordingly, in some embodiments, the contacting of the target gene result in binding of a DNA binding domain of a prime editor of the target SLC37A4 gene directed by the PEgRNA.

In some embodiments, contacting the target gene with the prime editing composition results in a nick in an edit strand of the target gene, e.g., a SLC37A4 gene by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene, e.g., a SLC37A4 gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5′ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, e.g., a SLC37A4 gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor. In some embodiments, the target gene, e.g., a SLC37A4 gene, is in a cell.

Accordingly, also provided herein are methods of modifying a cell, such as a human cell, a human primary cell, a human iPSC-derived cell, a liver cell, a hepatocyte, a cholangiocyte, a kidney cell, a proximal tubule cell, or a Müller cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell.

In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a retinal progenitor cell. In some embodiments, the cell is a retina precursor cell. In some embodiments, the cell is a fibroblast.

In some embodiments, the cell is a human progenitor cell. In some embodiments, the cell is a human stem cell. in some embodiments, the cell is an induced human pluripotent stem cell. In some embodiments, the cell is a human embryonic stem cell. In some embodiments, the cell is a human retinal progenitor cell. In some embodiments, the cell is a human retina precursor cell. In some embodiments, the cell is a human fibroblast.

In some embodiments, the cell is a primary cell. In some embodiments, the cell is a human primary cell. In some embodiments, the cell is a liver cell. In some embodiments, the cell is a hepatocyte. In some embodiments, the cell is a cholangiocyte. In some embodiments, the cell is a kidney cell. In some embodiments, the cell is a proximal tubule cell. In some embodiments, the cell is a Müller cell. In some embodiments, the cell is a primary human hepatocyte derived from an induced human pluripotent stem cell (iPSC). In some embodiments, the cell is a primary human cholangiocyte derived from an induced human pluripotent stem cell (iPSC). In some embodiments, the cell is a primary human renal proximal tubule cell derived from an induced human pluripotent stem cell (iPSC). In some embodiments, the cell is a primary human Müller cell derived from an induced human pluripotent stem cell (iPSC).

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a SLC37A4 gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37 (3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a SLC37A4 gene within the genome of a cell) to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 1% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 1% and an indel frequency of less than 0.1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 5% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 5% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 5% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 7.5% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 7.5% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 7.5% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 10% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 10% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 10% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 15% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 15% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 15% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 20% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 20% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 20% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 30% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 30% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 30% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 40% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 40% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 40% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 50% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 50% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 50% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 60% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 60% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 60% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 70% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 70% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 70% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 80% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 80% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 80% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 90% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 90% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 90% and an indel frequency of less than 0.1% in a target cell, e.g., a human primary cell, human iPSC, human fibroblast, human hepatocyte, human cholangiocyte, human proximal tubule cell, or human Müller cell.

In some embodiments, the prime editing methods disclosed herein have an editing efficiency of at least about 95% and an indel frequency of less than 1% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 95% and an indel frequency of less than 0.5% in a target cell, e.g., a human primary cell, human iPSC, human fibroblast, human hepatocyte, human cholangiocyte, human proximal tubule cell, or human Müller cell. In some embodiments, the prime editing methods disclosed herein have an editing efficiency of at least about 95% and an indel frequency of less than 0.1% in a target cell, e.g., a human primary cell, human iPSC, human fibroblast, human hepatocyte, human cholangiocyte, human proximal tubule cell, or human Müller cell. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a SLC37A4 gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a SLC37A4 gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing methods disclosed herein can be used to edit a target SLC37A4 gene. In some embodiments, the target SLC37A4 gene comprises a mutation compared to a wild type SLC37A4 gene. In some embodiments, the mutation is associated with Glycogen storage disease type 1B. In some embodiments, the target SLC37A4 gene comprises an editing target sequence that contains the mutation associated with Glycogen storage disease type 1B. In some embodiments, the mutation is in a coding region of the target SLC37A4 gene. In some embodiments, the mutation is in an exon of the target SLC37A4 gene. In some embodiments, the prime editing method comprises contacting a target SLC37A4 gene with a prime editing composition comprising a prime editor, a PEgRNA, and/or a ngRNA. In some embodiments, contacting the target SLC37A4 gene with the prime editing composition results in incorporation of one or more intended nucleotide edits in the target SLC37A4 gene. In some embodiments, the incorporation is in a region of the target SLC37A4 gene that corresponds to an editing target sequence in the SLC37A4 gene. In some embodiments, the one or more intended nucleotide edits comprises a single nucleotide substitution, an insertion, a deletion, or any combination thereof, compared to the endogenous sequence of the target SLC37A4 gene. In some embodiments, incorporation of the one or more intended nucleotide edits results in replacement of one or more mutations with the corresponding sequence that encodes a wild type G6PT1 set forth in SEQ ID NO: 1. In some embodiments, incorporation of the one or more intended nucleotide edits results in replacement of the one or more mutations with the corresponding sequence in a wild type SLC37A4 gene. In some embodiments, incorporation of the one more intended nucleotide edits results in correction of a mutation in the target SLC37A4 gene. In some embodiments, the target SLC37A4 gene comprises an editing target sequence that contains the mutation. In some embodiments, contacting the target SLC37A4 gene with the prime editing composition results in incorporation of one or more intended nucleotide edits in the target SLC37A4 gene, which corrects the mutation in the editing target sequence (or a double stranded region comprising the editing target sequence and the complementary sequence to the editing target sequence on a target strand) in the target SLC37A4 gene. In some embodiments, the mutation is in exon 8 of the target SLC37A4 gene. In some embodiments, the mutation results in a c. 1015G->T nucleotide substitution in the sequence encoding a G6PT1 protein and a G339C amino acid substitution in the G6PT1 protein. In some embodiments, the correction results in restoration of wild type expression, i.e., G at position 1015 in the sequence encoding the G6PT1 protein, and thereby a restoration of wild type G6PT1 with glycine at position 339. In some embodiments, the mutation results in a c. 1042-1043delCT in the sequence encoding a G6PT1 protein. In some embodiments, the correction results in restoration of wild type expression, i.e., CT insertion at position 1042 in the sequence encoding the G6PT1 protein.

In some embodiments, the target SLC37A4 gene is in a target cell. Accordingly, in one aspect provided herein is a method of editing a target cell comprising a target SLC37A4 gene that encodes a polypeptide that comprises one or more mutations relative to a wild type SLC37A4 gene. In some embodiments, the methods of the present disclosure comprise introducing a prime editing composition comprising a PEgRNA, a prime editor polypeptide, and/or a ngRNA into the target cell that has the target SLC37A4 gene to edit the target SLC37A4 gene, thereby generating an edited cell. In some embodiments, the target cell is a mammalian cell. In some embodiments, the target cell is a human cell. In some embodiments, the target cell is a progenitor cell. In some embodiments, the target cell is a stem cell. in some embodiments, the target cell is an induced pluripotent stem cell. In some embodiments, the target cell is an embryonic stem cell. In some embodiments, the target cell is a retinal progenitor cell. In some embodiments, the target cell is a retina precursor cell. In some embodiments, the target cell is a fibroblast. In some embodiments, the target cell is a human progenitor cell. In some embodiments, the target cell is a human stem cell. in some embodiments, the target cell is an induced human pluripotent stem cell. In some embodiments, the target cell is a human embryonic stem cell. In some embodiments, the target cell is a human retinal progenitor cell. In some embodiments, the target cell is a human retina precursor cell. In some embodiments, the target cell is a human fibroblast. In some embodiments, the target cell is a primary cell. In some embodiments, the target cell is a human primary cell. In some embodiments, the target cell is a liver cell. In some embodiments, the target cell is a hepatocyte. In some embodiments, the target cell is a cholangiocyte. In some embodiments, the target cell is a renal proximal tubule cell. In some embodiments, the target cell is a human cell from a liver. In some embodiments, the target cell is a human hepatocyte. In some embodiments, the target cell is a human Müller cell. In some embodiments, the target cell is a human cholangiocyte. In some embodiments, the target cell is a human renal proximal tubule cell. In some embodiments, the cell is a human cell from an inner ear. In some embodiments, the cell is a primary human hepatocyte derived from an induced human pluripotent stem cell (iPSC). In some embodiments, the cell is a primary human Müller cell derived from an induced human pluripotent stem cell (iPSC). In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target SLC37A4 gene that comprises one or more mutations restores wild type expression and function of G6PT1 encoded by the SLC37A4 gene. In some embodiments, the target SLC37A4 gene encodes a G339C amino acid substitution as compared to the wild type G6PT1 SLC37A4 protein prior to incorporation of the one or more intended nucleotide edits. In some embodiments, the target SLC37A4 gene encodes a L348fs amino acid frameshift mutation as compared to the wild type G6PT1 SLC37A4 protein prior to incorporation of the one or more intended nucleotide edits. In some embodiments, expression and/or function of G6PT1 may be measured when expressed in a target cell. In some embodiments, incorporation of the one or more intended nucleotide edits in the target SLC37A4 gene comprising one or more mutations lead to a fold change in a level of SLC37A4 gene expression, G6PT1 expression, or a combination thereof. In some embodiments, a change in the level of SLC37A4 expression can comprise a fold change of, e.g., 2-fold, 3-fold, 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target SLC37A4 gene that comprises one or more mutations restores wild type expression of G6PT1 by at least 10%, 20%, 30%, 40%, 50%, 60%, 70%, 80%, 90%, 95%, 099% or more as compared to wild type expression of the SLC37A4 protein in a suitable control cell that comprises a wild type SLC37A4 gene.

In some embodiments, a G6PT1 expression increase can be measured by a G6PT1 functional assay. In some embodiments, protein expression can be measured using a protein assay. In some embodiments, protein expression can be measured using antibody testing. In some embodiments, an antibody can comprise anti-G6PT1. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof.

### Methods of Treating Glycogen Storage Disease Type 1B

In some embodiments, provided herein are methods for treatment of a subject diagnosed with a disease associated with or caused by one or more pathogenic mutations that can be corrected by prime editing. In some embodiments, provided herein are methods for treating Glycogen storage disease type 1B that comprise administering to a subject a therapeutically effective amount of a prime editing composition, or a pharmaceutical composition comprising a prime editing composition as described herein. In some embodiments, administration of the prime editing composition results in incorporation of one or more intended nucleotide edits in the target gene in the subject. In some embodiments, administration of the prime editing composition results in correction of one or more pathogenic mutations, e.g., point mutations, insertions, or deletions, associated with Glycogen storage disease type 1B in the subject. In some embodiments, the target gene comprise an editing target sequence that contains the pathogenic mutation. In some embodiments, administration of the prime editing composition results in incorporation of one or more intended nucleotide edits in the target gene that corrects the pathogenic mutation in the editing target sequence (or a double stranded region comprising the editing target sequence and the complementary sequence to the editing target sequence on a target strand) of the target gene in the subject.

In some embodiments, the method provided herein comprises administering to a subject an effective amount of a prime editing composition, for example, a PEgRNA, a prime editor, and/or a ngRNA. In some embodiments, the method comprises administering to the subject an effective amount of a prime editing composition described herein, for example, polynucleotides, vectors, or constructs that encode prime editing composition components, or RNPs, LNPs, and/or polypeptides comprising prime editing composition components. Prime editing compositions can be administered to target the SLC37A4 gene in a subject, e.g., a human subject, suffering from, having, susceptible to, or at risk for Glycogen storage disease type 1B. Identifying a subject in need of such treatment can be in the judgment of a subject or a health care professional and can be subjective (e.g., opinion) or objective (e.g., measurable by a test or diagnostic method). In some embodiments, the subject has Glycogen storage disease type 1B.

In some embodiments, the subject has been diagnosed with Glycogen storage disease type 1B by sequencing of a SLC37A4 gene in the subject. In some embodiments, the subject comprises at least a copy of SLC37A4 gene that comprises one or more mutations compared to a wild type SLC37A4 gene. In some embodiments, the subject comprises at least a copy of SLC37A4 gene that comprises a mutation in a coding region of the SLC37A4 gene. In some embodiments, the subject comprises at least a copy of SLC37A4 gene that comprises a mutation in exon 8, as compared to a wild type SLC37A4 gene. In some embodiments, the subject comprises at least a copy of SLC37A4 gene that comprises mutation G339C of the SLC37A4 gene as compared to a wild type SLC37A4 gene. In some embodiments, the subject comprises at least a copy of SLC37A4 gene that comprises mutation L348fs of the SLC37A4 gene as compared to a wild type SLC37A4 gene.

In some embodiments, the method comprises directly administering prime editing compositions provided herein to a subject. The prime editing compositions described herein can be delivered with in any form as described herein, e.g., as LNPs, RNPs, polynucleotide vectors such as viral vectors, or mRNAs. The prime editing compositions can be formulated with any pharmaceutically acceptable carrier described herein or known in the art for administering directly to a subject. Components of a prime editing composition or a pharmaceutical composition thereof may be administered to the subject simultaneously or sequentially. For example, in some embodiments, the method comprises administering a prime editing composition, or pharmaceutical composition thereof, comprising a complex that comprises a prime editor fusion protein and a PEgRNA and/or a ngRNA, to a subject. In some embodiments, the method comprises administering a polynucleotide or vector encoding a prime editor to a subject simultaneously with a PEgRNA and/or a ngRNA. In some embodiments, the method comprises administering a polynucleotide or vector encoding a prime editor to a subject before administration with a PEgRNA and/or a ngRNA.

Suitable routes of administrating the prime editing compositions to a subject include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, the compositions described are administered intraperitoneally, intravenously, or by direct injection or direct infusion. In some embodiments, the compositions described herein are administered by direct injection. In some embodiments, the compositions described herein are administered by subretinal injection. In some embodiments, the compositions described herein are administered by injection to the fovea or parafoveal regions. In some embodiments, the compositions described herein are administered by injection to peripheral regions of the retina. In some embodiments, the compositions described herein are administered by injection through the round window. In some embodiments, the compositions described herein are administered to the retina. In some embodiments, the compositions described herein are administered to a subject by injection, by means of a catheter, by means of a suppository, or by means of an implant.

In some embodiments, the method comprises administering cells edited with a prime editing composition described herein to a subject. In some embodiments, the cells are allogeneic. In some embodiments, allogeneic cells are or have been contacted ex vivo with a prime editing composition or pharmaceutical composition thereof and are introduced into a human subject in need thereof. In some embodiments, the cells are autologous to the subject. In some embodiments, cells are removed from a subject and contacted ex vivo with a prime editing composition or pharmaceutical composition thereof and are re-introduced into the subject.

In some embodiments, cells are contacted ex vivo with one or more components of a prime editing composition. In some embodiments, the ex vivo-contacted cells are introduced into the subject, and the subject is administered in vivo with one or more components of a prime editing composition. For example, in some embodiments, cells are contacted ex vivo with a prime editor and introduced into a subject. In some embodiments, the subject is then administered with a PEgRNA and/or a ngRNA, or a polynucleotide encoding the PEgRNA and/or the ngRNA.

In some embodiments, cells contacted with the prime editing composition are determined for incorporation of the one or more intended nucleotide edits in the genome before re-introduction into the subject. In some embodiments, the cells are enriched for incorporation of the one or more intended nucleotide edits in the genome before re-introduction into the subject. In some embodiments, the edited cells are primary cells. In some embodiments, the edited cells are progenitor cells. In some embodiments, the edited cells are stem cells. In some embodiments, the edited cells are hepatocytes. In some embodiments, the edited cells are primary human cells. In some embodiments, the edited cells are human progenitor cells. In some embodiments, the edited cells are human stem cells. In some embodiments, the edited cells are human hepatocytes. In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject. The prime editing composition or components thereof may be introduced into a cell by any delivery approaches as described herein, including LNP administration, RNP administration, electroporation, nucleofection, transfection, viral transduction, microinjection, cell membrane disruption and diffusion, or any other approach known in the art.

The cells edited with prime editing can be introduced into the subject by any route known in the art. In some embodiments, the edited cells are administered to a subject by direct infusion. In some embodiments, the edited cells are administered to a subject by intravenous infusion. In some embodiments, the edited cells are administered to a subject as implants.

The pharmaceutical compositions, prime editing compositions, and cells, as described herein, can be administered in effective amounts. In some embodiments, the effective amount depends upon the mode of administration. In some embodiments, the effective amount depends upon the stage of the condition, the age and physical condition of the subject, the nature of concurrent therapy, if any, and like factors well-known to the medical practitioner.

The specific dose administered can be a uniform dose for each subject. Alternatively, a subject's dose can be tailored to the approximate body weight of the subject. Other factors in determining the appropriate dosage can include the disease or condition to be treated or prevented, the severity of the disease, the route of administration, and the age, sex and medical condition of the patient.

In embodiments wherein components of a prime editing composition are administered sequentially, the time between sequential administration can be at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days.

In some embodiments, a method of monitoring treatment progress is provided. In some embodiments, the method includes the step of determining a level of diagnostic marker, for example, correction of a mutation in SLC37A4 gene, or diagnostic measurement associated with Glycogen storage disease type 1B, in a subject suffering from Glycogen storage disease type 1B symptoms and has been administered an effective amount of a prime editing composition described herein. The level of the diagnostic marker determined in the method can be compared to known levels of the marker in either healthy normal controls or in other afflicted subjects to establish the subject's disease status.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target SLC37A4 gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid: nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid: nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and ψ 2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine, and octa-arginine. The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 5 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 3 below.

TABLE 5
Exemplary lipids for nanoparticle formulation or gene transfer

| | Abbre- | |
|---|---|---|
| Lipid | viation | Feature |
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N41-(2,3-Dioleyloxy)prophyliN,N,N- | DOTMA | Cationic |
| trimethylammonium chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3- | GAP- | Cationic |
| bis(dodecyloxy)-1-propanaminium bromide | DLRIE | |
| Cetyltrimethylammonium bromide | CTAB | Cationic |
| 6-Lauroxyhexyl omithinate | LHON | Cationic |
| 1-(2,3-Dioleoyloxypropy1)-2,4,6- | 2Oc | Cationic |
| trimethylpyridinium | | |
| 2,3-Dioleyloxy-N-P(spenninecarboxamido- | DOSPA | Cationic |
| ethy1J-N,Ndimethyl- | | |
| l-propanatninium trifluoroacetate | | |
| 1,2-Dioley1-3-trimethylamtnonium-propane | DOPA | Cationic |
| N-(2-Hydroxyethyl)-N,N-dimethy1-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermy1)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl- | CCS | Cationic |
| spenmine | | |
| N-t-Butyl-N0-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane-1,9- | CDAN | Cationic |
| diamine | | |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]- | DLin-KC2- | Cationic |
| dioxolane | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 6 below.

TABLE 6
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 7 below.

TABLE 7
Exemplary polynucleotide delivery methods

| | | Delivery | Duration | | |
|---|---|---|---|---|---|
| | | into Non- | of | Genome | Type of |
| Deliv- | | Dividing | Expres- | Inte- | Molecule |
| ery | Vector/Mode | Cells | sion | gration | Delivered |
| Physical | (e.g., | YES | Transient | NO | Nucleic |
| | electro- | | | | Acids and |
| | poration, | | | | Proteins |
| | particle gun, | | | | |
| | Calcium | | | | |
| | phosphate | | | | |
| | transfection) | | | | |
| Viral | Retrovirus | NO | Stable | YES | RNA |
| | Lentivirus | YES | Stable | YES/NO | RNA |
| | | | | with | |
| | | | | modifi- | |
| | | | | cation | |
| | Adenovirus | YES | Transient | NO | DNA |
| | Adeno- | YES | Stable | NO | DNA |
| | Associated | | | | |
| | Virus | | | | |
| | (AAV) | | | | |
| | Vaccinia | YES | Very | NO | DNA |
| | Virus | | Transient | | |
| | Herpes | YES | Stable | NO | DNA |
| | Simplex | | | | |
| | Virus | | | | |
| Non- | Cationic | YES | Transient | Depends | Nucleic |
| Viral | | | | on what is | acids and |
| | | | | delivered | Proteins |
| | Polymeric | YES | Transient | NO | Nucleic |
| | Nanoparticles | | | | Acids |
| Bio- | Attenuated | YES | Transient | NO | Nucleic |
| logical | Bacteria | | | | Acids |
| Non- | Engineered | YES | Transient | NO | Nucleic |
| Viral | Bacteriophages | | | | Acids |
| Deliv- | Mammalian | YES | Transient | NO | Nucleic |
| ery | Virus-like | | | | Acids |
| Vehi- | Particles | | | | |
| cles | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prime editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prime editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

### Example 1—Screening of PEgRNA for Editing of a Mutation Associated with Glycogen Storage Disease Type 1B

The following examples are provided for illustrative purposes only and are not intended to limit the scope of the claims provided herein.

PEgRNA assembly: PEgRNA libraries were assembled by one of three methods: in the first method, pooled synthesized DNA oligos encoding the PEgRNA and flanking U6 expression plasmid homology regions were cloned into U6 expression plasmids via Gibson cloning and sequencing of bacterial colonies via Sanger or Next-generation sequencing. In the second method, double-stranded linear DNA fragments encoding PEgRNA and homology sequences as above were individually Gibson-cloned into U6 expression plasmids. In the third method, for each PEgRNA, separate oligos encoding a protospacer, a gRNA scaffold, and PEgRNA extension (PBS and RTT) were ligated, and then cloned into a U6 expression plasmid as described in Anzalone et al., Nature. 2019 December; 576 (7785): 149-157. Bacterial colonies carrying sequence-verified plasmids were propagated in LB or TB. Plasmid DNA is purified by minipreps for mammalian transfection.

Chemically synthesized PEgRNAs were modified at the 5′ end and the 3′ end: the three 5′ most nucleotides were modified to phosphorothioated 2′-O-methyl nucleotides. The three consecutive nucleotides that precede the 3′ most nucleotide (i.e. three consecutive nucleotides immediately 5′ of the last nucleotide at the 3′ end) were also modified to phosphorothioated 2′-O-methyl nucleotides.

HEK cell culture and transfection: HEK293T cells were propagated in DMEM with 10% FBS. Prior to transfection, cells were seeded in 96-well plates and then transfected with Lipofectamine 2000 or MessengerMax according to the manufacturer's directions with DNA or mRNA encoding PE2 and PEgRNA (and ngRNA for PE3 experiments). Three days after transfection, gDNA was harvested in lysis buffer for high throughput sequencing and was sequenced using Miseq.

Lentiviral production and cell line generation: Generation of mutant cell line. Lentiviral transfer plasmids containing the SLC37A4 c.1015G->T mutation (G339C) with flanking sequences from the SLC37A4 gene on each side, and an IRES-Puromycin selection cassette, may be cloned behind an EF1α short promoter. HEK293T cells may be transiently transfected with the transfer plasmids and packaging plasmids containing VSV glycoprotein and lentiviral gag/pol coding sequences. After transfection, lentiviral particles may be harvested from the cell media and concentrated. HEK293T cells may be transduced using serial dilutions of the lentiviral particles described above. Cells generated at a dilution of MOI <1, as determined by survival following puromycin, are selected for expansion. A resulting HEK293T cell line carrying the c. 1015G->T mutation may be used to screen PEgRNAs.

### Installation of G339C Mutation by Prime Editing: Alternate Method for Generation of Mutant Cell Line.

PEgRNAs for NGG PAM recognition were designed to incorporate a SLC37A4 c. 1015G->T mutation in the wild type endogenous SLC37A4 gene in HEK293T cells by prime editing as a proxy to examine editing efficiency.

A wild type HEK293T cell line was expanded and transiently transfected with a plasmid encoding the PE2 fusion protein and a G339C mutation installation PEgRNA in arrayed 96-well plates for assessment of editing by high-throughput sequencing. Prior to transfection, cells were seeded in 96-well plates and then transfected with Lipofectamine 2000 or MessengerMax according to the manufacturer's directions with DNA or mRNA PE2 and PEgRNA. Three days after transfection, gDNA was harvested in lysis buffer for high throughput sequencing and sequenced using Miseq.

Glycogen storage disease type 1B mutation correction with PE2 system: A HEK293T cell line carrying the G339C mutation, such as one made by a method described above, was expanded and transiently transfected with a PE and PEgRNA in arrayed 96-well plates for assessment of editing by high-throughput sequencing. An exemplary PEgRNA spacer close to the G339C mutation is listed in Table 8. Spacer S01 has a sequence from the sense (or positive) strand.

Exemplary RTT sequences in PEgRNAs for spacers S01, S02, S03, S04, S05, S06, S07, S08, S09, and S10 are listed in Tables 9a, 10a, 11a, 12a, 13a, 14a, 15a, 16a, 17a, and 18a, respectively. Exemplary PBS sequences in PEgRNAs for spacers S01, S02, S03, S04, S05, S06, S07, S08, S09, and S10 are listed in Tables 9b, 10b, 11b, 12b, 13b, 14b, 15b, 16b, 17b, and 18b, respectively. Exemplary RTT/PBS combinations in PEgRNAs for spacers S01, S02, S03, S04, S05, S06, S07, S08, S09, and S10 are listed in Tables 9c, 10c, 11c, 12c, 13c, 14c, 15c, 16c, 17c, and 18c, respectively.

The PEgRNAs made according to these exemplary embodiments may contain, in order from 5′ to 3′, a spacer, a gRNA core (SEQ ID NO: 54) as discussed above, an RTT appropriate for the spacer, a PBS appropriate for the spacer, and a 3′ end modifier region (SEQ ID NO: 57) as discussed above. In some embodiments, a PEgRNA includes the sequence of spacer S01, an RTT sequence selected from the RTT sequences listed in Table 9a, and a PBS sequence selected from the PBS sequences listed in Table 9b (an “S01 PEgRNA”). In some embodiments, a PEgRNA includes the sequence of spacer S02, an RTT sequence selected from the RTT sequences listed in Table 10a, and a PBS sequence selected from the PBS sequences listed in Table 10b (an “S02 PEgRNA”). In some embodiments, a PEgRNA includes the sequence of spacer S03, an RTT sequence selected from the RTT sequences listed in Table 11a, and a PBS sequence selected from the PBS sequences listed in Table 11b (an “S03 PEgRNA”). In some embodiments, a PEgRNA includes the sequence of spacer S04, an RTT sequence selected from the RTT sequences listed in Table 12a, and a PBS sequence selected from the PBS sequences listed in Table 12b (an “S04 PEgRNA”). In some embodiments, a PEgRNA includes the sequence of spacer S05, an RTT sequence selected from the RTT sequences listed in Table 13a, and a PBS sequence selected from the PBS sequences listed in Table 13b (an “S05 PEgRNA”). In some embodiments, a PEgRNA includes the sequence of spacer S06, an RTT sequence selected from the RTT sequences listed in Table 14a, and a PBS sequence selected from the PBS sequences listed in Table 14b (an “S06 PEgRNA”). In some embodiments, a PEgRNA includes the sequence of spacer S07, an RTT sequence selected from the RTT sequences listed in Table 15a, and a PBS sequence selected from the PBS sequences listed in Table 15b (an “S07 PEgRNA”). In some embodiments, a PEgRNA includes the sequence of spacer S09, an RTT sequence selected from the RTT sequences listed in Table 16a, and a PBS sequence selected from the PBS sequences listed in Table 16b (an “S08 PEgRNA”). In some embodiments, a PEgRNA includes the sequence of spacer S09, an RTT sequence selected from the RTT sequences listed in Table 17a, and a PBS sequence selected from the PBS sequences listed in Table 17b (an “S09 PEgRNA”). In some embodiments, a PEgRNA includes the sequence of spacer S10, an RTT sequence selected from the RTT sequences listed in Table 18a, and a PBS sequence selected from the PBS sequences listed in Table 18b (an “S10 PEgRNA”).

The PEgRNA may also include a 5′ end modifier region as discussed above. The PEgRNAs made according to these exemplary embodiments may include chemically modified RNA nucleobases as discussed above. Specifically, in these PEgRNAs the first three 5′ residues may be phosphorothioated 2′O-methyl RNA bases, and the last three 3′ residues before the final residue (i.e., the three consecutive nucleotides immediately 5′ of the last nucleotide at the 3′ end) may be phosphorothioated 2′O-methyl RNA bases.

The results of several experiments measuring correction of the G339C mutation in HEK293T cells using various synthetic PEgRNAs alone are reported in Table 18d, Table 18e, Table 18f, and Table 18g. PEgRNAs are identified by “PEG-nnnn” numbers, for each of which RTT and PSB sequences are provided elsewhere. The data is reported as percentage of sampled cells in which sequencing identified the mutation as correctly repaired (“edit %”) or otherwise (i.e., incorrectly) modified (“indel %”).

The results of several experiments measuring correction of the L348fs mutation in HEK293T cells using various synthetic PEgRNAs alone are reported in Table 24d and Table 24e. PEgRNAs are identified by “PEG-nnnn” numbers, for each of which RTT and PSB sequences are provided elsewhere. The data is reported as percentage of sampled cells in which sequencing identified the mutation as correctly repaired (“edit %”) or otherwise (i.e., incorrectly) modified (“indel %”).

Glycogen storage disease type 1B G339C mutation correction with PE3 system: a second-nick guide RNA (“ngRNA”) that causes a nick on the opposite strand compared to the PEgRNA (i.e., on the non-edit strand) may be included in order improve efficiency and/or fidelity of prime editing as discussed above. Exemplary ngRNA negative-strand spacers are listed in Table 8a, and exemplary positive-strand spacers are listed in Table 8b. A ngRNA according to these exemplary embodiments will contain, in order from 5′ to 3′, a spacer, a gRNA core such as SEQ ID NO: 54 as discussed above, and optionally a 3′ end modifier region such as SEQ ID NO: 57 as discussed above. The ngRNA may also include a 5′ end modifier region as discussed above. The ngRNA may include chemically modified RNA nucleobases as discussed above. For example, in a ngRNA the first three 5′ residues may be phosphorothioated 2′O-methyl RNA bases, and the last three 3′ residues may be phosphorothioated 2′O-methyl RNA bases. A PEgRNA with a positive-strand spacer may be paired with negative-strand ngRNA. In some embodiments, a PE3 system may include an S01 PEgRNA and an ngRNA with a spacer having a sequence from Table 8A. In some embodiments, a PE3 system may include an S02 PEgRNA and an ngRNA with a spacer having a sequence from Table 8A. In some embodiments, a PE3 system may include an S03 PEgRNA and an ngRNA with a spacer having a sequence from Table 8A. In some embodiments, a PE3 system may include an S04 PEgRNA and an ngRNA with a spacer having a sequence from Table 8A. In some embodiments, a PE3 system may include an S05 PEgRNA and an ngRNA with a spacer having a sequence from Table 8A. In some embodiments, a PE3 system may include a PEgRNA comprising the spacer S06 and an ngRNA with a spacer having a sequence from Table 8A. In some embodiments, a PE3 system may include a PEgRNA comprising the spacer S07 and an ngRNA with a spacer having a sequence from Table 8B. In some embodiments, a PE3 system may include a PEgRNA comprising the spacer S08 and an ngRNA with a spacer having a sequence from Table 8B. In some embodiments, a PE3 system may include a PEgRNA comprising the spacer S09 and an ngRNA with a spacer having a sequence from Table 8B. In some embodiments, a PE3 system may include a PEgRNA comprising the spacer S10 and an ngRNA with a spacer having a sequence from Table 8B.

The results of several experiments measuring correction of the G339C mutation in HEK293T cells using various synthetic PEgRNAs in combination with various ngRNAs as laid out in the tables referenced above are reported in Table 18h, Table 18i, and Table 18j. PEgRNAs are identified by “PEG-nnnn” numbers, for each of which RTT and PSB sequences are provided elsewhere. ngRNAs are identified by sequence numbers from Table 8c and Table 8d. The data is reported as percentage of sampled cells in which sequencing identified the mutation as correctly repaired (“edit %”) or otherwise (i.e., incorrectly) modified (“indel %”).

Glycogen Storage Disease Type 1B Mutation Correction with PE3 System in iPSC:

A dose-response study was conducted using in human inducible pluripotent stem cells. iPSCs were mutated to carry the G339C mutation in the SLC37A4 gene and differentiated to a hepatocyte lineage. They were co-transfected mRNA encoding Prime Editor, RNA encoding PEgRNA and RNA encoding ngRNA targeting the SLC37A4 gene. Seventy-two hours following transfection gDNA were harvested. Next generation sequencing was used to calculate editing efficiency by quantifying the number of alleles with the desired sequence change. iPSC transfection: iPSCs were seeded in a 96-well plate the day prior to transfection. On transfection day, a mixture consisting of mRNA encoding Prime Editor, RNA encoding PEgRNA, and ngRNA were diluted in Optimem for a total volume of 6.5 μl. This mix was titrated together, keeping the ratio of mRNA, PEgRNA and ngRNA fixed. PEgRNA doses tested were 112, 74.7, 49.8, 33.2, 22.1, 14.7, 9.8, 6.6, 4.4, 2.9, 2.0 and 1.3 ng. ngRNA doses tested were 37.3, 24.9, 16.6, 11.1, 7.4, 4.9, 3.3, 2.2, 1.5, 1, 0.7 and 0.4 ng. Prime Editor mRNA doses tested were 337.4, 224.9, 150, 100, 66.6, 44.4, 29.6, 19.7, 13.1, 8.8, 5.8 and 3.9 ng. The mixture was then added to a dilution of transfection reagent consisting of Lipofectamine Stem reagent diluted in Optimem for a total volume of 6.5 ul. The two mixtures were mixed and incubated for 10 min at room temperature. 13 ul of transfection mixture were added to each well and swirled to ensure even distribution. Plates were returned to the incubator and cultured at 37 C with 5% CO₂.

Two trials of dose responses for various combinations of PEgRNA and ngRNA were tested in iPSC, with two replicates for each trial. The results are provided in Table 18k. The reported dose is the sum of mRNAm PEgRNA, and ngRNA.

As noted above, PEgRNAs comprising the spacers S07, S08, S09, or S10, when they also include an RTT sequence from Tables 15a, 16a, 17a, or 18a, respectively, can also correct the nearby c. 1042-1043delCT (L348fs) frameshift mutation. This is because the editing template is long enough to provide a corrective sequence template to cover both mutation sites.

Lentiviral production and cell line generation: Generation of mutant cell line. Lentiviral transfer plasmids containing the SLC37A4 c. 1042delCT mutation (L348fs) with flanking sequences from the SLC37A4 gene on each side, and an IRES-Puromycin selection cassette, may be cloned behind an EF1α short promoter. HEK293T cells may be transiently transfected with the transfer plasmids and packaging plasmids containing VSV glycoprotein and lentiviral gag/pol coding sequences. After transfection, lentiviral particles may be harvested from the cell media and concentrated. HEK293T cells may be transduced using serial dilutions of the lentiviral particles described above. Cells generated at a dilution of MOI <1, as determined by survival following puromycin, are selected for expansion. A resulting HEK293T cell line carrying the c. 1042delCT mutation may be used to screen PEgRNAs.

### Installation of L348fs Mutation by Prime Editing: Alternate Method for Generation of Mutant Cell Line.

PEgRNAs for NGG PAM recognition were designed to incorporate a SLC37A4 c. 1042delCT mutation in the wild type endogenous SLC37A4 gene in HEK293T cells by prime editing as a proxy to examine editing efficiency.

A wild type HEK293T cell line is expanded and transiently transfected with a plasmid encoding the PE2 fusion protein and a L348fs mutation installation PEgRNA in arrayed 96-well plates for assessment of editing by high-throughput sequencing. Prior to transfection, cells were seeded in 96-well plates and then transfected with Lipofectamine 2000 or MessengerMax according to the manufacturer's directions with DNA or mRNA PE2 and PEgRNA. Three days after transfection, gDNA was harvested in lysis buffer for high throughput sequencing and sequenced using Miseq.

Glycogen storage disease type 1B mutation correction with PE2 system: A HEK293T cell line carrying the L348fs mutation, such as one made by a method described above, is expanded and transiently transfected with a PE and PEgRNA in arrayed 96-well plates for assessment of editing by high-throughput sequencing. Six exemplary PEgRNA spacers close to the L348fs mutation are listed in Table 8. Spacers S11, S12, and S13 have sequences from the sense (or positive) strand, and spacers S14, S15, and S16 have sequences from the antisense (or negative) strand.

Exemplary RTT sequences in PEgRNAs for spacers S11, S12, S13, S14, S15, and S16 are listed in Tables 19a, 20a, 21a, 22a, 23a, and 24a, respectively. Exemplary PBS sequences in PEgRNAs for spacers $11, S12, S13, S14, S15, and S16 are listed in Tables 19b, 20b, 21b, 22b, 23b, and 24b, respectively. Exemplary RTT/PBS combinations in PEgRNAs for spacers S11, S12, S13, S14, S15, and S16 are listed in Tables 19c, 20c, 21c, 22c, 23c, and 24c, respectively.

The PEgRNAs made according to these exemplary embodiments may contain, in order from 5′ to 3′, a spacer, a gRNA core (e.g., SEQ ID NO: 54) as discussed above, an RTT appropriate for the spacer, a PBS appropriate for the spacer, and a 3′ end modifier region (e.g., SEQ ID NO: 57) as discussed above. In some embodiments, a PEgRNA includes the sequence of spacer S11, an RTT sequence selected from the RTT sequences listed in Table 19a, and the sequence of one PBS in Table 19b (an “S11 PEgRNA”). In some embodiments, a PEgRNA includes the sequence of spacer S12, an RTT sequence selected from the RTT sequences listed in Table 20a, and a PBS sequence selected from the PBS sequences listed in Table 20b (an “S12 PEgRNA”). In some embodiments, a PEgRNA includes the sequence of spacer S13, an RTT sequence selected from the RTT sequences listed in Table 21a, and a PBS sequence selected from the PBS sequences listed in PBS in Table 21b (an “S13 PEgRNA”). In some embodiments, a PEgRNA includes the sequence of spacer S14, an RTT sequence selected from the RTT sequences listed in Table 22a, and a PBS sequence selected from the PBS sequences listed in Table 22b (an “S14 PEgRNA”). In some embodiments, a PEgRNA includes the sequence of spacer S15, an RTT sequence selected from the RTT sequences listed in Table 23a, and a PBS sequence selected from the PBS sequences listed in Table 23b (an “S15 PEgRNA”). In some embodiments, a PEgRNA includes the sequence of spacer S16, an RTT sequence selected from the RTT sequences listed in Table 24a, and a PBS sequence selected from the PBS sequences listed in in Table 24b (an “S16 PEgRNA”).

The PEgRNA may also include a 5′ end modifier region as discussed above. The PEgRNAs made according to these exemplary embodiments may include chemically modified RNA nucleobases as discussed above. Specifically, in these PEgRNAs the first three 5′ residues may be phosphorothioated 2′O-methyl RNA bases, and the last three 3′ residues before the final residue (i.e., the three consecutive nucleotides immediately 5′ of the last nucleotide at the 3′ end) may be phosphorothioated 2′O-methyl RNA bases.

Glycogen storage disease type 1B mutation correction with PE3 system: a second-nick guide RNA (“ngRNA”) that causes a nick on the opposite strand compared to the PEgRNA (i.e., on the non-edit strand) may be included in order improve efficiency and/or fidelity of prime editing as discussed above. Exemplary ngRNA negative-strand spacers are listed in Table 8c, and exemplary positive-strand spacers are listed in Table 8d. ngRNAs according to these exemplary embodiments may contain, in order from 5′ to 3′, a spacer, a gRNA core such as SEQ ID NO: 54 as discussed above, and optionally a 3′ end modifier region such as SEQ ID NO: 57 as discussed above. The ngRNA may also include a 5′ end modifier region as discussed above. The ngRNA may include chemically modified RNA nucleobases as discussed above. For example, in a ngRNA the first three 5′ residues may be phosphorothioated 2′O-methyl RNA bases, and the last three 3′ residues may be phosphorothioated 2′O-methyl RNA bases. A PEgRNA with a positive-strand spacer may be paired with negative-strand ngRNA. In some embodiments, a PE3 system may include a PEgRNA comprising the spacer S11 and an ngRNA with a spacer having a sequence selected from the ngRNA spacer sequences provided in Table 8c. In some embodiments, a PE3 system may include a PEgRNA comprising the spacer S12 and an ngRNA with a spacer having a sequence selected from the ngRNA spacer sequences provided in Table 8c. In some embodiments, a PE3 system may include a PEgRNA comprising the spacer S13 and an ngRNA with a spacer having a sequence selected from the ngRNA spacer sequences provided in Table 8c. In some embodiments, a PE3 system may include a PEgRNA comprising the spacer S14 and an ngRNA with a spacer having a sequence selected from the ngRNA spacer sequences provided in Table 8d. In some embodiments, a PE3 system may include a PEgRNA comprising the spacer S15 and an ngRNA with a spacer having a sequence selected from the ngRNA spacer sequences provided in Table 8d. In some embodiments, a PE3 system may include a PEgRNA comprising the spacer S16 and an ngRNA with a spacer having a sequence selected from the ngRNA spacer sequences provided in Table 8d.

The results of several experiments measuring correction of the L348fs mutation in HEK293T cells using various synthetic PEgRNAs in combination with various ngRNAs as laid out in the tables referenced above are reported in Table 24f, Table 24g, Table 24h, and Table 24i. PEgRNAs are identified by “PEG-nnnn” numbers, for each of which RTT and PSB sequences are provided elsewhere. ngRNAs are identified by sequence numbers from Table 8c and Table 8d. The data is reported as percentage of sampled cells in which sequencing identified the mutation as correctly repaired (“edit %”) or otherwise (i.e., incorrectly) modified (“indel %”).

Glycogen Storage Disease Type 1B L348fs Mutation Correction with PE3 System in iPSC:

A dose-response study was conducted using in human inducible pluripotent stem cells. iPSCs were mutated to carry the L348fs mutation in the SLC37A4 gene and differentiated to a hepatocyte lineage. They were co-transfected mRNA encoding Prime Editor, RNA encoding PEgRNA and RNA encoding ngRNA targeting the SLC37A4 gene. Seventy-two hours following transfection gDNA were harvested. Next generation sequencing was used to calculate editing efficiency by quantifying the number of alleles with the desired sequence change. iPSC transfection: iPSCs were seeded in a 96-well plate the day prior to transfection. On transfection day, a mixture consisting of mRNA encoding Prime Editor, RNA encoding PEgRNA, and ngRNA were diluted in Optimem for a total volume of 6.5 μl. This mix was titrated together, keeping the ratio of mRNA, PEgRNA and ngRNA fixed. PEgRNA doses tested were 112, 74.7, 49.8, 33.2, 22.1, 14.7, 9.8, 6.6, 4.4, 2.9, 2.0 and 1.3 ng. ngRNA doses tested were 37.3, 24.9, 16.6, 11.1, 7.4, 4.9, 3.3, 2.2, 1.5, 1, 0.7 and 0.4 ng. Prime Editor mRNA doses tested were 337.4, 224.9, 150, 100, 66.6, 44.4, 29.6, 19.7, 13.1, 8.8, 5.8 and 3.9 ng. The mixture was then added to a dilution of transfection reagent consisting of Lipofectamine Stem reagent diluted in Optimem for a total volume of 6.5 ul. The two mixtures were mixed and incubated for 10 min at room temperature. 13 ul of transfection mixture were added to each well and swirled to ensure even distribution. Plates were returned to the incubator and cultured at 37 C with 5% CO₂.

Two trials of dose responses for various combinations of PEgRNA and ngRNA were tested in iPSC, with two replicates for each trial. The results are provided in Table 24j. The reported dose is the sum of mRNAm PEgRNA, and ngRNA.

As noted above, PEgRNAs comprising the S12 or S13 spacers, when they also include an RTT sequence from Tables 20a or 21a, respectively, can also correct the nearby c.1015G->T (G339C) transversion mutation. This is because the editing template is long enough to provide a corrective sequence template to cover both mutation sites.

### Sequences and Sequence Tables

Exemplary wild type moloney murine leukemia virus reverse transcriptase (SEQ ID NO: 50):
TLNIEDEYRLHETSKEPDVSLGSTWLSDFPQAWAETGGMGLAVRQAPLIIPLKATSTP
VSIKQYPMSQEARLGIKPHIQRLLDQGILVPCQSPWNTPLLPVKKPGTNDYRPVQDLR
EVNKRVEDIHPTVPNPYNLLSGLPPSHQWYTVLDLKDAFFCLRLHPTSQPLFAFEWR
DPEMGISGQLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLTEARKE
TVMGQPTPKTPRQLREFLGTAGFCRLWIPGFAEMAAPLYPLTKTGTLFNWGPDQQK
AYQEIKQALLTAPALGLPDLTKPFELFVDEKQGYAKGVLTQKLGPWRRPVAYLSKK
LDPVAAGWPPCLRMVAAIAVLTKDAGKLTMGQPLVILAPHAVEALVKQPPDRWLS
NARMTHYQALLLDTDRVQFGPVVALNPATLLPLPEEGLQHNCLDILAEAHGTRPDL
TDQPLPDADHTWYTDGSSLLQEGQRKAGAAVTTETEVIWAKALPAGTSAQRAELIA
LTQALKMAEGKKLNVYTDSRYAFATAHIHGEIYRRRGLLTSEGKEIKNKDEILALLK
ALFLPKRLSIIHCPGHQKGHSAEARGNRMADQAARKAAITETPDTSTLLIENSSP
Exemplary Streptococcus pyogenes Cas9 (SpCas9) amino acid sequence (SEQ ID NO: 51):
MDKKYSIGLDIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
Exemplary Staphylococcus lugdunensis Cas9 (Slu Cas9) amino acid sequence
WP_002460848.1 (SEQ ID NO: 52):
MKRNYILGLDIGITSVGYGIIDYETRDVIDAGVRLFKEANVENNEGRRSKRGARRLK
RRRRHRIQRVKKLLFDYNLLTDHSELSGINPYEARVKGLSQKLSEEEFSAALLHLAKR
RGVHNVNEVEEDTGNELSTKEQISRNSKALEEKYVAELQLERLKKDGEVRGSINRFK
TSDYVKEAKQLLKVQKAYHQLDQSFIDTYIDLLETRRTYYEGPGEGSPFGWKDIKE
WYEMLMGHCTYFPEELRSVKYAYNADLYNALNDLNNLVITRDENEKLEYYEKFQII
ENVFKQKKKPTLKQIAKEILVNEEDIKGYRVTSTGKPEFTNLKVYHDIKDITARKEIIE
NAELLDQIAKILTIYQSSEDIQEELTNLNSELTQEEIEQISNLKGYTGTHNLSLKAINLIL
DELWHTNDNQIAIFNRLKLVPKKVDLSQQKEIPTTLVDDFILSPVVKRSFIQSIKVINAI
IKKYGLPNDIIIELAREKNSKDAQKMINEMQKRNRQTNERIEEIIRTTGKENAKYLIEKI
KLHDMQEGKCLYSLEAIPLEDLLNNPFNYEVDHIIPRSVSFDNSFNNKVLVKQEEASK
KGNRTPFQYLSSSDSKISYETFKKHILNLAKGKGRISKTKKEYLLEERDINRFSVQKDF
INRNLVDTRYATRGLMNLLRSYFRVNNLDVKVKSINGGFTSFLRRKWKFKKERNKG
YKHHAEDALIIANADFIFKEWKKLDKAKKVMENQMFEEKQAESMPEIETEQEYKEIF
ITPHQIKHIKDFKDYKYSHRVDKKPNRELINDTLYSTRKDDKGNTLIVNNLNGLYDK
DNDKLKKLINKSPEKLLMYHHDPQTYQKLKLIMEQYGDEKNPLYKYYEETGNYLTK
YSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYNNDLIKINGELYR
VIGVNNDLLNRIEVNMIDITYREYLENMNDKRPPRIIKTIASKTQSIKKYSTDILGNLY
EVKSKKHPQIIKK
Exemplary prime editor fusion protein: SEQ ID NO: 53 (see Table 4).
SLC37A4/G6PT1 amino acid sequence (SEQ ID NO: 1):
MAAQGYGYYRTVIFSAMFGGYSLYYFNRKTFSFVMPSLVEEIPLDKDDLGFITSSQS
AAYAISKFVSGVLSDQMSARWLFSSGLLLVGLVNIFFAWSSTVPVFAALWFLNGLAQ
GLGWPPCGKVLRKWFEPSQFGTWWAILSTSMNLAGGLGPILATILAQSYSWRSTLAL
SGALCVVVSFLCLLLIHNEPADVGLRNLDPMPSEGKKGSLKEESTLQELLLSPYLWV
LSTGYLVVFGVKTCCTDWGQFFLIQEKGQSALVGSSYMSALEVGGLVGSIAAGYLS
DRAMAKAGLSNYGNPRHGLLLFMMAGMTVSMYLFRVTVTSDSPKLWILVLGAVFG
FSSYGPIALFGVIANESAPPNLCGTSHAIVGLMANVGGFLAGLPFSTIAKHYSWSTAF
WVAEVICAASTAAFFLLRNIRTKMGRVSKKAE
SLC37A4/G6PT1 cDNA sequence (SEQ ID NO: 2):
gtcctattcgggctcccgcctctgttcagttccagagacaattcatgggtcttggggccaccgaggcgctgtccctgaccaccagcacg
agacccctttctatcgcgccagtcctgtggtctccgcacctctccagctcctgcacccccggcccccgtggttcccagccgcacagtag
cgtgtcctgggtagcgtgaggacccacggggctgggcaggtgccacgagcccgccgcctcttcgccgcccgccgcctctcctcctc
tcccgcccgccgcctggccctcccctaccaggctgagcctctgggtgccagaagcgcggggcctccgggagaatacgtgcggtcg
cccgctccgcgtgcgcctacgccttctgctccagttgctttcccaattgagcggaaaagccggggcatgttgccggggccctgggcg
ggacggttgtgccctgcagcccgaagcccgccggggcaccttcccgcccacgagctgcccagtccctctgcttgcggcccctgcca
acgtcccacaggacactgggtccccttggagcctccccaggcttaatgattgtccagaaggcggctataaagggagcctgggaggct
gggggaggagggagcagaaaaaacccaactcagcagatctgggaactgtgagagcggcaagcaggaactgtggtcagaggctg
tgcgtcttggctggtagggcctgctcttttctaccATGgcagcccagggctatggctattatcgcactgtgatcttctcagccatgtttg
ggggctacagcctgtattacttcaatcgcaagaccttctcctttgtcatgccatcattggtggaagagatccctttggacaaggatgatttg
gggttcatcaccagcagccagtcggcagcttatgctatcagcaagtttgtcagtggggtgctgtctgaccagatgagtgctcgctggct
cttctcttctgggctgctcctggttggcctggtcaacatattctttgcctggagctccacagtacctgtctttgctgccctctggttcctta
atggcctggcccaggggctgggctggcccccatgtgggaaggtcctgcggaagtggtttgagccatctcagtttggcacttggtgggcca
tcctgtcaaccagcatgaacctggctggagggctgggccctatcctggcaaccatccttgcccagagctacagctggcgcagcacgc
tggccctatctggggcactgtgtgtggttgtctccttcctctgtctcctgctcatccacaatgaacctgctgatgttggactccgcaacctg
gaccccatgccctctgagggcaagaagggctccttgaaggaggagagcaccctgcaggagctgctgctgtccccttacctgtgggtg
ctctccactggttaccttgtggtgtttggagtaaagacctgctgtactgactggggccagttcttccttatccaggagaaaggacagtcag
cccttgtaggtagctcctacatgagtgccctggaagttgggggccttgtaggcagcatcgcagctggctacctgtcagaccgggccat
ggcaaaggcgggactgtccaactacgggaaccctcgccatggcctgttgctgttcatgatggctggcatgacagtgtccatgtacctct
tccgggtaacagtgaccagtgactcccccaagctctggatcctggtattgggagctgtattt [symbol] gtttctcctcgtatggccccattgccct
gtttggagtcatagccaacgagagtgcccctcccaacttgtgtggcacctcccacgccattgtgggactcatggccaatgtgggcggc
tttctggctgggctgcccttcagcaccattgccaagcactacagttggagcacagccttctgggtggctgaagtgatttgtgcggccag
cacggctgccttcttcctcctacgaaacatccgcaccaagatgggccgagtgtccaagaaggctgagtgaagagagtccaggttccg
gagcaccatcccacggtggccttccccctgcacgctctgcggggagaaaaggaggggcctgcctggctagccctgaacctttcactt
tccatttctgcgccttttctgtcacccgggggcgctggaagttatcagtggctagtgaggtcccagctccctgatcctatgctctatttaaa
agataacctttggccttagactccgttagctcctatttcctgccttcagacaaacaggaaacttctgcagtcaggaaggctcctgtaccctt
cttcttttcctaggccctgtcctgcccgcatcctaccccatccccacctgaagtgaggctatccctgcagctgcagggcactaatgaccc
ttgacttctgctgggtcctaagtcctctcagcagtgggtgactgctgttgccaatacctcagactccagggaaagagaggaggccatca
ttctcactgtaccactaggcgcagttggatataggtgggaagaaaaggtgacttgttatagaagattaaaactagatttgatactgaacac
tgtcagtgattcattttttcaaagtgagacagcttccttgggaaatattgtcaatacctgctctttccaccccaaaatggaaagacttcattt
ccctggaatggggaagctgaagtgtagatggactcctttaaaactcatgcctcctctgccttcacctgaacattgaatagttcaatgattatt
ttagagataaagctattgggttgtggacagattaaataacttgatggagggaa

TABLE 8
Exemplary Spacer sequences

| Name | SEQ ID NO: | Sequence |
|---|---|---|
| S01 | 60 | CAGGGCAAUGGGGCCAUACG |
| S02 | 61 | UGACUCCAAACAGGGCAAUG |
| S03 | 62 | AUGACUCCAAACAGGGCAAU |
| S04 | 63 | UAUGACUCCAAACAGGGCAA |
| S05 | 64 | GUUGGCUAUGACUCCAAACA |
| S06 | 65 | CGUUGGCUAUGACUCCAAAC |
| S07 | 66 | CAGCUCUGGAUCCUGGUAUU |
| S08 | 67 | GCAGCUCUGGAUCCUGGUAU |
| S09 | 68 | CCCUUGGCAGCUCUGGAUCC |
| S10 | 69 | CACUCUGCCCUUGGCAGCUC |
| S11 | 6043 | UCGUUGGCUAUGACUCCAAA |
| S12 | 6044 | GUUGGGAGGGGCACUCUCGU |
| S13 | 6045 | GGUGCCACACAAGUUGGGAG |
| S14 | 6046 | CGUAUGGCCCCAUUGCCGUU |
| S15 | 6047 | GUAUUUGGUUUCUCCUCGUA |
| S16 | 6048 | CUGGUAUUGGGAGCUGUAUU |

TABLE 8a
Exemplary negative strand nick-guide RNA spacers

| SEQ ID NO: | Spacer sequence |
|---|---|
| 332 | AUUGUGGGACUCAUGGCCAA |
| 333 | CCACGCCAUUGUGGGACUCA |
| 334 | GGCACCUCCCACGCCAUUGU |
| 335 | UGGCACCUCCCACGCCAUUG |
| 336 | AGUGCCCCUCCCAACUUGUG |
| 337 | UAUGGCCCCAUUGCCCUGUU |
| 338 | GUAUUUgGUUUCUCCUCGUA |
| 339 | CAGCUCUGGAUCCUGGUAUU |
| 340 | GCAGCUCUGGAUCCUGGUAU |
| 341 | CCCUUGGCAGCUCUGGAUCC |
| 342 | CACUCUGCCCUUGGCAGCUC |
| 343 | AUGCCACUCCACUCUGCCCU |
| 344 | GCAGGGGUGGGUAGGCAGGU |
| 345 | UUUGGCAGGGGUGGGUAGGC |
| 346 | UUGCUUUGGCAGGGGUGGGU |
| 347 | CUUCUUGCUUUGGCAGGGGU |
| 5001 | GUAUUUGGUUUCUCAUCGUA |
| 5002 | GUAUUUGGUUUCUCGUCGUA |
| 5003 | GUAUUUGGUUUCUCUUCGUA |
| 5004 | UAUGGUCCCAUUGCCCUGUU |
| 5005 | UAUGGGCCCAUUGCCCUGUU |
| 5006 | UAUGGACCCAUUGCCCUGUU |
| 5007 | UAUGGCCCUAUUGCCCUGUU |
| 5008 | UAUGGCCCGAUUGCCCUGUU |
| 5009 | UAUGGCCCAAUUGCCCUGUU |
| 5010 | UAUGGCCCCAUUGCUCUGUU |
| 5011 | UAUGGCCCCAUUGCACUGUU |
| 5012 | UAUGGCCCCAUUGCGCUGUU |
| 5013 | ACCAAGCCUUCUUCUUGCUU |
| 5014 | AGCCUUCUUCUUGCUUUGGC |
| 5015 | GCCUUCUUCUUGCUUUGGCA |
| 5016 | CCUUCUUCUUGCUUUGGCAG |
| 5017 | UCUUCUUGCUUUGGCAGGGG |

TABLE 8b
Exemplary positive strand nick-guide RNA spacers

| SEQ ID NO: | Spacer sequence |
|---|---|
| 348 | AUUGGCCAUGAGUCCCACAA |
| 349 | CCAUGAGUCCCACAAUGGCG |
| 350 | CAUGAGUCCCACAAUGGCGU |
| 351 | GAGUCCCACAAUGGCGUGGG |
| 352 | GUGGGAGGUGCCACACAAGU |
| 353 | UGGGAGGUGCCACACAAGUU |
| 354 | GAGGUGCCACACAAGUUGGG |
| 355 | AGGUGCCACACAAGUUGGGA |
| 356 | GGUGCCACACAAGUUGGGAG |
| 357 | GUUGGGAGGGGCACUCUCGU |
| 358 | CGUUGGCUAUGACUCCAAAC |
| 359 | GUUGGCUAUGACUCCAAACA |
| 360 | UAUGACUCCAAACAGGGCAA |
| 361 | AUGACUCCAAACAGGGCAAU |
| 362 | UGACUCCAAACAGGGCAAUG |
| 363 | CAGGGCAAUGGGGCCAUACG |
| 364 | CAAAUACAGCUCCCAAUACC |
| 365 | ACCAGGAUCCAGAGCUGCCA |
| 366 | CCAGGAUCCAGAGCUGCCAA |
| 367 | CAGAGCUGCCAAGGGCAGAG |
| 368 | CUGCCAAGGGCAGAGUGGAG |
| 369 | AGUGGAGUGGCAUUCAGAGU |
| 370 | CCCCUGCCAAAGCAAGAAGA |
| 5018 | CUAGGAUCCAGAGCUGCCAA |
| 5019 | CAAGGAUCCAGAGCUGCCAA |
| 5020 | CGAGGAUCCAGAGCUGCCAA |
| 5021 | ACUAGGAUCCAGAGCUGCCA |
| 5022 | ACAAGGAUCCAGAGCUGCCA |
| 5023 | ACGAGGAUCCAGAGCUGCCA |
| 5024 | UGACUCCAAACAGAGCAAUG |
| 5025 | UGACUCCAAACAGUGCAAUG |
| 5026 | UGACUCCAAACAGCGCAAUG |
| 5027 | AUGACUCCAAACAGAGCAAU |
| 5028 | AUGACUCCAAACAGUGCAAU |
| 5029 | AUGACUCCAAACAGCGCAAU |
| 5030 | CAAAUACAGCUCCUAAUACC |
| 5031 | CAAAUACAGCUCCGAAUACC |
| 5032 | CAAAUACAGCUCCAAAUACC |
| 5033 | CAAAUACAGCUCCCAAUACU |
| 5034 | CAAAUACAGCUCCCAAUACG |
| 5035 | CAAAUACAGCUCCCAAUACA |
| 6083 | GCCAAAGCAAGAAGAAGGCU |

TABLE 8c
Exemplary negative strand nick-guide RNA spacers

| SEQ ID NO: | Spacer sequence |
|---|---|
| 521 | AUUGUGGGACUCAUGGCCAA |
| 522 | CCACGCCAUUGUGGGACUCA |
| 523 | GGCACCUCCCACGCCAUUGU |
| 524 | UGGCACCUCCCACGCCAUUG |
| 525 | AGUGCCCCUCCCAACUUGUG |
| 526 | UAUGGCCCCAUUGCCcuGUU |
| 527 | GUAUUUGGUUUCUCCUCGUA |
| 528 | CUGGUAUUGGGAGCUGUAUU |
| 529 | CAGCUCUGGAUCCUGGUAUU |
| 530 | GCAGCUCUGGAUCCUGGUAU |
| 531 | CCCUUGGCAGCUCUGGAUCC |
| 532 | CACUCUGCCCUUGGCAGCUC |
| 533 | AUGCCACUCCACUCUGCCCU |
| 3201 | UAUGGCCCCAUUGCUCUGUU |
| 3202 | UAUGGCCCCAUUGCACUGUU |
| 3203 | UAUGGCCCCAUUGCGCUGUU |
| 3204 | ACCAAGCCUUCUUCUUGCUU |
| 3205 | AGCCUUCUUCUUGCUUUGGC |
| 3206 | GCCUUCUUCUUGCUUUGGCA |
| 3207 | CCUUCUUCUUGCUUUGGCAG |
| 3208 | UCUUCUUGCUUUGGCAGGGG |
| 3209 | GCAGGGGUGGGUAGGCAGGU |
| 3210 | UUUGGCAGGGGUGGGUAGGC |
| 3212 | GUAUUUGGUUUCUCAUCGUA |
| 3213 | GUAUUUGGUUUCUCGUCGUA |
| 3214 | GUAUUUGGUUUCUCUUCGUA |

TABLE 8d
Exemplary positive strand nick-guide RNA spacers

| SEQ ID NO: | Spacer sequence |
|---|---|
| 534 | UAGAAGUUAACACUUACCAU |
| 535 | AUUGGCCAUGAGUCCCACAA |
| 536 | CCAUGAGUCCCACAAUGGCG |
| 537 | CAUGAGUCCCACAAUGGCGU |
| 538 | GAGUCCCACAAUGGCGUGGG |
| 539 | GUGGGAGGUGCCACACAAGU |
| 540 | UGGGAGGUGCCACACAAGUU |
| 541 | GAGGUGCCACACAAGUUGGG |
| 542 | AGGUGCCACACAAGUUGGGA |
| 543 | GGUGCCACACAAGUUGGGAG |
| 544 | GUUGGGAGGGGCACUCUCGU |
| 545 | GUUGGCUAUGACUCCAAACa |
| 546 | UAUGACUCCAAACagGGCAA |
| 547 | AUGACUCCAAACagGGCAAU |
| 548 | UGACUCCAAACagGGCAAUG |
| 549 | CagGGCAAUGGGGCCAUACG |
| 550 | CAAAUACAGCUCCCAAUACC |
| 551 | ACCAGGAUCCAGAGCUGCCA |
| 552 | CCAGGAUCCAGAGCUGCCAA |
| 553 | CAGAGCUGCCAAGGGCAGAG |
| 554 | CUGCCAAGGGCAGAGUGGAG |
| 555 | AGUGGAGUGGCAUUCAGAGU |
| 3211 | CGUUGGCUAUGACUCCAAAC |
| 3215 | CCCCUGCCAAAGCAAGAAGA |

TABLE 9a
Exemplary RTT sequences for Spacer S01

| SEQ ID | | SEQ ID | |
|---|---|---|---|
| NO: | Sequence | NO: | Sequence |
| 70 | UgGUUUCUCCUCGU | 96 | GCUGUAUUUgGUUUCUcauCGU |
| 71 | UUgGUUUCaguUCGU | 97 | GCUGUAUUUgGUUUCUCCUCGU |
| 72 | UUgGUUUCUcuuCGU | 98 | AGCUGUAUUUgGUUUCUCCUCGU |
| 73 | UUgGUUUCUcguCGU | 99 | GAGCUGUAUUUgGUUUCUCCUCGU |
| 74 | UUgGUUUCagcUCGU | 100 | GGAGCUGUAUUUgGUUUCaguUCGU |
| 75 | UUgGUUUCUcauCGU | 101 | GGAGCUGUAUUUgGUUUCUcuuCGU |
| 76 | UUgGUUUCUCCUCGU | 102 | GGAGCUGUAUUUgGUUUCUcguCGU |
| 77 | UUUgGUUUCUCCUCGU | 103 | GGAGCUGUAUUUgGUUUCagcUCGU |
| 78 | AUUUgGUUUCaguUCGU | 104 | GGAGCUGUAUUUgGUUUCUcauCGU |
| 79 | AUUUgGUUUCUcuuCGU | 105 | GGAGCUGUAUUUgGUUUCUCCUCGU |
| 80 | AUUUgGUUUCUcguCGU | 106 | GGGAGCUGUAUUUgGUUUCUCCUCGU |
| 81 | AUUUgGUUUCagcUCGU | 107 | UGGGAGCUGUAUUUgGUUUCaguUCGU |
| 82 | AUUUgGUUUCUcauCGU | 108 | UGGGAGCUGUAUUUgGUUUCUcuuCGU |
| 83 | AUUUgGUUUCUCCUCGU | 109 | UGGGAGCUGUAUUUgGUUUCUcguCGU |
| 84 | UAUUUgGUUUCUCCUCGU | 110 | UGGGAGCUGUAUUUgGUUUCagcUCGU |
| 85 | GUAUUUgGUUUCaguUCGU | 111 | UGGGAGCUGUAUUUgGUUUCUcauCGU |
| 86 | GUAUUUgGUUUCUcuuCGU | 112 | UGGGAGCUGUAUUUgGUUUCUCCUCGU |
| 87 | GUAUUUgGUUUCUcguCGU | 113 | UUGGGAGCUGUAUUUgGUUUCUCCUCGU |
| 88 | GUAUUUgGUUUCagcUCGU | 114 | AUUGGGAGCUGUAUUUgGUUUCUCCUCGU |
| 89 | GUAUUUgGUUUCUcauCGU | 115 | UAUUGGGAGCUGUAUUUgGUUUCUCCUCGU |
| 90 | GUAUUUgGUUUCUCCUCGU | 116 | GUAUUGGGAGCUGUAUUUgGUUUCUCCUCGU |
| 91 | UGUAUUUgGUUUCUCCUCGU | 117 | GGUAUUGGGAGCUGUAUUUgGUUUCUCCUCGU |
| 92 | GCUGUAUUUgGUUUCaguUCGU | 118 | UGGUAUUGGGAGCUGUAUUUgGUUUCUCCUCGU |
| 93 | GCUGUAUUUgGUUUCUcuuCGU | 119 | UCCUGGUAUUGGGAGCUGUAUUUgGUUUCUCCUCGU |
| 94 | GCUGUAUUUgGUUUCUcguCGU | 120 | AUCCUGGUAUUGGGAGCUGUAUUUgGUUUCUCCUCGU |
| 95 | GCUGUAUUUgGUUUCagcUCGU | 3022 | CUGUAUUUgGUUUCUcguCGU |
| 3014 | UAUUUgGUUUCUcuuCGU | 3023 | CUGUAUUUGGUUUCUCCUCGU |
| 3012 | UAUUUgGUUUCUcguCGU | 3024 | CUGUAUUUGGUUUCUCAUCGU |
| 3013 | UAUUUGGUUUCUCAUCGU | 3101 | CUGUAUUUgGUUUCUCCUCGU |
| 3016 | GUAUUUgGUUUCUcguCGU | 3025 | GCUGUAUUUgGUUUCUcuuCGU |
| 3017 | GUAUUUGGUUUCUCAUCGU | 3026 | GCUGUAUUUgGUUUCUcguCGU |
| 3018 | UGUAUUUgGUUUCUcuuCGU | 3027 | GCUGUAUUUGGUUUCUCAUCGU |
| 3019 | UGUAUUUgGUUUCUcguCGU | 3028 | AGCUGUAUUUgGUUUCUcuuCGU |
| 3003 | UGUAUUUGGUUUCUCAUCGU | 3029 | AGCUGUAUUUgGUUUCUcguCGU |
| 3020 | UGUAUUUGGUUUCUCAUCGU | 3030 | AGCUGUAUUUGGUUUCUCAUCGU |
| 3021 | CUGUAUUUgGUUUCUcuuCGU | | |

TABLE 9b
Exemplary PBS sequences for Spacer S01

| | Sequence |
|---|---|
| SEQ REF NO: 242 | AUGGCCC |
| SEQ REF NO: 243 | AUGGCCCC |
| SEQ REF NO: 244 | AUGGCCCCA |
| SEQ ID NO: 245 | AUGGCCCCAU |
| SEQ ID NO: 246 | AUGGCCCCAUU |
| SEQ ID NO: 247 | AUGGCCCCAUUG |
| SEQ ID NO: 248 | AUGGCCCCAUUGC |
| SEQ ID NO: 249 | AUGGCCCCAUUGCC |
| SEQ ID NO: 250 | AUGGCCCCAUUGCCC |

TABLE 9c
Exemplary RTT/PBS Combinations with Spacer S01

| PEG # | RTT | PBS | PEG # | RTT | PBS | PEG # | RTT | PBS | PEG # | RTT | PBS |
|---|---|---|---|---|---|---|---|---|---|---|---|
| PEG-0001 | 70 | 242 | PEG-0039 | 84 | 244 | PEG-0077 | 98 | 246 | PEG-0115 | 112 | 248 |
| PEG-0002 | 70 | 243 | PEG-0040 | 84 | 245 | PEG-0078 | 98 | 247 | PEG-0116 | 112 | 249 |
| PEG-0003 | 70 | 244 | PEG-0041 | 84 | 246 | PEG-0079 | 98 | 248 | PEG-0117 | 112 | 250 |
| PEG-0004 | 70 | 245 | PEG-0042 | 84 | 247 | PEG-0080 | 98 | 249 | PEG-0118 | 113 | 242 |
| PEG-0005 | 70 | 246 | PEG-0043 | 84 | 248 | PEG-0081 | 98 | 250 | PEG-0119 | 113 | 243 |
| PEG-0006 | 70 | 247 | PEG-0044 | 84 | 249 | PEG-0082 | 99 | 242 | PEG-0120 | 113 | 244 |
| PEG-0007 | 70 | 248 | PEG-0045 | 84 | 250 | PEG-0083 | 99 | 243 | PEG-0121 | 113 | 245 |
| PEG-0008 | 70 | 249 | PEG-0693 | 85 | 243 | PEG-0084 | 99 | 244 | PEG-0122 | 113 | 246 |
| PEG-0009 | 70 | 250 | PEG-0694 | 85 | 245 | PEG-0085 | 99 | 245 | PEG-0123 | 113 | 247 |
| PEG-0685 | 71 | 243 | PEG-0695 | 85 | 247 | PEG-0086 | 99 | 246 | PEG-0124 | 113 | 248 |
| PEG-0686 | 71 | 245 | PEG-0696 | 85 | 249 | PEG-0087 | 99 | 247 | PEG-0125 | 113 | 249 |
| PEG-0687 | 71 | 247 | PEG-0621 | 86 | 243 | PEG-0088 | 99 | 248 | PEG-0126 | 113 | 250 |
| PEG-0688 | 71 | 249 | PEG-0622 | 86 | 245 | PEG-0089 | 99 | 249 | PEG-0127 | 114 | 242 |
| PEG-0613 | 72 | 243 | PEG-0623 | 86 | 247 | PEG-0090 | 99 | 250 | PEG-0128 | 114 | 243 |
| PEG-0614 | 72 | 245 | PEG-0624 | 86 | 249 | PEG-0701 | 100 | 243 | PEG-0129 | 114 | 244 |
| PEG-0615 | 72 | 247 | PEG-0669 | 87 | 243 | PEG-0702 | 100 | 245 | PEG-0130 | 114 | 245 |
| PEG-0616 | 72 | 249 | PEG-0670 | 87 | 245 | PEG-0703 | 100 | 247 | PEG-0131 | 114 | 246 |
| PEG-0661 | 73 | 243 | PEG-0671 | 87 | 247 | PEG-0704 | 100 | 249 | PEG-0132 | 114 | 247 |
| PEG-0662 | 73 | 245 | PEG-0672 | 87 | 249 | PEG-0629 | 101 | 243 | PEG-0133 | 114 | 248 |
| PEG-0663 | 73 | 247 | PEG-0717 | 88 | 243 | PEG-0630 | 101 | 245 | PEG-0134 | 114 | 249 |
| PEG-0664 | 73 | 249 | PEG-0718 | 88 | 245 | PEG-0631 | 101 | 247 | PEG-0135 | 114 | 250 |
| PEG-0709 | 74 | 243 | PEG-0719 | 88 | 247 | PEG-0632 | 101 | 249 | PEG-0136 | 115 | 242 |
| PEG-0710 | 74 | 245 | PEG-0720 | 88 | 249 | PEG-0677 | 102 | 243 | PEG-0137 | 115 | 243 |
| PEG-0711 | 74 | 247 | PEG-0645 | 89 | 243 | PEG-0678 | 102 | 245 | PEG-0138 | 115 | 244 |
| PEG-0712 | 74 | 249 | PEG-0646 | 89 | 245 | PEG-0679 | 102 | 247 | PEG-0139 | 115 | 245 |
| PEG-0637 | 75 | 243 | PEG-0647 | 89 | 247 | PEG-0680 | 102 | 249 | PEG-0140 | 115 | 246 |
| PEG-0638 | 75 | 245 | PEG-0648 | 89 | 249 | PEG-0725 | 103 | 243 | PEG-0141 | 115 | 247 |
| PEG-0639 | 75 | 247 | PEG-0046 | 90 | 242 | PEG-0726 | 103 | 245 | PEG-0142 | 115 | 248 |
| PEG-0640 | 75 | 249 | PEG-0047 | 90 | 243 | PEG-0727 | 103 | 247 | PEG-0143 | 115 | 249 |
| PEG-0010 | 76 | 242 | PEG-0048 | 90 | 244 | PEG-0728 | 103 | 249 | PEG-0144 | 115 | 250 |
| PEG-0011 | 76 | 243 | PEG-0049 | 90 | 245 | PEG-0653 | 104 | 243 | PEG-0145 | 116 | 242 |
| PEG-0012 | 76 | 244 | PEG-0050 | 90 | 246 | PEG-0654 | 104 | 245 | PEG-0146 | 116 | 243 |
| PEG-0013 | 76 | 245 | PEG-0051 | 90 | 247 | PEG-0655 | 104 | 247 | PEG-0147 | 116 | 244 |
| PEG-0014 | 76 | 246 | PEG-0052 | 90 | 248 | PEG-0656 | 104 | 249 | PEG-0148 | 116 | 245 |
| PEG-0015 | 76 | 247 | PEG-0053 | 90 | 249 | PEG-0091 | 105 | 242 | PEG-0149 | 116 | 246 |
| PEG-0016 | 76 | 248 | PEG-0054 | 90 | 250 | PEG-0092 | 105 | 243 | PEG-0150 | 116 | 247 |
| PEG-0017 | 76 | 249 | PEG-0055 | 91 | 242 | PEG-0093 | 105 | 244 | PEG-0151 | 116 | 248 |
| PEG-0018 | 76 | 250 | PEG-0056 | 91 | 243 | PEG-0094 | 105 | 245 | PEG-0152 | 116 | 249 |
| PEG-0019 | 77 | 242 | PEG-0057 | 91 | 244 | PEG-0095 | 105 | 246 | PEG-0153 | 116 | 250 |
| PEG-0020 | 77 | 243 | PEG-0058 | 91 | 245 | PEG-0096 | 105 | 247 | PEG-0154 | 117 | 242 |
| PEG-0021 | 77 | 244 | PEG-0059 | 91 | 246 | PEG-0097 | 105 | 248 | PEG-0155 | 117 | 243 |
| PEG-0022 | 77 | 245 | PEG-0060 | 91 | 247 | PEG-0098 | 105 | 249 | PEG-0156 | 117 | 244 |
| PEG-0023 | 77 | 246 | PEG-0061 | 91 | 248 | PEG-0099 | 105 | 250 | PEG-0157 | 117 | 245 |
| PEG-0024 | 77 | 247 | PEG-0062 | 91 | 249 | PEG-0100 | 106 | 242 | PEG-0158 | 117 | 246 |
| PEG-0025 | 77 | 248 | PEG-0063 | 91 | 250 | PEG-0101 | 106 | 243 | PEG-0159 | 117 | 247 |
| PEG-0026 | 77 | 249 | PEG-0697 | 92 | 243 | PEG-0102 | 106 | 244 | PEG-0160 | 117 | 248 |
| PEG-0027 | 77 | 250 | PEG-0698 | 92 | 245 | PEG-0103 | 106 | 245 | PEG-0161 | 117 | 249 |
| PEG-0689 | 78 | 243 | PEG-0699 | 92 | 247 | PEG-0104 | 106 | 246 | PEG-0162 | 117 | 250 |
| PEG-0690 | 78 | 245 | PEG-0700 | 92 | 249 | PEG-0105 | 106 | 247 | PEG-0163 | 118 | 242 |
| PEG-0691 | 78 | 247 | PEG-0625 | 93 | 243 | PEG-0106 | 106 | 248 | PEG-0164 | 118 | 243 |
| PEG-0692 | 78 | 249 | PEG-0626 | 93 | 245 | PEG-0107 | 106 | 249 | PEG-0165 | 118 | 244 |
| PEG-0617 | 79 | 243 | PEG-0627 | 93 | 247 | PEG-0108 | 106 | 250 | PEG-0166 | 118 | 245 |
| PEG-0618 | 79 | 245 | PEG-0628 | 93 | 249 | PEG-0705 | 107 | 243 | PEG-0167 | 118 | 246 |
| PEG-0619 | 79 | 247 | PEG-0673 | 94 | 243 | PEG-0706 | 107 | 245 | PEG-0168 | 118 | 247 |
| PEG-0620 | 79 | 249 | PEG-0674 | 94 | 245 | PEG-0707 | 107 | 247 | PEG-0169 | 118 | 248 |
| PEG-0665 | 80 | 243 | PEG-0675 | 94 | 247 | PEG-0708 | 107 | 249 | PEG-0170 | 118 | 249 |
| PEG-0666 | 80 | 245 | PEG-0676 | 94 | 249 | PEG-0633 | 108 | 243 | PEG-0171 | 118 | 250 |
| PEG-0667 | 80 | 247 | PEG-0721 | 95 | 243 | PEG-0634 | 108 | 245 | PEG-0172 | 119 | 242 |
| PEG-0668 | 80 | 249 | PEG-0722 | 95 | 245 | PEG-0635 | 108 | 247 | PEG-0173 | 119 | 243 |
| PEG-0713 | 81 | 243 | PEG-0723 | 95 | 247 | PEG-0636 | 108 | 249 | PEG-0174 | 119 | 244 |
| PEG-0714 | 81 | 245 | PEG-0724 | 95 | 249 | PEG-0681 | 109 | 243 | PEG-0175 | 119 | 245 |
| PEG-0715 | 81 | 247 | PEG-0649 | 96 | 243 | PEG-0682 | 109 | 245 | PEG-0176 | 119 | 246 |
| PEG-0716 | 81 | 249 | PEG-0650 | 96 | 245 | PEG-0683 | 109 | 247 | PEG-0177 | 119 | 247 |
| PEG-0641 | 82 | 243 | PEG-0651 | 96 | 247 | PEG-0684 | 109 | 249 | PEG-0178 | 119 | 248 |
| PEG-0642 | 82 | 245 | PEG-0652 | 96 | 249 | PEG-0729 | 110 | 243 | PEG-0179 | 119 | 249 |
| PEG-0643 | 82 | 247 | PEG-0064 | 97 | 242 | PEG-0730 | 110 | 245 | PEG-0180 | 119 | 250 |
| PEG-0644 | 82 | 249 | PEG-0065 | 97 | 243 | PEG-0731 | 110 | 247 | PEG-0181 | 120 | 242 |
| PEG-0028 | 83 | 242 | PEG-0066 | 97 | 244 | PEG-0732 | 110 | 249 | PEG-0182 | 120 | 243 |
| PEG-0029 | 83 | 243 | PEG-0067 | 97 | 245 | PEG-0657 | 111 | 243 | PEG-0183 | 120 | 244 |
| PEG-0030 | 83 | 244 | PEG-0068 | 97 | 246 | PEG-0658 | 111 | 245 | PEG-0184 | 120 | 245 |
| PEG-0031 | 83 | 245 | PEG-0069 | 97 | 247 | PEG-0659 | 111 | 247 | PEG-0185 | 120 | 246 |
| PEG-0032 | 83 | 246 | PEG-0070 | 97 | 248 | PEG-0660 | 111 | 249 | PEG-0186 | 120 | 247 |
| PEG-0033 | 83 | 247 | PEG-0071 | 97 | 249 | PEG-0109 | 112 | 242 | PEG-0187 | 120 | 248 |
| PEG-0034 | 83 | 248 | PEG-0072 | 97 | 250 | PEG-0110 | 112 | 243 | PEG-0188 | 120 | 249 |
| PEG-0035 | 83 | 249 | PEG-0073 | 98 | 242 | PEG-0111 | 112 | 244 | PEG-0189 | 120 | 250 |
| PEG-0036 | 83 | 250 | PEG-0074 | 98 | 243 | PEG-0112 | 112 | 245 | PEG-4216 | 3028 | 249 |
| PEG-0037 | 84 | 242 | PEG-0075 | 98 | 244 | PEG-0113 | 112 | 246 | PEG-4150 | 3029 | 249 |
| PEG-0038 | 84 | 243 | PEG-0076 | 98 | 245 | PEG-0114 | 112 | 247 | PEG-4183 | 3030 | 249 |
| PEG-4184 | 3014 | 243 | PEG-4125 | 94 | 244 | PEG-4200 | 3021 | 246 | PEG-4142 | 87 | 248 |
| PEG-4117 | 3012 | 243 | PEG-4158 | 96 | 244 | PEG-4134 | 3022 | 246 | PEG-4175 | 89 | 248 |
| PEG-4151 | 3013 | 243 | PEG-4193 | 3028 | 244 | PEG-4167 | 3024 | 246 | PEG-4209 | 3018 | 248 |
| PEG-4185 | 3018 | 243 | PEG-4126 | 3029 | 244 | PEG-4113 | 3101 | 246 | PEG-4143 | 3019 | 248 |
| PEG-4118 | 3019 | 243 | PEG-4159 | 3030 | 244 | PEG-4201 | 93 | 246 | PEG-4176 | 3003 | 248 |
| PEG-4006 | 3003 | 243 | PEG-4194 | 3014 | 245 | PEG-4135 | 94 | 246 | PEG-4210 | 3021 | 248 |
| PEG-4186 | 3021 | 243 | PEG-4127 | 3012 | 245 | PEG-4168 | 96 | 246 | PEG-4144 | 3022 | 248 |
| PEG-4119 | 3022 | 243 | PEG-4160 | 3013 | 245 | PEG-4202 | 3028 | 246 | PEG-4177 | 3024 | 248 |
| PEG-4152 | 3024 | 243 | PEG-4195 | 3018 | 245 | PEG-4136 | 3029 | 246 | PEG-4115 | 3101 | 248 |
| PEG-4110 | 3101 | 243 | PEG-4128 | 3019 | 245 | PEG-4169 | 3030 | 246 | PEG-4211 | 93 | 248 |
| PEG-4187 | 3028 | 243 | PEG-4161 | 3003 | 245 | PEG-4203 | 3014 | 247 | PEG-4145 | 94 | 248 |
| PEG-4120 | 3029 | 243 | PEG-4196 | 3021 | 245 | PEG-4137 | 3012 | 247 | PEG-4178 | 96 | 248 |
| PEG-4153 | 3030 | 243 | PEG-4129 | 3022 | 245 | PEG-4170 | 3013 | 247 | PEG-4212 | 3028 | 248 |
| PEG-4188 | 3014 | 244 | PEG-4162 | 3024 | 245 | PEG-4204 | 3018 | 247 | PEG-4146 | 3029 | 248 |
| PEG-4121 | 3012 | 244 | PEG-4112 | 3101 | 245 | PEG-4138 | 3019 | 247 | PEG-4179 | 3030 | 248 |
| PEG-4154 | 3013 | 244 | PEG-4197 | 3028 | 245 | PEG-4171 | 3003 | 247 | PEG-4213 | 3014 | 249 |
| PEG-4189 | 86 | 244 | PEG-4130 | 3029 | 245 | PEG-4205 | 3021 | 247 | PEG-4147 | 3012 | 249 |
| PEG-4122 | 87 | 244 | PEG-4163 | 3030 | 245 | PEG-4139 | 3022 | 247 | PEG-4180 | 3013 | 249 |
| PEG-4155 | 89 | 244 | PEG-4198 | 3014 | 246 | PEG-4172 | 3024 | 247 | PEG-4214 | 3018 | 249 |
| PEG-4190 | 3018 | 244 | PEG-4131 | 3012 | 246 | PEG-4114 | 3101 | 247 | PEG-4148 | 3019 | 249 |
| PEG-4123 | 3019 | 244 | PEG-4164 | 3013 | 246 | PEG-4206 | 3028 | 247 | PEG-4181 | 3003 | 249 |
| PEG-4156 | 3003 | 244 | PEG-4005 | 86 | 246 | PEG-4140 | 3029 | 247 | PEG-4215 | 3021 | 249 |
| PEG-4191 | 3021 | 244 | PEG-4132 | 87 | 246 | PEG-4173 | 3030 | 247 | PEG-4149 | 3022 | 249 |
| PEG-4124 | 3022 | 244 | PEG-4165 | 89 | 246 | PEG-4207 | 3014 | 248 | PEG-4182 | 3024 | 249 |
| PEG-4157 | 3024 | 244 | PEG-4199 | 3018 | 246 | PEG-4141 | 3012 | 248 | PEG-4116 | 3101 | 249 |
| PEG-4111 | 3101 | 244 | PEG-4133 | 3019 | 246 | PEG-4174 | 3013 | 248 | | | |
| PEG-4192 | 93 | 244 | PEG-4166 | 3003 | 246 | PEG-4208 | 86 | 248 | | | |

TABLE 10a
Exemplary RTT sequences for Spacer S02

| SEQ | | SEQ | |
|---|---|---|---|
| ID NO: | Sequence | ID NO: | Sequence |
| 121 | UgGUUUCUCCUCGUAUGGC | 134 | GCUGUAUUUgGUUUCUCCUCGUAUGGCCCCAU |
| | CCCAU | | |
| 122 | UUgGUUUCUCCUCGUAUGG | 135 | AGCUGUAUUUgGUUUCUCCUCGUAUGGuCCCAU |
| | CCCCAU | | |
| 123 | UUUgGUUUCUCCUCGUAUG | 136 | AGCUGUAUUUgGUUUCUCCUCGUAUGGgCCCAU |
| | GuCCCAU | | |
| 124 | UUUgGUUUCUCCUCGUAUG | 137 | AGCUGUAUUUgGUUUCUCCUCGUAUGGCCCCAU |
| | GgCCCAU | | |
| 125 | UUUgGUUUCUCCUCGUAUG | 138 | AGCUGUAUUUgGUUUCUCCUCGUAUGGaCCCAU |
| | GCCCCAU | | |
| 126 | UUUgGUUUCUCCUCGUAUG | 139 | GAGCUGUAUUUgGUUUCUCCUCGUAUGGCCCCA |
| | GaCCCAU | | U |
| 127 | AUUUgGUUUCUCCUCGUAU | 140 | GGAGCUGUAUUUgGUUUCUCCUCGUAUGGCCCC |
| | GGCCCCAU | | AU |
| 128 | UAUUUgGUUUCUCCUCGUA | 141 | GGGAGCUGUAUUUgGUUUCUCCUCGUAUGGuCC |
| | UGGCCCCAU | | CAU |
| 129 | GUAUUUgGUUUCUCCUCGU | 142 | GGGAGCUGUAUUUgGUUUCUCCUCGUAUGGgCC |
| | AUGGuCCCAU | | CAU |
| 130 | GUAUUUgGUUUCUCCUCGU | 143 | GGGAGCUGUAUUUgGUUUCUCCUCGUAUGGCCC |
| | AUGGgCCCAU | | CAU |
| 131 | GUAUUUgGUUUCUCCUCGU | 144 | GGGAGCUGUAUUUgGUUUCUCCUCGUAUGGaCC |
| | AUGGCCCCAU | | CAU |
| 132 | GUAUUUgGUUUCUCCUCGU | 145 | UGGGAGCUGUAUUUgGUUUCUCCUCGUAUGGCC |
| | AUGGaCCCAU | | CCAU |
| 133 | UGUAUUUgGUUUCUCCUCG | 3053 | GCUGUAUUUGGUUUCUCCUCGUAUGGGCCCAU |
| | UAUGGCCCCAU | | |
| 3035 | UAUUUGGUUUCUCCUCGUA | 3054 | GCUGUAUUUGGUUUCUCCUCGUAUGGCCCCAU |
| | UGGUCCCAU | | |
| 3036 | UAUUUGGUUUCUCCUCGUA | 3055 | GCUGUAUUUGGUUUCUCCUCGUAUGGACCCAU |
| | UGGGCCCAU | | |
| 3037 | UAUUUGGUUUCUCCUCGUA | 3056 | AGCUGUAUUUGGUUUCUCCUCGUAUGGUCCCAU |
| | UGGCCCCAU | | |
| 3038 | UAUUUGGUUUCUCCUCGUA | 3057 | AGCUGUAUUUGGUUUCUCCUCGUAUGGGCCCAU |
| | UGGACCCAU | | |
| 3039 | GUAUUUGGUUUCUCCUCGU | 3058 | AGCUGUAUUUGGUUUCUCCUCGUAUGGCCCCAU |
| | AUGGUCCCAU | | |
| 3040 | GUAUUUGGUUUCUCCUCGU | 3059 | AGCUGUAUUUGGUUUCUCCUCGUAUGGACCCAU |
| | AUGGGCCCAU | | |
| 3041 | GUAUUUGGUUUCUCCUCGU | 3060 | GAGCUGUAUUUGGUUUCUCCUCGUAUGGUCCCAU |
| | AUGGCCCCAU | | |
| 3042 | GUAUUUGGUUUCUCCUCGU | 3061 | GAGCUGUAUUUGGUUUCUCCUCGUAUGGGCCCAU |
| | AUGGACCCAU | | |
| 3043 | UGUAUUUGGUUUCUCCUCG | 3062 | GAGCUGUAUUUGGUUUCUCCUCGUAUGGCCCCAU |
| | UAUGGUCCCAU | | |
| 3004 | UGUAUUUGGUUUCUCCUCG | 3063 | GAGCUGUAUUUGGUUUCUCCUCGUAUGGACCCAU |
| | UAUGGGCCCAU | | |
| 3044 | UGUAUUUGGUUUCUCCUCG | 3064 | GGAGCUGUAUUUGGUUUCUCCUCGUAUGGUCCCAU |
| | UAUGGGCCCAU | | |
| 3045 | UGUAUUUGGUUUCUCCUCG | 3065 | GGAGCUGUAUUUGGUUUCUCCUCGUAUGGGCCCAU |
| | UAUGGCCCCAU | | |
| 3046 | UGUAUUUGGUUUCUCCUCG | 3066 | GGAGCUGUAUUUGGUUUCUCCUCGUAUGGCCCCAU |
| | UAUGGACCCAU | | |
| 3047 | CUGUAUUUGGUUUCUCCUC | 3005 | GGAGCUGUAUUUGGUUUCUCCUCGUAUGGACCCAU |
| | GUAUGGUCCCAU | | |

TABLE 10b
Exemplary PBS sequences for Spacer S02

| | Sequence |
|---|---|
| SEQ REF NO: 251 | UGCCCUG |
| SEQ REF NO: 252 | UGCCCUGU |
| SEQ REF NO: 253 | UGCCCUGUU |
| SEQ ID NO: 254 | UGCCCUGUUU |
| SEQ ID NO: 255 | UGCCCUGUUUG |
| SEQ ID NO: 256 | UGCCCUGUUUGG |
| SEQ ID NO: 257 | UGCCCUGUUUGGA |
| SEQ ID NO: 258 | UGCCCUGUUUGGAG |
| SEQ ID NO: 259 | UGCCCUGUUUGGAGU |

TABLE 10c
Exemplary RTT/PBS Combinations with Spacer S02

| PEG # | RTT | PBS | PEG # | RTT | PBS | PEG # | RTT | PBS | PEG # | RTT | PBS |
|---|---|---|---|---|---|---|---|---|---|---|---|
| PEG-0190 | 121 | 251 | PEG-0220 | 127 | 254 | PEG-0250 | 133 | 257 | PEG-0280 | 140 | 251 |
| PEG-0191 | 121 | 252 | PEG-0221 | 127 | 255 | PEG-0251 | 133 | 258 | PEG-0281 | 140 | 252 |
| PEG-0192 | 121 | 253 | PEG-0222 | 127 | 256 | PEG-0252 | 133 | 259 | PEG-0282 | 140 | 253 |
| PEG-0193 | 121 | 254 | PEG-0223 | 127 | 257 | PEG-0253 | 134 | 251 | PEG-0283 | 140 | 254 |
| PEG-0194 | 121 | 255 | PEG-0224 | 127 | 258 | PEG-0254 | 134 | 252 | PEG-0284 | 140 | 255 |
| PEG-0195 | 121 | 256 | PEG-0225 | 127 | 259 | PEG-0255 | 134 | 253 | PEG-0285 | 140 | 256 |
| PEG-0196 | 121 | 257 | PEG-0226 | 128 | 251 | PEG-0256 | 134 | 254 | PEG-0286 | 140 | 257 |
| PEG-0197 | 121 | 258 | PEG-0227 | 128 | 252 | PEG-0257 | 134 | 255 | PEG-0287 | 140 | 258 |
| PEG-0198 | 121 | 259 | PEG-0228 | 128 | 253 | PEG-0258 | 134 | 256 | PEG-0288 | 140 | 259 |
| PEG-0199 | 122 | 251 | PEG-0229 | 128 | 254 | PEG-0259 | 134 | 257 | PEG-0761 | 141 | 252 |
| PEG-0200 | 122 | 252 | PEG-0230 | 128 | 255 | PEG-0260 | 134 | 258 | PEG-0762 | 141 | 254 |
| PEG-0201 | 122 | 253 | PEG-0231 | 128 | 256 | PEG-0261 | 134 | 259 | PEG-0763 | 141 | 256 |
| PEG-0202 | 122 | 254 | PEG-0232 | 128 | 257 | PEG-0757 | 135 | 252 | PEG-0764 | 141 | 258 |
| PEG-0203 | 122 | 255 | PEG-0233 | 128 | 258 | PEG-0758 | 135 | 254 | PEG-0777 | 142 | 252 |
| PEG-0204 | 122 | 256 | PEG-0234 | 128 | 259 | PEG-0759 | 135 | 256 | PEG-0778 | 142 | 254 |
| PEG-0205 | 122 | 257 | PEG-0753 | 129 | 252 | PEG-0760 | 135 | 258 | PEG-0779 | 142 | 256 |
| PEG-0206 | 122 | 258 | PEG-0754 | 129 | 254 | PEG-0773 | 136 | 252 | PEG-0780 | 142 | 258 |
| PEG-0207 | 122 | 259 | PEG-0755 | 129 | 256 | PEG-0774 | 136 | 254 | PEG-0289 | 143 | 251 |
| PEG-0749 | 123 | 252 | PEG-0756 | 129 | 258 | PEG-0775 | 136 | 256 | PEG-0290 | 143 | 252 |
| PEG-0750 | 123 | 254 | PEG-0769 | 130 | 252 | PEG-0776 | 136 | 258 | PEG-0291 | 143 | 253 |
| PEG-0751 | 123 | 256 | PEG-0770 | 130 | 254 | PEG-0262 | 137 | 251 | PEG-0292 | 143 | 254 |
| PEG-0752 | 123 | 258 | PEG-0771 | 130 | 256 | PEG-0263 | 137 | 252 | PEG-0293 | 143 | 255 |
| PEG-0765 | 124 | 252 | PEG-0772 | 130 | 258 | PEG-0264 | 137 | 253 | PEG-0294 | 143 | 256 |
| PEG-0766 | 124 | 254 | PEG-0235 | 131 | 251 | PEG-0265 | 137 | 254 | PEG-0295 | 143 | 257 |
| PEG-0767 | 124 | 256 | PEG-0236 | 131 | 252 | PEG-0266 | 137 | 255 | PEG-0296 | 143 | 258 |
| PEG-0768 | 124 | 258 | PEG-0237 | 131 | 253 | PEG-0267 | 137 | 256 | PEG-0297 | 143 | 259 |
| PEG-0208 | 125 | 251 | PEG-0238 | 131 | 254 | PEG-0268 | 137 | 257 | PEG-0745 | 144 | 252 |
| PEG-0209 | 125 | 252 | PEG-0239 | 131 | 255 | PEG-0269 | 137 | 258 | PEG-0746 | 144 | 254 |
| PEG-0210 | 125 | 253 | PEG-0240 | 131 | 256 | PEG-0270 | 137 | 259 | PEG-0747 | 144 | 256 |
| PEG-0211 | 125 | 254 | PEG-0241 | 131 | 257 | PEG-0741 | 138 | 252 | PEG-0748 | 144 | 258 |
| PEG-0212 | 125 | 255 | PEG-0242 | 131 | 258 | PEG-0742 | 138 | 254 | PEG-0298 | 145 | 251 |
| PEG-0213 | 125 | 256 | PEG-0243 | 131 | 259 | PEG-0743 | 138 | 256 | PEG-0299 | 145 | 252 |
| PEG-0214 | 125 | 257 | PEG-0737 | 132 | 252 | PEG-0744 | 138 | 258 | PEG-0300 | 145 | 253 |
| PEG-0215 | 125 | 258 | PEG-0738 | 132 | 254 | PEG-0271 | 139 | 251 | PEG-0301 | 145 | 254 |
| PEG-0216 | 125 | 259 | PEG-0739 | 132 | 256 | PEG-0272 | 139 | 252 | PEG-0302 | 145 | 255 |
| PEG-0733 | 126 | 252 | PEG-0740 | 132 | 258 | PEG-0273 | 139 | 253 | PEG-0303 | 145 | 256 |
| PEG-0734 | 126 | 254 | PEG-0244 | 133 | 251 | PEG-0274 | 139 | 254 | PEG-0304 | 145 | 257 |
| PEG-0735 | 126 | 256 | PEG-0245 | 133 | 252 | PEG-0275 | 139 | 255 | PEG-0305 | 145 | 258 |
| PEG-0736 | 126 | 258 | PEG-0246 | 133 | 253 | PEG-0276 | 139 | 256 | PEG-0306 | 145 | 259 |
| PEG-0217 | 127 | 251 | PEG-0247 | 133 | 254 | PEG-0277 | 139 | 257 | PEG-4271 | 3004 | 257 |
| PEG-0218 | 127 | 252 | PEG-0248 | 133 | 255 | PEG-0278 | 139 | 258 | PEG-4334 | 3046 | 257 |
| PEG-0219 | 127 | 253 | PEG-0249 | 133 | 256 | PEG-0279 | 139 | 259 | PEG-4398 | 3047 | 257 |
| PEG-4350 | 3035 | 252 | PEG-4302 | 3063 | 253 | PEG-4381 | 3052 | 255 | PEG-4272 | 3048 | 257 |
| PEG-4224 | 3036 | 252 | PEG-4366 | 3064 | 253 | PEG-4255 | 3053 | 255 | PEG-4222 | 3049 | 257 |
| PEG-4287 | 3038 | 252 | PEG-4240 | 3065 | 253 | PEG-4318 | 3055 | 255 | PEG-4335 | 3050 | 257 |
| PEG-4351 | 129 | 252 | PEG-4303 | 3005 | 253 | PEG-4382 | 135 | 255 | PEG-4399 | 3052 | 257 |
| PEG-4225 | 130 | 252 | PEG-4367 | 141 | 253 | PEG-4256 | 136 | 255 | PEG-4273 | 3053 | 257 |
| PEG-4288 | 132 | 252 | PEG-4241 | 142 | 253 | PEG-4319 | 138 | 255 | PEG-4336 | 3055 | 257 |
| PEG-4352 | 3043 | 252 | PEG-4304 | 144 | 253 | PEG-4383 | 3060 | 255 | PEG-4400 | 135 | 257 |
| PEG-4226 | 3004 | 252 | PEG-4368 | 3035 | 254 | PEG-4257 | 3061 | 255 | PEG-4274 | 136 | 257 |
| PEG-4289 | 3046 | 252 | PEG-4242 | 3036 | 254 | PEG-4320 | 3063 | 255 | PEG-4337 | 138 | 257 |
| PEG-4353 | 3047 | 252 | PEG-4305 | 3038 | 254 | PEG-4384 | 3064 | 255 | PEG-4401 | 3060 | 257 |
| PEG-4227 | 3048 | 252 | PEG-4369 | 129 | 254 | PEG-4258 | 3065 | 255 | PEG-4275 | 3061 | 257 |
| PEG-4217 | 3049 | 252 | PEG-4243 | 130 | 254 | PEG-4321 | 3005 | 255 | PEG-4338 | 3063 | 257 |
| PEG-4290 | 3050 | 252 | PEG-4008 | 132 | 254 | PEG-4385 | 141 | 255 | PEG-4402 | 3064 | 257 |
| PEG-4354 | 3052 | 252 | PEG-4306 | 132 | 254 | PEG-4259 | 142 | 255 | PEG-4276 | 3065 | 257 |
| PEG-4228 | 3053 | 252 | PEG-4370 | 3043 | 254 | PEG-4322 | 144 | 255 | PEG-4339 | 3005 | 257 |
| PEG-4291 | 3055 | 252 | PEG-4244 | 3004 | 254 | PEG-4386 | 3035 | 256 | PEG-4403 | 141 | 257 |
| PEG-4355 | 135 | 252 | PEG-4307 | 3046 | 254 | PEG-4260 | 3036 | 256 | PEG-4277 | 142 | 257 |
| PEG-4229 | 136 | 252 | PEG-4371 | 3047 | 254 | PEG-4323 | 3038 | 256 | PEG-4340 | 144 | 257 |
| PEG-4292 | 138 | 252 | PEG-4245 | 3048 | 254 | PEG-4387 | 129 | 256 | PEG-4404 | 3035 | 258 |
| PEG-4356 | 3060 | 252 | PEG-4219 | 3049 | 254 | PEG-4261 | 130 | 256 | PEG-4278 | 3036 | 258 |
| PEG-4230 | 3061 | 252 | PEG-4308 | 3050 | 254 | PEG-4324 | 132 | 256 | PEG-4341 | 3038 | 258 |
| PEG-4293 | 3063 | 252 | PEG-4372 | 3052 | 254 | PEG-4388 | 3043 | 256 | PEG-4405 | 129 | 258 |
| PEG-4357 | 3064 | 252 | PEG-4246 | 3053 | 254 | PEG-4262 | 3004 | 256 | PEG-4279 | 130 | 258 |
| PEG-4231 | 3065 | 252 | PEG-4309 | 3055 | 254 | PEG-4325 | 3046 | 256 | PEG-4342 | 132 | 258 |
| PEG-4294 | 3005 | 252 | PEG-4373 | 135 | 254 | PEG-4389 | 3047 | 256 | PEG-4406 | 3043 | 258 |
| PEG-4358 | 141 | 252 | PEG-4247 | 136 | 254 | PEG-4263 | 3048 | 256 | PEG-4009 | 3004 | 258 |
| PEG-4232 | 142 | 252 | PEG-4310 | 138 | 254 | PEG-4221 | 3049 | 256 | PEG-4280 | 3004 | 258 |
| PEG-4295 | 144 | 252 | PEG-4374 | 3060 | 254 | PEG-4326 | 3050 | 256 | PEG-4343 | 3046 | 258 |
| PEG-4359 | 3035 | 253 | PEG-4248 | 3061 | 254 | PEG-4390 | 3052 | 256 | PEG-4407 | 3047 | 258 |
| PEG-4233 | 3036 | 253 | PEG-4311 | 3063 | 254 | PEG-4264 | 3053 | 256 | PEG-4281 | 3048 | 258 |
| PEG-4296 | 3038 | 253 | PEG-4375 | 3064 | 254 | PEG-4327 | 3055 | 256 | PEG-4223 | 3049 | 258 |
| PEG-4360 | 129 | 253 | PEG-4249 | 3065 | 254 | PEG-4391 | 135 | 256 | PEG-4344 | 3050 | 258 |
| PEG-4234 | 130 | 253 | PEG-4312 | 3005 | 254 | PEG-4265 | 136 | 256 | PEG-4408 | 3052 | 258 |
| PEG-4007 | 132 | 253 | PEG-4376 | 141 | 254 | PEG-4328 | 138 | 256 | PEG-4282 | 3053 | 258 |
| PEG-4297 | 132 | 253 | PEG-4250 | 142 | 254 | PEG-4392 | 3060 | 256 | PEG-4345 | 3055 | 258 |
| PEG-4361 | 3043 | 253 | PEG-4010 | 144 | 254 | PEG-4266 | 3061 | 256 | PEG-4409 | 135 | 258 |
| PEG-4235 | 3004 | 253 | PEG-4313 | 144 | 254 | PEG-4329 | 3063 | 256 | PEG-4283 | 136 | 258 |
| PEG-4298 | 3046 | 253 | PEG-4377 | 3035 | 255 | PEG-4393 | 3064 | 256 | PEG-4346 | 138 | 258 |
| PEG-4362 | 3047 | 253 | PEG-4251 | 3036 | 255 | PEG-4267 | 3065 | 256 | PEG-4410 | 3060 | 258 |
| PEG-4236 | 3048 | 253 | PEG-4314 | 3038 | 255 | PEG-4330 | 3005 | 256 | PEG-4284 | 3061 | 258 |
| PEG-4218 | 3049 | 253 | PEG-4378 | 129 | 255 | PEG-4394 | 141 | 256 | PEG-4347 | 3063 | 258 |
| PEG-4299 | 3050 | 253 | PEG-4252 | 130 | 255 | PEG-4268 | 142 | 256 | PEG-4411 | 3064 | 258 |
| PEG-4363 | 3052 | 253 | PEG-4315 | 132 | 255 | PEG-4331 | 144 | 256 | PEG-4285 | 3065 | 258 |
| PEG-4237 | 3053 | 253 | PEG-4379 | 3043 | 255 | PEG-4395 | 3035 | 257 | PEG-4348 | 3005 | 258 |
| PEG-4300 | 3055 | 253 | PEG-4253 | 3004 | 255 | PEG-4269 | 3036 | 257 | PEG-4412 | 141 | 258 |
| PEG-4364 | 135 | 253 | PEG-4316 | 3046 | 255 | PEG-4332 | 3038 | 257 | PEG-4286 | 142 | 258 |
| PEG-4238 | 136 | 253 | PEG-4380 | 3047 | 255 | PEG-4396 | 129 | 257 | PEG-4349 | 144 | 258 |
| PEG-4301 | 138 | 253 | PEG-4254 | 3048 | 255 | PEG-4270 | 130 | 257 | | | |
| PEG-4365 | 3060 | 253 | PEG-4220 | 3049 | 255 | PEG-4333 | 132 | 257 | | | |
| PEG-4239 | 3061 | 253 | PEG-4317 | 3050 | 255 | PEG-4397 | 3043 | 257 | | | |

TABLE 11a
Exemplary RTT sequences for Spacer S03

| SEQ ID | | SEQ ID | |
|---|---|---|---|
| NO: | Sequence | NO: | Sequence |
| 146 | UgGUUUCUCCUCGUAUGGC | 152 | UGUAUUUgGUUUCUCCUCGUAUGGCCCCAU |
| | CCCAUU | | U |
| 147 | UUgGUUUCUCCUCGUAUGG | 153 | GCUGUAUUUgGUUUCUCCUCGUAUGGCCCC |
| | CCCCAUU | | AUU |
| 148 | UUUgGUUUCUCCUCGUAUG | 154 | AGCUGUAUUUgGUUUCUCCUCGUAUGGCCC |
| | GCCCCAUU | | CAUU |
| 149 | AUUUgGUUUCUCCUCGUAU | 155 | GAGCUGUAUUUgGUUUCUCCUCGUAUGGCC |
| | GGCCCCAUU | | CCAUU |
| 150 | UAUUUgGUUUCUCCUCGUA | 156 | GGAGCUGUAUUUgGUUUCUCCUCGUAUGGC |
| | UGGCCCCAUU | | CCCAUU |
| 151 | GUAUUUgGUUUCUCCUCGU | 157 | GGGAGCUGUAUUUgGUUUCUCCUCGUAUGG |
| | AUGGCCCCAUU | | CCCCAUU |

TABLE 11b
Exemplary PBS sequences for Spacer S03

| | Sequence |
|---|---|
| SEQ REF NO: 260 | GCCCUGU |
| SEQ REF NO: 261 | GCCCUGUU |
| SEQ REF NO: 262 | GCCCUGUUU |
| SEQ ID NO: 263 | GCCCUGUUUG |
| SEQ ID NO: 264 | GCCCUGUUUGG |
| SEQ ID NO: 265 | GCCCUGUUUGGA |
| SEQ ID NO: 266 | GCCCUGUUUGGAG |
| SEQ ID NO: 267 | GCCCUGUUUGGAGU |
| SEQ ID NO: 268 | GCCCUGUUUGGAGUC |

TABLE 11c
Exemplary RTT/PBS Combinations with Spacer S03

| PEG # | RTT | PBS |
|---|---|---|
| PEG-0307 | 146 | 260 |
| PEG-0308 | 146 | 261 |
| PEG-0309 | 146 | 262 |
| PEG-0310 | 146 | 263 |
| PEG-0311 | 146 | 264 |
| PEG-0312 | 146 | 265 |
| PEG-0313 | 146 | 266 |
| PEG-0314 | 146 | 267 |
| PEG-0315 | 146 | 268 |
| PEG-0316 | 147 | 260 |
| PEG-0317 | 147 | 261 |
| PEG-0318 | 147 | 262 |
| PEG-0319 | 147 | 263 |
| PEG-0320 | 147 | 264 |
| PEG-0321 | 147 | 265 |
| PEG-0322 | 147 | 266 |
| PEG-0323 | 147 | 267 |
| PEG-0324 | 147 | 268 |
| PEG-0325 | 148 | 260 |
| PEG-0326 | 148 | 261 |
| PEG-0327 | 148 | 262 |
| PEG-0328 | 148 | 263 |
| PEG-0329 | 148 | 264 |
| PEG-0330 | 148 | 265 |
| PEG-0331 | 148 | 266 |
| PEG-0332 | 148 | 267 |
| PEG-0333 | 148 | 268 |
| PEG-0334 | 149 | 260 |
| PEG-0335 | 149 | 261 |
| PEG-0336 | 149 | 262 |
| PEG-0337 | 149 | 263 |
| PEG-0338 | 149 | 264 |
| PEG-0339 | 149 | 265 |
| PEG-0340 | 149 | 266 |
| PEG-0341 | 149 | 267 |
| PEG-0342 | 149 | 268 |
| PEG-0343 | 150 | 260 |
| PEG-0344 | 150 | 261 |
| PEG-0345 | 150 | 262 |
| PEG-0346 | 150 | 263 |
| PEG-0347 | 150 | 264 |
| PEG-0348 | 150 | 265 |
| PEG-0349 | 150 | 266 |
| PEG-0350 | 150 | 267 |
| PEG-0351 | 150 | 268 |
| PEG-0352 | 151 | 260 |
| PEG-0353 | 151 | 261 |
| PEG-0354 | 151 | 262 |
| PEG-0355 | 151 | 263 |
| PEG-0356 | 151 | 264 |
| PEG-0357 | 151 | 265 |
| PEG-0358 | 151 | 266 |
| PEG-0359 | 151 | 267 |
| PEG-0360 | 151 | 268 |
| PEG-0361 | 152 | 260 |
| PEG-0362 | 152 | 261 |
| PEG-0363 | 152 | 262 |
| PEG-0364 | 152 | 263 |
| PEG-0365 | 152 | 264 |
| PEG-0366 | 152 | 265 |
| PEG-0367 | 152 | 266 |
| PEG-0368 | 152 | 267 |
| PEG-0369 | 152 | 268 |
| PEG-0370 | 153 | 260 |
| PEG-0371 | 153 | 261 |
| PEG-0372 | 153 | 262 |
| PEG-0373 | 153 | 263 |
| PEG-0374 | 153 | 264 |
| PEG-0375 | 153 | 265 |
| PEG-0376 | 153 | 266 |
| PEG-0377 | 153 | 267 |
| PEG-0378 | 153 | 268 |
| PEG-0379 | 154 | 260 |
| PEG-0380 | 154 | 261 |
| PEG-0381 | 154 | 262 |
| PEG-0382 | 154 | 263 |
| PEG-0383 | 154 | 264 |
| PEG-0384 | 154 | 265 |
| PEG-0385 | 154 | 266 |
| PEG-0386 | 154 | 267 |
| PEG-0387 | 154 | 268 |
| PEG-0388 | 155 | 260 |
| PEG-0389 | 155 | 261 |
| PEG-0390 | 155 | 262 |
| PEG-391 | 155 | 263 |
| PEG-0392 | 155 | 264 |
| PEG-0393 | 155 | 265 |
| PEG-0394 | 155 | 266 |
| PEG-0395 | 155 | 267 |
| PEG-0396 | 155 | 268 |
| PEG-0397 | 156 | 260 |
| PEG-0398 | 156 | 261 |
| PEG-0399 | 156 | 262 |
| PEG-0400 | 156 | 263 |
| PEG-0401 | 156 | 264 |
| PEG-0402 | 156 | 265 |
| PEG-0403 | 156 | 266 |
| PEG-0404 | 156 | 267 |
| PEG-0405 | 156 | 268 |
| PEG-0406 | 157 | 260 |
| PEG-0407 | 157 | 261 |
| PEG-0408 | 157 | 262 |
| PEG-0409 | 157 | 263 |
| PEG-0410 | 157 | 264 |
| PEG-0411 | 157 | 265 |
| PEG-0412 | 157 | 266 |
| PEG-0413 | 157 | 267 |
| PEG-0414 | 157 | 268 |

TABLE 12a
Exemplary RTT sequences for Spacer S04

| SEQ ID NO: | Sequence | SEQ ID NO: | Sequence |
|---|---|---|---|
| 158 | UgGUUUCUCCUCGUAUGGC | 164 | UGUAUUUgGUUUCUCCUCGUAUGGCC |
| | CCCAUUG | | CCAUUG |
| 159 | UUgGUUUCUCCUCGUAUGG | 165 | GCUGUAUUUgGUUUCUCCUCGUAUGG |
| | CCCCAUUG | | CCCCAUUG |
| 160 | UUUgGUUUCUCCUCGUAUG | 166 | AGCUGUAUUUgGUUUCUCCUCGUAUG |
| | GCCCCAUUG | | GCCCCAUUG |
| 161 | AUUUgGUUUCUCCUCGUAU | 167 | GAGCUGUAUUUgGUUUCUCCUCGUAU |
| | GGCCCCAUUG | | GGCCCCAUUG |
| 162 | UAUUUgGUUUCUCCUCGUA | 168 | GGAGCUGUAUUUgGUUUCUCCUCGUA |
| | UGGCCCCAUUG | | UGGCCCCAUUG |
| 163 | GUAUUUgGUUUCUCCUCGU | 3001 | GGAGCUGUAUUUgGUUUCUCCUCGUA |
| | AUGGCCCCAUUG | | UGGCCCCAUUG |

TABLE 12b
Exemplary PBS sequences for Spacer S04

| | Sequence |
|---|---|
| SEQ REF NO: 269 | CCCUGUU |
| SEQ REF NO: 270 | CCCUGUUU |
| SEQ REF NO: 271 | CCCUGUUUG |
| SEQ ID NO: 272 | CCCUGUUUGG |
| SEQ ID NO: 273 | CCCUGUUUGGA |
| SEQ ID NO: 274 | CCCUGUUUGGAG |
| SEQ ID NO: 275 | CCCUGUUUGGAGU |
| SEQ ID NO: 276 | CCCUGUUUGGAGUC |
| SEQ ID NO: 277 | CCCUGUUUGGAGUCA |

TABLE 12c
Exemplary RTT/PBS Combinations with Spacer S04

| PEG # | RTT | PBS | PEG # | RTT | PBS | PEG # | RTT | PBS | PEG # | RTT | PBS |
|---|---|---|---|---|---|---|---|---|---|---|---|
| PEG-0415 | 158 | 269 | PEG-0440 | 160 | 276 | PEG-0465 | 163 | 274 | PEG-0490 | 166 | 272 |
| PEG-0416 | 158 | 270 | PEG-0441 | 160 | 277 | PEG-0466 | 163 | 275 | PEG-0491 | 166 | 273 |
| PEG-0417 | 158 | 271 | PEG-0442 | 161 | 269 | PEG-0467 | 163 | 276 | PEG-0492 | 166 | 274 |
| PEG-0418 | 158 | 272 | PEG-0443 | 161 | 270 | PEG-0468 | 163 | 277 | PEG-0493 | 166 | 275 |
| PEG-0419 | 158 | 273 | PEG-0444 | 161 | 271 | PEG-0469 | 164 | 269 | PEG-0494 | 166 | 276 |
| PEG-0420 | 158 | 274 | PEG-0445 | 161 | 272 | PEG-0470 | 164 | 270 | PEG-0495 | 166 | 277 |
| PEG-0421 | 158 | 275 | PEG-0446 | 161 | 273 | PEG-0471 | 164 | 271 | PEG-0496 | 167 | 269 |
| PEG-0422 | 158 | 276 | PEG-0447 | 161 | 274 | PEG-0472 | 164 | 272 | PEG-0497 | 167 | 270 |
| PEG-0423 | 158 | 277 | PEG-0448 | 161 | 275 | PEG-0473 | 164 | 273 | PEG-0498 | 167 | 271 |
| PEG-0424 | 159 | 269 | PEG-0449 | 161 | 276 | PEG-0474 | 164 | 274 | PEG-0499 | 167 | 272 |
| PEG-0425 | 159 | 270 | PEG-0450 | 161 | 277 | PEG-0475 | 164 | 275 | PEG-0500 | 167 | 273 |
| PEG-0426 | 159 | 271 | PEG-0451 | 162 | 269 | PEG-0476 | 164 | 276 | PEG-0501 | 167 | 274 |
| PEG-0427 | 159 | 272 | PEG-0452 | 162 | 270 | PEG-0477 | 164 | 277 | PEG-0502 | 167 | 275 |
| PEG-0428 | 159 | 273 | PEG-0453 | 162 | 271 | PEG-0478 | 165 | 269 | PEG-0503 | 167 | 276 |
| PEG-0429 | 159 | 274 | PEG-0454 | 162 | 272 | PEG-0479 | 165 | 270 | PEG-0504 | 167 | 277 |
| PEG-0430 | 159 | 275 | PEG-0455 | 162 | 273 | PEG-0480 | 165 | 271 | PEG-0505 | 168 | 269 |
| PEG-0431 | 159 | 276 | PEG-0456 | 162 | 274 | PEG-0481 | 165 | 272 | PEG-0506 | 168 | 270 |
| PEG-0432 | 159 | 277 | PEG-0457 | 162 | 275 | PEG-0482 | 165 | 273 | PEG-0507 | 168 | 271 |
| PEG-0433 | 160 | 269 | PEG-0458 | 162 | 276 | PEG-0483 | 165 | 274 | PEG-0508 | 168 | 272 |
| PEG-0434 | 160 | 270 | PEG-0459 | 162 | 277 | PEG-0484 | 165 | 275 | PEG-0509 | 168 | 273 |
| PEG-0435 | 160 | 271 | PEG-0460 | 163 | 269 | PEG-0485 | 165 | 276 | PEG-0510 | 168 | 274 |
| PEG-0436 | 160 | 272 | PEG-0461 | 163 | 270 | PEG-0486 | 165 | 277 | PEG-0511 | 168 | 275 |
| PEG-0437 | 160 | 273 | PEG-0462 | 163 | 271 | PEG-0487 | 166 | 269 | PEG-0512 | 168 | 276 |
| PEG-0438 | 160 | 274 | PEG-0463 | 163 | 272 | PEG-0488 | 166 | 270 | PEG-0513 | 168 | 277 |
| PEG-0439 | 160 | 275 | PEG-0464 | 163 | 273 | PEG-0489 | 166 | 271 | PEG-4001 | 3001 | 270 |
| PEG-4002 | 3001 | 272 | PEG-4003 | 3001 | 274 | PEG-4004 | 3001 | 276 | | | |

TABLE 13a
Exemplary RTT sequences for Spacer S05

| SEQ ID NO: | Sequence | SEQ ID NO: | Sequence |
|---|---|---|---|
| 169 | UgGUUUCUCCUCGUAUGGC | 172 | AUUUgGUUUCUCCUCGUAUGGCCCCAU |
| | CCCAUUGCCCUGU | | UGCCCUGU |
| 170 | UUgGUUUCUCCUCGUAUGG | 173 | UAUUUgGUUUCUCCUCGUAUGGCCCCA |
| | CCCCAUUGCCCUGU | | UUGCCCUGU |
| 171 | UUUgGUUUCUCCUCGUAUG | 174 | GUAUUUgGUUUCUCCUCGUAUGGCCCC |
| | GCCCCAUUGCCCUGU | | AUUGCCCUGU |

TABLE 13b
Exemplary PBS sequences for Spacer S05

| | Sequence |
|---|---|
| SEQ REF NO: 278 | UUGGAGU |
| SEQ REF NO: 279 | UUGGAGUC |
| SEQ REF NO: 280 | UUGGAGUCA |
| SEQ ID NO: 281 | UUGGAGUCAU |
| SEQ ID NO: 282 | UUGGAGUCAUA |
| SEQ ID NO: 283 | UUGGAGUCAUAG |
| SEQ ID NO: 284 | UUGGAGUCAUAGC |
| SEQ ID NO: 285 | UUGGAGUCAUAGCC |
| SEQ ID NO: 286 | UUGGAGUCAUAGCCA |

TABLE 13c
Exemplary RTT/PBS Combinations with Spacer S05

| PEG # | RTT | PBS |
|---|---|---|
| PEG-0514 | 169 | 278 |
| PEG-0515 | 169 | 279 |
| PEG-0516 | 169 | 280 |
| PEG-0517 | 169 | 281 |
| PEG-0518 | 169 | 282 |
| PEG-0519 | 169 | 283 |
| PEG-0520 | 169 | 284 |
| PEG-0521 | 169 | 285 |
| PEG-0522 | 169 | 286 |
| PEG-0523 | 170 | 278 |
| PEG-0524 | 170 | 279 |
| PEG-0525 | 170 | 280 |
| PEG-0526 | 170 | 281 |
| PEG-0527 | 170 | 282 |
| PEG-0528 | 170 | 283 |
| PEG-0529 | 170 | 284 |
| PEG-0530 | 170 | 285 |
| PEG-0531 | 170 | 286 |
| PEG-0532 | 171 | 278 |
| PEG-0533 | 171 | 279 |
| PEG-0534 | 171 | 280 |
| PEG-0535 | 171 | 281 |
| PEG-0536 | 171 | 282 |
| PEG-0537 | 171 | 283 |
| PEG-0538 | 171 | 284 |
| PEG-0539 | 171 | 285 |
| PEG-0540 | 171 | 286 |
| PEG-0541 | 172 | 278 |
| PEG-0542 | 172 | 279 |
| PEG-0543 | 172 | 280 |
| PEG-0544 | 172 | 281 |
| PEG-0545 | 172 | 282 |
| PEG-0546 | 172 | 283 |
| PEG-0547 | 172 | 284 |
| PEG-0548 | 172 | 285 |
| PEG-0549 | 172 | 286 |
| PEG-0550 | 173 | 278 |
| PEG-0551 | 173 | 279 |
| PEG-0552 | 173 | 280 |
| PEG-0553 | 173 | 281 |
| PEG-0554 | 173 | 282 |
| PEG-0555 | 173 | 283 |
| PEG-0556 | 173 | 284 |
| PEG-0557 | 173 | 285 |
| PEG-0558 | 173 | 286 |
| PEG-0559 | 174 | 278 |
| PEG-0560 | 174 | 279 |
| PEG-0561 | 174 | 280 |
| PEG-0562 | 174 | 281 |
| PEG-0563 | 174 | 282 |
| PEG-0564 | 174 | 283 |
| PEG-0565 | 174 | 284 |
| PEG-0566 | 174 | 285 |
| PEG-0567 | 174 | 286 |

TABLE 14a
Exemplary RTT sequences for Spacer S06

| SEQ ID | | SEQ ID | |
|---|---|---|---|
| NO: | Sequence | NO: | Sequence |
| 175 | UgGUUUCUCCUCGUAUGGCCCC | 178 | AUUUgGUUUCUCCUCGUAUGGCCCCAU |
| | AUUGCCCUGUU | | UGCCCUGUU |
| 176 | UUgGUUUCUCCUCGUAUGGCCC | 179 | UAUUUgGUUUCUCCUCGUAUGGCCCCA |
| | CAUUGCCCUGUU | | UUGCCCUGUU |
| 177 | UUUgGUUUCUCCUCGUAUGGCC | | |
| | CCAUUGCCCUGUU | | |

TABLE 14b
Exemplary PBS sequences for Spacer S06

| | Sequence |
|---|---|
| SEQ REF NO: 287 | UGGAGUC |
| SEQ REF NO: 288 | UGGAGUCA |
| SEQ REF NO: 289 | UGGAGUCAU |
| SEQ ID NO: 290 | UGGAGUCAUA |
| SEQ ID NO: 291 | UGGAGUCAUAG |
| SEQ ID NO: 292 | UGGAGUCAUAGC |
| SEQ ID NO: 293 | UGGAGUCAUAGCC |
| SEQ ID NO: 294 | UGGAGUCAUAGCCA |
| SEQ ID NO: 295 | UGGAGUCAUAGCCAA |

TABLE 14c
Exemplary RTT/PBS Combinations with Spacer S06

| PEG # | RTT | PBS |
|---|---|---|
| PEG-0568 | 175 | 287 |
| PEG-0569 | 175 | 288 |
| PEG-0570 | 175 | 289 |
| PEG-0571 | 175 | 290 |
| PEG-0572 | 175 | 291 |
| PEG-0573 | 175 | 292 |
| PEG-0574 | 175 | 293 |
| PEG-0575 | 175 | 294 |
| PEG-0576 | 175 | 295 |
| PEG-0577 | 176 | 287 |
| PEG-0578 | 176 | 288 |
| PEG-0579 | 176 | 289 |
| PEG-0580 | 176 | 290 |
| PEG-0581 | 176 | 291 |
| PEG-0582 | 176 | 292 |
| PEG-0583 | 176 | 293 |
| PEG-0584 | 176 | 294 |
| PEG-0585 | 176 | 295 |
| PEG-0586 | 177 | 287 |
| PEG-0587 | 177 | 288 |
| PEG-0588 | 177 | 289 |
| PEG-0589 | 177 | 290 |
| PEG-0590 | 177 | 291 |
| PEG-0591 | 177 | 292 |
| PEG-0592 | 177 | 293 |
| PEG-0593 | 177 | 294 |
| PEG-0594 | 177 | 295 |
| PEG-0595 | 178 | 287 |
| PEG-0596 | 178 | 288 |
| PEG-0597 | 178 | 289 |
| PEG-0598 | 178 | 290 |
| PEG-0599 | 178 | 291 |
| PEG-0600 | 178 | 292 |
| PEG-0601 | 178 | 293 |
| PEG-0602 | 178 | 294 |
| PEG-0603 | 178 | 295 |
| PEG-0604 | 179 | 287 |
| PEG-0605 | 179 | 288 |
| PEG-0606 | 179 | 289 |
| PEG-0607 | 179 | 290 |
| PEG-0608 | 179 | 291 |
| PEG-0609 | 179 | 292 |
| PEG-0610 | 179 | 293 |
| PEG-0611 | 179 | 294 |
| PEG-0612 | 179 | 295 |

TABLE 15a
Exemplary RTT sequences for Spacer S07

| SEQ ID | | SEQ ID | |
|---|---|---|---|
| NO: | Sequence | NO: | Sequence |
| 180 | ACCAAAUACAGCUCCCAAU | 188 | GAGGAGAAACCAAAUACAGCUCCCA |
| | | | AU |
| 181 | AACCAAAUACAGCUCCCAAU | 189 | ACGAGGAGAAACCAAAUACAGCUCC |
| | | | CAAU |
| 182 | AAACCAAAUACAGCUCCCAA | 190 | UACGAGGAGAAACCAAAUACAGCUC |
| | U | | CCAAU |
| 183 | GAAACCAAAUACAGCUCCCA | 191 | AUACGAGGAGAAACCAAAUACAGCU |
| | AU | | CCCAAU |
| 184 | AGAAACCAAAUACAGCUCCC | 192 | GCCAUACGAGGAGAAACCAAAUACA |
| | AAU | | GCUCCCAAU |
| 185 | GAGAAACCAAAUACAGCUCC | 193 | GGCCAUACGAGGAGAAACCAAAUAC |
| | CAAU | | AGCUCCCAAU |
| 186 | GGAGAAACCAAAUACAGCUC | 194 | GGGCCAUACGAGGAGAAACCAAAUA |
| | CCAAU | | CAGCUCCCAAU |
| 187 | AGGAGAAACCAAAUACAGCU | | |
| | CCCAAU | | |

TABLE 15b
Exemplary PBS sequences for Spacer S07

| | Sequence |
|---|---|
| SEQ REF NO: 296 | ACCAGGA |
| SEQ REF NO: 297 | ACCAGGAU |
| SEQ REF NO: 298 | ACCAGGAUC |
| SEQ ID NO: 299 | ACCAGGAUCC |
| SEQ ID NO: 300 | ACCAGGAUCCA |
| SEQ ID NO: 301 | ACCAGGAUCCAG |
| SEQ ID NO: 302 | ACCAGGAUCCAGA |
| SEQ ID NO: 303 | ACCAGGAUCCAGAG |
| SEQ ID NO: 304 | ACCAGGAUCCAGAGC |

TABLE 15c
Exemplary RTT/PBS Combinations with Spacer S07

| PEG # | RTT | PBS |
|---|---|---|
| PEG-0781 | 180 | 296 |
| PEG-0782 | 180 | 297 |
| PEG-0783 | 180 | 298 |
| PEG-0784 | 180 | 299 |
| PEG-0785 | 180 | 300 |
| PEG-0786 | 180 | 301 |
| PEG-0787 | 180 | 302 |
| PEG-0788 | 180 | 303 |
| PEG-0789 | 180 | 304 |
| PEG-0790 | 181 | 296 |
| PEG-0791 | 181 | 297 |
| PEG-0792 | 181 | 298 |
| PEG-0793 | 181 | 299 |
| PEG-0794 | 181 | 300 |
| PEG-0795 | 181 | 301 |
| PEG-0796 | 181 | 302 |
| PEG-0797 | 181 | 303 |
| PEG-0798 | 181 | 304 |
| PEG-0799 | 182 | 296 |
| PEG-0800 | 182 | 297 |
| PEG-0801 | 182 | 298 |
| PEG-0802 | 182 | 299 |
| PEG-0803 | 182 | 300 |
| PEG-0804 | 182 | 301 |
| PEG-0805 | 182 | 302 |
| PEG-0806 | 182 | 303 |
| PEG-0807 | 182 | 304 |
| PEG-0808 | 183 | 296 |
| PEG-0809 | 183 | 297 |
| PEG-0810 | 183 | 298 |
| PEG-0811 | 183 | 299 |
| PEG-0812 | 183 | 300 |
| PEG-0813 | 183 | 301 |
| PEG-0814 | 183 | 302 |
| PEG-0815 | 183 | 303 |
| PEG-0816 | 183 | 304 |
| PEG-0817 | 184 | 296 |
| PEG-0818 | 184 | 297 |
| PEG-0819 | 184 | 298 |
| PEG-0820 | 184 | 299 |
| PEG-0821 | 184 | 300 |
| PEG-0822 | 184 | 301 |
| PEG-0823 | 184 | 302 |
| PEG-0824 | 184 | 303 |
| PEG-0825 | 184 | 304 |
| PEG-0826 | 185 | 296 |
| PEG-0827 | 185 | 297 |
| PEG-0828 | 185 | 298 |
| PEG-0829 | 185 | 299 |
| PEG-0830 | 185 | 300 |
| PEG-0831 | 185 | 301 |
| PEG-0832 | 185 | 302 |
| PEG-0833 | 185 | 303 |
| PEG-0834 | 185 | 304 |
| PEG-0835 | 186 | 296 |
| PEG-0836 | 186 | 297 |
| PEG-0837 | 186 | 298 |
| PEG-0838 | 186 | 299 |
| PEG-0839 | 186 | 300 |
| PEG-0840 | 186 | 301 |
| PEG-0841 | 186 | 302 |
| PEG-0842 | 186 | 303 |
| PEG-0843 | 186 | 304 |
| PEG-0844 | 187 | 296 |
| PEG-0845 | 187 | 297 |
| PEG-0846 | 187 | 298 |
| PEG-0847 | 187 | 299 |
| PEG-0848 | 187 | 300 |
| PEG-0849 | 187 | 301 |
| PEG-0850 | 187 | 302 |
| PEG-0851 | 187 | 303 |
| PEG-0852 | 187 | 304 |
| PEG-0853 | 188 | 296 |
| PEG-0854 | 188 | 297 |
| PEG-0855 | 188 | 298 |
| PEG-0856 | 188 | 299 |
| PEG-0857 | 188 | 300 |
| PEG-0858 | 188 | 301 |
| PEG-0859 | 188 | 302 |
| PEG-0860 | 188 | 303 |
| PEG-0861 | 188 | 304 |
| PEG-0862 | 189 | 296 |
| PEG-0863 | 189 | 297 |
| PEG-0864 | 189 | 298 |
| PEG-0865 | 189 | 299 |
| PEG-0866 | 189 | 300 |
| PEG-0867 | 189 | 301 |
| PEG-0868 | 189 | 302 |
| PEG-0869 | 189 | 303 |
| PEG-0870 | 189 | 304 |
| PEG-0871 | 190 | 296 |
| PEG-0872 | 190 | 297 |
| PEG-0873 | 190 | 298 |
| PEG-0874 | 190 | 299 |
| PEG-0875 | 190 | 300 |
| PEG-0876 | 190 | 301 |
| PEG-0877 | 190 | 302 |
| PEG-0878 | 190 | 303 |
| PEG-0879 | 190 | 304 |
| PEG-0880 | 191 | 296 |
| PEG-0881 | 191 | 297 |
| PEG-0882 | 191 | 298 |
| PEG-0883 | 191 | 299 |
| PEG-0884 | 191 | 300 |
| PEG-0885 | 191 | 301 |
| PEG-0886 | 191 | 302 |
| PEG-0887 | 191 | 303 |
| PEG-0888 | 191 | 304 |
| PEG-0889 | 192 | 296 |
| PEG-0890 | 192 | 297 |
| PEG-0891 | 192 | 298 |
| PEG-0892 | 192 | 299 |
| PEG-0893 | 192 | 300 |
| PEG-0894 | 192 | 301 |
| PEG-0895 | 192 | 302 |
| PEG-0896 | 192 | 303 |
| PEG-0897 | 192 | 304 |
| PEG-0898 | 193 | 296 |
| PEG-0899 | 193 | 297 |
| PEG-0900 | 193 | 298 |
| PEG-0901 | 193 | 299 |
| PEG-0902 | 193 | 300 |
| PEG-0903 | 193 | 301 |
| PEG-0904 | 193 | 302 |
| PEG-0905 | 193 | 303 |
| PEG-0906 | 193 | 304 |
| PEG-0907 | 194 | 296 |
| PEG-0908 | 194 | 297 |
| PEG-0909 | 194 | 298 |
| PEG-0910 | 194 | 299 |
| PEG-0911 | 194 | 300 |
| PEG-0912 | 194 | 301 |
| PEG-0913 | 194 | 302 |
| PEG-0914 | 194 | 303 |
| PEG-0915 | 194 | 304 |
| PEG-0916 | 195 | 296 |
| PEG-0917 | 195 | 297 |
| PEG-0918 | 195 | 298 |
| PEG-0919 | 195 | 299 |
| PEG-0920 | 195 | 300 |
| PEG-0921 | 195 | 301 |
| PEG-0922 | 195 | 302 |
| PEG-0923 | 195 | 303 |
| PEG-0924 | 195 | 304 |

TABLE 16a
Exemplary RTT sequences for Spacer S08

| SEQ ID | | SEQ ID | |
|---|---|---|---|
| NO: | Sequence | NO: | Sequence |
| 196 | ACCAAAUACAGCU | 211 | GGAGAAACCAAAUACAGCUCCa |
| | CCCAAUA | | AAUA |
| 197 | AACCAAAUACAGC | 212 | AGGAGAAACCAAAUACAGCUCC |
| | UCCCAAUA | | CAAUA |
| 198 | AAACCAAAUACAG | 213 | GAGGAGAAACCAAAUACAGCUC |
| | CUCCCAAUA | | CCAAUA |
| 199 | AAACCAAAUACAG | 214 | GAGGAGAAACCAAAUACAGCUC |
| | CUCCuAAUA | | CuAAUA |
| 200 | AAACCAAAUACAG | 215 | GAGGAGAAACCAAAUACAGCUC |
| | CUCCgAAUA | | CgAAUA |
| 201 | AAACCAAAUACAG | 216 | GAGGAGAAACCAAAUACAGCUC |
| | CUCCaAAUA | | CaAAUA |
| 202 | GAAACCAAAUACA | 217 | ACGAGGAGAAACCAAAUACAGC |
| | GCUCCCAAUA | | UCCCAAUA |
| 203 | AGAAACCAAAUAC | 219 | UACGAGGAGAAACcAAAUACAG |
| | AGCUCCCAAUA | | CUCCuAAUA |
| 204 | AGAAACCAAAUAC | 220 | UACGAGGAGAAACCAAAUACAG |
| | AGCUCCuAAUA | | CUCCgAAUA |
| 205 | AGAAACCAAAUAC | 221 | UACGAGGAGAAACCAAAUACAG |
| | AGCUCCgAAUA | | CUCCaAAUA |
| 206 | AGAAACCAAAUAC | 222 | AUACGAGGAGAAACCAAAUACA |
| | AGCUCCaAAUA | | GCUCCCAAUA |
| 207 | GAGAAACCAAAUA | 223 | GCCAUACGAGGAGAAACCAAAU |
| | CAGCUCCCAAUA | | ACAGCUCCCAAUA |
| 208 | GGAGAAACCAAAU | 224 | GGCCAUACGAGGAGAAACCAAA |
| | ACAGCUCCCAAUA | | UACAGCUCCCAAUA |
| 209 | GGAGAAACCAAAU | 225 | GGGCCAUACGAGGAGAAACCAA |
| | ACAGCUCCuAAUA | | AUACAGCUCCCAAUA |
| 210 | GGAGAAACCAAAU | 3007 | GAGAAACCAAAUACAGCUCCG |
| | ACAGCUCCgAAUA | | AGUA |
| 6086 | GAAACCAAAUACA | 3008 | GAGAAACCAAAUACAGCUCCA |
| | GCUCCUAAUA | | AGUA |
| 3002 | GAAACCAAAUACA | 3009 | AGGAGAAACCAAAUACAGCUC |
| | GCUCCGAGUA | | CUAAUA |
| 6087 | GAAACCAAAUACA | 3010 | AGGAGAAACCAAAUACAGCUC |
| | GCUCCGAGUA | | CGAGUA |
| 6088 | GAAACCAAAUACA | 3011 | AGGAGAAACCAAAUACAGCUC |
| | GCUCCCAAUA | | CAAGUA |
| 6089 | GAAACCAAAUACA | 3007 | GAGAAACCAAAUACAGCUCCG |
| | GCUCCAAGUA | | AGUA |
| 3006 | GAGAAACCAAAUA | | |
| | CAGCUCCUAAUA | | |

TABLE 16b
Exemplary PBS sequences for Spacer S08

| | Sequence |
|---|---|
| SEQ REF NO: 305 | CCAGGAU |
| SEQ REF NO: 306 | CCAGGAUC |
| SEQ REF NO: 307 | CCAGGAUCC |
| SEQ ID NO: 308 | CCAGGAUCCA |
| SEQ ID NO: 309 | CCAGGAUCCAG |
| SEQ ID NO: 310 | CCAGGAUCCAGA |
| SEQ ID NO: 311 | CCAGGAUCCAGAG |
| SEQ ID NO: 312 | CCAGGAUCCAGAGC |
| SEQ ID NO: 313 | CCAGGAUCCAGAGCU |

TABLE 16c
Exemplary RTT/PBS Combinations with Spacer S08

| PEG # | RTT | PBS | PEG # | RTT | PBS | PEG # | RTT | PBS | PEG # | RTT | PBS |
|---|---|---|---|---|---|---|---|---|---|---|---|
| PEG-0925 | 196 | 305 | PEG-0962 | 203 | 306 | PEG-1255 | 211 | 312 | PEG-1240 | 219 | 306 |
| PEG-0926 | 196 | 306 | PEG-0963 | 203 | 307 | PEG-0988 | 212 | 305 | PEG-1241 | 219 | 308 |
| PEG-0927 | 196 | 307 | PEG-0964 | 203 | 308 | PEG-0989 | 212 | 306 | PEG-1242 | 219 | 310 |
| PEG-0928 | 196 | 308 | PEG-0965 | 203 | 309 | PEG-0990 | 212 | 307 | PEG-1243 | 219 | 312 |
| PEG-0929 | 196 | 309 | PEG-0966 | 203 | 310 | PEG-0991 | 212 | 308 | PEG-1220 | 220 | 306 |
| PEG-0930 | 196 | 310 | PEG-0967 | 203 | 311 | PEG-0992 | 212 | 309 | PEG-1221 | 220 | 308 |
| PEG-0931 | 196 | 311 | PEG-0968 | 203 | 312 | PEG-0993 | 212 | 310 | PEG-1222 | 220 | 310 |
| PEG-0932 | 196 | 312 | PEG-0969 | 203 | 313 | PEG-0994 | 212 | 311 | PEG-1223 | 220 | 312 |
| PEG-0933 | 196 | 313 | PEG-1228 | 204 | 306 | PEG-0995 | 212 | 312 | PEG-1260 | 221 | 306 |
| PEG-0934 | 197 | 305 | PEG-1229 | 204 | 308 | PEG-0996 | 212 | 313 | PEG-1261 | 221 | 308 |
| PEG-0935 | 197 | 306 | PEG-1230 | 204 | 310 | PEG-0997 | 213 | 305 | PEG-1262 | 221 | 310 |
| PEG-0936 | 197 | 307 | PEG-1231 | 204 | 312 | PEG-0998 | 213 | 306 | PEG-1263 | 221 | 312 |
| PEG-0937 | 197 | 308 | PEG-1208 | 205 | 306 | PEG-0999 | 213 | 307 | PEG-1024 | 222 | 305 |
| PEG-0938 | 197 | 309 | PEG-1209 | 205 | 308 | PEG-1000 | 213 | 308 | PEG-1025 | 222 | 306 |
| PEG-0939 | 197 | 310 | PEG-1210 | 205 | 310 | PEG-1001 | 213 | 309 | PEG-1026 | 222 | 307 |
| PEG-0940 | 197 | 311 | PEG-1211 | 205 | 312 | PEG-1002 | 213 | 310 | PEG-1027 | 222 | 308 |
| PEG-0941 | 197 | 312 | PEG-1248 | 206 | 306 | PEG-1003 | 213 | 311 | PEG-1028 | 222 | 309 |
| PEG-0942 | 197 | 313 | PEG-1249 | 206 | 308 | PEG-1004 | 213 | 312 | PEG-1029 | 222 | 310 |
| PEG-0943 | 198 | 305 | PEG-1250 | 206 | 310 | PEG-1005 | 213 | 313 | PEG-1030 | 222 | 311 |
| PEG-0944 | 198 | 306 | PEG-1251 | 206 | 312 | PEG-1236 | 214 | 306 | PEG-1031 | 222 | 312 |
| PEG-0945 | 198 | 307 | PEG-0970 | 207 | 305 | PEG-1237 | 214 | 308 | PEG-1032 | 222 | 313 |
| PEG-0946 | 198 | 308 | PEG-0971 | 207 | 306 | PEG-1238 | 214 | 310 | PEG-1033 | 223 | 305 |
| PEG-0947 | 198 | 309 | PEG-0972 | 207 | 307 | PEG-1239 | 214 | 312 | PEG-1034 | 223 | 306 |
| PEG-0948 | 198 | 310 | PEG-0973 | 207 | 308 | PEG-1216 | 215 | 306 | PEG-1035 | 223 | 307 |
| PEG-0949 | 198 | 311 | PEG-0974 | 207 | 309 | PEG-1217 | 215 | 308 | PEG-1036 | 223 | 308 |
| PEG-0950 | 198 | 312 | PEG-0975 | 207 | 310 | PEG-1218 | 215 | 310 | PEG-1037 | 223 | 309 |
| PEG-0951 | 198 | 313 | PEG-0976 | 207 | 311 | PEG-1219 | 215 | 312 | PEG-1038 | 223 | 310 |
| PEG-1224 | 199 | 306 | PEG-0977 | 207 | 312 | PEG-1256 | 216 | 306 | PEG-1039 | 223 | 311 |
| PEG-1225 | 199 | 308 | PEG-0978 | 207 | 313 | PEG-1257 | 216 | 308 | PEG-1040 | 223 | 312 |
| PEG-1226 | 199 | 310 | PEG-0979 | 208 | 305 | PEG-1258 | 216 | 310 | PEG-1041 | 223 | 313 |
| PEG-1227 | 199 | 312 | PEG-0980 | 208 | 306 | PEG-1259 | 216 | 312 | PEG-1042 | 224 | 305 |
| PEG-1204 | 200 | 306 | PEG-0981 | 208 | 307 | PEG-1006 | 217 | 305 | PEG-1043 | 224 | 306 |
| PEG-1205 | 200 | 308 | PEG-0982 | 208 | 308 | PEG-1007 | 217 | 306 | PEG-1044 | 224 | 307 |
| PEG-1206 | 200 | 310 | PEG-0983 | 208 | 309 | PEG-1008 | 217 | 307 | PEG-1045 | 224 | 308 |
| PEG-1207 | 200 | 312 | PEG-0984 | 208 | 310 | PEG-1009 | 217 | 308 | PEG-1046 | 224 | 309 |
| PEG-1244 | 201 | 306 | PEG-0985 | 208 | 311 | PEG-1010 | 217 | 309 | PEG-1047 | 224 | 310 |
| PEG-1245 | 201 | 308 | PEG-0986 | 208 | 312 | PEG-1011 | 217 | 310 | PEG-1048 | 224 | 311 |
| PEG-1246 | 201 | 310 | PEG-0987 | 208 | 313 | PEG-1012 | 217 | 311 | PEG-1049 | 224 | 312 |
| PEG-1247 | 201 | 312 | PEG-1232 | 209 | 306 | PEG-1013 | 217 | 312 | PEG-1050 | 224 | 313 |
| PEG-0952 | 202 | 305 | PEG-1233 | 209 | 308 | PEG-1014 | 217 | 313 | PEG-1051 | 225 | 305 |
| PEG-0953 | 202 | 306 | PEG-1234 | 209 | 310 | PEG-1015 | 218 | 305 | PEG-1052 | 225 | 306 |
| PEG-0954 | 202 | 307 | PEG-1235 | 209 | 312 | PEG-1016 | 218 | 306 | PEG-1053 | 225 | 307 |
| PEG-0955 | 202 | 308 | PEG-1212 | 210 | 306 | PEG-1017 | 218 | 307 | PEG-1054 | 225 | 308 |
| PEG-0956 | 202 | 309 | PEG-1213 | 210 | 308 | PEG-1018 | 218 | 308 | PEG-1055 | 225 | 309 |
| PEG-0957 | 202 | 310 | PEG-1214 | 210 | 310 | PEG-1019 | 218 | 309 | PEG-1056 | 225 | 310 |
| PEG-0958 | 202 | 311 | PEG-1215 | 210 | 312 | PEG-1020 | 218 | 310 | PEG-1057 | 225 | 311 |
| PEG-0959 | 202 | 312 | PEG-1252 | 211 | 306 | PEG-1021 | 218 | 311 | PEG-1058 | 225 | 312 |
| PEG-0960 | 202 | 313 | PEG-1253 | 211 | 308 | PEG-1022 | 218 | 312 | PEG-1059 | 225 | 313 |
| PEG-0961 | 203 | 305 | PEG-1254 | 211 | 310 | PEG-1023 | 218 | 313 | PEG-4038 | 204 | 311 |
| PEG-4013 | 3001 | 306 | PEG-4087 | 3010 | 307 | PEG-4062 | 3008 | 309 | PEG-4103 | 205 | 311 |
| PEG-4079 | 3002 | 306 | PEG-4054 | 3011 | 307 | PEG-4030 | 209 | 309 | PEG-4071 | 206 | 311 |
| PEG-4046 | 3005 | 306 | PEG-4022 | 214 | 307 | PEG-4095 | 210 | 309 | PEG-4039 | 3006 | 311 |
| PEG-4014 | 3006 | 306 | PEG-4088 | 215 | 307 | PEG-4063 | 211 | 309 | PEG-4104 | 3007 | 311 |
| PEG-4080 | 3007 | 306 | PEG-4055 | 216 | 307 | PEG-4031 | 3009 | 309 | PEG-4072 | 3008 | 311 |
| PEG-4047 | 3008 | 306 | PEG-4023 | 300 | 308 | PEG-4096 | 3010 | 309 | PEG-4040 | 209 | 311 |
| PEG-4015 | 3009 | 306 | PEG-4089 | 3002 | 308 | PEG-4064 | 3011 | 309 | PEG-4105 | 210 | 311 |
| PEG-4081 | 3010 | 306 | PEG-4056 | 3005 | 308 | PEG-4032 | 214 | 309 | PEG-4073 | 211 | 311 |
| PEG-4048 | 3011 | 306 | PEG-4024 | 3006 | 308 | PEG-4097 | 215 | 309 | PEG-4041 | 3009 | 311 |
| PEG-4016 | 199 | 307 | PEG-4090 | 3007 | 308 | PEG-4065 | 216 | 309 | PEG-4106 | 3010 | 311 |
| PEG-4082 | 200 | 307 | PEG-4057 | 3008 | 308 | PEG-4033 | 3001 | 310 | PEG-4074 | 3011 | 311 |
| PEG-4049 | 201 | 307 | PEG-4025 | 3009 | 308 | PEG-4098 | 3002 | 310 | PEG-4042 | 214 | 311 |
| PEG-4017 | 3001 | 307 | PEG-4091 | 3010 | 308 | PEG-4066 | 3005 | 310 | PEG-4107 | 215 | 311 |
| PEG-4083 | 3002 | 307 | PEG-4058 | 3011 | 308 | PEG-4034 | 3006 | 310 | PEG-4075 | 216 | 311 |
| PEG-4050 | 3005 | 307 | PEG-4026 | 199 | 309 | PEG-4099 | 3007 | 310 | PEG-4043 | 3001 | 312 |
| PEG-4018 | 204 | 307 | PEG-4092 | 200 | 309 | PEG-4067 | 3008 | 310 | PEG-4012 | 3002 | 312 |
| PEG-4084 | 205 | 307 | PEG-4059 | 201 | 309 | PEG-4035 | 3009 | 310 | PEG-4076 | 3005 | 312 |
| PEG-4051 | 206 | 307 | PEG-4027 | 3001 | 309 | PEG-4100 | 3010 | 310 | PEG-4044 | 3006 | 312 |
| PEG-4019 | 3006 | 307 | PEG-4011 | 3002 | 309 | PEG-4068 | 3011 | 310 | PEG-4108 | 3007 | 312 |
| PEG-4085 | 3007 | 307 | PEG-4060 | 3005 | 309 | PEG-4036 | 199 | 311 | PEG-4077 | 3008 | 312 |
| PEG-4052 | 3008 | 307 | PEG-4028 | 204 | 309 | PEG-4101 | 200 | 311 | PEG-4045 | 3009 | 312 |
| PEG-4020 | 209 | 307 | PEG-4093 | 205 | 309 | PEG-4069 | 201 | 311 | PEG-4109 | 3010 | 312 |
| PEG-4086 | 210 | 307 | PEG-4061 | 206 | 309 | PEG-4037 | 3001 | 311 | PEG-4078 | 3011 | 312 |
| PEG-4053 | 211 | 307 | PEG-4029 | 3006 | 309 | PEG-4102 | 3002 | 311 | | | |
| PEG-4021 | 3009 | 307 | PEG-4094 | 3007 | 309 | PEG-4070 | 3005 | 311 | | | |

TABLE 17a
Exemplary RTT sequences for Spacer S09

| SEQ | | SEQ ID | |
|---|---|---|---|
| ID NO: | Sequence | NO: | Sequence |
| 226 | ACCAAAUACAGCUCCCAAUACCAG | 232 | GGAGAAACcAAAUACAGCUCCCAAUA |
| | GA | | CCAGGA |
| 227 | AACCAAAUACAGCUCCCAAUACCAG | 233 | AGGAGAAACCAAAUACAGCUCCCAAU |
| | GA | | ACCAGGA |
| 228 | AAACcAAAUACAGCUCCCAAUACCA | 234 | GAGGAGAAACCAAAUACAGCUCCCAA |
| | GGA | | UACCAGGA |
| 229 | GAAACcAAAUACAGCUCCCAAUACC | 235 | ACGAGGAGAAACCAAAUACAGCUCCC |
| | AGGA | | AAUACCAGGA |
| 230 | AGAAACcAAAUACAGCUCCCAAUAC | 236 | UACGAGGAGAAACCAAAUACAGCUCC |
| | CAGGA | | CAAUACCAGGA |
| 231 | GAGAAACcAAAUACAGCUCCCAAUA | | |
| | CCAGGA | | |

TABLE 17b
Exemplary PBS sequences for Spacer S09

| | Sequence |
|---|---|
| SEQ REF NO: 314 | UCCAGAG |
| SEQ REF NO: 315 | UCCAGAGC |
| SEQ REF NO: 316 | UCCAGAGCU |
| SEQ ID NO: 317 | UCCAGAGCUG |
| SEQ ID NO: 318 | UCCAGAGCUGC |
| SEQ ID NO: 319 | UCCAGAGCUGCC |
| SEQ ID NO: 320 | UCCAGAGCUGCCA |
| SEQ ID NO: 321 | UCCAGAGCUGCCAA |
| SEQ ID NO: 322 | UCCAGAGCUGCCAAG |

TABLE 17c
Exemplary RTT/PBS Combinations with Spacer S09

| PEG # | RTT | PBS |
|---|---|---|
| PEG-1060 | 226 | 314 |
| PEG-1061 | 226 | 315 |
| PEG-1062 | 226 | 316 |
| PEG-1063 | 226 | 317 |
| PEG-1064 | 226 | 318 |
| PEG-1065 | 226 | 319 |
| PEG-1066 | 226 | 320 |
| PEG-1067 | 226 | 321 |
| PEG-1068 | 226 | 322 |
| PEG-1069 | 227 | 314 |
| PEG-1070 | 227 | 315 |
| PEG-1071 | 227 | 316 |
| PEG-1072 | 227 | 317 |
| PEG-1073 | 227 | 318 |
| PEG-1074 | 227 | 319 |
| PEG-1075 | 227 | 320 |
| PEG-1076 | 227 | 321 |
| PEG-1077 | 227 | 322 |
| PEG-1078 | 228 | 314 |
| PEG-1079 | 228 | 315 |
| PEG-1080 | 228 | 316 |
| PEG-1081 | 228 | 317 |
| PEG-1082 | 228 | 318 |
| PEG-1083 | 228 | 319 |
| PEG-1084 | 228 | 320 |
| PEG-1085 | 228 | 321 |
| PEG-1086 | 228 | 322 |
| PEG-1087 | 229 | 314 |
| PEG-1088 | 229 | 315 |
| PEG-1089 | 229 | 316 |
| PEG-1090 | 229 | 317 |
| PEG-1091 | 229 | 318 |
| PEG-1092 | 229 | 319 |
| PEG-1093 | 229 | 320 |
| PEG-1094 | 229 | 321 |
| PEG-1095 | 229 | 322 |
| PEG-1096 | 230 | 314 |
| PEG-1097 | 230 | 315 |
| PEG-1098 | 230 | 316 |
| PEG-1099 | 230 | 317 |
| PEG-1100 | 230 | 318 |
| PEG-1101 | 230 | 319 |
| PEG-1102 | 230 | 320 |
| PEG-1103 | 230 | 321 |
| PEG-1104 | 230 | 322 |
| PEG-1105 | 231 | 314 |
| PEG-1106 | 231 | 315 |
| PEG-1107 | 231 | 316 |
| PEG-1108 | 231 | 317 |
| PEG-1109 | 231 | 318 |
| PEG-1110 | 231 | 319 |
| PEG-1111 | 231 | 320 |
| PEG-1112 | 231 | 321 |
| PEG-1113 | 231 | 322 |
| PEG-1114 | 232 | 314 |
| PEG-1115 | 232 | 315 |
| PEG-1116 | 232 | 316 |
| PEG-1117 | 232 | 317 |
| PEG-1118 | 232 | 318 |
| PEG-1119 | 232 | 319 |
| PEG-1120 | 232 | 320 |
| PEG-1121 | 232 | 321 |
| PEG-1122 | 232 | 322 |
| PEG-1123 | 233 | 314 |
| PEG-1124 | 233 | 315 |
| PEG-1125 | 233 | 316 |
| PEG-1126 | 233 | 317 |
| PEG-1127 | 233 | 318 |
| PEG-1128 | 233 | 319 |
| PEG-1129 | 233 | 320 |
| PEG-1130 | 233 | 321 |
| PEG-1131 | 233 | 322 |
| PEG-1132 | 234 | 314 |
| PEG-1133 | 234 | 315 |
| PEG-1134 | 234 | 316 |
| PEG-1135 | 234 | 317 |
| PEG-1136 | 234 | 318 |
| PEG-1137 | 234 | 319 |
| PEG-1138 | 234 | 320 |
| PEG-1139 | 234 | 321 |
| PEG-1140 | 234 | 322 |
| PEG-1141 | 235 | 314 |
| PEG-1142 | 235 | 315 |
| PEG-1143 | 235 | 316 |
| PEG-1144 | 235 | 317 |
| PEG-1145 | 235 | 318 |
| PEG-1146 | 235 | 319 |
| PEG-1147 | 235 | 320 |
| PEG-1148 | 235 | 321 |
| PEG-1149 | 235 | 322 |
| PEG-1150 | 236 | 314 |
| PEG-1151 | 236 | 315 |
| PEG-1152 | 236 | 316 |
| PEG-1153 | 236 | 317 |
| PEG-1154 | 236 | 318 |
| PEG-1155 | 236 | 319 |
| PEG-1156 | 236 | 320 |
| PEG-1157 | 236 | 321 |
| PEG-1158 | 236 | 322 |

TABLE 18a
Exemplary RTT sequences for Spacer S10

| SEQ ID NO: | Sequence | SEQ ID NO: | Sequence |
|---|---|---|---|
| 237 | ACCAAAUACAGCUCCCAAU | 240 | GAAACCAAAUACAGCUCCCAAUACC |
| | ACCAGGAUCCAGAG | | AGGAUCCAGAG |
| 238 | AACCAAAUACAGCUCCCAA | 241 | AGAAACCAAAUACAGCUCCCAAUAC |
| | UACCAGGAUCCAGAG | | CAGGAUCCAGAG |
| 239 | AAACCAAAUACAGCUCCCA | | |
| | AUACCAGGAUCCAGAG | | |

TABLE 18b
Exemplary PBS sequences for Spacer S10

| | Sequence |
|---|---|
| SEQ REF NO: 323 | CUGCCAA |
| SEQ REF NO: 324 | CUGCCAAG |
| SEQ REF NO: 325 | CUGCCAAGG |
| SEQ ID NO: 326 | CUGCCAAGGG |
| SEQ ID NO: 327 | CUGCCAAGGGC |
| SEQ ID NO: 328 | CUGCCAAGGGCA |
| SEQ ID NO: 329 | CUGCCAAGGGCAG |
| SEQ ID NO: 330 | CUGCCAAGGGCAGA |
| SEQ ID NO: 331 | CUGCCAAGGGCAGAG |

TABLE 18c
Exemplary RTT/PBS Combinations with Spacer S10

| PEG # | RTT | PBS |
|---|---|---|
| PEG-1159 | 237 | 323 |
| PEG-1160 | 237 | 324 |
| PEG-1161 | 237 | 325 |
| PEG-1162 | 237 | 326 |
| PEG-1163 | 237 | 327 |
| PEG-1164 | 237 | 328 |
| PEG-1165 | 237 | 329 |
| PEG-1166 | 237 | 330 |
| PEG-1167 | 237 | 331 |
| PEG-1168 | 238 | 323 |
| PEG-1169 | 238 | 324 |
| PEG-1170 | 238 | 325 |
| PEG-1171 | 238 | 326 |
| PEG-1172 | 238 | 327 |
| PEG-1173 | 238 | 328 |
| PEG-1174 | 238 | 329 |
| PEG-1175 | 238 | 330 |
| PEG-1176 | 238 | 331 |
| PEG-1177 | 239 | 323 |
| PEG-1178 | 239 | 324 |
| PEG-1179 | 239 | 325 |
| PEG-1180 | 239 | 326 |
| PEG-1181 | 239 | 327 |
| PEG-1182 | 239 | 328 |
| PEG-1183 | 239 | 329 |
| PEG-1184 | 239 | 330 |
| PEG-1185 | 239 | 331 |
| PEG-1186 | 240 | 323 |
| PEG-1187 | 240 | 324 |
| PEG-1188 | 240 | 325 |
| PEG-1189 | 240 | 326 |
| PEG-1190 | 240 | 327 |
| PEG-1191 | 240 | 328 |
| PEG-1192 | 240 | 329 |
| PEG-1193 | 240 | 330 |
| PEG-1194 | 240 | 331 |
| PEG-1195 | 241 | 323 |
| PEG-1196 | 241 | 324 |
| PEG-1197 | 241 | 325 |
| PEG-1198 | 241 | 326 |
| PEG-1199 | 241 | 327 |
| PEG-1200 | 241 | 328 |
| PEG-1201 | 241 | 329 |
| PEG-1202 | 241 | 330 |
| PEG-1203 | 241 | 331 |

TABLE 18d
Correction of G339C using PE2 systems, experiment 1.

| PEG # | Edit % | Indel % |
|---|---|---|
| PEG-0011 | 1% | 1% |
| PEG-0011 | 1% | 1% |
| PEG-0013 | 0% | 1% |
| PEG-0013 | 1% | 1% |
| PEG-0015 | 0% | 0% |
| PEG-0015 | 7% | 15% |
| PEG-0017 | 0% | 0% |
| PEG-0017 | 1% | 1% |
| PEG-0029 | 17% | 1% |
| PEG-0029 | 24% | 15% |
| PEG-0031 | 1% | 0% |
| PEG-0031 | 18% | 2% |
| PEG-0033 | 19% | 2% |
| PEG-0033 | 20% | 2% |
| PEG-0035 | 22% | 2% |
| PEG-0035 | 1% | 0% |
| PEG-0047 | 26% | 1% |
| PEG-0047 | 40% | 15% |
| PEG-0049 | 38% | 2% |
| PEG-0049 | 31% | 4% |
| PEG-0051 | 1% | 0% |
| PEG-0051 | 56% | 2% |
| PEG-0053 | 7% | 1% |
| PEG-0053 | 33% | 4% |
| PEG-0065 | 39% | 2% |
| PEG-0065 | 46% | 3% |
| PEG-0067 | 41% | 2% |
| PEG-0067 | 33% | 1% |
| PEG-0069 | 39% | 4% |
| PEG-0069 | 41% | 6% |
| PEG-0071 | 29% | 2% |
| PEG-0071 | 0% | 0% |
| PEG-0092 | 19% | 2% |
| PEG-0092 | 19% | 2% |
| PEG-0094 | 27% | 2% |
| PEG-0094 | 19% | 1% |
| PEG-0096 | 24% | 3% |
| PEG-0096 | 23% | 1% |
| PEG-0098 | 1% | 0% |
| PEG-0098 | 1% | 0% |
| PEG-0110 | 25% | 4% |
| PEG-0110 | 24% | 2% |
| PEG-0112 | 28% | 2% |
| PEG-0112 | 28% | 1% |
| PEG-0114 | 30% | 4% |
| PEG-0114 | 35% | 2% |
| PEG-0116 | 1% | 0% |
| PEG-0116 | 19% | 1% |
| PEG-0209 | 3% | 1% |
| PEG-0211 | 2% | 1% |
| PEG-0213 | 2% | 1% |
| PEG-0215 | 3% | 1% |
| PEG-0236 | 10% | 1% |
| PEG-0238 | 21% | 3% |
| PEG-0240 | 4% | 0% |
| PEG-0242 | 3% | 0% |
| PEG-0263 | 9% | 2% |
| PEG-0265 | 16% | 2% |
| PEG-0267 | 8% | 1% |
| PEG-0269 | 6% | 0% |
| PEG-0290 | 8% | 1% |
| PEG-0292 | 21% | 2% |
| PEG-0326 | 2% | 1% |
| PEG-0328 | 2% | 0% |
| PEG-0330 | 2% | 1% |
| PEG-0332 | 2% | 1% |
| PEG-0353 | 13% | 1% |
| PEG-0355 | 14% | 1% |
| PEG-0357 | 16% | 1% |
| PEG-0359 | 10% | 1% |
| PEG-0380 | 14% | 1% |
| PEG-0382 | 6% | 0% |
| PEG-0384 | 11% | 1% |
| PEG-0386 | 15% | 1% |
| PEG-0407 | 22% | 1% |
| PEG-0409 | 14% | 1% |
| PEG-0411 | 14% | 1% |
| PEG-0413 | 21% | 1% |
| PEG-0434 | 1% | 1% |
| PEG-0436 | 1% | 0% |
| PEG-0438 | 1% | 1% |
| PEG-0440 | 2% | 1% |
| PEG-0461 | 1% | 0% |
| PEG-0463 | 3% | 1% |
| PEG-0465 | 8% | 1% |
| PEG-0467 | 7% | 1% |
| PEG-0488 | 2% | 0% |
| PEG-0490 | 3% | 0% |
| PEG-0492 | 6% | 1% |
| PEG-0494 | 7% | 0% |
| PEG-0613 | 0% | 1% |
| PEG-0614 | 1% | 1% |
| PEG-0615 | 2% | 3% |
| PEG-0616 | 0% | 0% |
| PEG-0617 | 22% | 3% |
| PEG-0618 | 23% | 4% |
| PEG-0619 | 22% | 3% |
| PEG-0620 | 1% | 0% |
| PEG-0621 | 49% | 3% |
| PEG-0622 | 1% | 0% |
| PEG-0623 | 40% | 2% |
| PEG-0624 | 0% | 0% |
| PEG-0625 | 36% | 2% |
| PEG-0626 | 45% | 2% |
| PEG-0627 | 40% | 4% |
| PEG-0628 | 30% | 1% |
| PEG-0629 | 22% | 3% |
| PEG-0630 | 26% | 1% |
| PEG-0631 | 28% | 5% |
| PEG-0632 | 28% | 1% |
| PEG-0633 | 32% | 3% |
| PEG-0634 | 1% | 0% |
| PEG-0635 | 34% | 3% |
| PEG-0636 | 21% | 1% |
| PEG-0637 | 1% | 1% |
| PEG-0638 | 1% | 1% |
| PEG-0639 | 1% | 1% |
| PEG-0640 | 1% | 0% |
| PEG-0641 | 23% | 2% |
| PEG-0642 | 22% | 2% |
| PEG-0643 | 1% | 0% |
| PEG-0644 | 22% | 1% |
| PEG-0645 | 51% | 4% |
| PEG-0646 | 51% | 3% |
| PEG-0647 | 42% | 5% |
| PEG-0648 | 1% | 0% |
| PEG-0649 | 38% | 3% |
| PEG-0650 | 32% | 2% |
| PEG-0651 | 35% | 3% |
| PEG-0652 | 35% | 2% |
| PEG-0653 | 26% | 3% |
| PEG-0654 | 21% | 1% |
| PEG-0655 | 24% | 1% |
| PEG-0656 | 22% | 2% |
| PEG-0657 | 24% | 1% |
| PEG-0658 | 19% | 1% |
| PEG-0659 | 32% | 3% |
| PEG-0660 | 20% | 1% |
| PEG-0661 | 0% | 1% |
| PEG-0662 | 1% | 2% |
| PEG-0663 | 0% | 2% |
| PEG-0664 | 0% | 0% |
| PEG-0665 | 21% | 3% |
| PEG-0666 | 17% | 2% |
| PEG-0667 | 15% | 1% |
| PEG-0668 | 0% | 0% |
| PEG-0669 | 37% | 2% |
| PEG-0670 | 1% | 0% |
| PEG-0671 | 0% | 0% |
| PEG-0672 | 0% | 0% |
| PEG-0673 | 33% | 2% |
| PEG-0674 | 23% | 1% |
| PEG-0675 | 31% | 4% |
| PEG-0676 | 23% | 1% |
| PEG-0677 | 18% | 2% |
| PEG-0678 | 25% | 1% |
| PEG-0679 | 27% | 2% |
| PEG-0680 | 20% | 1% |
| PEG-0681 | 24% | 3% |
| PEG-0682 | 1% | 0% |
| PEG-0683 | 31% | 3% |
| PEG-0684 | 16% | 0% |
| PEG-0733 | 3% | 1% |
| PEG-0734 | 2% | 1% |
| PEG-0735 | 4% | 2% |
| PEG-0736 | 6% | 2% |
| PEG-0737 | 34% | 2% |
| PEG-0738 | 33% | 2% |
| PEG-0739 | 21% | 1% |
| PEG-0740 | 24% | 2% |
| PEG-0741 | 13% | 2% |
| PEG-0742 | 16% | 1% |
| PEG-0743 | 14% | 1% |
| PEG-0744 | 19% | 3% |
| PEG-0745 | 11% | 1% |
| PEG-0746 | 13% | 1% |
| PEG-0747 | 16% | 2% |
| PEG-0748 | 28% | 2% |
| PEG-0749 | 4% | 1% |
| PEG-0750 | 5% | 2% |
| PEG-0751 | 5% | 1% |
| PEG-0752 | 4% | 2% |
| PEG-0753 | 25% | 1% |
| PEG-0754 | 20% | 1% |
| PEG-0755 | 30% | 1% |
| PEG-0756 | 32% | 2% |
| PEG-0757 | 9% | 1% |
| PEG-0758 | 7% | 1% |
| PEG-0759 | 9% | 1% |
| PEG-0760 | 16% | 1% |
| PEG-0761 | 27% | 1% |
| PEG-0762 | 20% | 1% |
| PEG-0763 | 14% | 1% |
| PEG-0764 | 20% | 1% |
| PEG-0765 | 1% | 1% |
| PEG-0766 | 1% | 1% |
| PEG-0767 | 3% | 2% |
| PEG-0768 | 4% | 3% |
| PEG-0769 | 4% | 0% |
| PEG-0770 | 3% | 0% |
| PEG-0771 | 15% | 1% |
| PEG-0772 | 25% | 3% |
| PEG-0773 | 3% | 0% |
| PEG-0774 | 4% | 0% |
| PEG-0775 | 13% | 2% |
| PEG-0776 | 22% | 4% |
| PEG-0777 | 7% | 1% |
| PEG-0778 | 6% | 1% |
| PEG-0779 | 10% | 1% |
| PEG-0780 | 21% | 2% |
| PEG-0800 | 20% | 1% |
| PEG-0802 | 24% | 2% |
| PEG-0804 | 24% | 2% |
| PEG-0806 | 26% | |
| PEG-0809 | 20% | 6% |
| PEG-0811 | 22% | 2% |
| PEG-0813 | 16% | 5% |
| PEG-0815 | 14% | 2% |
| PEG-0818 | 28% | 3% |
| PEG-0820 | 4% | 0% |
| PEG-0822 | 16% | 1% |
| PEG-0824 | 18% | 1% |
| PEG-0827 | 29% | 1% |
| PEG-0829 | 37% | 2% |
| PEG-0831 | 35% | 2% |
| PEG-0833 | 34% | 1% |
| PEG-0836 | 37% | 1% |
| PEG-0838 | 42% | 2% |
| PEG-0840 | 39% | 4% |
| PEG-0842 | 1% | 0% |
| PEG-0845 | 39% | 2% |
| PEG-0847 | 1% | 0% |
| PEG-0849 | 26% | 2% |
| PEG-0851 | 32% | 1% |
| PEG-0854 | 33% | 2% |
| PEG-0856 | 31% | 1% |
| PEG-0858 | 22% | 2% |
| PEG-0860 | 2% | 0% |
| PEG-0863 | 30% | 2% |
| PEG-0865 | 19% | 1% |
| PEG-0867 | 0% | 0% |
| PEG-0869 | 17% | 1% |
| PEG-0872 | 28% | 1% |
| PEG-0874 | 0% | 0% |
| PEG-0876 | 0% | 0% |
| PEG-0878 | 14% | 1% |
| PEG-0944 | 28% | 1% |
| PEG-0946 | 36% | 1% |
| PEG-0948 | 32% | 2% |
| PEG-0950 | 34% | 1% |
| PEG-0962 | 35% | 1% |
| PEG-0964 | 39% | 2% |
| PEG-0966 | 34% | 4% |
| PEG-0968 | 1% | 0% |
| PEG-0980 | 38% | 2% |
| PEG-0982 | 1% | 0% |
| PEG-0984 | 24% | 2% |
| PEG-0986 | 29% | 1% |
| PEG-0998 | 36% | 2% |
| PEG-1000 | 25% | 2% |
| PEG-1002 | 13% | 1% |
| PEG-1004 | 17% | 1% |
| PEG-1016 | 21% | 1% |
| PEG-1018 | 0% | 0% |
| PEG-1020 | 13% | 1% |
| PEG-1022 | 0% | 0% |
| PEG-1070 | 1% | 0% |
| PEG-1072 | 2% | 0% |
| PEG-1074 | 1% | 0% |
| PEG-1076 | 2% | 1% |
| PEG-1097 | 5% | 0% |
| PEG-1099 | 3% | 0% |
| PEG-1101 | 12% | 0% |
| PEG-1103 | 10% | 1% |
| PEG-1133 | 3% | 0% |
| PEG-1135 | 4% | 0% |
| PEG-1137 | 4% | 0% |
| PEG-1139 | 4% | 1% |
| PEG-1151 | 2% | 1% |
| PEG-1153 | 2% | 0% |
| PEG-1155 | 1% | 0% |
| PEG-1157 | 3% | 0% |
| PEG-1204 | 29% | 1% |
| PEG-1205 | 0% | 0% |
| PEG-1206 | 11% | 1% |
| PEG-1207 | 0% | 0% |
| PEG-1208 | 30% | |
| PEG-1209 | 7% | |
| PEG-1210 | 2% | |
| PEG-1211 | 1% | 0% |
| PEG-1212 | 40% | 2% |
| PEG-1213 | 42% | 2% |
| PEG-1214 | 28% | 2% |
| PEG-1215 | 32% | 1% |
| PEG-1216 | 41% | 3% |
| PEG-1217 | 37% | 1% |
| PEG-1218 | 21% | 2% |
| PEG-1219 | 21% | 1% |
| PEG-1220 | 30% | 1% |
| PEG-1221 | 7% | |
| PEG-1222 | 2% | 0% |
| PEG-1223 | 1% | 0% |
| PEG-1224 | 36% | 1% |
| PEG-1225 | 12% | 0% |
| PEG-1226 | 29% | 1% |
| PEG-1227 | 1% | 0% |
| PEG-1228 | 40% | 3% |
| PEG-1229 | 12% | 0% |
| PEG-1230 | 1% | 0% |
| PEG-1231 | 1% | 0% |
| PEG-1232 | 29% | 2% |
| PEG-1233 | 35% | 2% |
| PEG-1234 | 23% | 2% |
| PEG-1235 | 1% | 0% |
| PEG-1236 | 39% | 2% |
| PEG-1237 | 33% | 2% |
| PEG-1238 | 21% | 1% |
| PEG-1239 | 0% | 0% |
| PEG-1240 | 30% | 3% |
| PEG-1241 | 2% | 0% |
| PEG-1242 | 16% | 1% |
| PEG-1243 | 27% | 1% |
| PEG-1244 | 28% | 2% |
| PEG-1245 | 31% | 4% |
| PEG-1246 | 34% | 3% |
| PEG-1247 | 41% | 20% |
| PEG-1248 | 34% | 1% |
| PEG-1249 | 1% | 0% |
| PEG-1250 | 24% | 1% |
| PEG-1251 | 1% | 0% |
| PEG-1252 | 45% | 2% |
| PEG-1253 | 12% | 1% |
| PEG-1254 | 23% | 1% |
| PEG-1256 | 29% | 1% |
| PEG-1257 | 23% | 2% |
| PEG-1258 | 18% | 1% |
| PEG-1259 | 22% | 1% |
| PEG-1260 | 17% | 1% |
| PEG-1261 | 18% | 1% |
| PEG-1262 | 12% | 1% |
| PEG-1263 | 15% | 1% |
| PEG-4001 | 25% | 1% |
| PEG-4002 | 22% | 1% |
| PEG-4003 | 15% | 1% |
| PEG-4004 | 13% | 1% |

TABLE 18e
Correction of G339C using PE2 systems,
experiment 2 (indel % not reported).

| PEG # | Edit % |
|---|---|
| PEG-0011 | 0% |
| PEG-0011 | 3% |
| PEG-0013 | 0% |
| PEG-0013 | 0% |
| PEG-0015 | 0% |
| PEG-0015 | 2% |
| PEG-0017 | 0% |
| PEG-0017 | 1% |
| PEG-0029 | 5% |
| PEG-0029 | 11% |
| PEG-0031 | 0% |
| PEG-0031 | 10% |
| PEG-0033 | 10% |
| PEG-0033 | 5% |
| PEG-0035 | 5% |
| PEG-0035 | 0% |
| PEG-0047 | 15% |
| PEG-0047 | 11% |
| PEG-0049 | 20% |
| PEG-0049 | 13% |
| PEG-0051 | 0% |
| PEG-0051 | 49% |
| PEG-0053 | 6% |
| PEG-0053 | 30% |
| PEG-0065 | 19% |
| PEG-0065 | 18% |
| PEG-0067 | 17% |
| PEG-0067 | 12% |
| PEG-0069 | 19% |
| PEG-0069 | 14% |
| PEG-0071 | 13% |
| PEG-0071 | 0% |
| PEG-0092 | 7% |
| PEG-0092 | 7% |
| PEG-0094 | 10% |
| PEG-0094 | 4% |
| PEG-0096 | 11% |
| PEG-0096 | 9% |
| PEG-0098 | 0% |
| PEG-0098 | 0% |
| PEG-0110 | 10% |
| PEG-0110 | 5% |
| PEG-0112 | 11% |
| PEG-0112 | 4% |
| PEG-0114 | 15% |
| PEG-0114 | 14% |
| PEG-0116 | 0% |
| PEG-0116 | 12% |
| PEG-0209 | 1% |
| PEG-0211 | 1% |
| PEG-0213 | 1% |
| PEG-0215 | 1% |
| PEG-0236 | 5% |
| PEG-0238 | 4% |
| PEG-0240 | 4% |
| PEG-0242 | 6% |
| PEG-0263 | 3% |
| PEG-0265 | 2% |
| PEG-0267 | 5% |
| PEG-0269 | 7% |
| PEG-0290 | 7% |
| PEG-0292 | 7% |
| PEG-0326 | 1% |
| PEG-0328 | 1% |
| PEG-0330 | 1% |
| PEG-0332 | 1% |
| PEG-0353 | 3% |
| PEG-0355 | 5% |
| PEG-0357 | 5% |
| PEG-0359 | 4% |
| PEG-0380 | 3% |
| PEG-0382 | 1% |
| PEG-0384 | 5% |
| PEG-0386 | 6% |
| PEG-0407 | 12% |
| PEG-0409 | 12% |
| PEG-0411 | 9% |
| PEG-0413 | 11% |
| PEG-0434 | 0% |
| PEG-0436 | 0% |
| PEG-0438 | 2% |
| PEG-0440 | 0% |
| PEG-0461 | 3% |
| PEG-0463 | 3% |
| PEG-0465 | 6% |
| PEG-0467 | 4% |
| PEG-0488 | 1% |
| PEG-0490 | 1% |
| PEG-0492 | 4% |
| PEG-0494 | 3% |
| PEG-0613 | 0% |
| PEG-0614 | 1% |
| PEG-0615 | 1% |
| PEG-0616 | 0% |
| PEG-0617 | 10% |
| PEG-0618 | 5% |
| PEG-0619 | 5% |
| PEG-0620 | 0% |
| PEG-0621 | 19% |
| PEG-0622 | 0% |
| PEG-0623 | 19% |
| PEG-0624 | 1% |
| PEG-0625 | 19% |
| PEG-0626 | 21% |
| PEG-0627 | 19% |
| PEG-0628 | 10% |
| PEG-0629 | 10% |
| PEG-0630 | 8% |
| PEG-0631 | 10% |
| PEG-0632 | 11% |
| PEG-0633 | 10% |
| PEG-0634 | 0% |
| PEG-0635 | 14% |
| PEG-0636 | 16% |
| PEG-0637 | 0% |
| PEG-0638 | 1% |
| PEG-0639 | 1% |
| PEG-0640 | 0% |
| PEG-0641 | 12% |
| PEG-0642 | 11% |
| PEG-0643 | 0% |
| PEG-0644 | 8% |
| PEG-0645 | 12% |
| PEG-0646 | 22% |
| PEG-0647 | 15% |
| PEG-0648 | 0% |
| PEG-0649 | 18% |
| PEG-0650 | 12% |
| PEG-0651 | 15% |
| PEG-0652 | 11% |
| PEG-0653 | 8% |
| PEG-0654 | 8% |
| PEG-0655 | 8% |
| PEG-0656 | 8% |
| PEG-0657 | 9% |
| PEG-0658 | 7% |
| PEG-0659 | 17% |
| PEG-0660 | 16% |
| PEG-0661 | 1% |
| PEG-0662 | 0% |
| PEG-0663 | 0% |
| PEG-0664 | 0% |
| PEG-0665 | 3% |
| PEG-0666 | 4% |
| PEG-0667 | 5% |
| PEG-0668 | 0% |
| PEG-0669 | 13% |
| PEG-0670 | 0% |
| PEG-0671 | 0% |
| PEG-0672 | 0% |
| PEG-0673 | 17% |
| PEG-0674 | 7% |
| PEG-0675 | 12% |
| PEG-0676 | 9% |
| PEG-0677 | 7% |
| PEG-0678 | 6% |
| PEG-0679 | 10% |
| PEG-0680 | 8% |
| PEG-0681 | 8% |
| PEG-0682 | 0% |
| PEG-0683 | 19% |
| PEG-0684 | 10% |
| PEG-0733 | 2% |
| PEG-0734 | 1% |
| PEG-0735 | 3% |
| PEG-0736 | 3% |
| PEG-0737 | 12% |
| PEG-0738 | 13% |
| PEG-0739 | 13% |
| PEG-0740 | 10% |
| PEG-0741 | 4% |
| PEG-0742 | 3% |
| PEG-0743 | 2% |
| PEG-0744 | 11% |
| PEG-0745 | 11% |
| PEG-0746 | 9% |
| PEG-0747 | 10% |
| PEG-0748 | 19% |
| PEG-0749 | 2% |
| PEG-0750 | 0% |
| PEG-0751 | 1% |
| PEG-0752 | 1% |
| PEG-0753 | 3% |
| PEG-0754 | 4% |
| PEG-0755 | 6% |
| PEG-0756 | 6% |
| PEG-0757 | 3% |
| PEG-0758 | 4% |
| PEG-0759 | 9% |
| PEG-0760 | 9% |
| PEG-0761 | 8% |
| PEG-0762 | 4% |
| PEG-0763 | 5% |
| PEG-0764 | 5% |
| PEG-0765 | 0% |
| PEG-0766 | 1% |
| PEG-0767 | 0% |
| PEG-0768 | 1% |
| PEG-0769 | 1% |
| PEG-0770 | 3% |
| PEG-0771 | 6% |
| PEG-0772 | 10% |
| PEG-0773 | 1% |
| PEG-0774 | 1% |
| PEG-0775 | 3% |
| PEG-0776 | 10% |
| PEG-0777 | 1% |
| PEG-0778 | 1% |
| PEG-0779 | 2% |
| PEG-0780 | 5% |
| PEG-0800 | 24% |
| PEG-0802 | 0% |
| PEG-0804 | 16% |
| PEG-0806 | 0% |
| PEG-0809 | 19% |
| PEG-0811 | 0% |
| PEG-0813 | 0% |
| PEG-0815 | 0% |
| PEG-0818 | 21% |
| PEG-0820 | 17% |
| PEG-0822 | 21% |
| PEG-0824 | 0% |
| PEG-0827 | 22% |
| PEG-0829 | 14% |
| PEG-0831 | 13% |
| PEG-0833 | 19% |
| PEG-0836 | 27% |
| PEG-0838 | 13% |
| PEG-0840 | 20% |
| PEG-0842 | 14% |
| PEG-0845 | 20% |
| PEG-0847 | 23% |
| PEG-0849 | 0% |
| PEG-0851 | 23% |
| PEG-0854 | 19% |
| PEG-0856 | 20% |
| PEG-0858 | 14% |
| PEG-0860 | 0% |
| PEG-0863 | 9% |
| PEG-0865 | 9% |
| PEG-0867 | 0% |
| PEG-0869 | 5% |
| PEG-0872 | 15% |
| PEG-0874 | 0% |
| PEG-0876 | 0% |
| PEG-0878 | 15% |
| PEG-0944 | 12% |
| PEG-0946 | 19% |
| PEG-0948 | 14% |
| PEG-0950 | 13% |
| PEG-0962 | 14% |
| PEG-0964 | 19% |
| PEG-0966 | 19% |
| PEG-0968 | 0% |
| PEG-0980 | 17% |
| PEG-0982 | 0% |
| PEG-0984 | 13% |
| PEG-0986 | 24% |
| PEG-0998 | 12% |
| PEG-1000 | 10% |
| PEG-1002 | 4% |
| PEG-1004 | 5% |
| PEG-1016 | 8% |
| PEG-1018 | 0% |
| PEG-1020 | 2% |
| PEG-1022 | 0% |
| PEG-1070 | 0% |
| PEG-1072 | 0% |
| PEG-1074 | 1% |
| PEG-1076 | 1% |
| PEG-1097 | 2% |
| PEG-1099 | 2% |
| PEG-1101 | 2% |
| PEG-1103 | 4% |
| PEG-1133 | 1% |
| PEG-1135 | 5% |
| PEG-1137 | 3% |
| PEG-1139 | 4% |
| PEG-1151 | 0% |
| PEG-1153 | 2% |
| PEG-1155 | 1% |
| PEG-1157 | 1% |
| PEG-1204 | 16% |
| PEG-1205 | 0% |
| PEG-1206 | 8% |
| PEG-1207 | 0% |
| PEG-1208 | 20% |
| PEG-1209 | 0% |
| PEG-1210 | 20% |
| PEG-1211 | 15% |
| PEG-1212 | 17% |
| PEG-1213 | 18% |
| PEG-1214 | 11% |
| PEG-1215 | 12% |
| PEG-1216 | 18% |
| PEG-1217 | 10% |
| PEG-1218 | 10% |
| PEG-1219 | 13% |
| PEG-1220 | 16% |
| PEG-1221 | 0% |
| PEG-1222 | 1% |
| PEG-1223 | 0% |
| PEG-1224 | 14% |
| PEG-1225 | 2% |
| PEG-1226 | 10% |
| PEG-1227 | 0% |
| PEG-1228 | 16% |
| PEG-1229 | 3% |
| PEG-1230 | 0% |
| PEG-1231 | 0% |
| PEG-1232 | 15% |
| PEG-1233 | 15% |
| PEG-1234 | 6% |
| PEG-1235 | 0% |
| PEG-1236 | 8% |
| PEG-1237 | 8% |
| PEG-1238 | 3% |
| PEG-1239 | 0% |
| PEG-1240 | 7% |
| PEG-1241 | 1% |
| PEG-1242 | 10% |
| PEG-1243 | 19% |
| PEG-1244 | 13% |
| PEG-1245 | 13% |
| PEG-1246 | 24% |
| PEG-1247 | 19% |
| PEG-1248 | 0% |
| PEG-1249 | 8% |
| PEG-1250 | 0% |
| PEG-1251 | 16% |
| PEG-1252 | 6% |
| PEG-1253 | 16% |
| PEG-1254 | 0% |
| PEG-1256 | 21% |
| PEG-1257 | 11% |
| PEG-1258 | 4% |
| PEG-1259 | 4% |
| PEG-1260 | 5% |
| PEG-1261 | 4% |
| PEG-1262 | 4% |
| PEG-1263 | 5% |
| PEG-4001 | 7% |
| PEG-4002 | 6% |
| PEG-4003 | 4% |
| PEG-4004 | 3% |

TABLE 18f
Correction of G339C using PE2 systems, experiment 3.

| PEG # | Edit % | Indel % | PEG # | Edit % | Indel % | PEG # | Edit % | Indel % | PEG # | Edit % | Indel % |
|---|---|---|---|---|---|---|---|---|---|---|---|
| PEG-0038 | 16% | 2% | PEG-0953 | 15% | 1% | PEG-4084 | 11% | 1% | PEG-4253 | 3% | 1% |
| PEG-0039 | 17% | 6% | PEG-0954 | 15% | 1% | PEG-4085 | 8% | 1% | PEG-4254 | 2% | 1% |
| PEG-0040 | 14% | 2% | PEG-0955 | 25% | 1% | PEG-4086 | 7% | 0% | PEG-4255 | 2% | 0% |
| PEG-0041 | 19% | 5% | PEG-0956 | 25% | 1% | PEG-4087 | 9% | 1% | PEG-4256 | 1% | 0% |
| PEG-0042 | 17% | 3% | PEG-0957 | 21% | 1% | PEG-4088 | 7% | 1% | PEG-4257 | 2% | 0% |
| PEG-0043 | 16% | 3% | PEG-0958 | 10% | 0% | PEG-4089 | 11% | 1% | PEG-4258 | 2% | 0% |
| PEG-0044 | 9% | 1% | PEG-0959 | 9% | 1% | PEG-4090 | 16% | 1% | PEG-4259 | 2% | 1% |
| PEG-0047 | 31% | 5% | PEG-0962 | 13% | 0% | PEG-4091 | 20% | 1% | PEG-4260 | 5% | 1% |
| PEG-0048 | 27% | 8% | PEG-0963 | 14% | 1% | PEG-4092 | 11% | 1% | PEG-0771 | 5% | 1% |
| PEG-0049 | 35% | 6% | PEG-0964 | 26% | 1% | PEG-4093 | 22% | 2% | PEG-4262 | 7% | 1% |
| PEG-0050 | 21% | 7% | PEG-0965 | 29% | 1% | PEG-4094 | 18% | 2% | PEG-4263 | 5% | 1% |
| PEG-0051 | 25% | 4% | PEG-0966 | 15% | 1% | PEG-4095 | 29% | 1% | PEG-4264 | 4% | 1% |
| PEG-0052 | 18% | 3% | PEG-0967 | 8% | 0% | PEG-4096 | 18% | 1% | PEG-0775 | 3% | 0% |
| PEG-0053 | 17% | 2% | PEG-0968 | 12% | 1% | PEG-4097 | 14% | 1% | PEG-4266 | 2% | 0% |
| PEG-0056 | 23% | 5% | PEG-0971 | 11% | 0% | PEG-4099 | 18% | 1% | PEG-4267 | 3% | 1% |
| PEG-0057 | 22% | 8% | PEG-0972 | 12% | 1% | PEG-4100 | 16% | 1% | PEG-0779 | 3% | 1% |
| PEG-0058 | 27% | 10% | PEG-0973 | 25% | 1% | PEG-4101 | 15% | 1% | PEG-4269 | 4% | 1% |
| PEG-0059 | 16% | 5% | PEG-0974 | 8% | 0% | PEG-4102 | 13% | 0% | PEG-4270 | 5% | 1% |
| PEG-0060 | 19% | 3% | PEG-0975 | 8% | 1% | PEG-4103 | 15% | 0% | PEG-4271 | 5% | 2% |
| PEG-0061 | 18% | 2% | PEG-0976 | 6% | 0% | PEG-4104 | 10% | 1% | PEG-4272 | 4% | 1% |
| PEG-0062 | 14% | 2% | PEG-0977 | 6% | 0% | PEG-4105 | 10% | 0% | PEG-4273 | 2% | 1% |
| PEG-0065 | 14% | 4% | PEG-0980 | 11% | 0% | PEG-4106 | 8% | 1% | PEG-4274 | 2% | 0% |
| PEG-0066 | 13% | 3% | PEG-0981 | 11% | 1% | PEG-4107 | 8% | 0% | PEG-4275 | 3% | 0% |
| PEG-0067 | 16% | 3% | PEG-0982 | 27% | 1% | PEG-4108 | 9% | 0% | PEG-4276 | 2% | 1% |
| PEG-0068 | 13% | 2% | PEG-0983 | 6% | 0% | PEG-4109 | 5% | 0% | PEG-4277 | 2% | 0% |
| PEG-0069 | 12% | 2% | PEG-0984 | 9% | 0% | PEG-4110 | 16% | 4% | PEG-4278 | 8% | 2% |
| PEG-0070 | 10% | 1% | PEG-0985 | 8% | 0% | PEG-4111 | 15% | 3% | PEG-0772 | 8% | 2% |
| PEG-0071 | 7% | 1% | PEG-0986 | 6% | 0% | PEG-4112 | 18% | 5% | PEG-4009 | 10% | 3% |
| PEG-0074 | 10% | 2% | PEG-0989 | 12% | 1% | PEG-4113 | 10% | 2% | PEG-4281 | 7% | 1% |
| PEG-0075 | 8% | 1% | PEG-0990 | 14% | 1% | PEG-4114 | 13% | 2% | PEG-4282 | 6% | 1% |
| PEG-0076 | 9% | 1% | PEG-0991 | 18% | 1% | PEG-4116 | 9% | 1% | PEG-0776 | 6% | 1% |
| PEG-0077 | 12% | 2% | PEG-0992 | 3% | 0% | PEG-4117 | 18% | 1% | PEG-4284 | 5% | 1% |
| PEG-0078 | 7% | 1% | PEG-0993 | 8% | 1% | PEG-4118 | 30% | 1% | PEG-4285 | 6% | 1% |
| PEG-0079 | 5% | 1% | PEG-0994 | 4% | 0% | PEG-4119 | 24% | 1% | PEG-0780 | 5% | 1% |
| PEG-0080 | 5% | 1% | PEG-0995 | 4% | 0% | PEG-4120 | 21% | 1% | PEG-4287 | 6% | 1% |
| PEG-0227 | 5% | 1% | PEG-0998 | 8% | 1% | PEG-4121 | 18% | 1% | PEG-0737 | 10% | 1% |
| PEG-0228 | 4% | 0% | PEG-0999 | 15% | 1% | PEG-4122 | 31% | 1% | PEG-4289 | 9% | 2% |
| PEG-0229 | 4% | 0% | PEG-1000 | 19% | 1% | PEG-4123 | 26% | 1% | PEG-4290 | 6% | 1% |
| PEG-0230 | 5% | 1% | PEG-1001 | 6% | 0% | PEG-4124 | 24% | 1% | PEG-4291 | 4% | 1% |
| PEG-0231 | 4% | 1% | PEG-1002 | 6% | 1% | PEG-4125 | 21% | 1% | PEG-0741 | 4% | 1% |
| PEG-0232 | 3% | 1% | PEG-1003 | 4% | 0% | PEG-4126 | 19% | 1% | PEG-4293 | 6% | 1% |
| PEG-0233 | 5% | 1% | PEG-1004 | 4% | 0% | PEG-4127 | 23% | 1% | PEG-4294 | 4% | 1% |
| PEG-0236 | 6% | 1% | PEG-1204 | 15% | 1% | PEG-4128 | 27% | 1% | PEG-0745 | 12% | 2% |
| PEG-0237 | 4% | 0% | PEG-1205 | 8% | 1% | PEG-4129 | 22% | 1% | PEG-4296 | 12% | 2% |
| PEG-0238 | 2% | 0% | PEG-1206 | 16% | 1% | PEG-4130 | 22% | 2% | PEG-4007 | 14% | 2% |
| PEG-0239 | 5% | 1% | PEG-1208 | 16% | 1% | PEG-4131 | 26% | 1% | PEG-4298 | 12% | 2% |
| PEG-0240 | 3% | 1% | PEG-1209 | 14% | 1% | PEG-4132 | 44% | 2% | PEG-4299 | 9% | 1% |
| PEG-0241 | 2% | 0% | PEG-1211 | 22% | 1% | PEG-4133 | 44% | 2% | PEG-4300 | 5% | 1% |
| PEG-0242 | 6% | 1% | PEG-1212 | 10% | 1% | PEG-4134 | 34% | 1% | PEG-4301 | 4% | 1% |
| PEG-0245 | 6% | 0% | PEG-1213 | 23% | 2% | PEG-4135 | 26% | 1% | PEG-4302 | 3% | 1% |
| PEG-0246 | 4% | 0% | PEG-1214 | 25% | 1% | PEG-4136 | 17% | 1% | PEG-4303 | 5% | 1% |
| PEG-0247 | 3% | 0% | PEG-1215 | 7% | 1% | PEG-4137 | 23% | 1% | PEG-4304 | 5% | 1% |
| PEG-0248 | 5% | 0% | PEG-1216 | 7% | 1% | PEG-4138 | 28% | 2% | PEG-4305 | 8% | 1% |
| PEG-0249 | 4% | 1% | PEG-1217 | 29% | 2% | PEG-4139 | 25% | 1% | PEG-0738 | 10% | 1% |
| PEG-0250 | 5% | 1% | PEG-1218 | 10% | 1% | PEG-4140 | 13% | 1% | PEG-4307 | 10% | 1% |
| PEG-0251 | 8% | 1% | PEG-1219 | 5% | 1% | PEG-4141 | 22% | 1% | PEG-4308 | 6% | 1% |
| PEG-0254 | 3% | 0% | PEG-1224 | 26% | 1% | PEG-4142 | 38% | 1% | PEG-4309 | 4% | 1% |
| PEG-0255 | 4% | 0% | PEG-1225 | 28% | 1% | PEG-4143 | 32% | 1% | PEG-0742 | 4% | 1% |
| PEG-0256 | 2% | 0% | PEG-1226 | 15% | 1% | PEG-4144 | 22% | 1% | PEG-4311 | 3% | 1% |
| PEG-0257 | 2% | 0% | PEG-1227 | 11% | 1% | PEG-4145 | 21% | 1% | PEG-4312 | 4% | 1% |
| PEG-0258 | 2% | 0% | PEG-1228 | 23% | 1% | PEG-4146 | 13% | 1% | PEG-0746 | 5% | 1% |
| PEG-0259 | 4% | 0% | PEG-1229 | 34% | 1% | PEG-4147 | 18% | 1% | PEG-4314 | 9% | 2% |
| PEG-0260 | 6% | 0% | PEG-1230 | 19% | 1% | PEG-4148 | 33% | 1% | PEG-4315 | 7% | 1% |
| PEG-0263 | 3% | 0% | PEG-1231 | 10% | 1% | PEG-4149 | 23% | 1% | PEG-4316 | 10% | 2% |
| PEG-0264 | 4% | 0% | PEG-1232 | 18% | 1% | PEG-4150 | 16% | 1% | PEG-4317 | 5% | 1% |
| PEG-0265 | 3% | 0% | PEG-1233 | 20% | 1% | PEG-4151 | 33% | 2% | PEG-4318 | 6% | 1% |
| PEG-0266 | 3% | 0% | PEG-1234 | 8% | 1% | PEG-4152 | 34% | 1% | PEG-4319 | 4% | 1% |
| PEG-0267 | 1% | 0% | PEG-1235 | 6% | 1% | PEG-4153 | 19% | 2% | PEG-4320 | 2% | 1% |
| PEG-0268 | 4% | 0% | PEG-1236 | 13% | 1% | PEG-4154 | 30% | 2% | PEG-4321 | 3% | 1% |
| PEG-0269 | 6% | 1% | PEG-1237 | 22% | 1% | PEG-4155 | 41% | 2% | PEG-4322 | 4% | 1% |
| PEG-0272 | 3% | 0% | PEG-1238 | 8% | 1% | PEG-4156 | 39% | 2% | PEG-4323 | 6% | 2% |
| PEG-0274 | 6% | 0% | PEG-1239 | 4% | 1% | PEG-4157 | 35% | 3% | PEG-0739 | 8% | 1% |
| PEG-0275 | 3% | 0% | PEG-1244 | 13% | 2% | PEG-4158 | 36% | 2% | PEG-4325 | 8% | 1% |
| PEG-0276 | 2% | 0% | PEG-1245 | 23% | 1% | PEG-4159 | 30% | 1% | PEG-4326 | 5% | 1% |
| PEG-0277 | 4% | 1% | PEG-1246 | 10% | 1% | PEG-4160 | 38% | 1% | PEG-4327 | 3% | 1% |
| PEG-0278 | 5% | 1% | PEG-1247 | 6% | 1% | PEG-4161 | 44% | 3% | PEG-0743 | 3% | 1% |
| PEG-0281 | 14% | 1% | PEG-1248 | 7% | 1% | PEG-4162 | 30% | 2% | PEG-4329 | 4% | 1% |
| PEG-0282 | 10% | 1% | PEG-1249 | 26% | 1% | PEG-4163 | 26% | 1% | PEG-4330 | 6% | 1% |
| PEG-0283 | 19% | 1% | PEG-1250 | 15% | 1% | PEG-4164 | 28% | 1% | PEG-0747 | 6% | 1% |
| PEG-0284 | 11% | 1% | PEG-1251 | 9% | 1% | PEG-4165 | 38% | 2% | PEG-4332 | 12% | 2% |
| PEG-0285 | 5% | 0% | PEG-1252 | 10% | 1% | PEG-4166 | 31% | 2% | PEG-4333 | 11% | 2% |
| PEG-0286 | 7% | 1% | PEG-1253 | 22% | 2% | PEG-4167 | 28% | 2% | PEG-4334 | 12% | 2% |
| PEG-0287 | 7% | 1% | PEG-1254 | 14% | 1% | PEG-4168 | 32% | 2% | PEG-4335 | 10% | 2% |
| PEG-0290 | 20% | 1% | PEG-1255 | 6% | 1% | PEG-4169 | 24% | 1% | PEG-4336 | 7% | 1% |
| PEG-0291 | 16% | 1% | PEG-1256 | 7% | 1% | PEG-4170 | 28% | 1% | PEG-4337 | 5% | 1% |
| PEG-0292 | 32% | 2% | PEG-1257 | 22% | 1% | PEG-4171 | 34% | 1% | PEG-4338 | 6% | 1% |
| PEG-0293 | 17% | 1% | PEG-1258 | 9% | 1% | PEG-4172 | 26% | 1% | PEG-4339 | 8% | 2% |
| PEG-0294 | 7% | 0% | PEG-1259 | 3% | 1% | PEG-4173 | 14% | 1% | PEG-4340 | 7% | 1% |
| PEG-0295 | 10% | 1% | PEG-4005 | 23% | 1% | PEG-4174 | 20% | 1% | PEG-4341 | 10% | 2% |
| PEG-0296 | 11% | 1% | PEG-4006 | 46% | 3% | PEG-4175 | 30% | 1% | PEG-0740 | 10% | 2% |
| PEG-0621 | 36% | 1% | PEG-4011 | 25% | 1% | PEG-4176 | 31% | 1% | PEG-4343 | 11% | 1% |
| PEG-0622 | 39% | 1% | PEG-4012 | 26% | 1% | PEG-4177 | 28% | 1% | PEG-4344 | 9% | 1% |
| PEG-0623 | 18% | 1% | PEG-4013 | 27% | 1% | PEG-4178 | 21% | 1% | PEG-4345 | 5% | 1% |
| PEG-0624 | 39% | 1% | PEG-4014 | 14% | 1% | PEG-4179 | 13% | 1% | PEG-0744 | 5% | 1% |
| PEG-0625 | 26% | 2% | PEG-4015 | 17% | 1% | PEG-4180 | 25% | 1% | PEG-4347 | 5% | 1% |
| PEG-0626 | 26% | 8% | PEG-4016 | 21% | 1% | PEG-4181 | 37% | 1% | PEG-4348 | 6% | 2% |
| PEG-0627 | 6% | 0% | PEG-4017 | 17% | 1% | PEG-4182 | 24% | 1% | PEG-0748 | 0% | 0% |
| PEG-0628 | 26% | 1% | PEG-4018 | 16% | 1% | PEG-4183 | 21% | 1% | PEG-4350 | 6% | 1% |
| PEG-0645 | 52% | 3% | PEG-4019 | 13% | 1% | PEG-4184 | 19% | 1% | PEG-0753 | 5% | 1% |
| PEG-0646 | 50% | 1% | PEG-4020 | 19% | 1% | PEG-4185 | 36% | 1% | PEG-4352 | 6% | 1% |
| PEG-0647 | 39% | 1% | PEG-4021 | 18% | 1% | PEG-4186 | 27% | 1% | PEG-4353 | 3% | 0% |
| PEG-0648 | 40% | 1% | PEG-4022 | 19% | 1% | PEG-4187 | 17% | 1% | PEG-4354 | 3% | 0% |
| PEG-0649 | 27% | 3% | PEG-4023 | 31% | 1% | PEG-4188 | 31% | 1% | PEG-0757 | 2% | 0% |
| PEG-0650 | 32% | 2% | PEG-4024 | 21% | 1% | PEG-4189 | 42% | 1% | PEG-4356 | 2% | 0% |
| PEG-0651 | 20% | 1% | PEG-4025 | 22% | 1% | PEG-4190 | 31% | 1% | PEG-4357 | 4% | 1% |
| PEG-0652 | 28% | 1% | PEG-4026 | 19% | 2% | PEG-4191 | 21% | 1% | PEG-0761 | 8% | 1% |
| PEG-0669 | 32% | 1% | PEG-4027 | 24% | 1% | PEG-4192 | 23% | 1% | PEG-4359 | 6% | 1% |
| PEG-0670 | 36% | 1% | PEG-4028 | 10% | 0% | PEG-4193 | 13% | 1% | PEG-4360 | 8% | 1% |
| PEG-0671 | 35% | 1% | PEG-4029 | 8% | 1% | PEG-4194 | 29% | 1% | PEG-4361 | 7% | 1% |
| PEG-0672 | 29% | 1% | PEG-4030 | 7% | 1% | PEG-4195 | 37% | 2% | PEG-4362 | 4% | 1% |
| PEG-0673 | 12% | 48% | PEG-4031 | 10% | 0% | PEG-4196 | 16% | 1% | PEG-4363 | 3% | 1% |
| PEG-0674 | 25% | 1% | PEG-4032 | 7% | 1% | PEG-4197 | 15% | 1% | PEG-4364 | 3% | 1% |
| PEG-0675 | 22% | 1% | PEG-4033 | 12% | 0% | PEG-4198 | 16% | 1% | PEG-4365 | 2% | 0% |
| PEG-0676 | 22% | 1% | PEG-4034 | 7% | 1% | PEG-4199 | 21% | 1% | PEG-4366 | 3% | 0% |
| PEG-0800 | 21% | 2% | PEG-4035 | 5% | 0% | PEG-4200 | 14% | 1% | PEG-4367 | 10% | 1% |
| PEG-0801 | 32% | 5% | PEG-4036 | 16% | 1% | PEG-4201 | 13% | 1% | PEG-4368 | 7% | 1% |
| PEG-0802 | 23% | 7% | PEG-4037 | 10% | 0% | PEG-4202 | 10% | 0% | PEG-0754 | 7% | 1% |
| PEG-0803 | 19% | 2% | PEG-4038 | 9% | 0% | PEG-4203 | 16% | 1% | PEG-4370 | 5% | 1% |
| PEG-0804 | 19% | 3% | PEG-4039 | 9% | 0% | PEG-4204 | 13% | 0% | PEG-4371 | 3% | 1% |
| PEG-0805 | 25% | 3% | PEG-4040 | 9% | 1% | PEG-4205 | 10% | 0% | PEG-4372 | 2% | 1% |
| PEG-0806 | 8% | 1% | PEG-4041 | 9% | 0% | PEG-4206 | 7% | 0% | PEG-0758 | 1% | 0% |
| PEG-0809 | 27% | 2% | PEG-4042 | 6% | 0% | PEG-4207 | 18% | 0% | PEG-4374 | 2% | 1% |
| PEG-0810 | 28% | 6% | PEG-4043 | 11% | 1% | PEG-4208 | 33% | 1% | PEG-4375 | 3% | 1% |
| PEG-0811 | 26% | 5% | PEG-4044 | 5% | 1% | PEG-4209 | 29% | 1% | PEG-0762 | 10% | 1% |
| PEG-0812 | 23% | 3% | PEG-4045 | 3% | 0% | PEG-4210 | 20% | 0% | PEG-4377 | 5% | 1% |
| PEG-0813 | 27% | 3% | PEG-4046 | 10% | 1% | PEG-4211 | 19% | 1% | PEG-4378 | 4% | 1% |
| PEG-0814 | 23% | 2% | PEG-4047 | 6% | 1% | PEG-4212 | 11% | 0% | PEG-4379 | 5% | 0% |
| PEG-0815 | 10% | 1% | PEG-4048 | 7% | 0% | PEG-4213 | 26% | 1% | PEG-4380 | 3% | 0% |
| PEG-0818 | 27% | 3% | PEG-4049 | 8% | 2% | PEG-4214 | 42% | 1% | PEG-4381 | 2% | 0% |
| PEG-0819 | 28% | 5% | PEG-4050 | 11% | 1% | PEG-4215 | 28% | 1% | PEG-4382 | 1% | 0% |
| PEG-0820 | 26% | 4% | PEG-4051 | 12% | 1% | PEG-4216 | 16% | 1% | PEG-4383 | 1% | 0% |
| PEG-0821 | 19% | 1% | PEG-4052 | 14% | 1% | PEG-4217 | 4% | 0% | PEG-4384 | 2% | 0% |
| PEG-0822 | 26% | 3% | PEG-4053 | 19% | 1% | PEG-4218 | 4% | 0% | PEG-4385 | 4% | 1% |
| PEG-0823 | 16% | 1% | PEG-4054 | 21% | 1% | PEG-4219 | 2% | 0% | PEG-4386 | 4% | 1% |
| PEG-0824 | 13% | 2% | PEG-4055 | 15% | 1% | PEG-4220 | 3% | 0% | PEG-0755 | 5% | 1% |
| PEG-0827 | 21% | 3% | PEG-4056 | 21% | 2% | PEG-4221 | 3% | 0% | PEG-4388 | 4% | 1% |
| PEG-0828 | 27% | 4% | PEG-4057 | 22% | 1% | PEG-4222 | 4% | 1% | PEG-4389 | 6% | 1% |
| PEG-0829 | 22% | 3% | PEG-4058 | 28% | 1% | PEG-4223 | 6% | 0% | PEG-4390 | 5% | 1% |
| PEG-0830 | 25% | 2% | PEG-4059 | 19% | 1% | PEG-4224 | 3% | 0% | PEG-0759 | 3% | 1% |
| PEG-0831 | 19% | 3% | PEG-4060 | 10% | 1% | PEG-0769 | 3% | 0% | PEG-4392 | 4% | 1% |
| PEG-0832 | 14% | 1% | PEG-4061 | 11% | 1% | PEG-4226 | 3% | 1% | PEG-4393 | 5% | 0% |
| PEG-0833 | 10% | 1% | PEG-4062 | 8% | 1% | PEG-4227 | 2% | 1% | PEG-0763 | 6% | 1% |
| PEG-0836 | 24% | 3% | PEG-4063 | 10% | 1% | PEG-4230 | 1% | 0% | PEG-4395 | 7% | 1% |
| PEG-0837 | 31% | 4% | PEG-4064 | 11% | 1% | PEG-4231 | 2% | 1% | PEG-4396 | 8% | 1% |
| PEG-0838 | 18% | 3% | PEG-4065 | 4% | 1% | PEG-0777 | 3% | 0% | PEG-4397 | 8% | 1% |
| PEG-0839 | 20% | 3% | PEG-4066 | 11% | 1% | PEG-4233 | 2% | 0% | PEG-4398 | 8% | 2% |
| PEG-0840 | 17% | 2% | PEG-4067 | 15% | 1% | PEG-4235 | 3% | 0% | PEG-4399 | 4% | 1% |
| PEG-0841 | 13% | 1% | PEG-4068 | 13% | 1% | PEG-4236 | 2% | 0% | PEG-4400 | 4% | 1% |
| PEG-0842 | 9% | 2% | PEG-4069 | 7% | 1% | PEG-4237 | 1% | 0% | PEG-4401 | 1% | 0% |
| PEG-0845 | 22% | 2% | PEG-4070 | 10% | 1% | PEG-4238 | 1% | 0% | PEG-4402 | 2% | 0% |
| PEG-0846 | 20% | 3% | PEG-4071 | 7% | 0% | PEG-4239 | 1% | 0% | PEG-4403 | 3% | 1% |
| PEG-0847 | 16% | 1% | PEG-4072 | 8% | 0% | PEG-4240 | 1% | 0% | PEG-4404 | 2% | 1% |
| PEG-0848 | 17% | 2% | PEG-4073 | 6% | 0% | PEG-4241 | 2% | 0% | PEG-0756 | 2% | 1% |
| PEG-0849 | 20% | 3% | PEG-4074 | 4% | 0% | PEG-4242 | 2% | 0% | PEG-4406 | 4% | 1% |
| PEG-0850 | 8% | 1% | PEG-4075 | 3% | 0% | PEG-0770 | 1% | 0% | PEG-4407 | 2% | 0% |
| PEG-0851 | 9% | 1% | PEG-4076 | 9% | 1% | PEG-4244 | 2% | 1% | PEG-4408 | 2% | 0% |
| PEG-0944 | 21% | 1% | PEG-4077 | 8% | 1% | PEG-4245 | 2% | 0% | PEG-0760 | 2% | 1% |
| PEG-0945 | 16% | 1% | PEG-4078 | 5% | 1% | PEG-4246 | 2% | 0% | PEG-4410 | 2% | 1% |
| PEG-0946 | 30% | 1% | PEG-4079 | 8% | 1% | PEG-0774 | 2% | 1% | PEG-4411 | 3% | 1% |
| PEG-0947 | 21% | 1% | PEG-4080 | 5% | 1% | PEG-4248 | 1% | 0% | PEG-0764 | 4% | 1% |
| PEG-0948 | 15% | 1% | PEG-4081 | 12% | 1% | PEG-0778 | 3% | 0% | | | |
| PEG-0949 | 14% | 1% | PEG-4082 | 16% | 2% | PEG-4251 | 3% | 1% | | | |
| PEG-0950 | 9% | 1% | PEG-4083 | 14% | 1% | PEG-4252 | 3% | 1% | | | |

TABLE 18g
Correction of G339C using PE2 systems, experiment 4.

| PEG # | Edit % | Indel % | PEG # | Edit % | Indel % | PEG # | Edit % | Indel % | PEG # | Edit % | Indel % |
|---|---|---|---|---|---|---|---|---|---|---|---|
| PEG-0038 | 14.7% | 2.7% | PEG-0954 | 10.1% | 0.5% | PEG-4085 | 13.7% | 1.2% | PEG-4249 | 0.1% | 0.2% |
| PEG-0039 | 19.3% | 7.7% | PEG-0955 | 14.0% | 0.2% | PEG-4086 | 10.6% | 0.7% | PEG-0778 | 0.0% | 0.2% |
| PEG-0040 | 19.1% | 3.3% | PEG-0956 | 8.5% | 0.4% | PEG-4087 | 10.4% | 0.8% | PEG-4251 | 1.9% | 0.5% |
| PEG-0041 | 18.0% | 4.8% | PEG-0957 | 13.4% | 0.3% | PEG-4088 | 9.2% | 0.6% | PEG-4252 | 0.1% | 0.2% |
| PEG-0042 | 12.5% | 1.7% | PEG-0958 | 6.5% | 0.2% | PEG-4089 | 11.2% | 0.3% | PEG-4253 | 0.0% | 0.2% |
| PEG-0043 | 16.0% | 3.1% | PEG-0959 | 3.4% | 0.3% | PEG-4090 | 10.2% | 0.4% | PEG-4254 | 0.1% | 0.2% |
| PEG-0044 | 11.7% | 1.4% | PEG-0962 | 16.4% | 0.8% | PEG-4091 | 12.8% | 0.7% | PEG-4255 | 1.0% | 0.3% |
| PEG-0047 | 29.0% | 5.0% | PEG-0963 | 10.1% | 0.7% | PEG-4092 | 8.9% | 0.7% | PEG-4256 | 0.8% | 0.3% |
| PEG-0048 | 24.3% | 7.1% | PEG-0964 | 11.3% | 0.7% | PEG-4093 | 12.2% | 0.8% | PEG-4257 | 0.9% | 0.3% |
| PEG-0049 | 31.3% | 5.4% | PEG-0965 | 10.2% | 0.8% | PEG-4094 | 9.0% | 0.6% | PEG-4258 | 0.1% | 0.2% |
| PEG-0050 | 25.3% | 8.2% | PEG-0966 | 9.4% | 0.4% | PEG-4095 | 10.8% | 0.4% | PEG-4259 | 0.0% | 0.2% |
| PEG-0051 | 16.5% | 2.0% | PEG-0967 | 5.5% | 0.3% | PEG-4096 | 6.6% | 0.7% | PEG-4260 | 2.2% | 0.9% |
| PEG-0052 | 19.0% | 2.8% | PEG-0968 | 4.5% | 0.3% | PEG-4097 | 6.2% | 0.3% | PEG-0771 | 2.4% | 0.6% |
| PEG-0053 | 18.2% | 2.2% | PEG-0971 | 7.5% | 0.5% | PEG-4098 | 8.3% | 0.6% | PEG-4262 | 3.6% | 0.7% |
| PEG-0056 | 24.9% | 5.5% | PEG-0972 | 7.6% | 0.5% | PEG-4099 | 9.0% | 0.4% | PEG-4263 | 0.0% | 0.1% |
| PEG-0057 | 18.9% | 7.0% | PEG-0973 | 10.2% | 0.5% | PEG-4100 | 13.6% | 0.6% | PEG-4264 | 0.0% | 0.2% |
| PEG-0058 | 20.5% | 6.6% | PEG-0974 | 7.9% | 0.4% | PEG-4101 | 10.1% | 0.5% | PEG-0775 | 1.9% | 0.4% |
| PEG-0059 | 17.6% | 6.2% | PEG-0975 | 4.2% | 0.3% | PEG-4102 | 11.7% | 0.5% | PEG-4266 | 1.4% | 0.3% |
| PEG-0060 | 15.7% | 2.2% | PEG-0976 | 3.7% | 0.2% | PEG-4103 | 14.4% | 0.5% | PEG-4267 | 1.5% | 0.3% |
| PEG-0061 | 17.4% | 2.2% | PEG-0977 | 2.6% | 0.3% | PEG-4104 | 6.0% | 0.3% | PEG-0779 | 1.3% | 0.2% |
| PEG-0062 | 13.2% | 1.8% | PEG-0980 | 10.7% | 0.5% | PEG-4105 | 5.2% | 0.3% | PEG-4269 | 4.3% | 1.1% |
| PEG-0065 | 13.6% | 5.7% | PEG-0981 | 10.1% | 0.4% | PEG-4106 | 3.0% | 0.3% | PEG-4270 | 4.1% | 1.0% |
| PEG-0066 | 13.9% | 3.0% | PEG-0982 | 7.9% | 0.3% | PEG-4107 | 2.8% | 0.2% | PEG-4271 | 4.1% | 1.2% |
| PEG-0067 | 17.8% | 3.8% | PEG-0983 | 6.9% | 0.4% | PEG-4108 | 10.4% | 0.6% | PEG-4272 | 3.3% | 0.5% |
| PEG-0068 | 14.3% | 2.5% | PEG-0984 | 4.4% | 0.2% | PEG-4109 | 9.7% | 0.7% | PEG-4273 | 1.8% | 0.3% |
| PEG-0069 | 9.3% | 1.5% | PEG-0985 | 2.4% | 0.1% | PEG-4110 | 17.4% | 5.0% | PEG-4274 | 2.7% | 0.5% |
| PEG-0070 | 9.5% | 1.2% | PEG-0986 | 2.5% | 0.3% | PEG-4111 | 14.0% | 3.8% | PEG-4275 | 2.2% | 0.3% |
| PEG-0071 | 6.7% | 1.3% | PEG-0989 | 8.1% | 0.4% | PEG-4112 | 13.6% | 4.0% | PEG-4276 | 2.2% | 0.6% |
| PEG-0074 | 9.3% | 3.1% | PEG-0990 | 6.3% | 0.2% | PEG-4113 | 13.3% | 2.2% | PEG-4277 | 1.5% | 0.5% |
| PEG-0075 | 8.1% | 1.3% | PEG-0991 | 5.0% | 0.3% | PEG-4114 | 9.7% | 1.4% | PEG-4278 | 7.5% | 2.6% |
| PEG-0076 | 12.2% | 1.6% | PEG-0992 | 5.3% | 0.4% | PEG-4115 | 9.7% | 1.2% | PEG-0772 | 6.2% | 1.7% |
| PEG-0077 | 6.8% | 0.7% | PEG-0993 | 4.4% | 0.3% | PEG-4116 | 8.1% | 0.8% | PEG-4009 | 5.4% | 0.9% |
| PEG-0078 | 7.6% | 1.0% | PEG-0994 | 2.1% | 0.3% | PEG-4117 | 21.4% | 0.6% | PEG-4281 | 4.4% | 0.6% |
| PEG-0079 | 3.1% | 0.4% | PEG-0995 | 2.9% | 0.2% | PEG-4118 | 25.2% | 1.1% | PEG-4282 | 5.3% | 0.6% |
| PEG-0080 | 4.6% | 0.7% | PEG-0998 | 4.2% | 0.3% | PEG-4119 | 17.6% | 0.6% | PEG-0776 | 4.1% | 0.6% |
| PEG-0227 | 2.0% | 0.3% | PEG-0999 | 8.2% | 0.4% | PEG-4120 | 6.6% | 0.5% | PEG-4284 | 3.6% | 1.0% |
| PEG-0228 | 3.0% | 0.4% | PEG-1000 | 5.8% | 0.5% | PEG-4121 | 18.4% | 0.8% | PEG-4285 | 3.7% | 0.5% |
| PEG-0229 | 3.2% | 0.3% | PEG-1001 | 3.8% | 0.3% | PEG-4122 | 37.5% | 1.3% | PEG-0780 | 4.4% | 0.8% |
| PEG-0230 | 3.3% | 0.5% | PEG-1002 | 4.6% | 0.4% | PEG-4123 | 28.3% | 1.2% | PEG-4287 | 6.5% | 1.4% |
| PEG-0231 | 1.8% | 0.6% | PEG-1003 | 1.5% | 0.1% | PEG-4124 | 13.8% | 0.8% | PEG-0737 | 7.4% | 0.7% |
| PEG-0232 | 2.1% | 0.4% | PEG-1004 | 2.8% | 0.2% | PEG-4125 | 12.1% | 0.5% | PEG-4289 | 7.5% | 1.5% |
| PEG-0233 | 2.7% | 0.3% | PEG-1204 | 7.7% | 0.8% | PEG-4126 | 11.3% | 0.7% | PEG-4290 | 5.8% | 0.8% |
| PEG-0236 | 2.0% | 0.3% | PEG-1205 | 9.2% | 0.8% | PEG-4127 | 16.5% | 0.9% | PEG-4291 | 5.6% | 0.8% |
| PEG-0237 | 5.0% | 0.3% | PEG-1206 | 8.5% | 0.7% | PEG-4128 | 15.8% | 0.9% | PEG-0741 | 2.8% | 0.6% |
| PEG-0238 | 2.1% | 0.2% | PEG-1207 | 8.4% | 0.6% | PEG-4129 | 14.2% | 0.6% | PEG-4293 | 3.4% | 0.4% |
| PEG-0239 | 4.0% | 0.5% | PEG-1208 | 5.8% | 0.4% | PEG-4130 | 15.1% | 1.1% | PEG-4294 | 2.4% | 0.9% |
| PEG-0240 | 3.3% | 0.4% | PEG-1209 | 12.5% | 0.9% | PEG-4131 | 20.4% | 0.5% | PEG-0745 | 6.1% | 0.8% |
| PEG-0241 | 2.3% | 0.3% | PEG-1210 | 11.3% | 0.8% | PEG-4132 | 33.6% | 1.4% | PEG-4296 | 7.7% | 1.3% |
| PEG-0242 | 3.4% | 0.3% | PEG-1211 | 6.6% | 0.3% | PEG-4133 | 23.0% | 0.9% | PEG-4007 | 7.9% | 1.2% |
| PEG-0245 | 4.7% | 0.3% | PEG-1212 | 5.6% | 0.4% | PEG-4134 | 16.3% | 0.9% | PEG-4298 | 8.1% | 1.2% |
| PEG-0246 | 4.5% | 0.6% | PEG-1213 | 15.0% | 1.1% | PEG-4135 | 17.6% | 0.8% | PEG-4299 | 4.3% | 0.9% |
| PEG-0247 | 2.2% | 0.3% | PEG-1214 | 7.4% | 0.4% | PEG-4136 | 18.1% | 1.1% | PEG-4300 | 3.0% | 0.4% |
| PEG-0248 | 3.0% | 0.4% | PEG-1215 | 14.0% | 0.9% | PEG-4137 | 23.7% | 0.9% | PEG-4301 | 1.8% | 0.7% |
| PEG-0249 | 4.8% | 0.4% | PEG-1216 | 4.1% | 0.3% | PEG-4138 | 25.6% | 0.6% | PEG-4302 | 2.2% | 0.5% |
| PEG-0250 | 1.3% | 0.3% | PEG-1217 | 6.4% | 0.3% | PEG-4139 | 13.4% | 0.6% | PEG-4303 | 3.1% | 0.7% |
| PEG-0251 | 3.5% | 0.4% | PEG-1218 | 8.7% | 0.5% | PEG-4140 | 12.3% | 0.6% | PEG-4304 | 3.5% | 0.6% |
| PEG-0254 | 1.5% | 0.3% | PEG-1219 | 4.0% | 0.3% | PEG-4141 | 10.9% | 0.9% | PEG-4305 | 7.6% | 1.2% |
| PEG-0255 | 1.4% | 0.2% | PEG-1224 | 20.9% | 0.8% | PEG-4142 | 22.3% | 1.0% | PEG-0738 | 7.9% | 1.1% |
| PEG-0256 | 1.3% | 0.1% | PEG-1225 | 13.3% | 0.7% | PEG-4143 | 19.4% | 0.8% | PEG-4307 | 7.2% | 1.4% |
| PEG-0257 | 1.1% | 0.2% | PEG-1226 | 6.7% | 0.4% | PEG-4144 | 15.5% | 0.5% | PEG-4308 | 4.9% | 0.5% |
| PEG-0258 | 2.3% | 0.3% | PEG-1227 | 10.3% | 0.5% | PEG-4145 | 14.6% | 0.7% | PEG-4309 | 3.4% | 0.8% |
| PEG-0259 | 2.5% | 0.3% | PEG-1228 | 16.7% | 1.0% | PEG-4146 | 6.9% | 0.5% | PEG-0742 | 2.2% | 0.6% |
| PEG-0260 | 3.5% | 0.3% | PEG-1229 | 9.9% | 0.5% | PEG-4147 | 18.5% | 0.6% | PEG-4311 | 2.0% | 0.4% |
| PEG-0263 | 2.8% | 0.4% | PEG-1230 | 2.8% | 0.3% | PEG-4148 | 18.0% | 0.5% | PEG-4312 | 2.4% | 0.3% |
| PEG-0264 | 3.2% | 0.4% | PEG-1231 | 6.6% | 0.7% | PEG-4149 | 15.1% | 0.4% | PEG-0746 | 3.0% | 0.5% |
| PEG-0265 | 1.6% | 0.2% | PEG-1232 | 12.0% | 0.9% | PEG-4150 | 6.8% | 0.4% | PEG-4314 | 7.0% | 1.2% |
| PEG-0267 | 1.3% | 0.3% | PEG-1233 | 4.8% | 0.3% | PEG-4151 | 34.1% | 1.5% | PEG-4315 | 7.7% | 0.9% |
| PEG-0268 | 2.1% | 0.4% | PEG-1234 | 2.3% | 0.2% | PEG-4152 | 29.4% | 1.4% | PEG-4316 | 7.7% | 1.1% |
| PEG-0269 | 3.8% | 0.3% | PEG-1235 | 7.0% | 0.4% | PEG-4153 | 6.9% | 0.9% | PEG-4317 | 5.8% | 1.0% |
| PEG-0272 | 1.9% | 0.2% | PEG-1236 | 7.8% | 0.7% | PEG-4154 | 10.3% | 0.4% | PEG-4318 | 3.8% | 0.6% |
| PEG-0273 | 3.6% | 0.4% | PEG-1237 | 7.4% | 0.5% | PEG-4155 | 43.2% | 2.6% | PEG-4319 | 1.6% | 0.3% |
| PEG-0274 | 2.7% | 0.3% | PEG-1238 | 3.2% | 0.4% | PEG-4156 | 25.1% | 1.9% | PEG-4320 | 0.9% | 0.4% |
| PEG-0275 | 1.7% | 0.3% | PEG-1239 | 1.4% | 0.2% | PEG-4157 | 25.1% | 2.0% | PEG-4321 | 1.3% | 0.4% |
| PEG-0276 | 1.3% | 0.3% | PEG-1244 | 7.9% | 1.1% | PEG-4158 | 21.6% | 1.0% | PEG-4322 | 2.3% | 0.6% |
| PEG-0277 | 2.0% | 0.2% | PEG-1245 | 10.3% | 0.8% | PEG-4159 | 17.0% | 1.1% | PEG-4323 | 3.8% | 1.4% |
| PEG-0278 | 4.3% | 0.5% | PEG-1246 | 8.9% | 0.8% | PEG-4160 | 22.1% | 0.7% | PEG-0739 | 3.5% | 0.8% |
| PEG-0281 | 7.6% | 0.8% | PEG-1247 | 5.9% | 0.6% | PEG-4161 | 26.8% | 1.9% | PEG-4325 | 4.2% | 0.8% |
| PEG-0282 | 6.8% | 0.6% | PEG-1248 | 3.5% | 0.6% | PEG-4162 | 23.4% | 1.3% | PEG-4326 | 3.0% | 0.5% |
| PEG-0283 | 11.5% | 0.9% | PEG-1249 | 10.5% | 0.4% | PEG-4163 | 1.3% | 0.3% | PEG-4327 | 1.8% | 0.5% |
| PEG-0284 | 4.0% | 0.3% | PEG-1250 | 10.0% | 0.4% | PEG-4164 | 4.8% | 0.4% | PEG-0743 | 2.4% | 0.7% |
| PEG-0285 | 4.8% | 0.4% | PEG-1251 | 4.4% | 0.5% | PEG-4165 | 31.1% | 1.6% | PEG-4329 | 9.6% | |
| PEG-0286 | 3.3% | 0.3% | PEG-1252 | 3.6% | 0.3% | PEG-4166 | 21.3% | 1.5% | PEG-4330 | 10.6% | |
| PEG-0287 | 6.9% | 0.9% | PEG-1253 | 8.5% | 0.7% | PEG-4167 | 21.1% | 0.8% | PEG-0747 | 8.7% | |
| PEG-0290 | 14.3% | 0.6% | PEG-1254 | 9.9% | 0.8% | PEG-4168 | 18.5% | 1.2% | PEG-4332 | 10.8% | |
| PEG-0291 | 13.2% | 0.7% | PEG-1255 | 2.9% | 0.4% | PEG-4169 | 11.7% | 0.6% | PEG-4333 | 10.1% | |
| PEG-0292 | 20.1% | 1.0% | PEG-1256 | 3.1% | 0.4% | PEG-4170 | 17.6% | 0.5% | PEG-4334 | 14.2% | |
| PEG-0293 | 7.3% | 0.7% | PEG-1257 | 10.1% | 0.7% | PEG-4171 | 21.7% | 1.2% | PEG-4335 | 9.7% | |
| PEG-0294 | 6.0% | 0.5% | PEG-1258 | 3.7% | 0.6% | PEG-4172 | 16.2% | 0.6% | PEG-4336 | 9.2% | |
| PEG-0295 | 5.6% | 0.4% | PEG-1259 | 1.6% | 0.5% | PEG-4173 | 6.9% | 0.4% | PEG-4337 | 8.7% | |
| PEG-0296 | 9.7% | 0.6% | PEG-4005 | 24.1% | 0.9% | PEG-4174 | 14.2% | 0.7% | PEG-4338 | 10.7% | |
| PEG-0621 | 25.0% | 0.8% | PEG-4006 | 43.2% | 2.5% | PEG-4175 | 13.7% | 0.7% | PEG-4339 | 8.1% | |
| PEG-0622 | 33.8% | 1.0% | PEG-4011 | 18.7% | 0.5% | PEG-4176 | 12.9% | 0.6% | PEG-4340 | 12.8% | |
| PEG-0623 | 18.0% | 0.4% | PEG-4012 | 13.1% | 0.9% | PEG-4177 | 12.9% | 0.8% | PEG-4341 | 13.6% | |
| PEG-0624 | 12.4% | 0.4% | PEG-4013 | 18.1% | 0.4% | PEG-4178 | 9.9% | 0.4% | PEG-0740 | 15.5% | |
| PEG-0625 | 15.0% | 1.2% | PEG-4014 | 14.7% | 0.6% | PEG-4179 | 4.9% | 0.4% | PEG-4343 | 15.5% | |
| PEG-0626 | 23.5% | 1.3% | PEG-4015 | 11.3% | 0.6% | PEG-4180 | 14.3% | 0.7% | PEG-4344 | 14.1% | |
| PEG-0627 | 6.3% | 0.3% | PEG-4016 | 11.2% | 0.7% | PEG-4181 | 17.5% | 0.8% | PEG-4345 | 11.0% | |
| PEG-0628 | 3.5% | 0.2% | PEG-4017 | 9.0% | 0.7% | PEG-4182 | 10.5% | 0.6% | PEG-0744 | 13.5% | |
| PEG-0645 | 54.2% | 1.9% | PEG-4018 | 8.6% | 0.6% | PEG-4183 | 14.6% | 0.8% | PEG-4347 | 11.4% | |
| PEG-0646 | 38.2% | 1.1% | PEG-4019 | 9.1% | 0.6% | PEG-4184 | 15.9% | 0.6% | PEG-4348 | 14.6% | |
| PEG-0647 | 24.4% | 1.2% | PEG-4020 | 7.9% | 0.5% | PEG-4185 | 21.6% | 0.6% | PEG-0748 | 9.2% | |
| PEG-0648 | 30.7% | 0.9% | PEG-4021 | 7.5% | 0.3% | PEG-4186 | 12.3% | 0.7% | PEG-4350 | 13.5% | |
| PEG-0649 | 32.6% | 4.1% | PEG-4022 | 3.6% | 0.4% | PEG-4187 | 7.5% | 0.5% | PEG-0753 | 11.8% | |
| PEG-0650 | 20.3% | 1.1% | PEG-4023 | 10.6% | 0.4% | PEG-4188 | 12.7% | 0.3% | PEG-4352 | 11.2% | |
| PEG-0651 | 11.1% | 0.6% | PEG-4025 | 4.3% | 0.4% | PEG-4189 | 23.7% | 0.4% | PEG-4353 | 11.4% | |
| PEG-0652 | 16.1% | 0.9% | PEG-4026 | 7.0% | 0.5% | PEG-4190 | 19.9% | 0.8% | PEG-4354 | 11.3% | |
| PEG-0669 | 29.4% | 1.4% | PEG-4027 | 8.1% | 0.8% | PEG-4191 | 11.2% | 0.7% | PEG-0757 | 10.6% | |
| PEG-0670 | 21.3% | 0.5% | PEG-4028 | 7.2% | 0.3% | PEG-4192 | 11.5% | 0.6% | PEG-4356 | 14.9% | |
| PEG-0671 | 29.6% | 1.2% | PEG-4029 | 4.0% | 0.3% | PEG-4193 | 9.8% | 0.8% | PEG-4357 | 13.6% | |
| PEG-0672 | 21.2% | 0.6% | PEG-4030 | 2.3% | 0.2% | PEG-4194 | 18.5% | 0.6% | PEG-0761 | 11.9% | |
| PEG-0673 | 19.0% | 1.1% | PEG-4031 | 3.7% | 0.3% | PEG-4195 | 21.5% | 1.0% | PEG-4359 | 13.9% | |
| PEG-0674 | 18.2% | 0.6% | PEG-4032 | 2.9% | 0.4% | PEG-4196 | 16.9% | 0.6% | PEG-4360 | 14.7% | |
| PEG-0675 | 22.1% | 1.0% | PEG-4033 | 3.3% | 0.1% | PEG-4197 | 14.5% | 0.6% | PEG-4361 | 11.2% | |
| PEG-0676 | 15.0% | 0.6% | PEG-4034 | 1.7% | 0.2% | PEG-4198 | 20.3% | 0.9% | PEG-4362 | 10.1% | |
| PEG-0800 | 19.2% | 2.3% | PEG-4035 | 3.2% | 0.2% | PEG-4199 | 22.9% | 1.1% | PEG-4363 | 10.5% | |
| PEG-0801 | 24.2% | 2.7% | PEG-4036 | 11.2% | 0.7% | PEG-4200 | 8.9% | 0.4% | PEG-4364 | 11.6% | |
| PEG-0802 | 21.0% | 6.5% | PEG-4037 | 10.2% | 0.3% | PEG-4201 | 9.9% | 0.4% | PEG-4365 | 9.3% | |
| PEG-0803 | 23.9% | 2.7% | PEG-4038 | 8.3% | 0.4% | PEG-4202 | 7.0% | 0.4% | PEG-4366 | 7.9% | |
| PEG-0804 | 21.3% | 3.0% | PEG-4039 | 4.5% | 0.4% | PEG-4203 | 13.6% | 0.5% | PEG-4367 | 12.6% | |
| PEG-0805 | 16.1% | 1.7% | PEG-4040 | 3.4% | 0.3% | PEG-4204 | 13.4% | 0.8% | PEG-4368 | 8.2% | |
| PEG-0806 | 12.4% | 1.3% | PEG-4041 | 3.2% | 0.3% | PEG-4205 | 8.0% | 0.4% | PEG-0754 | 4.0% | |
| PEG-0809 | 23.2% | 2.6% | PEG-4042 | 3.4% | 0.2% | PEG-4206 | 4.0% | 0.3% | PEG-4370 | 3.7% | |
| PEG-0810 | 20.5% | 4.6% | PEG-4043 | 9.4% | 0.7% | PEG-4207 | 6.7% | 0.4% | PEG-4371 | 2.1% | |
| PEG-0811 | 25.4% | 5.7% | PEG-4044 | 4.6% | 0.5% | PEG-4208 | 12.3% | 0.4% | PEG-4372 | 1.6% | |
| PEG-0812 | 20.4% | 2.3% | PEG-4045 | 2.1% | 0.4% | PEG-4209 | 9.7% | 0.4% | PEG-0758 | 1.6% | |
| PEG-0813 | 25.0% | 3.1% | PEG-4046 | 6.6% | 0.5% | PEG-4210 | 7.6% | 0.3% | PEG-4374 | 1.6% | |
| PEG-0814 | 13.2% | 1.3% | PEG-4047 | 2.2% | 0.3% | PEG-4211 | 4.8% | 0.4% | PEG-4375 | 1.6% | |
| PEG-0815 | 13.0% | 1.4% | PEG-4048 | 3.0% | 0.4% | PEG-4212 | 2.0% | 0.2% | PEG-0762 | 3.5% | |
| PEG-0818 | 23.2% | 2.0% | PEG-4049 | 4.2% | 0.7% | PEG-4213 | 9.5% | 0.3% | PEG-4377 | 4.8% | |
| PEG-0819 | 27.1% | 4.1% | PEG-4050 | 6.5% | 0.4% | PEG-4214 | 10.9% | 0.3% | PEG-4378 | 4.3% | |
| PEG-0820 | 26.0% | 2.7% | PEG-4051 | 6.2% | 0.5% | PEG-4215 | 6.6% | 0.4% | PEG-4379 | 4.4% | |
| PEG-0821 | 23.9% | 1.5% | PEG-4052 | 8.4% | 0.6% | PEG-4216 | 1.9% | 0.2% | PEG-4380 | 2.6% | |
| PEG-0822 | 23.3% | 2.2% | PEG-4053 | 8.2% | 0.6% | PEG-4217 | 2.8% | 0.3% | PEG-4381 | 3.1% | |
| PEG-0823 | 13.5% | 1.0% | PEG-4054 | 9.4% | 0.7% | PEG-4218 | 5.0% | 0.5% | PEG-4382 | 2.7% | |
| PEG-0824 | 15.5% | 1.7% | PEG-4055 | 6.7% | 0.5% | PEG-4219 | 1.3% | 0.2% | PEG-4383 | 1.9% | |
| PEG-0827 | 17.9% | 2.4% | PEG-4056 | 9.3% | 0.8% | PEG-4220 | 1.6% | 0.2% | PEG-4384 | 3.7% | |
| PEG-0828 | 28.0% | 3.0% | PEG-4057 | 9.2% | 0.7% | PEG-4221 | 3.0% | 0.3% | PEG-4385 | 4.3% | |
| PEG-0829 | 22.8% | 2.5% | PEG-4058 | 7.3% | 0.4% | PEG-4222 | 1.9% | 0.2% | PEG-4386 | 3.4% | |
| PEG-0830 | 26.1% | 1.7% | PEG-4059 | 6.3% | 0.7% | PEG-4223 | 3.5% | 0.3% | PEG-0755 | 3.5% | |
| PEG-0831 | 21.3% | 2.2% | PEG-4060 | 5.9% | 0.5% | PEG-4224 | 2.1% | 0.4% | PEG-4388 | 2.4% | |
| PEG-0832 | 17.1% | 1.3% | PEG-4061 | 8.8% | 0.3% | PEG-0769 | 2.7% | 0.4% | PEG-4389 | 2.5% | |
| PEG-0833 | 11.9% | 1.3% | PEG-4062 | 6.6% | 0.7% | PEG-4226 | 2.5% | 0.5% | PEG-4390 | 2.1% | |
| PEG-0836 | 15.4% | 1.8% | PEG-4063 | 5.9% | 0.8% | PEG-4227 | 1.9% | 0.5% | PEG-0759 | 2.7% | |
| PEG-0837 | 24.8% | 3.3% | PEG-4064 | 8.3% | 0.4% | PEG-4228 | 1.0% | 0.2% | PEG-4392 | 1.7% | |
| PEG-0838 | 18.1% | 2.1% | PEG-4065 | 3.0% | 0.4% | PEG-0773 | 0.7% | 0.2% | PEG-4393 | 2.1% | |
| PEG-0839 | 26.1% | 2.7% | PEG-4066 | 10.5% | 0.8% | PEG-4230 | 1.0% | 0.3% | PEG-0763 | 2.2% | |
| PEG-0840 | 15.5% | 1.9% | PEG-4067 | 12.1% | 1.0% | PEG-4231 | 1.4% | 0.4% | PEG-4395 | 3.3% | |
| PEG-0841 | 14.9% | 1.5% | PEG-4068 | 6.9% | 0.4% | PEG-0777 | 2.8% | 0.5% | PEG-4396 | 3.1% | |
| PEG-0842 | 9.9% | 0.9% | PEG-4069 | 5.8% | 0.6% | PEG-4233 | 1.5% | 0.4% | PEG-4397 | 2.9% | |
| PEG-0845 | 14.7% | 1.4% | PEG-4070 | 6.7% | 0.4% | PEG-4234 | 1.6% | 0.3% | PEG-4398 | 2.3% | |
| PEG-0846 | 16.1% | 2.4% | PEG-4071 | 7.0% | 0.2% | PEG-4235 | 1.8% | 0.4% | PEG-4399 | 1.0% | |
| PEG-0847 | 20.6% | 1.8% | PEG-4072 | 5.9% | 0.4% | PEG-4236 | 1.4% | 0.3% | PEG-4400 | 0.9% | |
| PEG-0848 | 18.2% | 2.7% | PEG-4073 | 4.5% | 0.3% | PEG-4237 | 0.9% | 0.4% | PEG-4401 | 1.1% | |
| PEG-0849 | 14.7% | 1.9% | PEG-4074 | 2.4% | 0.3% | PEG-4238 | 0.7% | 0.1% | PEG-4402 | 1.4% | |
| PEG-0850 | 9.9% | 1.0% | PEG-4075 | 2.1% | 0.3% | PEG-4239 | 0.6% | 0.3% | PEG-4403 | 1.7% | |
| PEG-0851 | 9.8% | 1.3% | PEG-4076 | 5.1% | 0.6% | PEG-4240 | 0.7% | 0.3% | PEG-4404 | 1.1% | |
| PEG-0944 | 15.2% | 0.8% | PEG-4077 | 2.7% | 0.5% | PEG-4241 | 1.4% | 0.3% | PEG-0756 | 1.1% | |
| PEG-0945 | 8.7% | 0.6% | PEG-4078 | 2.7% | 0.3% | PEG-4242 | 1.0% | 0.4% | PEG-4406 | 1.5% | |
| PEG-0946 | 13.5% | 0.2% | PEG-4079 | 5.3% | 0.5% | PEG-0770 | 1.0% | 0.3% | PEG-4407 | 1.2% | |
| PEG-0947 | 9.8% | 0.6% | PEG-4080 | 2.8% | 0.6% | PEG-4244 | 1.3% | 0.4% | PEG-4408 | 1.2% | |
| PEG-0948 | 11.7% | 0.7% | PEG-4081 | 5.1% | 0.2% | PEG-4245 | 1.5% | 0.3% | PEG-0760 | 0.7% | |
| PEG-0949 | 10.5% | 0.7% | PEG-4082 | 7.3% | 0.8% | PEG-4246 | 0.1% | 0.2% | PEG-4410 | 0.9% | |
| PEG-0950 | 5.5% | 0.4% | PEG-4083 | 5.2% | 0.6% | PEG-0774 | 0.2% | 0.2% | PEG-4411 | 1.2% | |
| PEG-0953 | 16.0% | 0.6% | PEG-4084 | 14.8% | 0.7% | PEG-4248 | 0.1% | 0.2% | PEG-0764 | 1.3% | |

TABLE 18h
Correction of G339C using PE3 systems, experiment 1.

| PEG# | ng # | edit % | indel % | PEG# | ng # | edit % | indel % | PEG# | ng # | edit % | indel % |
|---|---|---|---|---|---|---|---|---|---|---|---|
| PEG-0829 | 370 | 30% | 1% | PEG-0829 | 357 | 35% | 6% | PEG-0625 | 5015 | 13% | 6% |
| PEG-0962 | 370 | 27% | 3% | PEG-0838 | 357 | 28% | 5% | PEG-0754 | 5015 | 13% | 1% |
| PEG-1208 | 370 | 26% | 1% | PEG-1208 | 357 | 26% | 11% | PEG-0649 | 5015 | 10% | 6% |
| PEG-1228 | 370 | 19% | 1% | PEG-0962 | 357 | 23% | 8% | PEG-0673 | 5015 | 8% | 5% |
| PEG-1248 | 370 | 19% | 1% | PEG-1228 | 357 | 21% | 12% | PEG-0649 | 5014 | 30% | 1% |
| PEG-0838 | 370 | 17% | 1% | PEG-1248 | 357 | 19% | 9% | PEG-0625 | 5014 | 25% | 1% |
| PEG-0649 | 338 | 51% | 4% | PEG-0838 | 356 | 33% | 8% | PEG-0738 | 5014 | 23% | 1% |
| PEG-0625 | 338 | 41% | 5% | PEG-0829 | 356 | 32% | 4% | PEG-0065 | 5014 | 17% | 1% |
| PEG-0065 | 338 | 30% | 5% | PEG-1208 | 356 | 32% | 1% | PEG-0673 | 5014 | 17% | 1% |
| PEG-0673 | 338 | 29% | 3% | PEG-0962 | 356 | 31% | 2% | PEG-0754 | 5014 | 11% | 1% |
| PEG-0738 | 338 | 29% | 2% | PEG-1228 | 356 | 30% | 2% | PEG-0649 | 333 | 45% | 4% |
| PEG-0754 | 338 | 8% | 1% | PEG-1248 | 356 | 21% | 2% | PEG-0625 | 333 | 31% | 3% |
| PEG-0649 | 5003 | 45% | 3% | PEG-0838 | 355 | 32% | 4% | PEG-0065 | 333 | 29% | 5% |
| PEG-0625 | 5003 | 37% | 4% | PEG-0829 | 355 | 30% | 3% | PEG-0673 | 333 | 25% | 3% |
| PEG-0738 | 5003 | 31% | 2% | PEG-0962 | 355 | 27% | 2% | PEG-0738 | 333 | 16% | 2% |
| PEG-0673 | 5003 | 29% | 2% | PEG-1228 | 355 | 25% | 2% | PEG-0754 | 333 | 7% | 1% |
| PEG-0065 | 5003 | 27% | 3% | PEG-1208 | 355 | 24% | 2% | PEG-0649 | 5013 | 33% | 2% |
| PEG-0754 | 5003 | 10% | 1% | PEG-1248 | 355 | 16% | 1% | PEG-0065 | 5013 | 26% | 1% |
| PEG-0649 | 5002 | 45% | 3% | PEG-0838 | 354 | 37% | 5% | PEG-0625 | 5013 | 26% | 1% |
| PEG-0625 | 5002 | 35% | 3% | PEG-0829 | 354 | 36% | 2% | PEG-0673 | 5013 | 25% | 1% |
| PEG-0673 | 5002 | 27% | 3% | PEG-0962 | 354 | 27% | 2% | PEG-0738 | 5013 | 25% | 2% |
| PEG-0738 | 5002 | 27% | 2% | PEG-1228 | 354 | 26% | 1% | PEG-0754 | 5013 | 17% | 1% |
| PEG-0065 | 5002 | 24% | 2% | PEG-1208 | 354 | 25% | 1% | PEG-1248 | 364 | 29% | 2% |
| PEG-0754 | 5002 | 11% | 1% | PEG-1248 | 354 | 18% | 1% | PEG-0962 | 364 | 27% | 1% |
| PEG-0649 | 5001 | 45% | 3% | PEG-0838 | 353 | 35% | 9% | PEG-1208 | 364 | 25% | 1% |
| PEG-0625 | 5001 | 34% | 3% | PEG-0829 | 353 | 34% | 2% | PEG-1228 | 364 | 24% | 2% |
| PEG-0065 | 5001 | 30% | 1% | PEG-1208 | 353 | 27% | 1% | PEG-0829 | 364 | 20% | 2% |
| PEG-0738 | 5001 | 27% | 1% | PEG-0962 | 353 | 26% | 1% | PEG-0838 | 364 | 12% | 2% |
| PEG-0673 | 5001 | 26% | 2% | PEG-1228 | 353 | 25% | 1% | PEG-1208 | 361 | 25% | 6% |
| PEG-0754 | 5001 | 9% | 1% | PEG-1248 | 353 | 17% | 1% | PEG-0962 | 361 | 24% | 7% |
| PEG-1208 | 363 | 27% | 2% | PEG-0829 | 352 | 34% | 2% | PEG-1228 | 361 | 23% | 7% |
| PEG-0962 | 363 | 23% | 3% | PEG-1228 | 352 | 26% | 2% | PEG-0829 | 361 | 22% | 10% |
| PEG-1248 | 363 | 22% | 3% | PEG-1208 | 352 | 25% | 2% | PEG-1248 | 361 | 21% | 6% |
| PEG-1228 | 363 | 21% | 3% | PEG-0838 | 352 | 24% | 2% | PEG-0838 | 361 | 19% | 7% |
| PEG-0829 | 363 | 20% | 9% | PEG-0962 | 352 | 22% | 1% | PEG-1248 | 359 | 25% | 13% |
| PEG-0838 | 363 | 17% | 7% | PEG-1248 | 352 | 16% | 1% | PEG-1228 | 359 | 24% | 12% |
| PEG-0625 | 340 | 40% | 5% | PEG-0649 | 336 | 42% | 3% | PEG-0829 | 359 | 23% | 6% |
| PEG-0649 | 340 | 35% | 3% | PEG-0065 | 336 | 31% | 4% | PEG-0962 | 359 | 22% | 9% |
| PEG-0065 | 340 | 24% | 5% | PEG-0625 | 336 | 30% | 2% | PEG-1208 | 359 | 19% | 6% |
| PEG-0673 | 340 | 22% | 2% | PEG-0673 | 336 | 22% | 2% | PEG-0838 | 359 | 18% | 4% |
| PEG-0738 | 340 | 19% | 2% | PEG-0738 | 336 | 14% | 1% | PEG-1208 | 369 | 29% | 2% |
| PEG-0754 | 340 | 15% | 2% | PEG-0754 | 336 | 4% | 1% | PEG-0962 | 369 | 27% | 3% |
| PEG-1208 | 362 | 28% | 3% | PEG-0625 | 344 | 34% | 6% | PEG-1228 | 369 | 27% | 2% |
| PEG-0962 | 362 | 26% | 1% | PEG-0649 | 344 | 34% | 6% | PEG-1248 | 369 | 20% | 2% |
| PEG-0829 | 362 | 25% | 11% | PEG-0738 | 344 | 26% | 2% | PEG-0829 | 369 | 9% | 4% |
| PEG-1228 | 362 | 25% | 4% | PEG-0673 | 344 | 23% | 6% | PEG-0838 | 369 | 4% | 4% |
| PEG-1248 | 362 | 25% | 4% | PEG-0065 | 344 | 20% | 6% | PEG-0829 | 350 | 31% | 7% |
| PEG-0838 | 362 | 18% | 8% | PEG-0754 | 344 | 17% | 1% | PEG-0838 | 350 | 27% | 5% |
| PEG-0962 | 360 | 25% | 6% | PEG-0649 | 345 | 37% | 5% | PEG-1208 | 350 | 19% | 2% |
| PEG-1248 | 360 | 22% | 5% | PEG-0625 | 345 | 33% | 6% | PEG-1228 | 350 | 15% | 2% |
| PEG-0829 | 360 | 21% | 9% | PEG-0738 | 345 | 33% | 2% | PEG-0962 | 350 | 13% | 2% |
| PEG-1208 | 360 | 21% | 5% | PEG-0673 | 345 | 21% | 5% | PEG-1248 | 350 | 12% | 1% |
| PEG-1228 | 360 | 19% | 7% | PEG-0754 | 345 | 17% | 1% | PEG-0649 | 339 | 47% | 5% |
| PEG-0838 | 360 | 16% | 10% | PEG-0065 | 345 | 15% | 5% | PEG-0673 | 339 | 34% | 5% |
| PEG-0649 | 337 | 43% | 3% | PEG-0829 | 351 | 26% | 2% | PEG-0625 | 339 | 32% | 3% |
| PEG-0625 | 337 | 31% | 3% | PEG-0838 | 351 | 25% | 3% | PEG-0738 | 339 | 30% | 6% |
| PEG-0065 | 337 | 28% | 1% | PEG-1208 | 351 | 16% | 6% | PEG-0065 | 339 | 24% | 4% |
| PEG-0673 | 337 | 24% | 1% | PEG-1228 | 351 | 13% | 5% | PEG-0754 | 339 | 13% | 5% |
| PEG-0738 | 337 | 21% | 2% | PEG-0962 | 351 | 11% | 7% | PEG-0649 | 341 | 46% | 4% |
| PEG-0754 | 337 | 8% | 1% | PEG-1248 | 351 | 8% | 2% | PEG-0065 | 341 | 33% | 6% |
| PEG-0829 | 358 | 27% | 4% | PEG-0829 | 349 | 30% | 3% | PEG-0625 | 341 | 30% | 3% |
| PEG-0838 | 358 | 25% | 5% | PEG-0838 | 349 | 30% | 4% | PEG-0673 | 341 | 29% | 4% |
| PEG-1208 | 358 | 23% | 10% | PEG-0962 | 349 | 11% | 3% | PEG-0738 | 341 | 26% | 4% |
| PEG-1228 | 358 | 21% | 13% | PEG-1208 | 349 | 9% | 3% | PEG-0754 | 341 | 11% | 2% |
| PEG-1248 | 358 | 20° | 11% | PEG-1228 | 349 | 9% | 3% | PEG-0649 | 342 | 31% | 7% |
| PEG-0962 | 358 | 19% | 9% | PEG-1248 | 349 | 8% | 3% | PEG-0625 | 342 | 25% | 6% |
| PEG-0829 | 365 | 30% | 5% | PEG-0649 | 335 | 25% | 2% | PEG-0065 | 342 | 22% | 8% |
| PEG-1248 | 365 | 29% | 3% | PEG-0065 | 335 | 23% | 2% | PEG-0738 | 342 | 17% | 2% |
| PEG-0962 | 365 | 28% | 2% | PEG-0673 | 335 | 22% | 1% | PEG-0673 | 342 | 14% | 6% |
| PEG-1208 | 365 | 28% | 3% | PEG-0625 | 335 | 20% | 1% | PEG-0754 | 342 | 11% | 1% |
| PEG-1228 | 365 | 24% | 3% | PEG-0738 | 335 | 18% | 1% | PEG-0738 | 346 | 31% | 2% |
| PEG-0838 | 365 | 12% | 1% | PEG-0754 | 335 | 9% | 1% | PEG-0649 | 346 | 30% | 2% |
| PEG-0962 | 366 | 28% | 1% | PEG-0738 | 347 | 31% | 2% | PEG-0625 | 346 | 26% | 2% |
| PEG-1208 | 366 | 27% | 2% | PEG-0649 | 347 | 27% | 3% | PEG-0065 | 346 | 19% | 2% |
| PEG-1248 | 366 | 24% | 2% | PEG-0625 | 347 | 20% | 3% | PEG-0673 | 346 | 18% | 1% |
| PEG-0829 | 366 | 23% | 2% | PEG-0673 | 347 | 15% | 3% | PEG-0754 | 346 | 15% | 1% |
| PEG-1228 | 366 | 23% | 3% | PEG-0754 | 347 | 14% | 1% | PEG-0649 | 334 | 34% | 2% |
| PEG-0838 | 366 | 9% | 3% | PEG-0065 | 347 | 12% | 3% | PEG-0625 | 334 | 24% | 2% |
| PEG-0625 | 343 | 36% | 6% | PEG-0738 | 5017 | 34% | 3% | PEG-0738 | 334 | 24% | 4% |
| PEG-0649 | 343 | 33% | 7% | PEG-0649 | 5017 | 23% | 3% | PEG-0065 | 334 | 22% | 3% |
| PEG-0738 | 343 | 24° | 3% | PEG-0673 | 5017 | 22% | 2% | PEG-0673 | 334 | 20% | 2% |
| PEG-0065 | 343 | 18% | 6% | PEG-0065 | 5017 | 16% | 2% | PEG-0754 | 334 | 7% | 3% |
| PEG-0673 | 343 | 17% | 5% | PEG-0754 | 5017 | 16% | 1% | PEG-0649 | none | 43% | 3% |
| PEG-0754 | 343 | 14% | 2% | PEG-0625 | 5017 | 14% | 3% | PEG-0829 | none | 37% | 1% |
| PEG-0962 | 367 | 37% | 3% | PEG-0829 | 348 | 28% | 3% | PEG-0962 | none | 31% | 2% |
| PEG-1208 | 367 | 27% | 2% | PEG-0838 | 348 | 18% | 2% | PEG-1248 | none | 28% | 3% |
| PEG-1228 | 367 | 25% | 3% | PEG-1208 | 348 | 8% | 2% | PEG-0065 | none | 27% | 2% |
| PEG-1248 | 367 | 24% | 2% | PEG-1248 | 348 | 7% | 1% | PEG-0625 | none | 27% | 2% |
| PEG-0829 | 367 | 22% | 2% | PEG-1228 | 348 | 6% | 1% | PEG-1208 | none | 24% | 3% |
| PEG-0838 | 367 | 9% | 2% | PEG-0962 | 348 | 5% | 1% | PEG-0673 | none | 21% | 1% |
| PEG-1208 | 368 | 30% | 3% | PEG-0738 | 5016 | 36% | 3% | PEG-0838 | none | 21% | 0% |
| PEG-0962 | 368 | 29% | 2% | PEG-0649 | 5016 | 35% | 4% | PEG-1228 | none | 19% | 3% |
| PEG-1248 | 368 | 28% | 3% | PEG-0625 | 5016 | 23% | 3% | PEG-0738 | none | 18% | 1% |
| PEG-1228 | 368 | 23% | 3% | PEG-0673 | 5016 | 21% | 4% | PEG-0754 | none | 5% | 1% |
| PEG-0829 | 368 | 19% | 1% | PEG-0754 | 5016 | 16% | 1% | | | | |
| PEG-0838 | 368 | 12% | 2% | PEG-0738 | 5015 | 31% | 2% | | | | |

TABLE 18i
Correction of G339C using PE3 systems, experiment 2.

| PEG# | ng # | edit % | Indel % | PEG# | ng # | edit % | indel % | PEG# | ng # | edit % | indel % |
|---|---|---|---|---|---|---|---|---|---|---|---|
| PEG-0065 | 333 | 5% | 8% | PEG-1208 | 352 | 25% | 1% | PEG-0065 | 5001 | 22% | 1% |
| PEG-0625 | 333 | 8% | 8% | PEG-1228 | 352 | 28% | 1% | PEG-0625 | 5001 | 27% | 1% |
| PEG-0649 | 333 | 8% | 8% | PEG-1248 | 352 | 17% | 1% | PEG-0649 | 5001 | 30% | 2% |
| PEG-0673 | 333 | 7% | 6% | PEG-0829 | 353 | 27% | 4% | PEG-0673 | 5001 | 17% | 1% |
| PEG-0738 | 333 | 15% | 6% | PEG-0838 | 353 | 27% | 3% | PEG-0738 | 5001 | 22% | 1% |
| PEG-0754 | 333 | 7% | 4% | PEG-0962 | 353 | 17% | 1% | PEG-0754 | 5001 | 12% | 1% |
| PEG-0065 | 334 | 11% | 7% | PEG-1208 | 353 | 29% | 2% | PEG-0065 | 5002 | 21% | 1% |
| PEG-0625 | 334 | 11% | 7% | PEG-1228 | 353 | 33% | 2% | PEG-0625 | 5002 | 26% | 1% |
| PEG-0649 | 334 | 12% | 10% | PEG-1248 | 353 | 22% | 2% | PEG-0649 | 5002 | 27% | 2% |
| PEG-0673 | 334 | 8% | 5% | PEG-0829 | 354 | 26% | 2% | PEG-0673 | 5002 | 17% | 1% |
| PEG-0738 | 334 | 16% | 5% | PEG-0838 | 354 | 26% | 2% | PEG-0738 | 5002 | 22% | 2% |
| PEG-0754 | 334 | 7% | 4% | PEG-0962 | 354 | 20% | 2% | PEG-0754 | 5002 | 10% | 1% |
| PEG-0065 | 335 | 12% | 6% | PEG-1208 | 354 | 24% | 2% | PEG-0065 | 5003 | 20% | 1% |
| PEG-0625 | 335 | 15% | 7% | PEG-1228 | 354 | 31% | 2% | PEG-0625 | 5003 | 26% | 2% |
| PEG-0649 | 335 | 19% | 9% | PEG-1248 | 354 | 23% | 2% | PEG-0649 | 5003 | 28% | 2% |
| PEG-0673 | 335 | 10% | 5% | PEG-0829 | 355 | 29% | 2% | PEG-0673 | 5003 | 17% | 2% |
| PEG-0738 | 335 | 17% | 4% | PEG-0838 | 355 | 25% | 2% | PEG-0738 | 5003 | 22% | 2% |
| PEG-0754 | 335 | 7% | 3% | PEG-0962 | 355 | 21% | 1% | PEG-0754 | 5003 | 5% | 1% |
| PEG-0065 | 336 | 11% | 10% | PEG-1208 | 355 | 26% | 1% | PEG-0625 | 5013 | 26% | 2% |
| PEG-0625 | 336 | 13% | 15% | PEG-1228 | 355 | 32% | 2% | PEG-0649 | 5013 | 27% | 3% |
| PEG-0649 | 336 | 15% | 17% | PEG-1248 | 355 | 22% | 1% | PEG-0673 | 5013 | 28% | 3% |
| PEG-0673 | 336 | 9% | 10% | PEG-0829 | 356 | 30% | 5% | PEG-0738 | 5013 | 23% | 2% |
| PEG-0738 | 336 | 17% | 9% | PEG-0838 | 356 | 30% | 4% | PEG-0754 | 5013 | 16% | 2% |
| PEG-0754 | 336 | 7% | 6% | PEG-0962 | 356 | 23% | 2% | PEG-0065 | 5014 | 20% | 1% |
| PEG-0065 | 337 | 20% | 3% | PEG-1208 | 356 | 29% | 2% | PEG-0625 | 5014 | 25% | 2% |
| PEG-0625 | 337 | 26% | 3% | PEG-1228 | 356 | 40% | 3% | PEG-0649 | 5014 | 25% | 2% |
| PEG-0649 | 337 | 27% | 4% | PEG-1248 | 356 | 27% | 2% | PEG-0673 | 5014 | 24% | 2% |
| PEG-0673 | 337 | 19% | 2% | PEG-0829 | 357 | 29% | 5% | PEG-0738 | 5014 | 26% | 2% |
| PEG-0738 | 337 | 19% | 2% | PEG-0838 | 357 | 29% | 5% | PEG-0754 | 5014 | 16% | 1% |
| PEG-0754 | 337 | 8% | 1% | PEG-0962 | 357 | 21% | 3% | PEG-0065 | 5015 | 22% | 2% |
| PEG-0065 | 338 | 27% | 2% | PEG-1208 | 357 | 30% | 2% | PEG-0625 | 5015 | 32% | 3% |
| PEG-0625 | 338 | 28% | 2% | PEG-1228 | 357 | 37% | 4% | PEG-0649 | 5015 | 31% | 3% |
| PEG-0649 | 338 | 32% | 3% | PEG-1248 | 357 | 30% | 2% | PEG-0673 | 5015 | 29% | 2% |
| PEG-0673 | 338 | 21% | 1% | PEG-0829 | 358 | 24% | 6% | PEG-0738 | 5015 | 33% | 2% |
| PEG-0738 | 338 | 26% | 2% | PEG-0838 | 358 | 24% | 6% | PEG-0754 | 5015 | 15% | 1% |
| PEG-0754 | 338 | 11% | 1% | PEG-0962 | 358 | 16% | 3% | PEG-0065 | 5016 | 25% | 2% |
| PEG-0065 | 339 | 19% | 11% | PEG-1208 | 358 | 28% | 3% | PEG-0625 | 5016 | 34% | 3% |
| PEG-0625 | 339 | 22% | 16% | PEG-1228 | 358 | 33% | 4% | PEG-0649 | 5016 | 31% | 4% |
| PEG-0649 | 339 | 21% | 18% | PEG-1248 | 358 | 27% | 4% | PEG-0673 | 5016 | 32% | 3% |
| PEG-0673 | 339 | 20% | 12% | PEG-0829 | 359 | 20% | 6% | PEG-0738 | 5016 | 29% | 2% |
| PEG-0738 | 339 | 26% | 6% | PEG-0838 | 359 | 22% | 9% | PEG-0754 | 5016 | 19% | 1% |
| PEG-0754 | 339 | 15% | 5% | PEG-0962 | 359 | 21% | 4% | PEG-0065 | 5017 | 23% | 2% |
| PEG-0065 | 340 | 21% | 16% | PEG-1208 | 359 | 30% | 3% | PEG-0625 | 5017 | 29% | 3% |
| PEG-0625 | 340 | 21% | 15% | PEG-1228 | 359 | 24% | 4% | PEG-0649 | 5017 | 29% | 5% |
| PEG-0649 | 340 | 15% | 15% | PEG-1248 | 359 | 23% | 6% | PEG-0673 | 5017 | 28% | 3% |
| PEG-0673 | 340 | 20% | 13% | PEG-0829 | 360 | 13% | 10% | PEG-0738 | 5017 | 27% | 2% |
| PEG-0738 | 340 | 23% | 4% | PEG-0838 | 360 | 19% | 18% | PEG-0754 | 5017 | 19% | 1% |
| PEG-0754 | 340 | 15% | 4% | PEG-0962 | 360 | 13% | 8% | PEG-0829 | 5030 | 24% | 1% |
| PEG-0065 | 341 | 25% | 14% | PEG-1208 | 360 | 28% | 9% | PEG-0838 | 5030 | 17% | 1% |
| PEG-0625 | 341 | 26% | 12% | PEG-1228 | 360 | 25% | 7% | PEG-0962 | 5030 | 16% | 1% |
| PEG-0649 | 341 | 25% | 16% | PEG-1248 | 360 | 17% | 7% | PEG-1208 | 5030 | 24% | 1% |
| PEG-0673 | 341 | 21% | 11% | PEG-0829 | 361 | 21% | 9% | PEG-1228 | 5030 | 20% | 1% |
| PEG-0738 | 341 | 29% | 4% | PEG-0838 | 361 | 20% | 12% | PEG-1248 | 5030 | 19% | 1% |
| PEG-0754 | 341 | 16% | 3% | PEG-0962 | 361 | 19% | 8% | PEG-0065 | none | 24% | 2% |
| PEG-0065 | 342 | 18% | 5% | PEG-1208 | 361 | 29% | 6% | PEG-0065 | none | 20% | 1% |
| PEG-0625 | 342 | 23% | 7% | PEG-1228 | 361 | 27% | 6% | PEG-0625 | none | 28% | 2% |
| PEG-0649 | 342 | 23% | 8% | PEG-1248 | 361 | 19% | 7% | PEG-0625 | none | 24% | 2% |
| PEG-0673 | 342 | 18% | 5% | PEG-0829 | 362 | 18% | 10% | PEG-0649 | none | 24% | 2% |
| PEG-0738 | 342 | 22% | 3% | PEG-0838 | 362 | 20% | 12% | PEG-0649 | none | 24% | 2% |
| PEG-0754 | 342 | 19% | 2% | PEG-0962 | 362 | 18% | 8% | PEG-0673 | none | 24% | 1% |
| PEG-0065 | 343 | 20% | 5% | PEG-1208 | 362 | 31% | 5% | PEG-0673 | none | 23% | 1% |
| PEG-0625 | 343 | 25% | 7% | PEG-1228 | 362 | 30% | 6% | PEG-0738 | none | 26% | 2% |
| PEG-0649 | 343 | 24% | 8% | PEG-1248 | 362 | 24% | 7% | PEG-0738 | none | 25% | 1% |
| PEG-0673 | 343 | 21% | 5% | PEG-0829 | 363 | 15% | 11% | PEG-0738 | none | 24% | 2% |
| PEG-0738 | 343 | 27% | 2% | PEG-0838 | 363 | 20% | 15% | PEG-0738 | none | 22% | 2% |
| PEG-0754 | 343 | 16% | 2% | PEG-0962 | 363 | 17% | 12% | PEG-0738 | none | 18% | 3% |
| PEG-0065 | 344 | 23% | 3% | PEG-1208 | 363 | 28% | 8% | PEG-0738 | none | 16% | 1% |
| PEG-0625 | 344 | 26% | 3% | PEG-1228 | 363 | 31% | 8% | PEG-0754 | none | 11% | 1% |
| PEG-0649 | 344 | 31% | 4% | PEG-1248 | 363 | 24% | 13% | PEG-0754 | none | 10% | 1% |
| PEG-0673 | 344 | 23% | 3% | PEG-0829 | 364 | 20% | 5% | PEG-0754 | none | 10% | 1% |
| PEG-0738 | 344 | 29% | 2% | PEG-0838 | 364 | 15% | 4% | PEG-0754 | none | 9% | 1% |
| PEG-0754 | 344 | 18% | 2% | PEG-0962 | 364 | 14% | 2% | PEG-0754 | none | 9% | 1% |
| PEG-0065 | 345 | 20% | 2% | PEG-1208 | 364 | 17% | 1% | PEG-0754 | none | 8% | 1% |
| PEG-0625 | 345 | 26% | 2% | PEG-1228 | 364 | 17% | 1% | PEG-0829 | none | 29% | 1% |
| PEG-0649 | 345 | 28% | 4% | PEG-1248 | 364 | 19% | 1% | PEG-0829 | none | 27% | 1% |
| PEG-0673 | 345 | 23% | 2% | PEG-0829 | 365 | 22% | 3% | PEG-0829 | none | 20% | 1% |
| PEG-0738 | 345 | 29% | 2% | PEG-0838 | 365 | 18% | 3% | PEG-0829 | none | 20% | 1% |
| PEG-0754 | 345 | 22% | 1% | PEG-0962 | 365 | 18% | 3% | PEG-0829 | none | 14% | 1% |
| PEG-0065 | 346 | 22% | 2% | PEG-1208 | 365 | 26% | 2% | PEG-0829 | none | 13% | 1% |
| PEG-0625 | 346 | 27% | 2% | PEG-1228 | 365 | 27% | 2% | PEG-0838 | none | 26% | 1% |
| PEG-0649 | 346 | 28% | 4% | PEG-1248 | 365 | 22% | 3% | PEG-0838 | none | 23% | 1% |
| PEG-0673 | 346 | 28% | 3% | PEG-0829 | 366 | 17% | 5% | PEG-0838 | none | 18% | 0% |
| PEG-0738 | 346 | 27% | 3% | PEG-0838 | 366 | 14% | 6% | PEG-0838 | none | 17% | 1% |
| PEG-0754 | 346 | 19% | 1% | PEG-0962 | 366 | 15% | 4% | PEG-0838 | none | 16% | 1% |
| PEG-0065 | 347 | 23% | 2% | PEG-1208 | 366 | 20% | 3% | PEG-0838 | none | 14% | 1% |
| PEG-0625 | 347 | 31% | 3% | PEG-1228 | 366 | 24% | 4% | PEG-0962 | none | 24% | 1% |
| PEG-0649 | 347 | 30% | 4% | PEG-1248 | 366 | 18% | 6% | PEG-0962 | none | 20% | 1% |
| PEG-0673 | 347 | 28% | 3% | PEG-0829 | 367 | 18% | 4% | PEG-0962 | none | 19% | 1% |
| PEG-0738 | 347 | 27% | 2% | PEG-0838 | 367 | 15% | 4% | PEG-0962 | none | 18% | 0% |
| PEG-0754 | 347 | 18% | 1% | PEG-0962 | 367 | 15% | 3% | PEG-0962 | none | 16% | 1% |
| PEG-0829 | 348 | 26% | 3% | PEG-1208 | 367 | 15% | 6% | PEG-0962 | none | 14% | 1% |
| PEG-0838 | 348 | 22% | 2% | PEG-1228 | 367 | 20% | 4% | PEG-0962 | none | 13% | 1% |
| PEG-0962 | 348 | 18% | 1% | PEG-1248 | 367 | 18% | 5% | PEG-0962 | none | 13% | 1% |
| PEG-1208 | 348 | 22% | 1% | PEG-0829 | 368 | 15% | 8% | PEG-1208 | none | 30% | 1% |
| PEG-1228 | 348 | 26% | 2% | PEG-0838 | 368 | 15% | 8% | PEG-1208 | none | 30% | 1% |
| PEG-1248 | 348 | 20% | 2% | PEG-0962 | 368 | 16% | 9% | PEG-1208 | none | 24% | 1% |
| PEG-0829 | 349 | 30% | 2% | PEG-1208 | 368 | 17% | 5% | PEG-1208 | none | 23% | 1% |
| PEG-0838 | 349 | 25% | 3% | PEG-1228 | 368 | 23% | 7% | PEG-1208 | none | 23% | 1% |
| PEG-0962 | 349 | 19% | 1% | PEG-1248 | 368 | 18% | 12% | PEG-1208 | none | 22% | 1% |
| PEG-1208 | 349 | 23% | 1% | PEG-0829 | 369 | 12% | 3% | PEG-1208 | none | 21% | 1% |
| PEG-1228 | 349 | 30% | 2% | PEG-0838 | 369 | 12% | 5% | PEG-1208 | none | 20% | 1% |
| PEG-1248 | 349 | 20% | 1% | PEG-0962 | 369 | 10% | 4% | PEG-1228 | none | 26% | 1% |
| PEG-0829 | 350 | 27% | 4% | PEG-1208 | 369 | 18% | 4% | PEG-1228 | none | 25% | 1% |
| PEG-0838 | 350 | 27% | 4% | PEG-1228 | 369 | 17% | 4% | PEG-1228 | none | 24% | 1% |
| PEG-0962 | 350 | 19% | 2% | PEG-1248 | 369 | 12% | 5% | PEG-1228 | none | 23% | 1% |
| PEG-1208 | 350 | 25% | 3% | PEG-0065 | 370 | 9% | 11% | PEG-1228 | none | 23% | 1% |
| PEG-1228 | 350 | 31% | 3% | PEG-0625 | 370 | 7% | 9% | PEG-1228 | none | 22% | 1% |
| PEG-1248 | 350 | 20% | 2% | PEG-0649 | 370 | 8% | 10% | PEG-1228 | none | 19% | 1% |
| PEG-0829 | 351 | 22% | 2% | PEG-0673 | 370 | 6% | 7% | PEG-1228 | none | 19% | 1% |
| PEG-0838 | 351 | 19% | 2% | PEG-0738 | 370 | 9% | 5% | PEG-1248 | none | 21% | 1% |
| PEG-0962 | 351 | 13% | 1% | PEG-0754 | 370 | 4% | 2% | PEG-1248 | none | 21% | 1% |
| PEG-1208 | 351 | 22% | 1% | PEG-0829 | 370 | 23% | 1% | PEG-1248 | none | 21% | 1% |
| PEG-1228 | 351 | 26% | 2% | PEG-0838 | 370 | 17% | 1% | PEG-1248 | none | 19% | 1% |
| PEG-1248 | 351 | 18% | 1% | PEG-0962 | 370 | 15% | 1% | PEG-1248 | none | 18% | 1% |
| PEG-0829 | 352 | 29% | 2% | PEG-1208 | 370 | 22% | 1% | PEG-1248 | none | 17% | 1% |
| PEG-0838 | 352 | 22% | 1% | PEG-1228 | 370 | 21% | 2% | PEG-1248 | none | 17% | 1% |
| PEG-0962 | 352 | 13% | 1% | PEG-1248 | 370 | 20% | 1% | PEG-1248 | none | 15% | 1% |

TABLE 18j
Correction of G339C using PE3 systems, experiment 3.

| | | First trial | | | | | | Second trial | | | | | |
|---|---|---|---|---|---|---|---|---|---|---|---|---|---|
| | | Replicate A | | Replicate B | | Replicate C | | Replicate A | | Replicate B | | Replicate C | |
| PEG# | ng# | edit % | indel % | edit % | indel % | edit % | indel % | edit % | indel % | edit % | indel % | edit % | indel % |
| 4005 | 5003 | 40.1 | 1.1 | 40.7 | 40.7 | 44.5 | 1.2 | 40.9 | 1.3 | 43.3 | 1.4 | 42.2 | 1.2 |
| 625 | 5003 | 29.1 | 1.9 | 34.1 | 34.1 | 35.4 | 2.4 | 33.6 | 1.6 | 34.7 | 2.1 | 40.3 | 2.1 |
| 670 | 5002 | 40.4 | 1.1 | 45.0 | 45.0 | 46.5 | 1.2 | 44.8 | 1.2 | 44.4 | 0.9 | 46.2 | 1.1 |
| 673 | 5002 | 35.4 | 1.1 | 34.6 | 34.6 | 37.0 | 1.5 | 34.8 | 1.3 | 37.1 | 1.3 | 38.6 | 1.2 |
| 674 | 5002 | 27.5 | 1.0 | 35.8 | 35.8 | 34.2 | 1.2 | 37.3 | 1.3 | 38.4 | 1.3 | 39.9 | 1.5 |
| 49 | 338 | 43.0 | 6.8 | 47.2 | 47.2 | 45.4 | 7.7 | 43.4 | 6.9 | 45.4 | 8.3 | 49.0 | 9.1 |
| 47 | 338 | 38.2 | 5.8 | 48.3 | 48.3 | 44.8 | 6.3 | 43.9 | 6.7 | 48.3 | 6.5 | 49.7 | 7.8 |
| 56 | 338 | 35.7 | 6.7 | 42.3 | 42.3 | 42.0 | 8.6 | 43.1 | 8.1 | 43.2 | 9.0 | 45.4 | 10.2 |
| 50 | 338 | 34.1 | 9.3 | 31.1 | 31.1 | 34.5 | 10.0 | 41.7 | 13.4 | 41.4 | 13.1 | 44.5 | 13.3 |
| 65 | 338 | 24.7 | 7.9 | 28.2 | 28.2 | 24.1 | 9.3 | 23.9 | 6.6 | 25.2 | 7.4 | 27.6 | 13.9 |
| 67 | 338 | 26.1 | 4.2 | 26.0 | 26.0 | 26.3 | 4.3 | 22.7 | 3.9 | 25.3 | 3.7 | 25.8 | 3.7 |
| 645 | 5001 | 39.9 | 1.9 | 42.3 | 42.3 | 45.6 | 2.1 | 47.4 | 1.3 | 43.6 | 2.0 | 47.3 | 1.9 |
| 646 | 5001 | 41.2 | 1.0 | 40.5 | 40.5 | 43.6 | 1.0 | 40.4 | 0.9 | 41.6 | 0.8 | 43.0 | 1.0 |
| 4006 | 5001 | 34.1 | 2.0 | 40.7 | 40.7 | 45.7 | 2.7 | 40.1 | 2.6 | 41.8 | 2.9 | 44.6 | 2.0 |
| 649 | 5001 | 32.2 | 3.3 | 29.3 | 29.3 | 29.9 | 3.2 | 34.6 | 3.6 | 35.7 | 3.4 | 39.1 | 3.9 |
| 645 | 364 | 54.0 | 1.5 | 52.8 | 52.8 | 53.3 | 1.6 | 59.8 | 1.6 | 56.7 | 1.7 | 60.9 | 1.4 |
| 670 | 364 | 49.3 | 0.8 | 55.9 | 55.9 | 54.6 | 1.2 | 53.3 | 1.0 | 54.6 | 1.2 | 57.2 | 0.9 |
| 646 | 364 | 56.1 | 0.7 | 50.8 | 50.8 | 49.4 | 1.0 | 56.9 | 0.9 | 53.6 | 0.8 | 55.3 | 1.0 |
| 4006 | 364 | 46.9 | 1.5 | 53.0 | 53.0 | 51.2 | 2.1 | 47.8 | 1.6 | 48.9 | 1.8 | 52.5 | 1.9 |
| 4005 | 364 | 52.8 | 1.1 | 49.8 | 49.8 | 47.6 | 1.1 | 48.0 | 0.9 | 49.7 | 1.1 | 52.1 | 1.1 |
| 649 | 364 | 37.3 | 2.3 | 35.5 | 35.5 | 31.2 | 2.3 | 41.3 | 2.3 | 40.3 | 2.0 | 41.4 | 2.1 |
| 674 | 364 | 32.0 | 1.1 | 38.2 | 38.2 | 32.0 | 1.2 | 35.7 | 1.1 | 39.1 | 1.1 | 40.8 | 1.4 |
| 673 | 364 | 31.3 | 1.2 | 32.7 | 32.7 | 37.8 | 1.5 | 31.4 | 1.0 | 35.3 | 0.9 | 35.0 | 1.3 |
| 625 | 364 | 21.8 | 1.0 | 33.5 | 33.5 | 34.7 | 1.3 | 38.9 | 1.2 | 36.7 | 0.9 | 36.8 | 1.5 |
| 49 | 364 | 29.2 | 4.8 | 29.8 | 29.8 | 32.1 | 5.9 | 31.2 | 5.4 | 30.7 | 5.2 | 31.5 | 5.6 |
| 47 | 364 | 26.6 | 4.2 | 28.7 | 28.7 | 30.0 | 4.6 | 31.7 | 5.2 | 30.1 | 5.0 | 30.3 | 4.8 |
| 50 | 364 | 24.5 | 7.5 | 23.4 | 23.4 | 24.3 | 7.2 | 24.4 | 8.4 | 24.5 | 8.9 | 24.2 | 8.7 |
| 56 | 364 | 21.8 | 4.3 | 22.1 | 22.1 | 23.4 | 4.9 | 21.2 | 4.6 | 20.9 | 5.2 | 21.8 | 4.4 |
| 65 | 364 | 9.9 | 3.4 | 11.3 | 11.3 | 13.1 | 5.7 | 10.2 | 4.5 | 10.8 | 3.9 | 11.5 | 4.7 |
| 67 | 364 | 10.0 | 1.8 | 12.1 | 12.1 | 13.5 | 2.9 | 9.6 | 2.5 | 11.7 | 2.2 | 9.3 | 2.6 |
| 4009 | 5005 | 12.2 | 1.9 | 10.9 | 10.9 | 9.5 | 1.6 | 14.3 | 2.5 | 14.9 | 2.4 | 15.3 | 2.6 |
| 292 | 337 | 31.7 | 2.1 | 34.1 | 34.1 | 31.4 | 1.9 | 36.9 | 2.8 | 39.0 | 3.2 | 39.9 | 3.4 |
| 251 | 337 | 11.4 | 0.7 | 11.6 | 11.6 | 11.6 | 0.6 | 13.8 | 0.8 | 13.2 | 1.3 | 13.2 | 1.0 |
| 237 | 337 | 5.2 | 0.3 | 7.4 | 7.4 | 7.2 | 0.3 | 9.6 | 0.6 | 10.1 | 0.3 | 11.8 | 0.8 |
| 238 | 337 | 8.1 | 0.4 | 7.5 | 7.5 | 5.5 | 0.3 | 8.5 | 0.4 | 9.3 | 0.6 | 9.1 | 0.4 |
| 4007 | 5006 | 15.3 | 1.3 | 16.3 | 16.3 | 15.6 | 1.5 | 20.2 | 2.2 | 19.6 | 2.2 | 21.0 | 2.1 |
| 738 | 5006 | 12.0 | 1.2 | 11.3 | 11.3 | 14.8 | 1.6 | 16.1 | 1.5 | 16.2 | 1.6 | 17.8 | 2.1 |
| 746 | 5006 | 6.9 | 0.8 | 5.0 | 5.0 | 7.1 | 0.9 | 7.3 | 0.6 | 9.3 | 1.4 | 10.9 | 1.4 |
| 1224 | 365 | 45.2 | 0.8 | 46.7 | 46.7 | 49.5 | 1.0 | 48.2 | 1.0 | 51.4 | 1.0 | 55.7 | 0.7 |
| 1225 | 365 | 32.3 | 0.6 | 35.7 | 35.7 | 38.8 | 0.8 | 38.0 | 0.7 | 41.8 | 1.0 | 42.6 | 0.9 |
| 944 | 365 | 26.5 | 0.9 | 33.7 | 33.7 | 36.3 | 1.4 | 40.0 | 1.4 | 39.7 | 2.0 | 38.8 | 1.3 |
| 4012 | 365 | 28.5 | 1.3 | 27.4 | 27.4 | 28.7 | 1.1 | 33.1 | 1.4 | 31.9 | 1.5 | 35.2 | 1.4 |
| 962 | 365 | 27.3 | 0.7 | 27.8 | 27.8 | 24.4 | 0.6 | 33.3 | 1.0 | 31.3 | 0.8 | 34.0 | 1.5 |
| 946 | 365 | 25.4 | 0.6 | 27.2 | 27.2 | 29.8 | 0.6 | 30.2 | 0.6 | 28.3 | 0.7 | 32.2 | 0.8 |
| 4011 | 365 | 29.0 | 0.6 | 24.7 | 24.7 | 26.8 | 0.6 | 32.3 | 0.6 | 28.8 | 0.8 | 28.6 | 0.5 |
| 956 | 365 | 24.2 | 0.7 | 26.5 | 26.5 | 27.8 | 0.6 | 29.0 | 0.8 | 30.6 | 1.0 | 29.0 | 1.0 |
| 1215 | 365 | 17.9 | 0.7 | 25.5 | 25.5 | 30.2 | 1.3 | 29.0 | 1.2 | 31.8 | 1.2 | 30.5 | 1.6 |
| 959 | 365 | 19.7 | 0.5 | 18.8 | 18.8 | 19.6 | 0.6 | 27.9 | 1.0 | 27.8 | 1.3 | 30.2 | 1.1 |
| 1217 | 365 | 15.9 | 0.8 | 21.2 | 21.2 | 23.2 | 1.1 | 24.9 | 1.9 | 24.1 | 1.8 | 28.1 | 1.4 |
| 1208 | 365 | 17.0 | 0.9 | 18.7 | 18.7 | 20.8 | 0.9 | 24.5 | 0.7 | 24.0 | 0.9 | 27.0 | 0.9 |
| 1000 | 365 | 10.6 | 0.4 | 17.3 | 17.3 | 18.1 | 0.8 | 18.9 | 0.7 | 21.4 | 0.9 | 20.6 | 0.7 |
| 986 | 365 | 9.8 | 0.5 | 11.0 | 11.0 | 13.1 | 0.6 | 15.7 | 0.8 | 16.1 | 0.9 | 17.8 | 1.2 |
| 1224 | 356 | 49.0 | 2.9 | 55.7 | 55.7 | 55.5 | 3.6 | 60.6 | 3.3 | 60.0 | 4.3 | 65.4 | 5.2 |
| 1225 | 356 | 43.1 | 1.7 | 50.6 | 50.6 | 51.5 | 3.3 | 51.3 | 2.2 | 54.8 | 3.1 | 51.5 | 2.8 |
| 944 | 356 | 29.4 | 2.9 | 37.5 | 37.5 | 36.8 | 5.2 | 38.6 | 4.0 | 42.2 | 6.3 | 41.6 | 3.6 |
| 4012 | 356 | 33.0 | 3.7 | 32.2 | 32.2 | 33.9 | 3.8 | 41.1 | 4.1 | 38.8 | 4.0 | 43.7 | 5.7 |
| 946 | 356 | 28.9 | 2.6 | 32.7 | 32.7 | 33.3 | 3.0 | 35.2 | 2.9 | 36.3 | 3.0 | 38.6 | 3.3 |
| 4011 | 356 | 33.6 | 2.6 | 28.2 | 28.2 | 31.0 | 2.0 | 35.5 | 1.8 | 37.8 | 2.6 | 38.9 | 2.5 |
| 962 | 356 | 24.9 | 1.7 | 26.7 | 26.7 | 28.7 | 1.6 | 37.6 | 3.5 | 37.1 | 3.6 | 39.8 | 4.6 |
| 1215 | 356 | 20.5 | 1.4 | 27.3 | 27.3 | 32.5 | 3.4 | 31.1 | 3.0 | 37.0 | 3.7 | 36.3 | 3.8 |
| 956 | 356 | 22.8 | 1.4 | 31.2 | 31.2 | 30.4 | 2.2 | 31.2 | 1.8 | 34.2 | 3.1 | 34.1 | 2.5 |
| 1217 | 356 | 21.6 | 1.9 | 27.0 | 27.0 | 29.5 | 3.7 | 32.8 | 4.6 | 33.6 | 3.7 | 38.7 | 4.6 |
| 1208 | 356 | 19.2 | 1.8 | 23.8 | 23.8 | 28.7 | 3.1 | 31.9 | 2.7 | 32.8 | 3.5 | 37.5 | 4.8 |
| 959 | 356 | 18.8 | 1.1 | 17.7 | 17.7 | 22.4 | 1.3 | 30.7 | 3.0 | 31.9 | 3.2 | 33.3 | 4.0 |
| 1000 | 356 | 10.4 | 0.8 | 20.3 | 20.3 | 22.0 | 1.8 | 20.4 | 1.5 | 25.5 | 2.7 | 24.0 | 2.5 |
| 986 | 356 | 12.5 | 1.4 | 15.5 | 15.5 | 16.8 | 1.8 | 16.3 | 1.8 | 17.9 | 2.2 | 21.1 | 2.3 |
| 801 | 354 | 40.3 | 9.5 | 41.5 | 41.5 | 42.2 | 10.9 | 42.7 | 10.2 | 47.4 | 9.9 | 45.1 | 10.8 |
| 837 | 354 | 31.5 | 5.4 | 37.6 | 37.6 | 38.0 | 7.5 | 37.9 | 9.5 | 41.1 | 9.5 | 43.6 | 9.1 |
| 819 | 354 | 36.6 | 6.9 | 36.1 | 36.1 | 38.7 | 8.7 | 37.8 | 7.1 | 40.0 | 7.7 | 40.4 | 6.8 |
| 828 | 354 | 29.3 | 4.9 | 37.8 | 37.8 | 39.4 | 7.6 | 41.3 | 7.5 | 39.9 | 8.3 | 40.2 | 7.5 |
| 820 | 354 | 28.3 | 5.5 | 36.4 | 36.4 | 36.2 | 7.7 | 40.1 | 8.5 | 41.2 | 10.2 | 43.1 | 10.9 |
| 838 | 354 | 32.2 | 5.6 | 31.5 | 31.5 | 35.5 | 6.8 | 36.1 | 5.7 | 37.0 | 6.7 | 36.3 | 6.5 |
| 829 | 354 | 24.7 | 3.6 | 28.6 | 28.6 | 30.9 | 4.5 | 33.1 | 6.6 | 35.1 | 7.1 | 35.8 | 9.0 |
| 805 | 354 | 21.8 | 2.5 | 23.6 | 23.6 | 24.7 | 3.4 | 23.6 | 2.3 | 26.9 | 3.0 | 30.8 | 5.4 |
| 801 | 353 | 36.4 | 14.1 | 36.9 | 36.9 | 38.2 | 17.1 | 43.0 | 14.3 | 43.7 | 15.2 | 44.6 | 14.5 |
| 837 | 353 | 31.2 | 7.5 | 35.2 | 35.2 | 35.2 | 10.4 | 38.7 | 10.6 | 41.0 | 12.6 | 39.4 | 13.2 |
| 819 | 353 | 31.0 | 7.9 | 36.7 | 36.7 | 36.2 | 13.2 | 37.6 | 9.9 | 38.4 | 11.2 | 39.0 | 9.7 |
| 820 | 353 | 29.8 | 8.0 | 35.3 | 35.3 | 35.6 | 10.9 | 39.0 | 12.8 | 39.1 | 13.9 | 40.0 | 15.1 |
| 828 | 353 | 25.0 | 5.0 | 35.4 | 35.4 | 36.7 | 10.5 | 38.4 | 9.8 | 39.5 | 10.5 | 40.9 | 11.0 |
| 838 | 353 | 33.0 | 9.0 | 31.9 | 31.9 | 33.4 | 9.6 | 35.9 | 8.2 | 37.0 | 9.6 | 38.0 | 11.7 |
| 829 | 353 | 26.1 | 4.9 | 30.0 | 30.0 | 31.0 | 6.8 | 36.7 | 10.8 | 35.1 | 9.5 | 37.8 | 11.7 |
| 805 | 353 | 23.9 | 5.1 | 21.8 | 21.8 | 23.6 | 5.0 | 26.5 | 4.9 | 27.6 | 6.7 | 29.9 | 6.9 |
| 292 | 5016 | 37.8 | 2.9 | 39.4 | 39.4 | 42.1 | 3.2 | 48.6 | 3.4 | 51.2 | 4.2 | | |
| 4007 | 5016 | 24.9 | 2.3 | 22.6 | 22.6 | 23.5 | 2.2 | 35.6 | 3.2 | 30.4 | 3.3 | 37.7 | 3.4 |
| 738 | 5016 | 18.5 | 1.8 | 19.4 | 19.4 | 23.6 | 2.5 | 27.5 | 2.8 | 27.2 | 3.5 | 29.4 | 3.1 |
| 251 | 5016 | 18.3 | 1.6 | 19.2 | 19.2 | 18.1 | 1.6 | 20.7 | 1.2 | 20.8 | 2.1 | 24.4 | 2.6 |
| 746 | 5016 | 13.4 | 1.5 | 12.4 | 12.4 | 13.8 | 1.7 | 16.7 | 2.1 | 20.7 | 1.8 | 24.4 | 2.2 |
| 4009 | 5016 | 14.1 | 2.7 | 11.9 | 11.9 | 11.0 | 1.9 | 19.6 | 4.3 | 19.8 | 4.1 | 17.7 | 3.9 |
| 237 | 5016 | 7.4 | 0.6 | 9.4 | 9.4 | 11.4 | 1.0 | 16.0 | 1.4 | 16.3 | 1.4 | 16.6 | 1.9 |
| 238 | 5016 | 7.3 | 0.5 | 6.8 | 6.8 | 6.0 | 0.4 | 11.7 | 1.0 | 13.3 | 1.2 | 14.5 | 1.2 |

TABLE 18k
Correction of G339C using PE3 systems in iPSC.

| | | | | Trial 1 | | | | Trial 2 | | | |
|---|---|---|---|---|---|---|---|---|---|---|---|
| | | | | Replcate A | | Replicate B | | Replcate A | | Replicate B | |
| PEG # | ng# | Dose | | Edit % | Indel % | Edit % | Indel % | Edit % | Indel % | Edit % | Indel % |
| 649 | 338 | 486.7 | ng | 5 | 0.27 | 6.78 | 0.28 | | | 18.58 | 0.79 |
| 649 | 338 | 324.5 | ng | 7.51 | 0.42 | 7.06 | 0.23 | 16.64 | 0.39 | 15.77 | 0.53 |
| 649 | 338 | 216.4 | ng | 4.37 | 0.28 | 4.55 | 0.21 | 12.99 | 0.56 | 14.13 | 0.61 |
| 649 | 338 | 144.3 | ng | 2.97 | 0.26 | 2.8 | 0.19 | 9.06 | 0.27 | 7.41 | 0.56 |
| 649 | 338 | 96.1 | ng | 1.62 | 0.18 | 1.17 | 0.16 | 3.95 | 0.1 | 3.88 | 0.28 |
| 649 | 338 | 64 | ng | 0.92 | 0.05 | 0.56 | 0.08 | 1.97 | 0.05 | 1.76 | 0.1 |
| 649 | 338 | 42.7 | ng | 0.35 | 0.1 | 0.22 | 0.12 | 0.59 | 0.14 | 0.88 | 0.12 |
| 649 | 338 | 28.5 | ng | 0.28 | 0.14 | 0.26 | 0.1 | 0.54 | 0.1 | 0.34 | 0.1 |
| 649 | 338 | 19 | ng | 0.12 | 0.08 | 0.12 | 0.04 | 0.14 | 0.07 | 0.2 | 0.16 |
| 649 | 338 | 12.7 | ng | 0.11 | 0.11 | 0.06 | 0.05 | 0.12 | 0.02 | 0.06 | 0.09 |
| 649 | 338 | 8.5 | ng | 0.07 | 0.12 | 0.11 | 0.06 | 0.15 | 0.03 | 0.15 | 0.13 |
| 649 | 338 | 5.6 | ng | 0.05 | 0.05 | 0.02 | 0.08 | 0.05 | 0.04 | | |
| 1208 | 367 | 486.7 | ng | 22.36 | 0.39 | 27.82 | 0.46 | | | | |
| 1208 | 367 | 324.5 | ng | 14.11 | 0.35 | 21.48 | 0.39 | 38.23 | 0.67 | 43.95 | 0.77 |
| 1208 | 367 | 216.4 | ng | 18.79 | 0.32 | 15.39 | 0.27 | 40.64 | 0.8 | 39.13 | 0.67 |
| 1208 | 367 | 144.3 | ng | 10.14 | 0.18 | 8.54 | 0.16 | 30.55 | 0.4 | 26.28 | 0.5 |
| 1208 | 367 | 96.1 | ng | 7.11 | 0.16 | 3.81 | 0.11 | 14.35 | 0.2 | 13.82 | 0.23 |
| 1208 | 367 | 64 | ng | 2.56 | 0.08 | 1.58 | 0.1 | 8.01 | 0.13 | 6.94 | 0.11 |
| 1208 | 367 | 42.7 | ng | 1.39 | 0.09 | 1.31 | 0.01 | 1.73 | 0.13 | 2.95 | 0.12 |
| 1208 | 367 | 28.5 | ng | 0.44 | 0.04 | 0.61 | 0.05 | 1.5 | 0.03 | 1.35 | 0.07 |
| 1208 | 367 | 19 | ng | 0.31 | 0.05 | 0.37 | 0.05 | 0.52 | 0.08 | 0.88 | 0.14 |
| 1208 | 367 | 12.7 | ng | 0.31 | 0.05 | 0.21 | 0.03 | 0.28 | 0.07 | 0.32 | 0.08 |
| 1208 | 367 | 8.5 | ng | 0.13 | 0.07 | 0.06 | 0.03 | 0.07 | 0.05 | 0.21 | 0.05 |
| 1208 | 367 | 5.6 | ng | 0.11 | 0.06 | 0.06 | 0.07 | 0.11 | 0.06 | 0.17 | 0.13 |
| 829 | 354 | 486.7 | ng | 17.99 | 1.18 | 18.26 | 1 | | | 29.01 | 2.45 |
| 829 | 354 | 324.5 | ng | 17.41 | 1.06 | 14.54 | 0.58 | 17.92 | 3.71 | 24.63 | 2.06 |
| 829 | 354 | 216.4 | ng | 10.4 | 0.32 | 9.74 | 0.33 | 24.91 | 1.07 | 22.04 | 1.17 |
| 829 | 354 | 144.3 | ng | 7.15 | 0.31 | 3.82 | 0.11 | 16.84 | 0.42 | 12.1 | 0.45 |
| 829 | 354 | 96.1 | ng | 4.54 | 0.16 | 1.99 | 0.04 | 12.95 | 0.21 | 6.01 | 0.28 |
| 829 | 354 | 64 | ng | 1.54 | 0.04 | 1.01 | 0.03 | 3.62 | 0.06 | 2.86 | 0.17 |
| 829 | 354 | 42.7 | ng | 0.74 | 0.06 | 0.32 | 0.03 | 2.12 | 0.1 | 1.39 | 0.07 |
| 829 | 354 | 28.5 | ng | 0.32 | 0.06 | 0.26 | 0.03 | 0.48 | 0.07 | 0.53 | 0.12 |
| 829 | 354 | 19 | ng | 0.13 | 0.06 | 0.11 | 0.07 | 0.36 | 0.08 | 0.39 | 0.13 |
| 829 | 354 | 12.7 | ng | 0.14 | 0.06 | 0.07 | 0.08 | 0.14 | 0.12 | 0.15 | 0.1 |
| 829 | 354 | 8.5 | ng | 0.05 | 0.08 | 0.03 | 0.03 | 0.17 | 0.12 | 0.14 | 0.11 |
| 829 | 354 | 5.6 | ng | 0.11 | 0.07 | 0.03 | 0.08 | 0.01 | 0.08 | 0.09 | 0.04 |
| 738 | 5017 | 486.7 | ng | 5.99 | 1.34 | 3.47 | 0.58 | | | 6.87 | 1.61 |
| 738 | 5017 | 324.5 | ng | 1.89 | 0.57 | 2.64 | 0.59 | 2.34 | 0.54 | 4.41 | 1.06 |
| 738 | 5017 | 216.4 | ng | 2.46 | 0.37 | 1.5 | 0.11 | 7.12 | 1.48 | 2.85 | 0.62 |
| 738 | 5017 | 144.3 | ng | 1.29 | 0.43 | 0.34 | 0.09 | 2.9 | 0.67 | 1.5 | 0.38 |
| 738 | 5017 | 96.1 | ng | 0.41 | 0.12 | 0.35 | 0.05 | 2.26 | 0.06 | 0.73 | 0.22 |
| 738 | 5017 | 64 | ng | 0.2 | 0.05 | 0.02 | 0.06 | 0.61 | 0.34 | 0.22 | 0.11 |
| 738 | 5017 | 42.7 | ng | 0.08 | 0.08 | 0.06 | 0.03 | 0.05 | 0.05 | 0.04 | 0.09 |
| 738 | 5017 | 28.5 | ng | 0.05 | 0.04 | 0.03 | 0.07 | 0.06 | 0.06 | 0.01 | 0.16 |
| 738 | 5017 | 19 | ng | 0.07 | 0.09 | 0.01 | 0.08 | 0.07 | 0.08 | 0.04 | 0.11 |
| 738 | 5017 | 12.7 | ng | 0.07 | 0.08 | 0.03 | 0.07 | 0.02 | 0.1 | 0.05 | 0.09 |
| 738 | 5017 | 8.5 | ng | 0.04 | 0.05 | 0.04 | 0.02 | 0.02 | 0.09 | 0.03 | 0.1 |
| 738 | 5017 | 5.6 | ng | 0.01 | 0.05 | 0.01 | 0.04 | 0.01 | 0.1 | 0.04 | 0.11 |
| 649 | none | 486.7 | ng | 11.35 | 0.75 | 7.3 | 0.37 | | | 9.94 | 0.41 |
| 649 | none | 324.5 | ng | 8.86 | 0.59 | 6.86 | 0.34 | 3.47 | 0.81 | 12.18 | 0.55 |
| 649 | none | 216.4 | ng | 8.35 | 0.48 | 3.83 | 0.36 | 13.48 | 0.7 | 9.21 | 0.51 |
| 649 | none | 144.3 | ng | 4.03 | 0.26 | 1.77 | 0.09 | 8.48 | 0.4 | 5.71 | 0.3 |
| 649 | none | 96.1 | ng | 1.75 | 0.2 | 0.9 | 0.11 | 6.55 | 0.38 | 2.48 | 0.22 |
| 649 | none | 64 | ng | 0.66 | 0.09 | 0.59 | 0.08 | 1.7 | 0.03 | 1.13 | 0.22 |
| 649 | none | 42.7 | ng | 0.3 | 0.05 | 0.44 | 0.03 | 0.89 | 0.26 | 0.53 | 0.13 |
| 649 | none | 28.5 | ng | 0.16 | 0.06 | 0.29 | 0.1 | 0.25 | 0.09 | 0.38 | 0.07 |
| 649 | none | 19 | ng | 0.08 | 0.06 | 0.14 | 0.02 | 0.33 | 0.07 | 0.14 | 0.17 |
| 649 | none | 12.7 | ng | 0.06 | 0.09 | 0.04 | 0.05 | 0.21 | 0.06 | 0.08 | 0.11 |
| 649 | none | 8.5 | ng | 0.02 | 0.08 | 0.05 | 0.09 | 0.14 | 0.06 | 0.06 | 0.08 |
| 649 | none | 5.6 | ng | 0.06 | 0.1 | 0.02 | 0.03 | 0.17 | 0.15 | 0.03 | 0.05 |
| 1208 | none | 486.7 | ng | 11.7 | 0.46 | 9.52 | 0.22 | | | 14.19 | 0.29 |
| 1208 | none | 324.5 | ng | 6.55 | 0.14 | 7.66 | 0.17 | 6.99 | 0.98 | 13.12 | 0.54 |
| 1208 | none | 216.4 | ng | 8.78 | 0.16 | 7.67 | 0.14 | 11.23 | 0.6 | 9.4 | 0.33 |
| 1208 | none | 144.3 | ng | 5.84 | 0.16 | 3.03 | 0.1 | 7.7 | 0.28 | 7.98 | 0.25 |
| 1208 | none | 96.1 | ng | 3.99 | 0.1 | 2.15 | 0.07 | 5.76 | 0.14 | 4.99 | 0.15 |
| 1208 | none | 64 | ng | 1.79 | 0.08 | 1.22 | 0.06 | 5.9 | 0.18 | 2.48 | 0.13 |
| 1208 | none | 42.7 | ng | 0.92 | 0.07 | 0.62 | 0.09 | 2.86 | 0.09 | 1.41 | 0.16 |
| 1208 | none | 28.5 | ng | 0.55 | 0.1 | 0.38 | 0.03 | 0.73 | 0.15 | 0.59 | 0.18 |
| 1208 | none | 19 | ng | 0.31 | 0.07 | 0.19 | 0.07 | 0.13 | 0.07 | 0.21 | 0.1 |
| 1208 | none | 12.7 | ng | 0.11 | 0.04 | 0.09 | 0.07 | 0.16 | 0.13 | 0.18 | 0.13 |
| 1208 | none | 8.5 | ng | 0.13 | 0.08 | 0.09 | 0.08 | 0.33 | 0.05 | 0.09 | 0.1 |
| 1208 | none | 5.6 | ng | 0.06 | 0.09 | 0.06 | 0.02 | 0.04 | 0.02 | 0.08 | 0.1 |
| 829 | none | 486.7 | ng | 15.4 | 0.55 | 12.76 | 0.39 | | | 21.84 | 0.7 |
| 829 | none | 324.5 | ng | 13.72 | 0.5 | 8.86 | 0.28 | 16.18 | 1.05 | 19.2 | 0.77 |
| 829 | none | 216.4 | ng | 7.72 | 0.16 | 5.86 | 0.13 | 19.99 | 0.99 | 12.65 | 0.23 |
| 829 | none | 144.3 | ng | 5.29 | 0.15 | 3.07 | 0.09 | 13.99 | 0.41 | 11.92 | 0.48 |
| 829 | none | 96.1 | ng | 2.64 | 0.06 | 1.52 | 0.05 | 12.2 | 0.31 | 6.54 | 0.24 |
| 829 | none | 64 | ng | 1.24 | 0.08 | 0.65 | 0.04 | 7.69 | 0.23 | 2.74 | 0.18 |
| 829 | none | 42.7 | ng | 0.56 | 0.04 | 0.3 | 0.05 | 2.82 | 0.09 | 1.1 | 0.08 |
| 829 | none | 28.5 | ng | 0.36 | 0.06 | 0.12 | 0.06 | 0.58 | 0.05 | 0.45 | 0.15 |
| 829 | none | 19 | ng | 0.19 | 0.06 | 0.11 | 0.07 | 0.54 | 0.11 | 0.17 | 0.08 |
| 829 | none | 12.7 | ng | 0.18 | 0.08 | 0.05 | 0.07 | 0.33 | 0.09 | 0.26 | 0.13 |
| 829 | none | 8.5 | ng | 0.07 | 0.09 | 0.05 | 0.05 | 0.06 | 0.09 | 0.15 | 0.07 |
| 829 | none | 5.6 | ng | 0.02 | 0.05 | 0.04 | 0.06 | 0.14 | 0.1 | 0.05 | 0.1 |
| 738 | none | 486.7 | ng | 1.84 | 0.28 | 1.84 | 0.08 | | | 3.38 | 0.5 |
| 738 | none | 324.5 | ng | 2.22 | 0.23 | 1.11 | 0.08 | 3.48 | 0.23 | 2.23 | 0.17 |
| 738 | none | 216.4 | ng | 1.8 | 0.18 | 0.61 | 0.15 | 2.13 | 0.64 | 1.41 | 0.17 |
| 738 | none | 144.3 | ng | 0.56 | 0.12 | 0.41 | 0.09 | 1.69 | 0.34 | 0.7 | 0.22 |
| 738 | none | 96.1 | ng | 0.3 | 0.11 | 0.14 | 0.07 | 0.44 | 0.14 | 0.44 | 0.1 |
| 738 | none | 64 | ng | 0.15 | 0.07 | 0.05 | 0.04 | 0.53 | 0.11 | 0.24 | 0.08 |
| 738 | none | 42.7 | ng | 0.01 | 0.06 | 0.04 | 0.1 | 0.03 | 0.08 | 0.04 | 0.05 |
| 738 | none | 28.5 | ng | 0.03 | 0.08 | 0.03 | 0.03 | 0.02 | 0.05 | 0.1 | 0.12 |
| 738 | none | 19 | ng | 0.04 | 0.05 | 0.02 | 0.1 | 0.03 | 0.16 | 0.04 | 0.13 |
| 738 | none | 12.7 | ng | 0.03 | 0.06 | 0.01 | 0.07 | 0.03 | 0.08 | 0.02 | 0.15 |
| 738 | none | 8.5 | ng | 0.05 | 0.08 | 0.03 | 0.05 | 0.03 | 0.1 | 0.03 | 0.19 |
| 738 | none | 5.6 | ng | 0.06 | 0.03 | 0.01 | 0.09 | 0.02 | 0.02 | 0.04 | 0.15 |

TABLE 181
Partial list of sequences

| SEQ ID | |
|---|---|
| NO. | Sequence |
| 54 | guuuuAGAGCUAGAAAUAGCAAGUUAAAAUAAGGCUAGUCCGUUAUCAACU |
| | UGAAAAAGUGGCACCGAGUCGgugc |
| 57 | AACAUUGACGCGUCUCUACGUGGGGGCGCG |
| 60 | CAGGGCAAUGGGGCCAUACG |
| 61 | UGACUCCAAACAGGGCAAUG |
| 62 | AUGACUCCAAACAGGGCAAU |
| 63 | UAUGACUCCAAACAGGGCAA |
| 64 | GUUGGCUAUGACUCCAAACA |
| 65 | CGUUGGCUAUGACUCCAAAC |
| 66 | CAGCUCUGGAUCCUGGUAUU |
| 67 | GCAGCUCUGGAUCCUGGUAU |
| 68 | CCCUUGGCAGCUCUGGAUCC |
| 69 | CACUCUGCCCUUGGCAGCUC |
| 70 | UgGUUUCUCCUCGU |
| 71 | UUgGUUUCaguUCGU |
| 72 | UUgGUUUCUcuuCGU |
| 73 | UUgGUUUCUcguCGU |
| 74 | UUgGUUUCagcUCGU |
| 75 | UUgGUUUCUcauCGU |
| 76 | UUgGUUUCUCCUCGU |
| 77 | UUUgGUUUCUCCUCGU |
| 78 | AUUUgGUUUCaguUCGU |
| 79 | AUUUgGUUUCUcuuCGU |
| 80 | AUUUgGUUUCUcguCGU |
| 81 | AUUUgGUUUCagcUCGU |
| 82 | AUUUgGUUUCUcauCGU |
| 83 | AUUUgGUUUCUCCUCGU |
| 84 | UAUUUgGUUUCUCCUCGU |
| 85 | GUAUUUgGUUUCaguUCGU |
| 86 | GUAUUUgGUUUCUcuuCGU |
| 87 | GUAUUUgGUUUCUcguCGU |
| 88 | GUAUUUgGUUUCagcUCGU |
| 89 | GUAUUUgGUUUCUcauCGU |
| 90 | GUAUUUgGUUUCUCCUCGU |
| 91 | UGUAUUUgGUUUCUCCUCGU |
| 92 | GCUGUAUUUgGUUUCaguUCGU |
| 93 | GCUGUAUUUgGUUUCUcuuCGU |
| 94 | GCUGUAUUUgGUUUCUcguCGU |
| 95 | GCUGUAUUUgGUUUCagcUCGU |
| 96 | GCUGUAUUUgGUUUCUcauCGU |
| 97 | GCUGUAUUUgGUUUCUCCUCGU |
| 98 | AGCUGUAUUUgGUUUCUCCUCGU |
| 99 | GAGCUGUAUUUgGUUUCUCCUCGU |
| 100 | GGAGCUGUAUUUgGUUUCaguUCGU |
| 101 | GGAGCUGUAUUUgGUUUCUcuuCGU |
| 102 | GGAGCUGUAUUUgGUUUCUcguCGU |
| 103 | GGAGCUGUAUUUgGUUUCagcUCGU |
| 104 | GGAGCUGUAUUUgGUUUCUcauCGU |
| 105 | GGAGCUGUAUUUgGUUUCUCCUCGU |
| 106 | GGGAGCUGUAUUUgGUUUCUCCUCGU |
| 107 | UGGGAGCUGUAUUUgGUUUCaguUCGU |
| 108 | UGGGAGCUGUAUUUgGUUUCUcuuCGU |
| 109 | UGGGAGCUGUAUUUgGUUUCUcguCGU |
| 110 | UGGGAGCUGUAUUUgGUUUCagcUCGU |
| 111 | UGGGAGCUGUAUUUgGUUUCUcauCGU |
| 112 | UGGGAGCUGUAUUUgGUUUCUCCUCGU |
| 113 | UUGGGAGCUGUAUUUgGUUUCUCCUCGU |
| 114 | AUUGGGAGCUGUAUUUgGUUUCUCCUCGU |
| 115 | UAUUGGGAGCUGUAUUUgGUUUCUCCUCGU |
| 116 | GUAUUGGGAGCUGUAUUUgGUUUCUCCUCGU |
| 117 | GGUAUUGGGAGCUGUAUUUgGUUUCUCCUCGU |
| 118 | UGGUAUUGGGAGCUGUAUUUgGUUUCUCCUCGU |
| 119 | UCCUGGUAUUGGGAGCUGUAUUUgGUUUCUCCUCGU |
| 120 | AUCCUGGUAUUGGGAGCUGUAUUUgGUUUCUCCUCGU |
| 121 | UgGUUUCUCCUCGUAUGGCCCCAU |
| 122 | UUgGUUUCUCCUCGUAUGGCCCCAU |
| 123 | UUUgGUUUCUCCUCGUAUGGuCCCAU |
| 124 | UUUgGUUUCUCCUCGUAUGGgCCCAU |
| 125 | UUUgGUUUCUCCUCGUAUGGCCCCAU |
| 126 | UUUgGUUUCUCCUCGUAUGGaCCCAU |
| 127 | AUUUgGUUUCUCCUCGUAUGGCCCCAU |
| 128 | UAUUUgGUUUCUCCUCGUAUGGCCCCAU |
| 129 | GUAUUUgGUUUCUCCUCGUAUGGuCCCAU |
| 130 | GUAUUUgGUUUCUCCUCGUAUGGgCCCAU |
| 131 | GUAUUUgGUUUCUCCUCGUAUGGCCCCAU |
| 132 | GUAUUUgGUUUCUCCUCGUAUGGaCCCAU |
| 133 | UGUAUUUgGUUUCUCCUCGUAUGGCCCCAU |
| 134 | GCUGUAUUUgGUUUCUCCUCGUAUGGCCCCAU |
| 135 | AGCUGUAUUUgGUUUCUCCUCGUAUGGuCCCAU |
| 136 | AGCUGUAUUUgGUUUCUCCUCGUAUGGgCCCAU |
| 137 | AGCUGUAUUUgGUUUCUCCUCGUAUGGCCCCAU |
| 138 | AGCUGUAUUUgGUUUCUCCUCGUAUGGaCCCAU |
| 139 | GAGCUGUAUUUgGUUUCUCCUCGUAUGGCCCCAU |
| 140 | GGAGCUGUAUUUgGUUUCUCCUCGUAUGGCCCCAU |
| 141 | GGGAGCUGUAUUUgGUUUCUCCUCGUAUGGuCCCAU |
| 142 | GGGAGCUGUAUUUgGUUUCUCCUCGUAUGGgCCCAU |
| 143 | GGGAGCUGUAUUUgGUUUCUCCUCGUAUGGCCCCAU |
| 144 | GGGAGCUGUAUUUgGUUUCUCCUCGUAUGGaCCCAU |
| 145 | UGGGAGCUGUAUUUgGUUUCUCCUCGUAUGGCCCCAU |
| 146 | UgGUUUCUCCUCGUAUGGCCCCAUU |
| 147 | UUgGUUUCUCCUCGUAUGGCCCCAUU |
| 148 | UUUgGUUUCUCCUCGUAUGGCCCCAUU |
| 149 | AUUUgGUUUCUCCUCGUAUGGCCCCAUU |
| 150 | UAUUUgGUUUCUCCUCGUAUGGCCCCAUU |
| 151 | GUAUUUgGUUUCUCCUCGUAUGGCCCCAUU |
| 152 | UGUAUUUgGUUUCUCCUCGUAUGGCCCCAUU |
| 153 | GCUGUAUUUgGUUUCUCCUCGUAUGGCCCCAUU |
| 154 | AGCUGUAUUUgGUUUCUCCUCGUAUGGCCCCAUU |
| 155 | GAGCUGUAUUUgGUUUCUCCUCGUAUGGCCCCAUU |
| 156 | GGAGCUGUAUUUgGUUUCUCCUCGUAUGGCCCCAUU |
| 157 | GGGAGCUGUAUUUgGUUUCUCCUCGUAUGGCCCCAUU |
| 158 | UgGUUUCUCCUCGUAUGGCCCCAUUG |
| 159 | UUgGUUUCUCCUCGUAUGGCCCCAUUG |
| 160 | UUUgGUUUCUCCUCGUAUGGCCCCAUUG |
| 161 | AUUUgGUUUCUCCUCGUAUGGCCCCAUUG |
| 162 | UAUUUgGUUUCUCCUCGUAUGGCCCCAUUG |
| 163 | GUAUUUgGUUUCUCCUCGUAUGGCCCCAUUG |
| 164 | UGUAUUUgGUUUCUCCUCGUAUGGCCCCAUUG |
| 165 | GCUGUAUUUgGUUUCUCCUCGUAUGGCCCCAUUG |
| 166 | AGCUGUAUUUgGUUUCUCCUCGUAUGGCCCCAUUG |
| 167 | GAGCUGUAUUUgGUUUCUCCUCGUAUGGCCCCAUUG |
| 168 | GGAGCUGUAUUUgGUUUCUCCUCGUAUGGCCCCAUUG |
| 169 | UgGUUUCUCCUCGUAUGGCCCCAUUGCCCUGU |
| 170 | UUgGUUUCUCCUCGUAUGGCCCCAUUGCCCUGU |
| 171 | UUUgGUUUCUCCUCGUAUGGCCCCAUUGCCCUGU |
| 172 | AUUUgGUUUCUCCUCGUAUGGCCCCAUUGCCCUGU |
| 173 | UAUUUgGUUUCUCCUCGUAUGGCCCCAUUGCCCUGU |
| 174 | GUAUUUgGUUUCUCCUCGUAUGGCCCCAUUGCCCUGU |
| 175 | UgGUUUCUCCUCGUAUGGCCCCAUUGCCCUGUU |
| 176 | UUgGUUUCUCCUCGUAUGGCCCCAUUGCCCUGUU |
| 177 | UUUgGUUUCUCCUCGUAUGGCCCCAUUGCCCUGUU |
| 178 | AUUUgGUUUCUCCUCGUAUGGCCCCAUUGCCCUGUU |
| 179 | UAUUUgGUUUCUCCUCGUAUGGCCCCAUUGCCCUGUU |
| 180 | ACCAAAUACAGCUCCCAAU |
| 181 | AACCAAAUACAGCUCCCAAU |
| 182 | AAACCAAAUACAGCUCCCAAU |
| 183 | GAAACCAAAUACAGCUCCCAAU |
| 184 | AGAAACCAAAUACAGCUCCCAAU |
| 185 | GAGAAACCAAAUACAGCUCCCAAU |
| 186 | GGAGAAACCAAAUACAGCUCCCAAU |
| 187 | AGGAGAAACCAAAUACAGCUCCCAAU |
| 188 | GAGGAGAAACcAAAUACAGCUCCCAAU |
| 189 | ACGAGGAGAAACCAAAUACAGCUCCCAAU |
| 190 | UACGAGGAGAAACCAAAUACAGCUCCCAAU |
| 191 | AUACGAGGAGAAACCAAAUACAGCUCCCAAU |
| 192 | GCCAUACGAGGAGAAACCAAAUACAGCUCCCAAU |
| 193 | GGCCAUACGAGGAGAAACCAAAUACAGCUCCCAAU |
| 194 | GGGCCAUACGAGGAGAAACCAAAUACAGCUCCCAAU |
| 195 | GGGGCCAUACGAGGAGAAACcAAAUACAGCUCCCAAU |
| 196 | ACcAAAUACAGCUCCCAAUA |
| 197 | AACCAAAUACAGCUCCCAAUA |
| 198 | AAACCAAAUACAGCUCCCAAUA |
| 199 | AAACCAAAUACAGCUCCuAAUA |
| 200 | AAACCAAAUACAGCUCCgAAUA |
| 201 | AAACCAAAUACAGCUCCaAAUA |
| 202 | GAAACCAAAUACAGCUCCCAAUA |
| 203 | AGAAACCAAAUACAGCUCCCAAUA |
| 204 | AGAAACCAAAUACAGCUCCuAAUA |
| 205 | AGAAACCAAAUACAGCUCCgAAUA |
| 206 | AGAAACCAAAUACAGCUCCaAAUA |
| 207 | GAGAAACCAAAUACAGCUCCCAAUA |
| 208 | GGAGAAACCAAAUACAGCUCCCAAUA |
| 209 | GGAGAAACCAAAUACAGCUCCuAAUA |
| 210 | GGAGAAACCAAAUACAGCUCCgAAUA |
| 211 | GGAGAAACCAAAUACAGCUCCaAAUA |
| 212 | AGGAGAAACCAAAUACAGCUCCCAAUA |
| 213 | GAGGAGAAACCAAAUACAGCUCCCAAUA |
| 214 | GAGGAGAAACCAAAUACAGCUCCuAAUA |
| 215 | GAGGAGAAACCAAAUACAGCUCCgAAUA |
| 216 | GAGGAGAAACCAAAUACAGCUCCaAAUA |
| 217 | ACGAGGAGAAACCAAAUACAGCUCCCAAUA |
| 218 | UACGAGGAGAAACCAAAUACAGCUCCCAAUA |
| 219 | UACGAGGAGAAACCAAAUACAGCUCCuAAUA |
| 220 | UACGAGGAGAAACCAAAUACAGCUCCgAAUA |
| 221 | UACGAGGAGAAACCAAAUACAGCUCCaAAUA |
| 222 | AUACGAGGAGAAACCAAAUACAGCUCCCAAUA |
| 223 | GCCAUACGAGGAGAAACCAAAUACAGCUCCCAAUA |
| 224 | GGCCAUACGAGGAGAAACCAAAUACAGCUCCCAAUA |
| 225 | GGGCCAUACGAGGAGAAACcAAAUACAGCUCCCAAUA |
| 226 | ACCAAAUACAGCUCCCAAUACCAGGA |
| 227 | AACCAAAUACAGCUCCCAAUACCAGGA |
| 228 | AAACCAAAUACAGCUCCCAAUACCAGGA |
| 229 | GAAACCAAAUACAGCUCCCAAUACCAGGA |
| 230 | AGAAACCAAAUACAGCUCCCAAUACCAGGA |
| 231 | GAGAAACCAAAUACAGCUCCCAAUACCAGGA |
| 232 | GGAGAAACCAAAUACAGCUCCCAAUACCAGGA |
| 233 | AGGAGAAACCAAAUACAGCUCCCAAUACCAGGA |
| 234 | GAGGAGAAACCAAAUACAGCUCCCAAUACCAGGA |
| 235 | ACGAGGAGAAACCAAAUACAGCUCCCAAUACCAGGA |
| 236 | UACGAGGAGAAACCAAAUACAGCUCCCAAUACCAGGA |
| 237 | ACCAAAUACAGCUCCCAAUACCAGGAUCCAGAG |
| 238 | AACCAAAUACAGCUCCCAAUACCAGGAUCCAGAG |
| 239 | AAACCAAAUACAGCUCCCAAUACCAGGAUCCAGAG |
| 240 | GAAACCAAAUACAGCUCCCAAUACCAGGAUCCAGAG |
| 241 | AGAAACCAAAUACAGCUCCCAAUACCAGGAUCCAGAG |
| | AUGGCCC (SEQ REF NO: 242) |
| | AUGGCCCC (SEQ REF NO: 243) |
| | AUGGCCCCA (SEQ REF NO: 244) |
| 245 | AUGGCCCCAU |
| 246 | AUGGCCCCAUU |
| 247 | AUGGCCCCAUUG |
| 248 | AUGGCCCCAUUGC |
| 249 | AUGGCCCCAUUGCC |
| 250 | AUGGCCCCAUUGCCC |
| | UGCCCUG (SEQ REF NO: 251) |
| | UGCCCUGU (SEQ REF NO: 252) |
| | UGCCCUGUU (SEQ REF NO: 253) |
| 254 | UGCCCUGUUU |
| 255 | UGCCCUGUUUG |
| 256 | UGCCCUGUUUGG |
| 257 | UGCCCUGUUUGGA |
| 258 | UGCCCUGUUUGGAG |
| 259 | UGCCCUGUUUGGAGU |
| | GCCCUGU (SEQ REF NO: 260) |
| | GCCCUGUU (SEQ REF NO: 261) |
| | GCCCUGUUU (SEQ REF NO: 262) |
| 263 | GCCCUGUUUG |
| 264 | GCCCUGUUUGG |
| 265 | GCCCUGUUUGGA |
| 266 | GCCCUGUUUGGAG |
| 267 | GCCCUGUUUGGAGU |
| 268 | GCCCUGUUUGGAGUC |
| | CCCUGUU (SEQ REF NO: 269) |
| | CCCUGUUU (SEQ REF NO: 270) |
| | CCCUGUUUG (SEQ REF NO: 271) |
| 272 | CCCUGUUUGG |
| 273 | CCCUGUUUGGA |
| 274 | CCCUGUUUGGAG |
| 275 | CCCUGUUUGGAGU |
| 276 | CCCUGUUUGGAGUC |
| 277 | CCCUGUUUGGAGUCA |
| | UUGGAGU (SEQ REF NO: 243) |
| | UUGGAGUC (SEQ REF NO: 243) |
| | UUGGAGUCA (SEQ REF NO: 243) |
| 281 | UUGGAGUCAU |
| 282 | UUGGAGUCAUA |
| 283 | UUGGAGUCAUAG |
| 284 | UUGGAGUCAUAGC |
| 285 | UUGGAGUCAUAGCC |
| 286 | UUGGAGUCAUAGCCA |
| | UGGAGUC (SEQ REF NO: 287) |
| | UGGAGUCA (SEQ REF NO: 288) |
| | UGGAGUCAU (SEQ REF NO: 289) |
| 290 | UGGAGUCAUA |
| 291 | UGGAGUCAUAG |
| 292 | UGGAGUCAUAGC |
| 293 | UGGAGUCAUAGCC |
| 294 | UGGAGUCAUAGCCA |
| 295 | UGGAGUCAUAGCCAA |
| | ACCAGGA (SEQ REF NO: 296) |
| | ACCAGGAU (SEQ REF NO: 297) |
| | ACCAGGAUC (SEQ REF NO: 298) |
| 299 | ACCAGGAUCC |
| 300 | ACCAGGAUCCA |
| 301 | ACCAGGAUCCAG |
| 302 | ACCAGGAUCCAGA |
| 303 | ACCAGGAUCCAGAG |
| 304 | ACCAGGAUCCAGAGC |
| | CCAGGAU (SEQ REF NO: 305) |
| | CCAGGAUC (SEQ REF NO: 306) |
| | CCAGGAUCC (SEQ REF NO: 307) |
| 308 | CCAGGAUCCA |
| 309 | CCAGGAUCCAG |
| 310 | CCAGGAUCCAGA |
| 311 | CCAGGAUCCAGAG |
| 312 | CCAGGAUCCAGAGC |
| 313 | CCAGGAUCCAGAGCU |
| | UCCAGAG (SEQ REF NO: 314) |
| | UCCAGAGC (SEQ REF NO: 315) |
| | UCCAGAGCU (SEQ REF NO: 316) |
| 317 | UCCAGAGCUG |
| 318 | UCCAGAGCUGC |
| 319 | UCCAGAGCUGCC |
| 320 | UCCAGAGCUGCCA |
| 321 | UCCAGAGCUGCCAA |
| 322 | UCCAGAGCUGCCAAG |
| | CUGCCAA (SEQ REF NO: 323) |
| | CUGCCAAG (SEQ REF NO: 324) |
| | CUGCCAAGG (SEQ REF NO: 325) |
| 326 | CUGCCAAGGG |
| 327 | CUGCCAAGGGC |
| 328 | CUGCCAAGGGCA |
| 329 | CUGCCAAGGGCAG |
| 330 | CUGCCAAGGGCAGA |
| 331 | CUGCCAAGGGCAGAG |
| 332 | AUUGUGGGACUCAUGGCCAA |
| 333 | CCACGCCAUUGUGGGACUCA |
| 334 | GGCACCUCCCACGCCAUUGU |
| 335 | UGGCACCUCCCACGCCAUUG |
| 336 | AGUGCCCCUCCCAACUUGUG |
| 337 | UAUGGCCCCAUUGCCCUGUU |
| 338 | GUAUUUgGUUUCUCCUCGUA |
| 339 | CAGCUCUGGAUCCUGGUAUU |
| 340 | GCAGCUCUGGAUCCUGGUAU |
| 341 | CCCUUGGCAGCUCUGGAUCC |
| 342 | CACUCUGCCCUUGGCAGCUC |
| 343 | AUGCCACUCCACUCUGCCCU |
| 344 | GCAGGGGUGGGUAGGCAGGU |
| 345 | UUUGGCAGGGGUGGGUAGGC |
| 346 | UUGCUUUGGCAGGGGUGGGU |
| 347 | CUUCUUGCUUUGGCAGGGGU |
| 348 | AUUGGCCAUGAGUCCCACAA |
| 349 | CCAUGAGUCCCACAAUGGCG |
| 350 | CAUGAGUCCCACAAUGGCGU |
| 351 | GAGUCCCACAAUGGCGUGGG |
| 352 | GUGGGAGGUGCCACACAAGU |
| 353 | UGGGAGGUGCCACACAAGUU |
| 354 | GAGGUGCCACACAAGUUGGG |
| 355 | AGGUGCCACACAAGUUGGGA |
| 356 | GGUGCCACACAAGUUGGGAG |
| 357 | GUUGGGAGGGGCACUCUCGU |
| 358 | CGUUGGCUAUGACUCCAAAC |
| 359 | GUUGGCUAUGACUCCAAACA |
| 360 | UAUGACUCCAAACAGGGCAA |
| 361 | AUGACUCCAAACAGGGCAAU |
| 362 | UGACUCCAAACAGGGCAAUG |
| 363 | CAGGGCAAUGGGGCCAUACG |
| 364 | CAAAUACAGCUCCCAAUACC |
| 365 | ACCAGGAUCCAGAGCUGCCA |
| 366 | CCAGGAUCCAGAGCUGCCAA |
| 367 | CAGAGCUGCCAAGGGCAGAG |
| 368 | CUGCCAAGGGCAGAGUGGAG |
| 369 | AGUGGAGUGGCAUUCAGAGU |
| 370 | CCCCUGCCAAAGCAAGAAGA |
| 3001 | GAAACCAAAUACAGCUCCUAAUA |
| 3001 | GGAGCUGUAUUUgGUUUCUCCUCGUAUGGCCCCAUUG |
| 3002 | GAAACCAAAUACAGCUCCGAGUA |
| 3003 | GAAACCAAAUACAGCUCCGAGUA |
| 3003 | UGUAUUUGGUUUCUCAUCGU |
| 3004 | GAAACCAAAUACAGCUCCCAAUA |
| 3004 | UGUAUUUGGUUUCUCCUCGUAUGGGCCCAU |
| 3005 | GAAACCAAAUACAGCUCCAAGUA |
| 3005 | GGAGCUGUAUUUGGUUUCUCCUCGUAUGGACCCAU |
| 3006 | GAGAAACCAAAUACAGCUCCUAAUA |
| 3007 | GAGAAACCAAAUACAGCUCCGAGUA |
| 3008 | GAGAAACCAAAUACAGCUCCAAGUA |
| 3009 | AGGAGAAACCAAAUACAGCUCCUAAUA |
| 3010 | AGGAGAAACCAAAUACAGCUCCGAGUA |
| 3011 | AGGAGAAACCAAAUACAGCUCCAAGUA |
| 3012 | UAUUUgGUUUCUcguCGU |
| 3013 | UAUUUGGUUUCUCAUCGU |
| 3014 | UAUUUgGUUUCUcuuCGU |
| 3016 | GUAUUUgGUUUCUcguCGU |
| 3017 | GUAUUUGGUUUCUCAUCGU |
| 3018 | UGUAUUUgGUUUCUcuuCGU |
| 3019 | UGUAUUUgGUUUCUcguCGU |
| 3020 | UGUAUUUGGUUUCUCAUCGU |
| 3021 | CUGUAUUUgGUUUCUcuuCGU |
| 3022 | CUGUAUUUgGUUUCUcguCGU |
| 3023 | CUGUAUUUGGUUUCUCCUCGU |
| 3024 | CUGUAUUUGGUUUCUCAUCGU |
| 3025 | GCUGUAUUUgGUUUCUcuuCGU |
| 3026 | GCUGUAUUUgGUUUCUcguCGU |
| 3027 | GCUGUAUUUGGUUUCUCAUCGU |
| 3028 | AGCUGUAUUUgGUUUCUcuuCGU |
| 3029 | AGCUGUAUUUgGUUUCUcguCGU |
| 3030 | AGCUGUAUUUGGUUUCUCAUCGU |
| 3035 | UAUUUGGUUUCUCCUCGUAUGGUCCCAU |
| 3036 | UAUUUGGUUUCUCCUCGUAUGGGCCCAU |
| 3037 | UAUUUGGUUUCUCCUCGUAUGGCCCCAU |
| 3038 | UAUUUGGUUUCUCCUCGUAUGGACCCAU |
| 3039 | GUAUUUGGUUUCUCCUCGUAUGGUCCCAU |
| 3040 | GUAUUUGGUUUCUCCUCGUAUGGGCCCAU |
| 3041 | GUAUUUGGUUUCUCCUCGUAUGGCCCCAU |
| 3042 | GUAUUUGGUUUCUCCUCGUAUGGACCCAU |
| 3043 | UGUAUUUGGUUUCUCCUCGUAUGGUCCCAU |
| 3044 | UGUAUUUGGUUUCUCCUCGUAUGGGCCCAU |
| 3045 | UGUAUUUGGUUUCUCCUCGUAUGGCCCCAU |
| 3046 | UGUAUUUGGUUUCUCCUCGUAUGGACCCAU |
| 3047 | CUGUAUUUGGUUUCUCCUCGUAUGGUCCCAU |
| 3048 | CUGUAUUUGGUUUCUCCUCGUAUGGGCCCAU |
| 3049 | CUGUAUUUGGUUUCUCCUCGUAUGGCCCCAU |
| 3050 | CUGUAUUUGGUUUCUCCUCGUAUGGACCCAU |
| 3051 | CUGUAUUUgGUUUCUCCUCGUAUGGCCCCAU |
| 3052 | GCUGUAUUUGGUUUCUCCUCGUAUGGUCCCAU |
| 3053 | GCUGUAUUUGGUUUCUCCUCGUAUGGGCCCAU |
| 3054 | GCUGUAUUUGGUUUCUCCUCGUAUGGCCCCAU |
| 3055 | GCUGUAUUUGGUUUCUCCUCGUAUGGACCCAU |
| 3056 | AGCUGUAUUUGGUUUCUCCUCGUAUGGUCCCAU |
| 3057 | AGCUGUAUUUGGUUUCUCCUCGUAUGGGCCCAU |
| 3058 | AGCUGUAUUUGGUUUCUCCUCGUAUGGCCCCAU |
| 3059 | AGCUGUAUUUGGUUUCUCCUCGUAUGGACCCAU |
| 3060 | GAGCUGUAUUUGGUUUCUCCUCGUAUGGUCCCAU |
| 3061 | GAGCUGUAUUUGGUUUCUCCUCGUAUGGGCCCAU |
| 3062 | GAGCUGUAUUUGGUUUCUCCUCGUAUGGCCCCAU |
| 3063 | GAGCUGUAUUUGGUUUCUCCUCGUAUGGACCCAU |
| 3064 | GGAGCUGUAUUUGGUUUCUCCUCGUAUGGUCCCAU |
| 3065 | GGAGCUGUAUUUGGUUUCUCCUCGUAUGGGCCCAU |
| 3066 | GGAGCUGUAUUUGGUUUCUCCUCGUAUGGCCCCAU |
| 3067 | GGAGCUGUAUUUGGUUUCUCCUCGUAUGGACCCAU |
| 3068 | GGGAGCUGUAUUUGGUUUCUCCUCGUAUGGUCCCAU |
| 3069 | GGGAGCUGUAUUUGGUUUCUCCUCGUAUGGGCCCAU |
| 3070 | GGGAGCUGUAUUUGGUUUCUCCUCGUAUGGCCCCAU |
| 3071 | GGGAGCUGUAUUUGGUUUCUCCUCGUAUGGACCCAU |
| 3101 | CUGUAUUUgGUUUCUCCUCGU |
| 5001 | GUAUUUGGUUUCUCAUCGUA |
| 5002 | GUAUUUGGUUUCUCGUCGUA |
| 5003 | GUAUUUGGUUUCUCUUCGUA |
| 5004 | UAUGGUCCCAUUGCCCUGUU |
| 5005 | UAUGGGCCCAUUGCCCUGUU |
| 5006 | UAUGGACCCAUUGCCCUGUU |
| 5007 | UAUGGCCCUAUUGCCCUGUU |
| 5008 | UAUGGCCCGAUUGCCCUGUU |
| 5009 | UAUGGCCCAAUUGCCCUGUU |
| 5010 | UAUGGCCCCAUUGCUCUGUU |
| 5011 | UAUGGCCCCAUUGCACUGUU |
| 5012 | UAUGGCCCCAUUGCGCUGUU |
| 5013 | ACCAAGCCUUCUUCUUGCUU |
| 5014 | AGCCUUCUUCUUGCUUUGGC |
| 5015 | GCCUUCUUCUUGCUUUGGCA |
| 5016 | CCUUCUUCUUGCUUUGGCAG |
| 5017 | UCUUCUUGCUUUGGCAGGGG |
| 5018 | CUAGGAUCCAGAGCUGCCAA |
| 5019 | CAAGGAUCCAGAGCUGCCAA |
| 5020 | CGAGGAUCCAGAGCUGCCAA |
| 5021 | ACUAGGAUCCAGAGCUGCCA |
| 5022 | ACAAGGAUCCAGAGCUGCCA |
| 5023 | ACGAGGAUCCAGAGCUGCCA |
| 5024 | UGACUCCAAACAGAGCAAUG |
| 5025 | UGACUCCAAACAGUGCAAUG |
| 5026 | UGACUCCAAACAGCGCAAUG |
| 5027 | AUGACUCCAAACAGAGCAAU |
| 5028 | AUGACUCCAAACAGUGCAAU |
| 5029 | AUGACUCCAAACAGCGCAAU |
| 5030 | CAAAUACAGCUCCUAAUACC |
| 5030 | GCCAAAGCAAGAAGAAGGCU |
| 5031 | CAAAUACAGCUCCGAAUACC |
| 5032 | CAAAUACAGCUCCAAAUACC |
| 5033 | CAAAUACAGCUCCCAAUACU |
| 5034 | CAAAUACAGCUCCCAAUACG |
| 5035 | CAAAUACAGCUCCCAAUACA |

TABLE 19a
Exemplary RTT sequences for Spacer S11

| SEQ REF | | SEQ REF | |
|---|---|---|---|
| NO: | Sequence | NO: | Sequence |
| 6090 | UGCCcuGUUU | 6111 | UGGCCCCAUUGCacuGUUU |
| 6091 | UGCacuGUUU | 6112 | UGGCCCCAUUGCgcuGUUU |
| 6092 | UGCgcuGUUU | 6113 | UGGCCCCAUUGCucuGUUU |
| 6093 | UGCucuGUUU | 6114 | AUGGCCCCAUUGCCcuGUUU |
| 6094 | UUGCCcuGUUU | 6115 | AUGGCCCCAUUGCacuGUUU |
| 6095 | UUGCacuGUUU | 6116 | AUGGCCCCAUUGCgcuGUUU |
| 6096 | UUGCgcuGUUU | 6117 | AUGGCCCCAUUGCucuGUUU |
| 6097 | UUGCucuGUUU | 6118 | UAUGGCCCCAUUGCCcuGUUU |
| 6098 | AUUGCCcuGUUU | 6119 | GUAUGGCCCCAUUGCCcuGUUU |
| 6099 | AUUGCacuGUUU | 407 | UCGUAUGGCCCCAUUGCCcuGUUU |
| 6100 | AUUGCgcuGUUU | 408 | UCCUCGUAUGGCCCCAUUGCCcuGUU |
| | | | U |
| 6101 | AUUGCucuGUUU | 409 | UCUCCUCGUAUGGCCCCAUUGCCcuG |
| | | | UUU |
| 6102 | GCCCCAUUGCCcuGUUU | 410 | UUCUCCUCGUAUGGCCCCAUUGCCcu |
| | | | GUUU |
| 6103 | GCCCCAUUGCacuGUUU | 411 | UUUCUCCUCGUAUGGCCCCAUUGCCC |
| | | | uGUUU |
| 6104 | GCCCCAUUGCgcuGUUU | 412 | GUUUCUCCUCGUAUGGCCCCAUUGCC |
| | | | cuGUUU |
| 6105 | GCCCCAUUGCucuGUUU | 413 | GGUUUCUCCUCGUAUGGCCCCAUUGC |
| | | | CcuGUUU |
| 6106 | GGCCCCAUUGCCcuGUUU | 414 | UGGUUUCUCCUCGUAUGGCCCCAUUG |
| | | | CCcuGUUU |
| 6107 | GGCCCCAUUGCacuGUUU | 415 | UUGGUUUCUCCUCGUAUGGCCCCAUU |
| | | | GCCcuGUUU |
| 6108 | GGCCCCAUUGCgcuGUUU | 416 | UUUGGUUUCUCCUCGUAUGGCCCCAU |
| | | | UGCCcuGUUU |
| 6109 | GGCCCCAUUGCucuGUUU | 417 | AUUUGGUUUCUCCUCGUAUGGCCCCA |
| | | | UUGCCcuGUUU |
| 6110 | UGGCCCCAUUGCCcuGUUU | 6012 | CCCAUUGCgcuGUUU |
| 6005 | CAUUGCucuGUUU | 6013 | CCCAUUGCacuGUUU |
| 6006 | CAUUGCgcuGUUU | 6020 | CCCAUUGCCcuGUUU |
| 6007 | CAUUGCacuGUUU | 6014 | CCCCAUUGCucuGUUU |
| 6018 | CAUUGCCcuGUUU | 6015 | CCCCAUUGCgcuGUUU |
| 6008 | CCAUUGCucuGUUU | 6016 | CCCCAUUGCacuGUUU |
| 6009 | CCAUUGCgcuGUUU | 6017 | CCCCAUUGCCcuGUUU |
| 6010 | CCAUUGCacuGUUU | | |
| 6019 | CCAUUGCCcuGUUU | | |

TABLE 19b
Exemplary PBS sequences for Spacer S11

| | Sequence |
|---|---|
| SEQ REF NO: 467 | GGAGUCA |
| SEQ REF NO: 468 | GGAGUCAU |
| SEQ REF NO: 469 | GGAGUCAUA |
| SEQ ID NO: 470 | GGAGUCAUAG |
| SEQ ID NO: 471 | GGAGUCAUAGC |
| SEQ ID NO: 472 | GGAGUCAUAGCC |
| SEQ ID NO: 473 | GGAGUCAUAGCCA |
| SEQ ID NO: 474 | GGAGUCAUAGCCAA |
| SEQ ID NO: 475 | GGAGUCAUAGCCAAC |

TABLE 19c
Exemplary RTT/PBS Combinations with Spacer S11

| PEG# | RTT | PBS |
|---|---|---|
| 1704 | 377 | 467 |
| 1713 | 381 | 467 |
| 1722 | 385 | 467 |
| 1731 | 389 | 467 |
| 1740 | 393 | 467 |
| 1749 | 397 | 467 |
| 1758 | 401 | 467 |
| 1767 | 405 | 467 |
| 1776 | 406 | 467 |
| 1785 | 407 | 467 |
| 1794 | 408 | 467 |
| 1803 | 409 | 467 |
| 1812 | 410 | 467 |
| 1821 | 411 | 467 |
| 1830 | 412 | 467 |
| 1839 | 413 | 467 |
| 1848 | 414 | 467 |
| 1857 | 415 | 467 |
| 1866 | 416 | 467 |
| 1875 | 417 | 467 |
| 1974 | 380 | 468 |
| 2030 | 379 | 468 |
| 2002 | 378 | 468 |
| 1705 | 377 | 468 |
| 1978 | 384 | 468 |
| 2034 | 383 | 468 |
| 2006 | 382 | 468 |
| 1714 | 381 | 468 |
| 1982 | 388 | 468 |
| 2038 | 387 | 468 |
| 2010 | 386 | 468 |
| 1723 | 385 | 468 |
| 1986 | 392 | 468 |
| 2042 | 391 | 468 |
| 2014 | 390 | 468 |
| 1732 | 389 | 468 |
| 1990 | 396 | 468 |
| 2046 | 395 | 468 |
| 2018 | 394 | 468 |
| 1741 | 393 | 468 |
| 1994 | 400 | 468 |
| 2050 | 399 | 468 |
| 2022 | 398 | 468 |
| 1750 | 397 | 468 |
| 1998 | 404 | 468 |
| 2054 | 403 | 468 |
| 2026 | 402 | 468 |
| 1759 | 401 | 468 |
| 1768 | 405 | 468 |
| 1777 | 406 | 468 |
| 1786 | 407 | 468 |
| 1795 | 408 | 468 |
| 1804 | 409 | 468 |
| 1813 | 410 | 468 |
| 1822 | 411 | 468 |
| 1831 | 412 | 468 |
| 1840 | 413 | 468 |
| 1849 | 414 | 468 |
| 1858 | 415 | 468 |
| 1867 | 416 | 468 |
| 1876 | 417 | 468 |
| 1706 | 377 | 469 |
| 1715 | 381 | 469 |
| 1724 | 385 | 469 |
| 1733 | 389 | 469 |
| 1742 | 393 | 469 |
| 1751 | 397 | 469 |
| 1760 | 401 | 469 |
| 1769 | 405 | 469 |
| 1778 | 406 | 469 |
| 1787 | 407 | 469 |
| 1796 | 408 | 469 |
| 1805 | 409 | 469 |
| 1814 | 410 | 469 |
| 1823 | 411 | 469 |
| 1832 | 412 | 469 |
| 1841 | 413 | 469 |
| 1850 | 414 | 469 |
| 1859 | 415 | 469 |
| 1868 | 416 | 469 |
| 1877 | 417 | 469 |
| 1975 | 380 | 470 |
| 2031 | 379 | 470 |
| 2003 | 378 | 470 |
| 1707 | 377 | 470 |
| 1979 | 384 | 470 |
| 2035 | 383 | 470 |
| 2007 | 382 | 470 |
| 1716 | 381 | 470 |
| 1983 | 388 | 470 |
| 2039 | 387 | 470 |
| 2011 | 386 | 470 |
| 1725 | 385 | 470 |
| 1987 | 392 | 470 |
| 2043 | 391 | 470 |
| 2015 | 390 | 470 |
| 1734 | 389 | 470 |
| 1991 | 396 | 470 |
| 2047 | 395 | 470 |
| 2019 | 394 | 470 |
| 1743 | 393 | 470 |
| 1995 | 400 | 470 |
| 2051 | 399 | 470 |
| 2023 | 398 | 470 |
| 1752 | 397 | 470 |
| 1999 | 404 | 470 |
| 2055 | 403 | 470 |
| 2027 | 402 | 470 |
| 1761 | 401 | 470 |
| 1770 | 405 | 470 |
| 1779 | 406 | 470 |
| 1788 | 407 | 470 |
| 1797 | 408 | 470 |
| 1806 | 409 | 470 |
| 1815 | 410 | 470 |
| 1824 | 411 | 470 |
| 1833 | 412 | 470 |
| 1842 | 413 | 470 |
| 1851 | 414 | 470 |
| 1860 | 415 | 470 |
| 1869 | 416 | 470 |
| 1878 | 417 | 470 |
| 1708 | 377 | 471 |
| 1717 | 381 | 471 |
| 2429 | 388 | 471 |
| 2430 | 387 | 471 |
| 2431 | 386 | 471 |
| 1726 | 385 | 471 |
| 2432 | 6005 | 471 |
| 2433 | 6006 | 471 |
| 2434 | 6007 | 471 |
| 2435 | 6008 | 471 |
| 2436 | 6009 | 471 |
| 2437 | 6010 | 471 |
| 2438 | 6011 | 471 |
| 2439 | 6012 | 471 |
| 2440 | 6013 | 471 |
| 2441 | 6014 | 471 |
| 2442 | 6015 | 471 |
| 2443 | 6016 | 471 |
| 2444 | 392 | 471 |
| 2445 | 391 | 471 |
| 2446 | 390 | 471 |
| 1735 | 389 | 471 |
| 2447 | 396 | 471 |
| 2448 | 395 | 471 |
| 2449 | 394 | 471 |
| 1744 | 393 | 471 |
| 2450 | 400 | 471 |
| 2451 | 399 | 471 |
| 2452 | 398 | 471 |
| 1753 | 397 | 471 |
| 1762 | 401 | 471 |
| 1771 | 405 | 471 |
| 1780 | 406 | 471 |
| 1789 | 407 | 471 |
| 1798 | 408 | 471 |
| 1807 | 409 | 471 |
| 1816 | 410 | 471 |
| 1825 | 411 | 471 |
| 1834 | 412 | 471 |
| 1843 | 413 | 471 |
| 1852 | 414 | 471 |
| 1861 | 415 | 471 |
| 1870 | 416 | 471 |
| 1879 | 417 | 471 |
| 1976 | 380 | 472 |
| 2032 | 379 | 472 |
| 2004 | 378 | 472 |
| 1709 | 377 | 472 |
| 1980 | 384 | 472 |
| 2036 | 383 | 472 |
| 2008 | 382 | 472 |
| 1718 | 381 | 472 |
| 1984 | 388 | 472 |
| 2040 | 387 | 472 |
| 2012 | 386 | 472 |
| 1727 | 385 | 472 |
| 2453 | 6005 | 472 |
| 2454 | 6006 | 472 |
| 2455 | 6007 | 472 |
| 2538 | 6018 | 472 |
| 2456 | 6008 | 472 |
| 2457 | 6009 | 472 |
| 2458 | 6010 | 472 |
| 2459 | 6011 | 472 |
| 2460 | 6012 | 472 |
| 2461 | 6013 | 472 |
| 2462 | 6014 | 472 |
| 2463 | 6015 | 472 |
| 2464 | 6016 | 472 |
| 2465 | 6017 | 472 |
| 1988 | 392 | 472 |
| 2044 | 391 | 472 |
| 2016 | 390 | 472 |
| 1736 | 389 | 472 |
| 1992 | 396 | 472 |
| 2048 | 395 | 472 |
| 2020 | 394 | 472 |
| 1745 | 393 | 472 |
| 1996 | 400 | 472 |
| 2052 | 399 | 472 |
| 2024 | 398 | 472 |
| 1754 | 397 | 472 |
| 2000 | 404 | 472 |
| 2056 | 403 | 472 |
| 2028 | 402 | 472 |
| 1763 | 401 | 472 |
| 1772 | 405 | 472 |
| 1781 | 406 | 472 |
| 1790 | 407 | 472 |
| 1799 | 408 | 472 |
| 1808 | 409 | 472 |
| 1817 | 410 | 472 |
| 1826 | 411 | 472 |
| 1835 | 412 | 472 |
| 1844 | 413 | 472 |
| 1853 | 414 | 472 |
| 1862 | 415 | 472 |
| 1871 | 416 | 472 |
| 1880 | 417 | 472 |
| 1710 | 377 | 473 |
| 1719 | 381 | 473 |
| 2466 | 388 | 473 |
| 2467 | 387 | 473 |
| 2468 | 386 | 473 |
| 1728 | 385 | 473 |
| 2469 | 6005 | 473 |
| 2470 | 6006 | 473 |
| 2471 | 6007 | 473 |
| 2472 | 6018 | 473 |
| 2473 | 6008 | 473 |
| 2474 | 6009 | 473 |
| 2475 | 6010 | 473 |
| 2476 | 6019 | 473 |
| 2477 | 6011 | 473 |
| 2478 | 6012 | 473 |
| 2479 | 6013 | 473 |
| 2480 | 6020 | 473 |
| 2481 | 6014 | 473 |
| 2482 | 6015 | 473 |
| 2483 | 6016 | 473 |
| 2484 | 6017 | 473 |
| 2485 | 392 | 473 |
| 2486 | 391 | 473 |
| 2487 | 390 | 473 |
| 1737 | 389 | 473 |
| 2488 | 396 | 473 |
| 2489 | 395 | 473 |
| 2490 | 394 | 473 |
| 1746 | 393 | 473 |
| 2491 | 400 | 473 |
| 2492 | 399 | 473 |
| 2493 | 398 | 473 |
| 1755 | 397 | 473 |
| 1764 | 401 | 473 |
| 1773 | 405 | 473 |
| 1782 | 406 | 473 |
| 1791 | 407 | 473 |
| 1800 | 408 | 473 |
| 1809 | 409 | 473 |
| 1818 | 410 | 473 |
| 1827 | 411 | 473 |
| 1836 | 412 | 473 |
| 1845 | 413 | 473 |
| 1854 | 414 | 473 |
| 1863 | 415 | 473 |
| 1872 | 416 | 473 |
| 1881 | 417 | 473 |
| 1977 | 380 | 474 |
| 2033 | 379 | 474 |
| 2005 | 378 | 474 |
| 1711 | 377 | 474 |
| 1981 | 384 | 474 |
| 2037 | 383 | 474 |
| 2009 | 382 | 474 |
| 1720 | 381 | 474 |
| 1985 | 388 | 474 |
| 2041 | 387 | 474 |
| 2013 | 386 | 474 |
| 1729 | 385 | 474 |
| 2494 | 6005 | 474 |
| 2495 | 6006 | 474 |
| 2496 | 6007 | 474 |
| 2497 | 6018 | 474 |
| 2498 | 6008 | 474 |
| 2499 | 6009 | 474 |
| 2500 | 6010 | 474 |
| 2501 | 6019 | 474 |
| 2502 | 6011 | 474 |
| 2503 | 6012 | 474 |
| 2504 | 6013 | 474 |
| 2505 | 6020 | 474 |
| 2506 | 6014 | 474 |
| 2507 | 6015 | 474 |
| 2508 | 6016 | 474 |
| 2509 | 6017 | 474 |
| 1989 | 392 | 474 |
| 2045 | 391 | 474 |
| 2017 | 390 | 474 |
| 1738 | 389 | 474 |
| 1993 | 396 | 474 |
| 2049 | 395 | 474 |
| 2021 | 394 | 474 |
| 1747 | 393 | 474 |
| 1997 | 400 | 474 |
| 2053 | 399 | 474 |
| 2025 | 398 | 474 |
| 1756 | 397 | 474 |
| 2001 | 404 | 474 |
| 2057 | 403 | 474 |
| 2029 | 402 | 474 |
| 1765 | 401 | 474 |
| 1774 | 405 | 474 |
| 1783 | 406 | 474 |
| 1792 | 407 | 474 |
| 1801 | 408 | 474 |
| 1810 | 409 | 474 |
| 1819 | 410 | 474 |
| 1828 | 411 | 474 |
| 1837 | 412 | 474 |
| 1846 | 413 | 474 |
| 1855 | 414 | 474 |
| 1864 | 415 | 474 |
| 1873 | 416 | 474 |
| 1882 | 417 | 474 |
| 1712 | 377 | 475 |
| 1721 | 381 | 475 |
| 2510 | 388 | 475 |
| 2511 | 387 | 475 |
| 2512 | 386 | 475 |
| 1730 | 385 | 475 |
| 2513 | 6005 | 475 |
| 2514 | 6006 | 475 |
| 2515 | 6007 | 475 |
| 2516 | 6018 | 475 |
| 2517 | 6008 | 475 |
| 2518 | 6009 | 475 |
| 2519 | 6010 | 475 |
| 2520 | 6019 | 475 |
| 2521 | 6011 | 475 |
| 2522 | 6012 | 475 |
| 2523 | 6013 | 475 |
| 2524 | 6020 | 475 |
| 2525 | 6014 | 475 |
| 2526 | 6015 | 475 |
| 2527 | 6016 | 475 |
| 2528 | 6017 | 475 |
| 2529 | 392 | 475 |
| 2530 | 391 | 475 |
| 2531 | 390 | 475 |
| 1739 | 389 | 475 |
| 2532 | 396 | 475 |
| 2533 | 395 | 475 |
| 2534 | 394 | 475 |
| 1748 | 393 | 475 |
| 2535 | 400 | 475 |
| 2536 | 399 | 475 |
| 2537 | 398 | 475 |
| 1757 | 397 | 475 |
| 1766 | 401 | 475 |
| 1775 | 405 | 475 |
| 1784 | 406 | 475 |
| 1793 | 407 | 475 |
| 1802 | 408 | 475 |
| 1811 | 409 | 475 |
| 1820 | 410 | 475 |
| 1829 | 411 | 475 |
| 1838 | 412 | 475 |
| 1847 | 413 | 475 |
| 1856 | 414 | 475 |
| 1865 | 415 | 475 |
| 1874 | 416 | 475 |
| 1883 | 417 | 475 |

TABLE 20a
Exemplary RTT sequences for Spacer S12

| SEQ ID NO: | Sequence |
|---|---|
| 418 | GCCcuGUUUGGAGUCAUAG |
| | CCAACG |
| 419 | UGCCcuGUUUGGAGUCAUA |
| | GCCAACG |
| 420 | UUGCCcuGUUUGGAGUCAU |
| | AGCCAACG |
| 421 | AUUGCCcuGUUUGGAGUCA |
| | UAGCCAACG |
| 422 | GCCCCAUUGCCcuGUUUGG |
| | AGUCAUAGCCAACG |
| 423 | GGCCCCAUUGCCcuGUUUGGAG |
| | UCAUAGCCAACG |
| 424 | UGGCCCCAUUGCCcuGUUUGGA |
| | GUCAUAGCCAACG |
| 425 | AUGGCCCCAUUGCCcuGUUUGG |
| | AGUCAUAGCCAACG |
| 426 | UAUGGCCCCAUUGCCcuGUUUG |
| | GAGUCAUAGCCAACG |

TABLE 20b
Exemplary PBS sequences for Spacer S12

| | Sequence |
|---|---|
| SEQ REF NO: 476 | AGAGUGC |
| SEQ REF NO: 477 | AGAGUGCC |
| SEQ REF NO: 478 | AGAGUGCCC |
| SEQ ID NO: 479 | AGAGUGCCCC |
| SEQ ID NO: 480 | AGAGUGCCCCU |
| SEQ ID NO: 481 | AGAGUGCCCCUC |
| SEQ ID NO: 482 | AGAGUGCCCCUCC |
| SEQ ID NO: 483 | AGAGUGCCCCUCCC |
| SEQ ID NO: 484 | AGAGUGCCCCUCCCA |

TABLE 20c
Exemplary RTT/PBS Combinations with Spacer S12

| PEG # | RTT | PBS |
|---|---|---|
| 1884 | 418 | 476 |
| 1893 | 419 | 476 |
| 1902 | 420 | 476 |
| 1911 | 421 | 476 |
| 1920 | 422 | 476 |
| 1929 | 423 | 476 |
| 1938 | 424 | 476 |
| 1947 | 425 | 476 |
| 1956 | 426 | 476 |
| 1885 | 418 | 477 |
| 1894 | 419 | 477 |
| 1903 | 420 | 477 |
| 1912 | 421 | 477 |
| 1921 | 422 | 477 |
| 1930 | 423 | 477 |
| 1939 | 424 | 477 |
| 1948 | 425 | 477 |
| 1957 | 426 | 477 |
| 1886 | 418 | 478 |
| 1895 | 419 | 478 |
| 1904 | 420 | 478 |
| 1913 | 421 | 478 |
| 1922 | 422 | 478 |
| 1931 | 423 | 478 |
| 1940 | 424 | 478 |
| 1949 | 425 | 478 |
| 1958 | 426 | 478 |
| 1887 | 418 | 479 |
| 1896 | 419 | 479 |
| 1905 | 420 | 479 |
| 1914 | 421 | 479 |
| 1923 | 422 | 479 |
| 1932 | 423 | 479 |
| 1941 | 424 | 479 |
| 1950 | 425 | 479 |
| 1959 | 426 | 479 |
| 1888 | 418 | 480 |
| 1897 | 419 | 480 |
| 1906 | 420 | 480 |
| 1915 | 421 | 480 |
| 1924 | 422 | 480 |
| 1933 | 423 | 480 |
| 1942 | 424 | 480 |
| 1951 | 425 | 480 |
| 1960 | 426 | 480 |
| 1889 | 418 | 481 |
| 1898 | 419 | 481 |
| 1907 | 420 | 481 |
| 1916 | 421 | 481 |
| 1925 | 422 | 481 |
| 1934 | 423 | 481 |
| 1943 | 424 | 481 |
| 1952 | 425 | 481 |
| 1961 | 426 | 481 |
| 1890 | 418 | 482 |
| 1899 | 419 | 482 |
| 1908 | 420 | 482 |
| 1917 | 421 | 482 |
| 1926 | 422 | 482 |
| 1935 | 423 | 482 |
| 1944 | 424 | 482 |
| 1953 | 425 | 482 |
| 1962 | 426 | 482 |
| 1891 | 418 | 483 |
| 1900 | 419 | 483 |
| 1909 | 420 | 483 |
| 1918 | 421 | 483 |
| 1927 | 422 | 483 |
| 1936 | 423 | 483 |
| 1945 | 424 | 483 |
| 1954 | 425 | 483 |
| 1963 | 426 | 483 |
| 1892 | 418 | 484 |
| 1901 | 419 | 484 |
| 1910 | 420 | 484 |
| 1919 | 421 | 484 |
| 1928 | 422 | 484 |
| 1937 | 423 | 484 |
| 1946 | 424 | 484 |
| 1955 | 425 | 484 |
| 1964 | 426 | 484 |

TABLE 21a
Exemplary RTT sequence for Spacer S13

| SEQ | |
|---|---|
| ID NO: | Sequence |
| 427 | GCCcuGUUUGGAGUCAUAGCCAACGAGAGUGCCCCUC |

TABLE 21b
Exemplary PBS sequences for Spacer S13

| | Sequence |
|---|---|
| SEQ REF NO: 485 | CCAACUU |
| SEQ REF NO: 486 | CCAACUUG |
| SEQ REF NO: 487 | CCAACUUGU |
| SEQ ID NO: 488 | CCAACUUGUG |
| SEQ ID NO: 489 | CCAACUUGUGU |
| SEQ ID NO: 490 | CCAACUUGUGUG |
| SEQ ID NO: 491 | CCAACUUGUGUGG |
| SEQ ID NO: 492 | CCAACUUGUGUGGC |
| SEQ ID NO: 493 | CCAACUUGUGUGGCA |

TABLE 21c
Exemplary RTT/PBS Combinations with Spacer S13

| PEG # | RTT | PBS |
|---|---|---|
| 1965 | 427 | 485 |
| 1966 | 427 | 486 |
| 1967 | 427 | 487 |
| 1968 | 427 | 488 |
| 1969 | 427 | 489 |
| 1970 | 427 | 490 |
| 1971 | 427 | 491 |
| 1972 | 427 | 492 |
| 1973 | 427 | 493 |

TABLE 22a
Exemplary RTT sequences for Spacer S14

| SEQ ID NO: | Sequence | SEQ ID NO: | Sequence |
|---|---|---|---|
| 428 | ACUCCAAACag | 439 | UCUCGUUGGCUAUGACUCCAAACag |
| 429 | GACUCCAAACag | 440 | ACUCUCGUUGGCUAUGACUCCAAACag |
| 430 | UGACUCCAAACag | 441 | GCACUCUCGUUGGCUAUGACUCCAAAC |
| | | | ag |
| 431 | AUGACUCCAAACag | 442 | GGCACUCUCGUUGGCUAUGACUCCAAA |
| | | | Cag |
| 432 | UAUGACUCCAAACag | 443 | GGGCACUCUCGUUGGCUAUGACUCCAA |
| | | | ACag |
| 433 | GCUAUGACUCCAAACag | 444 | GGGGCACUCUCGUUGGCUAUGACUCCA |
| | | | AACag |
| 434 | GGCUAUGACUCCAAACag | 445 | AGGGGCACUCUCGUUGGCUAUGACUCC |
| | | | AAACag |
| 435 | UGGCUAUGACUCCAAACag | 446 | GAGGGGCACUCUCGUUGGCUAUGACUC |
| | | | CAAACag |
| 436 | UUGGCUAUGACUCCAAACa | 447 | GGAGGGGCACUCUCGUUGGCUAUGACU |
| | g | | CCAAACag |
| 437 | GUUGGCUAUGACUCCAAAC | 448 | GGGAGGGGCACUCUCGUUGGCUAUGAC |
| | ag | | UCCAAACag |
| 438 | UCGUUGGCUAUGACUCCAA | 449 | UGGGAGGGGCACUCUCGUUGGCUAUGA |
| | ACag | | CUCCAAACag |
| 6001 | CUAUGACUCCAAACag | | |

TABLE 22b
Exemplary PBS sequences for Spacer S14

| | Sequence |
|---|---|
| SEQ REF NO: 494 | GGCAAUG |
| SEQ REF NO: 495 | GGCAAUGG |
| SEQ REF NO: 496 | GGCAAUGGG |
| SEQ ID NO: 497 | GGCAAUGGGG |
| SEQ ID NO: 498 | GGCAAUGGGGC |
| SEQ ID NO: 499 | GGCAAUGGGGCC |
| SEQ ID NO: 500 | GGCAAUGGGGCCA |
| SEQ ID NO: 501 | GGCAAUGGGGCCAU |
| SEQ ID NO: 502 | GGCAAUGGGGCCAUA |

TABLE 22c
Exemplary RTT/PBS Combinations with Spacer S14

| PEG # | RTT | PBS |
|---|---|---|
| 2058 | 428 | 494 |
| 2067 | 429 | 494 |
| 2076 | 430 | 494 |
| 2085 | 431 | 494 |
| 2094 | 432 | 494 |
| 2103 | 433 | 494 |
| 2112 | 434 | 494 |
| 2121 | 435 | 494 |
| 2130 | 436 | 494 |
| 2139 | 437 | 494 |
| 2148 | 438 | 494 |
| 2157 | 439 | 494 |
| 2166 | 440 | 494 |
| 2175 | 441 | 494 |
| 2184 | 442 | 494 |
| 2193 | 443 | 494 |
| 2202 | 444 | 494 |
| 2211 | 445 | 494 |
| 2220 | 446 | 494 |
| 2229 | 447 | 494 |
| 2238 | 448 | 494 |
| 2247 | 449 | 494 |
| 2059 | 428 | 495 |
| 2068 | 429 | 495 |
| 2077 | 430 | 495 |
| 2086 | 431 | 495 |
| 2095 | 432 | 495 |
| 2104 | 433 | 495 |
| 2113 | 434 | 495 |
| 2122 | 435 | 495 |
| 2131 | 436 | 495 |
| 2140 | 437 | 495 |
| 2149 | 438 | 495 |
| 2158 | 439 | 495 |
| 2167 | 440 | 495 |
| 2176 | 441 | 495 |
| 2185 | 442 | 495 |
| 2194 | 443 | 495 |
| 2203 | 444 | 495 |
| 2212 | 445 | 495 |
| 2221 | 446 | 495 |
| 2230 | 447 | 495 |
| 2239 | 448 | 495 |
| 2248 | 449 | 495 |
| 2060 | 428 | 496 |
| 2069 | 429 | 496 |
| 2078 | 430 | 496 |
| 2087 | 431 | 496 |
| 2096 | 432 | 496 |
| 2105 | 433 | 496 |
| 2114 | 434 | 496 |
| 2123 | 435 | 496 |
| 2132 | 436 | 496 |
| 2141 | 437 | 496 |
| 2150 | 438 | 496 |
| 2159 | 439 | 496 |
| 2168 | 440 | 496 |
| 2177 | 441 | 496 |
| 2186 | 442 | 496 |
| 2195 | 443 | 496 |
| 2204 | 444 | 496 |
| 2213 | 445 | 496 |
| 2222 | 446 | 496 |
| 2231 | 447 | 496 |
| 2240 | 448 | 496 |
| 2249 | 449 | 496 |
| 2061 | 428 | 497 |
| 2070 | 429 | 497 |
| 2079 | 430 | 497 |
| 2088 | 431 | 497 |
| 2097 | 432 | 497 |
| 2409 | 6001 | 497 |
| 2106 | 433 | 497 |
| 2115 | 434 | 497 |
| 2124 | 435 | 497 |
| 2133 | 436 | 497 |
| 2142 | 437 | 497 |
| 2151 | 438 | 497 |
| 2160 | 439 | 497 |
| 2169 | 440 | 497 |
| 2178 | 441 | 497 |
| 2187 | 442 | 497 |
| 2196 | 443 | 497 |
| 2205 | 444 | 497 |
| 2214 | 445 | 497 |
| 2223 | 446 | 497 |
| 2232 | 447 | 497 |
| 2241 | 448 | 497 |
| 2250 | 449 | 497 |
| 2062 | 428 | 498 |
| 2071 | 429 | 498 |
| 2080 | 430 | 498 |
| 2089 | 431 | 498 |
| 2098 | 432 | 498 |
| 2410 | 6001 | 498 |
| 2107 | 433 | 498 |
| 2116 | 434 | 498 |
| 2125 | 435 | 498 |
| 2134 | 436 | 498 |
| 2143 | 437 | 498 |
| 2152 | 438 | 498 |
| 2161 | 439 | 498 |
| 2170 | 440 | 498 |
| 2179 | 441 | 498 |
| 2188 | 442 | 498 |
| 2197 | 443 | 498 |
| 2206 | 444 | 498 |
| 2215 | 445 | 498 |
| 2224 | 446 | 498 |
| 2233 | 447 | 498 |
| 2242 | 448 | 498 |
| 2251 | 449 | 498 |
| 2063 | 428 | 499 |
| 2072 | 429 | 499 |
| 2081 | 430 | 499 |
| 2090 | 431 | 499 |
| 2099 | 432 | 499 |
| 2411 | 6001 | 499 |
| 2108 | 433 | 499 |
| 2117 | 434 | 499 |
| 2126 | 435 | 499 |
| 2135 | 436 | 499 |
| 2144 | 437 | 499 |
| 2153 | 438 | 499 |
| 2162 | 439 | 499 |
| 2171 | 440 | 499 |
| 2180 | 441 | 499 |
| 2189 | 442 | 499 |
| 2198 | 443 | 499 |
| 2207 | 444 | 499 |
| 2216 | 445 | 499 |
| 2225 | 446 | 499 |
| 2234 | 447 | 499 |
| 2243 | 448 | 499 |
| 2252 | 449 | 499 |
| 2064 | 428 | 500 |
| 2073 | 429 | 500 |
| 2082 | 430 | 500 |
| 2091 | 431 | 500 |
| 2100 | 432 | 500 |
| 2412 | 6001 | 500 |
| 2109 | 433 | 500 |
| 2118 | 434 | 500 |
| 2127 | 435 | 500 |
| 2136 | 436 | 500 |
| 2145 | 437 | 500 |
| 2154 | 438 | 500 |
| 2163 | 439 | 500 |
| 2172 | 440 | 500 |
| 2181 | 441 | 500 |
| 2190 | 442 | 500 |
| 2199 | 443 | 500 |
| 2208 | 444 | 500 |
| 2217 | 445 | 500 |
| 2226 | 446 | 500 |
| 2235 | 447 | 500 |
| 2244 | 448 | 500 |
| 2253 | 449 | 500 |
| 2065 | 428 | 501 |
| 2074 | 429 | 501 |
| 2083 | 430 | 501 |
| 2092 | 431 | 501 |
| 2101 | 432 | 501 |
| 2413 | 6001 | 501 |
| 2110 | 433 | 501 |
| 2119 | 434 | 501 |
| 2128 | 435 | 501 |
| 2137 | 436 | 501 |
| 2146 | 437 | 501 |
| 2155 | 438 | 501 |
| 2164 | 439 | 501 |
| 2173 | 440 | 501 |
| 2182 | 441 | 501 |
| 2191 | 442 | 501 |
| 2200 | 443 | 501 |
| 2209 | 444 | 501 |
| 2218 | 445 | 501 |
| 2227 | 446 | 501 |
| 2236 | 447 | 501 |
| 2245 | 448 | 501 |
| 2254 | 449 | 501 |
| 2066 | 428 | 502 |
| 2075 | 429 | 502 |
| 2084 | 430 | 502 |
| 2093 | 431 | 502 |
| 2102 | 432 | 502 |
| 2111 | 433 | 502 |
| 2120 | 434 | 502 |
| 2129 | 435 | 502 |
| 2138 | 436 | 502 |
| 2147 | 437 | 502 |
| 2156 | 438 | 502 |
| 2165 | 439 | 502 |
| 2174 | 440 | 502 |
| 2183 | 441 | 502 |
| 2192 | 442 | 502 |
| 2201 | 443 | 502 |
| 2210 | 444 | 502 |
| 2219 | 445 | 502 |
| 2228 | 446 | 502 |
| 2237 | 447 | 502 |
| 2246 | 448 | 502 |
| 2255 | 449 | 502 |

TABLE 23a
Exemplary RTT sequences for Spacer S15

| SEQ ID NO: | Sequence | SEQ ID NO: | Sequence |
|---|---|---|---|
| 450 | ACagGGCAAUGGGGCCAUA | 457 | AUGACUCCAAACagGGCAAUGGGGC |
| | C | | CAUAC |
| 451 | AACagGGCAAUGGGGCCAU | 458 | UAUGACUCCAAACagGGCAAUGGGG |
| | AC | | CCAUAC |
| 452 | AAACagGGCAAUGGGGCCA | 459 | GCUAUGACUCCAAACagGGCAAUGG |
| | UAC | | GGCCAUAC |
| 453 | UCCAAACagGGCAAUGGGG | 460 | GGCUAUGACUCCAAACagGGCAAUG |
| | CCAUAC | | GGGCCAUAC |
| 454 | ACUCCAAACagGGCAAUGG | 461 | UGGCUAUGACUCCAAACagGGCAAU |
| | GGCCAUAC | | GGGGCCAUAC |
| 455 | GACUCCAAACagGGCAAUG | 462 | UUGGCUAUGACUCCAAACagGGCAA |
| | GGGCCAUAC | | UGGGGCCAUAC |
| 456 | UGACUCCAAACagGGCAAU | 463 | GUUGGCUAUGACUCCAAACagGGCA |
| | GGGGCCAUAC | | AUGGGGCCAUAC |
| 6003 | CCAAACagGGCAAUGGGGC | 6004 | CUCCAAACagGGCAAUGGGGCCAUA |
| | CAUAC | | C |

TABLE 23b
Exemplary PBS sequences for Spacer S15

| | Sequence |
|---|---|
| SEQ REF NO: 503 | GAGGAGA |
| SEQ REF NO: 504 | GAGGAGAA |
| SEQ REF NO: 505 | GAGGAGAAA |
| SEQ ID NO: 506 | GAGGAGAAAC |
| SEQ ID NO: 507 | GAGGAGAAACC |
| SEQ ID NO: 508 | GAGGAGAAACCA |
| SEQ ID NO: 509 | GAGGAGAAACCAA |
| SEQ ID NO: 510 | GAGGAGAAACCAAA |
| SEQ ID NO: 511 | GAGGAGAAACCAAAU |

TABLE 23c
Exemplary RTT/PBS Combinations with Spacer S15

| PEG # | RTT | PBS |
|---|---|---|
| 2256 | 450 | 503 |
| 2265 | 451 | 503 |
| 2274 | 452 | 503 |
| 2283 | 453 | 503 |
| 2292 | 454 | 503 |
| 2301 | 455 | 503 |
| 2310 | 456 | 503 |
| 2319 | 457 | 503 |
| 2328 | 458 | 503 |
| 2337 | 459 | 503 |
| 2346 | 460 | 503 |
| 2355 | 461 | 503 |
| 2364 | 462 | 503 |
| 2373 | 463 | 503 |
| 2257 | 450 | 504 |
| 2266 | 451 | 504 |
| 2275 | 452 | 504 |
| 2284 | 453 | 504 |
| 2293 | 454 | 504 |
| 2302 | 455 | 504 |
| 2311 | 456 | 504 |
| 2320 | 457 | 504 |
| 2329 | 458 | 504 |
| 2338 | 459 | 504 |
| 2347 | 460 | 504 |
| 2356 | 461 | 504 |
| 2365 | 462 | 504 |
| 2374 | 463 | 504 |
| 2258 | 450 | 505 |
| 2267 | 451 | 505 |
| 2276 | 452 | 505 |
| 2422 | 6003 | 505 |
| 2285 | 453 | 505 |
| 2423 | 453 | 505 |
| 2424 | 6004 | 505 |
| 2294 | 454 | 505 |
| 2425 | 454 | 505 |
| 2303 | 455 | 505 |
| 2426 | 455 | 505 |
| 2312 | 456 | 505 |
| 2427 | 456 | 505 |
| 2321 | 457 | 505 |
| 2428 | 457 | 505 |
| 2330 | 458 | 505 |
| 2339 | 459 | 505 |
| 2348 | 460 | 505 |
| 2357 | 461 | 505 |
| 2366 | 462 | 505 |
| 2375 | 463 | 505 |
| 2259 | 450 | 506 |
| 2268 | 451 | 506 |
| 2277 | 452 | 506 |
| 2414 | 6003 | 506 |
| 2286 | 453 | 506 |
| 2415 | 6004 | 506 |
| 2295 | 454 | 506 |
| 2304 | 455 | 506 |
| 2313 | 456 | 506 |
| 2322 | 457 | 506 |
| 2331 | 458 | 506 |
| 2340 | 459 | 506 |
| 2349 | 460 | 506 |
| 2358 | 461 | 506 |
| 2367 | 462 | 506 |
| 2376 | 463 | 506 |
| 2260 | 450 | 507 |
| 2269 | 451 | 507 |
| 2278 | 452 | 507 |
| 2416 | 6003 | 507 |
| 2287 | 453 | 507 |
| 2417 | 6004 | 507 |
| 2296 | 454 | 507 |
| 2305 | 455 | 507 |
| 2314 | 456 | 507 |
| 2323 | 457 | 507 |
| 2332 | 458 | 507 |
| 2341 | 459 | 507 |
| 2350 | 460 | 507 |
| 2359 | 461 | 507 |
| 2368 | 462 | 507 |
| 2377 | 463 | 507 |
| 2261 | 450 | 508 |
| 2270 | 451 | 508 |
| 2279 | 452 | 508 |
| 2418 | 6003 | 508 |
| 2288 | 453 | 508 |
| 2419 | 6004 | 508 |
| 2297 | 454 | 508 |
| 2306 | 455 | 508 |
| 2315 | 456 | 508 |
| 2324 | 457 | 508 |
| 2333 | 458 | 508 |
| 2342 | 459 | 508 |
| 2351 | 460 | 508 |
| 2360 | 461 | 508 |
| 2369 | 462 | 508 |
| 2378 | 463 | 508 |
| 2262 | 450 | 509 |
| 2271 | 451 | 509 |
| 2280 | 452 | 509 |
| 2420 | 6003 | 509 |
| 2289 | 453 | 509 |
| 2421 | 6004 | 509 |
| 2298 | 454 | 509 |
| 2307 | 455 | 509 |
| 2316 | 456 | 509 |
| 2325 | 457 | 509 |
| 2334 | 458 | 509 |
| 2343 | 459 | 509 |
| 2352 | 460 | 509 |
| 2361 | 461 | 509 |
| 2370 | 462 | 509 |
| 2379 | 463 | 509 |
| 2263 | 450 | 510 |
| 2272 | 451 | 510 |
| 2281 | 452 | 510 |
| 2290 | 453 | 510 |
| 2299 | 454 | 510 |
| 2308 | 455 | 510 |
| 2317 | 456 | 510 |
| 2326 | 457 | 510 |
| 2335 | 458 | 510 |
| 2344 | 459 | 510 |
| 2353 | 460 | 510 |
| 2362 | 461 | 510 |
| 2371 | 462 | 510 |
| 2380 | 463 | 510 |
| 2264 | 450 | 511 |
| 2273 | 451 | 511 |
| 2282 | 452 | 511 |
| 2291 | 453 | 511 |
| 2300 | 454 | 511 |
| 2309 | 455 | 511 |
| 2318 | 456 | 511 |
| 2327 | 457 | 511 |
| 2336 | 458 | 511 |
| 2345 | 459 | 511 |
| 2354 | 460 | 511 |
| 2363 | 461 | 511 |
| 2372 | 462 | 511 |
| 2381 | 463 | 511 |

TABLE 24a
Exemplary RTT sequences for Spacer S16

| SEQ ID NO: | Sequence |
|---|---|
| 464 | ACagGGCAAUGGGGCCAUA |
| | CGAGGAGAAACCAAAU |
| 465 | AACagGGCAAUGGGGCCAU |
| | ACGAGGAGAAACCAAAU |
| 466 | AAACagGGCAAUGGGGCCA |
| | UACGAGGAGAAACCAAAU |

TABLE 24b
Exemplary PBS sequences for Spacer S16

| | Sequence |
|---|---|
| SEQ REF NO: 512 | ACAGCUC |
| SEQ REF NO: 513 | ACAGCUCC |
| SEQ REF NO: 514 | ACAGCUCCC |
| SEQ ID NO: 515 | ACAGCUCCCA |
| SEQ ID NO: 516 | ACAGCUCCCAA |
| SEQ ID NO: 517 | ACAGCUCCCAAU |
| SEQ ID NO: 518 | ACAGCUCCCAAUA |
| SEQ ID NO: 519 | ACAGCUCCCAAUAC |
| SEQ ID NO: 520 | ACAGCUCCCAAUACC |

TABLE 24c
Exemplary RTT/PBS Combinations with Spacer S16

| PEG # | RTT | PBS |
|---|---|---|
| 2382 | 464 | 512 |
| 2391 | 465 | 512 |
| 2400 | 466 | 512 |
| 2383 | 464 | 513 |
| 2392 | 465 | 513 |
| 2401 | 466 | 513 |
| 2384 | 464 | 514 |
| 2393 | 465 | 514 |
| 2402 | 466 | 514 |
| 2385 | 464 | 515 |
| 2394 | 465 | 515 |
| 2403 | 466 | 515 |
| 2386 | 464 | 516 |
| 2395 | 465 | 516 |
| 2404 | 466 | 516 |
| 2387 | 464 | 517 |
| 2396 | 465 | 517 |
| 2405 | 466 | 517 |
| 2388 | 464 | 518 |
| 2397 | 465 | 518 |
| 2406 | 466 | 518 |
| 2389 | 464 | 519 |
| 2398 | 465 | 519 |
| 2407 | 466 | 519 |
| 2390 | 464 | 520 |
| 2399 | 465 | 520 |
| 2408 | 466 | 520 |

TABLE 24d
Correction of L348fs using PE2 systems, experiment 1.

| | First trial | | | | | | | |
|---|---|---|---|---|---|---|---|---|
| | Replicate A | | Replicate B | | Replicate C | | Second trial | |
| | edit | indel | edit | indel | edit | indel | edit | indel |
| Peg# | % | % | % | % | % | % | % | % |
| 2059 | 19.3% | 1.8% | 11.7% | 0.6% | 18.1% | 0.9% | 7.6% | 0.5% |
| 2068 | 20.4% | 1.3% | 10.2% | 0.6% | 14.8% | 0.7% | 8.2% | 0.5% |
| 2077 | 26.5% | 1.9% | 15.2% | 0.9% | 20.9% | 0.8% | 10.5% | 0.7% |
| 2086 | 22.6% | 1.3% | 20.7% | 1.7% | 18.4% | 1.4% | 11.5% | 0.5% |
| 2095 | 18.8% | 2.9% | 15.9% | 1.7% | 15.1% | 2.1% | 11.6% | 1.0% |
| 2104 | 16.1% | 2.5% | 12.9% | 1.5% | 11.6% | 1.8% | 11.9% | 1.0% |
| 2113 | 22.4% | 2.3% | 13.5% | 0.8% | 18.3% | 1.2% | 7.2% | 0.5% |
| 2061 | 41.8% | 2.1% | 24.4% | 1.0% | 34.5% | 1.7% | 12.7% | 0.6% |
| 2070 | 37.9% | 1.2% | 21.1% | 0.9% | 31.7% | 1.3% | 11.2% | 0.6% |
| 2079 | 40.4% | 1.9% | 21.8% | 1.3% | 33.4% | 1.7% | 11.7% | 0.7% |
| 2088 | 44.3% | 2.2% | 34.6% | 2.1% | 36.0% | 3.1% | 19.7% | 1.0% |
| 2097 | 43.1% | 2.8% | 21.3% | 2.1% | 36.7% | 2.9% | 19.5% | 1.3% |
| 2106 | 0.0% | 0.1% | 0.0% | 0.1% | 0.0% | 0.2% | 4.3% | 0.4% |
| 2115 | 40.2% | 3.4% | 23.8% | 1.7% | 32.6% | 3.0% | 14.3% | 0.6% |
| 2063 | 44.2% | 2.8% | 24.5% | 1.2% | 34.9% | 2.0% | 14.9% | 0.7% |
| 2072 | 40.2% | 1.6% | 22.2% | 1.0% | 33.7% | 1.8% | 12.7% | 0.7% |
| 2081 | 45.8% | 1.8% | 24.6% | 1.4% | 38.8% | 1.7% | 16.2% | 1.7% |
| 2090 | 48.5% | 2.4% | 35.9% | 3.3% | 44.3% | 3.2% | 20.7% | 1.2% |
| 2099 | 0.0% | 0.1% | 0.0% | 0.1% | 0.0% | 0.1% | 5.0% | 0.4% |
| 2108 | 45.5% | 5.7% | 30.8% | 2.5% | 35.6% | 4.5% | 16.2% | 1.6% |
| 2117 | 41.0% | 6.3% | 31.2% | 3.1% | 40.5% | 6.0% | 14.3% | 0.9% |
| 2065 | 43.1% | 3.0% | 22.5% | 1.3% | 38.4% | 2.0% | 13.1% | 0.6% |
| 2074 | 41.0% | 1.9% | 21.3% | 1.7% | 38.0% | 2.1% | 11.2% | 0.6% |
| 2083 | 43.7% | 1.9% | 23.0% | 1.2% | 38.8% | 1.7% | 15.2% | 0.8% |
| 2092 | 0.0% | 0.1% | 0.0% | 0.1% | 0.0% | 0.1% | 1.6% | 0.3% |
| 2101 | 49.1% | 2.4% | 30.5% | 1.6% | 41.5% | 2.2% | 17.5% | 0.9% |
| 2110 | 34.1% | 2.1% | 20.6% | 1.4% | 27.0% | 2.7% | 13.1% | 0.8% |
| 2119 | 39.1% | 3.7% | 28.7% | 2.3% | 35.4% | 5.4% | 14.0% | 0.9% |
| 2257 | 0.0% | 0.9% | 0.0% | 0.3% | 0.0% | 0.1% | 6.4% | 0.4% |
| 2284 | 8.3% | 0.4% | 2.7% | 0.4% | 6.3% | 0.4% | 2.3% | 0.3% |
| 2311 | 9.1% | 0.8% | 4.1% | 0.4% | 8.1% | 0.6% | 4.5% | 0.4% |
| 2338 | 7.7% | 0.9% | 3.5% | 0.3% | 7.7% | 0.6% | 6.4% | 0.4% |
| 2259 | 0.0% | 0.3% | 0.0% | 0.5% | 0.0% | 1.2% | 11.0% | 0.4% |
| 2286 | 18.7% | 1.3% | 6.6% | 0.3% | 13.8% | 0.8% | 6.0% | 0.4% |
| 2313 | 16.6% | 1.2% | 5.7% | 0.2% | 12.6% | 0.4% | 6.5% | 0.5% |
| 2340 | 11.2% | 0.7% | 4.1% | 0.4% | 8.4% | 0.4% | 6.7% | 0.3% |
| 2261 | 0.0% | 0.1% | 0.0% | 0.1% | 0.0% | 0.1% | 2.0% | 0.4% |
| 2288 | 0.0% | 0.1% | 0.0% | 0.1% | 0.0% | 0.1% | 3.2% | 0.3% |
| 2315 | 10.1% | 0.6% | 3.5% | 0.3% | 7.1% | 0.4% | 5.0% | 0.4% |
| 2342 | 13.1% | 1.2% | 3.8% | 0.3% | 7.1% | 0.8% | 5.3% | 0.4% |
| 2263 | 0.0% | 0.1% | 0.0% | 0.1% | 0.0% | 0.1% | 1.9% | 0.4% |
| 2290 | 7.0% | 0.1% | 1.7% | 0.2% | 6.6% | 0.3% | 5.1% | 0.3% |
| 2317 | 0.0% | 0.1% | 0.0% | 0.1% | 0.0% | 0.1% | 0.6% | 0.3% |
| 2344 | 5.3% | 0.3% | 2.4% | 0.4% | 4.8% | 0.4% | 3.6% | 0.4% |
| 1974 | 0.0% | 0.2% | 0.0% | 0.2% | 0.1% | 0.9% | 4.4% | 0.4% |
| 2030 | 0.0% | 0.1% | 0.0% | 0.3% | 0.1% | 0.1% | 7.4% | 0.3% |
| 2002 | 0.0% | 0.2% | 0.0% | 0.3% | 0.0% | 0.5% | 5.6% | 0.4% |
| 1705 | 0.2% | 0.7% | 0.2% | 0.2% | 0.3% | 0.1% | 8.0% | 0.3% |
| 1978 | 0.2% | 0.7% | 0.6% | 0.3% | 0.5% | 0.3% | 8.3% | 0.4% |
| 2034 | 0.6% | 0.8% | 0.2% | 0.4% | 0.3% | 0.5% | 7.9% | 0.3% |
| 2006 | 0.8% | 1.0% | 0.3% | 0.4% | 3.4% | 0.9% | 11.3% | 0.3% |
| 1714 | 0.7% | 0.1% | 0.7% | 0.3% | 0.9% | 0.6% | 4.0% | 0.4% |
| 1982 | 36.6% | 0.7% | 11.7% | 0.5% | 26.3% | 0.6% | 12.3% | 0.3% |
| 2038 | 26.7% | 0.6% | 9.9% | 0.5% | 23.2% | 0.4% | 3.4% | 0.4% |
| 2010 | 52.6% | 1.8% | 14.3% | 0.7% | 42.3% | 0.3% | 16.6% | 0.3% |
| 1723 | 7.2% | 0.3% | 6.7% | 0.8% | 9.2% | 0.3% | 6.3% | 0.3% |
| 1986 | 10.4% | 1.0% | 5.4% | 0.6% | 8.6% | 0.6% | 10.3% | 0.4% |
| 2042 | 24.1% | 0.2% | 6.0% | 0.4% | 14.9% | 0.5% | 5.7% | 0.4% |
| 2014 | 17.6% | 1.0% | 6.2% | 0.6% | 12.2% | 0.2% | 2.5% | 0.3% |
| 1732 | 0.9% | 0.1% | 2.0% | 0.2% | 1.2% | 0.9% | 4.0% | 0.4% |
| 1990 | 4.6% | 0.5% | 2.8% | 0.2% | 4.2% | 0.2% | 3.8% | 0.4% |
| 2046 | 22.1% | 0.7% | 5.8% | 0.3% | 11.4% | 0.2% | 6.4% | 0.4% |
| 2018 | 10.5% | 0.7% | 5.4% | 0.5% | 9.0% | 0.4% | 5.0% | 0.3% |
| 1741 | 3.3% | 1.4% | 2.5% | 0.7% | 2.3% | 1.0% | 9.7% | 0.3% |
| 1994 | 9.7% | 0.3% | 3.4% | 0.7% | 6.6% | 0.4% | 6.0% | 0.4% |
| 2050 | 17.4% | 0.5% | 11.3% | 0.5% | 12.7% | 0.3% | 4.7% | 0.4% |
| 2022 | 10.1% | 0.6% | 4.1% | 0.4% | 10.0% | 0.4% | 8.6% | 0.4% |
| 1750 | 1.9% | 0.5% | 1.6% | 0.4% | 2.1% | 0.3% | 1.2% | 0.4% |
| 1998 | 3.7% | 0.5% | 1.9% | 0.3% | 2.5% | 0.5% | 8.1% | 0.3% |
| 2054 | 10.2% | 0.7% | 5.0% | 0.4% | 7.7% | 0.6% | 7.4% | 0.5% |
| 2026 | 2.2% | 0.0% | 1.3% | 0.2% | 1.7% | 0.1% | 4.9% | 0.4% |
| 1759 | 2.1% | 0.7% | 1.1% | 0.6% | 1.8% | 0.3% | 4.0% | 0.4% |
| 1975 | 0.0% | 0.5% | 0.0% | 0.3% | 0.1% | 0.6% | 6.2% | 0.5% |
| 2031 | 0.0% | 0.5% | 0.0% | 0.5% | 0.0% | 0.5% | 8.7% | 0.3% |
| 2003 | 0.0% | 0.0% | 0.0% | 0.2% | 0.0% | 0.5% | 7.4% | 0.3% |
| 1707 | 0.2% | 0.1% | 0.0% | 0.1% | 0.1% | 0.1% | 2.5% | 0.4% |
| 1979 | 1.4% | 0.7% | 0.8% | 0.5% | 1.2% | 0.5% | 9.0% | 0.3% |
| 2035 | 0.1% | 0.1% | 0.0% | 0.3% | 0.1% | 0.4% | 7.5% | 0.4% |
| 2007 | 0.7% | 0.2% | 0.4% | 0.2% | 0.4% | 0.2% | 5.4% | 0.3% |
| 1716 | 1.2% | 0.8% | 1.4% | 0.4% | 1.2% | 0.6% | 4.2% | 0.3% |
| 1983 | 0.0% | 0.1% | 0.1% | 0.1% | 0.0% | 0.1% | 3.1% | 0.3% |
| 2039 | 38.9% | 2.2% | 15.3% | 0.5% | 34.5% | 1.1% | 7.5% | 0.4% |
| 2011 | 0.0% | 0.1% | 0.0% | 0.0% | 0.0% | 0.1% | 6.4% | 0.3% |
| 1725 | 0.2% | 0.1% | 0.0% | 0.1% | 0.0% | 0.1% | 0.1% | 0.4% |
| 1987 | 26.9% | 0.5% | 14.1% | 0.5% | 22.7% | 0.8% | 15.7% | 0.4% |
| 2043 | 28.3% | 0.7% | 9.4% | 0.6% | 22.1% | 0.6% | 8.8% | 0.4% |
| 2015 | 17.9% | 0.4% | 7.2% | 0.3% | 12.9% | 0.4% | 3.3% | 0.3% |
| 1734 | 3.0% | 0.5% | 2.1% | 0.2% | 3.4% | 0.6% | 7.1% | 0.3% |
| 1991 | 21.3% | 0.7% | 10.7% | 0.5% | 16.7% | 0.5% | 6.9% | 0.3% |
| 2047 | 0.0% | 0.1% | 0.0% | 0.1% | 0.0% | 0.1% | 3.1% | 0.4% |
| 2019 | 28.3% | 0.7% | 9.4% | 0.4% | 20.7% | 0.8% | 8.7% | 0.4% |
| 1743 | 0.0% | 0.1% | 0.0% | 0.1% | 0.0% | 0.1% | 5.4% | 0.3% |
| 1995 | 11.9% | 0.6% | 5.9% | 0.2% | 11.0% | 0.7% | 8.5% | 0.3% |
| 2051 | 35.5% | 1.3% | 18.3% | 0.8% | 31.3% | 0.6% | 12.0% | 0.6% |
| 2023 | 21.8% | 1.0% | 6.8% | 0.5% | 12.6% | 0.5% | 5.7% | 0.3% |
| 1752 | 9.9% | 2.6% | 6.1% | 1.3% | 8.3% | 3.5% | 4.7% | 0.4% |
| 1999 | 0.0% | 0.1% | 0.0% | 0.1% | 0.0% | 0.1% | 1.0% | 0.4% |
| 2055 | 15.7% | 0.4% | 9.1% | 0.6% | 11.2% | 0.4% | 7.5% | 0.4% |
| 2027 | 13.5% | 0.6% | 6.6% | 0.4% | 11.8% | 0.5% | 10.2% | 0.4% |
| 1761 | 5.7% | 0.4% | 3.8% | 0.4% | 3.8% | 0.6% | 6.7% | 0.3% |
| 1976 | 0.4% | 0.4% | 0.0% | 0.2% | 0.8% | 0.4% | 7.0% | 0.4% |
| 2032 | 0.0% | 0.1% | 0.0% | 0.1% | 0.0% | 0.1% | 5.3% | 0.3% |
| 2004 | 0.0% | 0.1% | 0.0% | 0.1% | 0.0% | 0.1% | 3.6% | 0.3% |
| 1709 | 0.0% | 1.0% | 0.1% | 0.3% | 0.0% | 0.4% | 6.1% | 0.4% |
| 1980 | 6.6% | 1.3% | 2.1% | 0.7% | 3.7% | 0.7% | 10.8% | 0.4% |
| 2036 | 0.0% | 0.2% | 0.0% | 0.1% | 0.0% | 0.1% | 4.0% | 0.3% |
| 2008 | 9.9% | 9.1% | 2.8% | 2.3% | 9.0% | 5.3% | 4.8% | 3.8% |
| 1718 | 4.4% | 0.8% | 2.3% | 0.3% | 5.0% | 0.9% | 6.9% | 0.4% |
| 1984 | 68.9% | 2.1% | 34.7% | 0.8% | 59.5% | 1.6% | 25.1% | 0.5% |
| 2040 | 58.8% | 0.8% | 25.4% | 0.6% | 52.3% | 0.7% | 14.4% | 0.4% |
| 2012 | 0.0% | 0.1% | 0.0% | 0.1% | 0.0% | 0.0% | 5.1% | 0.3% |
| 1727 | 46.4% | 1.4% | 34.6% | 0.9% | 43.6% | 0.7% | 18.9% | 0.4% |
| 1988 | 0.0% | 0.1% | 0.0% | 0.1% | 0.0% | 0.1% | 5.8% | 0.4% |
| 2044 | 67.0% | 1.3% | 32.8% | 1.1% | 61.6% | 0.8% | 17.8% | 0.6% |
| 2016 | 65.9% | 1.4% | 35.2% | 1.2% | 57.9% | 1.4% | 17.1% | 0.6% |
| 1736 | 33.6% | 1.5% | 23.6% | 0.7% | 28.4% | 2.0% | 12.4% | 0.3% |
| 1992 | 58.6% | 1.1% | 26.3% | 0.4% | 51.6% | 1.2% | 9.6% | 0.4% |
| 2048 | 59.2% | 1.2% | 17.1% | 0.6% | 46.9% | 1.2% | 12.2% | 0.5% |
| 2020 | 65.6% | 0.3% | 28.2% | 0.8% | 61.4% | 1.0% | 15.4% | 0.7% |
| 1745 | 22.6% | 0.9% | 18.9% | 0.8% | 20.4% | 0.8% | 11.4% | 0.5% |
| 1996 | 56.1% | 1.5% | 23.0% | 0.6% | 49.8% | 1.4% | 9.9% | 0.5% |
| 2052 | 0.0% | 0.1% | 0.0% | 0.1% | 0.0% | 0.1% | 3.9% | 0.4% |
| 2024 | 54.0% | 1.2% | 25.6% | 1.2% | 49.0% | 1.1% | 18.2% | 0.6% |
| 1754 | 0.2% | 0.1% | 0.1% | 0.2% | 0.2% | 0.2% | 2.5% | 0.4% |
| 2000 | 48.4% | 0.5% | 22.8% | 0.7% | 41.6% | 0.9% | 14.3% | 0.4% |
| 2056 | 46.5% | 1.3% | 19.8% | 0.7% | 39.3% | 0.7% | 12.0% | 0.4% |
| 2028 | 0.0% | 0.1% | 0.0% | 0.1% | 0.0% | 0.1% | 4.2% | 0.5% |
| 1763 | 27.1% | 1.2% | 17.7% | 0.9% | 25.0% | 2.5% | 11.7% | 0.6% |
| 1977 | 0.0% | 0.1% | 0.0% | 0.1% | 0.0% | 0.1% | 0.5% | 0.4% |
| 2033 | 0.1% | 1.7% | 0.0% | 2.2% | 0.1% | 2.5% | 3.1% | 0.5% |
| 2005 | 0.0% | 0.3% | 0.0% | 0.1% | 0.0% | 0.2% | 3.3% | 0.3% |
| 1711 | 0.2% | 0.4% | 0.0% | 0.4% | 0.0% | 0.6% | 5.1% | 0.4% |
| 1981 | 8.5% | 1.2% | 3.8% | 0.5% | 7.1% | 0.4% | 10.6% | 0.4% |
| 2037 | 0.0% | 0.1% | 0.0% | 0.1% | 0.0% | 0.1% | 5.4% | 0.3% |
| 2009 | 0.0% | 0.1% | 0.0% | 0.1% | 0.0% | 0.1% | 5.3% | 0.4% |
| 1720 | 2.7% | 1.1% | 2.2% | 0.4% | 2.5% | 0.7% | 4.7% | 0.4% |
| 1985 | 64.1% | 0.9% | 23.4% | 0.5% | 61.8% | 0.8% | 20.4% | 0.5% |
| 2041 | 67.0% | 1.4% | 26.8% | 0.8% | 56.4% | 0.9% | 17.0% | 0.5% |
| 2013 | 0.0% | 0.1% | 0.0% | 0.1% | 0.0% | 0.1% | 4.2% | 0.3% |
| 1729 | 49.2% | 1.5% | 33.3% | 0.9% | 43.6% | 1.2% | 19.5% | 0.5% |
| 1989 | 71.0% | 0.9% | 39.7% | 0.9% | 62.6% | 0.7% | 27.2% | 0.5% |
| 2045 | 66.3% | 0.9% | 29.6% | 0.6% | 61.3% | 0.8% | 17.6% | 0.5% |
| 2017 | 72.6% | 1.0% | 42.1% | 1.1% | 69.5% | 1.3% | 18.6% | 0.5% |
| 1738 | 0.0% | 0.2% | 0.0% | 0.1% | 0.0% | 0.1% | 1.3% | 0.3% |
| 1993 | 70.9% | 1.4% | 39.9% | 1.0% | 67.7% | 1.6% | 18.5% | 0.5% |
| 2049 | 0.0% | 0.1% | 0.0% | 0.1% | 0.0% | 0.1% | 0.7% | 0.3% |
| 2021 | 73.8% | 1.3% | 38.5% | 0.8% | 69.7% | 1.8% | 16.3% | 0.6% |
| 1747 | 0.0% | 0.1% | 0.0% | 0.1% | 0.0% | 0.1% | 5.2% | 0.3% |
| 1997 | 0.0% | 0.1% | 0.0% | 0.1% | 0.0% | 0.1% | −7.8% | 0.3% |
| 2053 | 70.4% | 2.3% | 35.2% | 1.4% | 59.7% | 1.7% | 21.1% | 0.6% |
| 2025 | 0.0% | 0.1% | 0.0% | 0.1% | 0.0% | 0.1% | 0.2% | 0.4% |
| 1756 | 39.6% | 2.3% | 24.2% | 0.8% | 33.3% | 1.6% | 12.3% | 0.5% |
| 2001 | 58.2% | 1.0% | 29.8% | 0.6% | 53.1% | 1.0% | 17.1% | 0.7% |
| 2057 | 0.0% | 0.1% | 0.0% | 0.1% | 0.0% | 0.1% | −0.2% | 0.5% |
| 2029 | 0.0% | 0.1% | 0.0% | 0.1% | 0.0% | 0.1% | 3.2% | 0.3% |
| 1765 | 37.3% | 2.2% | 25.8% | 0.6% | 35.7% | 2.2% | 15.0% | 0.6% |

TABLE 24e
Correction of L348fs using PE2 systems, experiment 2.

| PEG # | edit % | indel % | PEG # | edit % | indel % | PEG # | edit % | indel % | PEG # | edit % | indel % |
|---|---|---|---|---|---|---|---|---|---|---|---|
| 1728 | 35% | 1% | 2091 | 38% | 4% | 2321 | 7% | 1% | 2485 | 63% | 2% |
| 1729 | 41% | 1% | 2092 | 34% | 4% | 2429 | 47% | 1% | 2486 | 54% | 2% |
| 1730 | 38% | 1% | 2097 | 26% | 4% | 2430 | 24% | 1% | 2487 | 59% | 2% |
| 1736 | 29% | 1% | 2098 | 30% | 5% | 2431 | 47% | 1% | 2488 | 57% | 1% |
| 1737 | 31% | 2% | 2099 | 32% | 9% | 2432 | 43% | 1% | 2489 | 47% | 2% |
| 1738 | 23% | 1% | 2100 | 33% | 6% | 2433 | 41% | 1% | 2490 | 46% | 2% |
| 1739 | 30% | 2% | 2101 | 29% | 7% | 2434 | 43% | 1% | 2491 | 58% | 1% |
| 1745 | 20% | 2% | 2106 | 23% | 5% | 2435 | 40% | 1% | 2492 | 35% | 2% |
| 1746 | 22% | 2% | 2107 | 32% | 6% | 2436 | 40% | 1% | 2493 | 43% | 1% |
| 1747 | 24% | 2% | 2108 | 31% | 13% | 2437 | 38% | 1% | 2494 | 47% | 1% |
| 1748 | 31% | 2% | 2109 | 34% | 7% | 2438 | 38% | 1% | 2495 | 41% | 2% |
| 1754 | 18% | 2% | 2110 | 33% | 5% | 2439 | 47% | 2% | 2496 | 45% | 1% |
| 1756 | 21% | 2% | 2115 | 22% | 4% | 2440 | 42% | 1% | 2497 | 27% | 1% |
| 1757 | 26% | 1% | 2116 | 28% | 5% | 2441 | 40% | 1% | 2498 | 39% | 1% |
| 1984 | 51% | 1% | 2117 | 31% | 6% | 2442 | 47% | 2% | 2499 | 38% | 2% |
| 1985 | 58% | 1% | 2118 | 30% | 7% | 2443 | 42% | 2% | 2500 | 41% | 1% |
| 1988 | 42% | 1% | 2119 | 27% | 8% | 2444 | 36% | 1% | 2501 | 25% | 1% |
| 1989 | 40% | 1% | 2286 | 15% | 2% | 2445 | 46% | 1% | 2502 | 41% | 1% |
| 1992 | 30% | 1% | 2287 | 7% | 1% | 2446 | 51% | 1% | 2503 | 47% | 1% |
| 1993 | 37% | 1% | 2288 | 8% | 1% | 2447 | 29% | 1% | 2505 | 23% | 1% |
| 1996 | 32% | 1% | 2289 | 5% | 0% | 2448 | 42% | 1% | 2506 | 30% | 1% |
| 1997 | 41% | 1% | 2295 | 9% | 2% | 2449 | 41% | 1% | 2507 | 44% | 2% |
| 2012 | 56% | 1% | 2296 | 4% | 0% | 2450 | 25% | 2% | 2508 | 19% | 1% |
| 2013 | 57% | 2% | 2297 | 4% | 1% | 2451 | 32% | 2% | 2509 | 19% | 1% |
| 2016 | 1% | 0% | 2298 | 3% | 0% | 2452 | 30% | 1% | 2510 | 56% | 1% |
| 2017 | 30% | 2% | 2304 | 0% | 0% | 2453 | 52% | 1% | 2511 | 43% | 2% |
| 2020 | 34% | 1% | 2305 | 4% | 1% | 2454 | 47% | 1% | 2512 | 41% | 2% |
| 2021 | 37% | 3% | 2306 | 3% | 1% | 2455 | 47% | 1% | 2513 | 48% | 1% |
| 2024 | 34% | 2% | 2307 | 3% | 1% | 2456 | 47% | 1% | 2514 | 41% | 1% |
| 2025 | 28% | 2% | 2313 | 10% | 1% | 2457 | 47% | 1% | 2515 | 29% | 1% |
| 2040 | 47% | 1% | 2314 | 6% | 1% | 2458 | 45% | 2% | 2516 | 20% | 1% |
| 2041 | 37% | 2% | 2315 | 4% | 0% | 2459 | 48% | 1% | 2517 | 46% | 1% |
| 2044 | 48% | 2% | 2316 | 4% | 1% | 2460 | 49% | 2% | 2518 | 37% | 1% |
| 2045 | 45% | 1% | 2322 | 9% | 0% | 2461 | 44% | 1% | 2519 | 25% | 1% |
| 2048 | 43% | 2% | 2323 | 7% | 0% | 2462 | 47% | 1% | 2520 | 15% | 1% |
| 2049 | 46% | 1% | 2324 | 4% | 1% | 2463 | 42% | 2% | 2521 | 50% | 1% |
| 2052 | 40% | 2% | 2325 | 5% | 0% | 2465 | 28% | 2% | 2522 | 34% | 1% |
| 2053 | 43% | 2% | 2409 | 18% | 3% | 2466 | 43% | 1% | 2523 | 22% | 1% |
| 2061 | 28% | 2% | 2410 | 25% | 5% | 2467 | 45% | 2% | 2524 | 17% | 1% |
| 2062 | 24% | 2% | 2411 | 35% | 8% | 2468 | 65% | 2% | 2525 | 43% | 1% |
| 2063 | 33% | 3% | 2412 | 27% | 5% | 2469 | 34% | 1% | 2526 | 30% | 2% |
| 2064 | 26% | 2% | 2413 | 25% | 4% | 2470 | 45% | 2% | 2527 | 19% | 2% |
| 2065 | 28% | 2% | 2414 | 6% | 1% | 2471 | 60% | 1% | 2528 | 19% | 1% |
| 2070 | 30% | 2% | 2415 | 8% | 2% | 2472 | 22% | 1% | 2529 | 39% | 2% |
| 2071 | 27% | 2% | 2416 | 3% | 1% | 2473 | 41% | 1% | 2530 | 48% | 2% |
| 2072 | 34% | 3% | 2417 | 4% | 1% | 2474 | 46% | 2% | 2531 | 29% | 2% |
| 2073 | 29% | 2% | 2418 | 4% | 1% | 2475 | 60% | 2% | 2532 | 38% | 1% |
| 2074 | 30% | 2% | 2419 | 4% | 0% | 2476 | 24% | 2% | 2533 | 46% | 2% |
| 2079 | 22% | 2% | 2420 | 4% | 1% | 2477 | 34% | 1% | 2534 | 26% | 2% |
| 2080 | 25% | 2% | 2421 | 3% | 0% | 2478 | 48% | 2% | 2535 | 35% | 1% |
| 2081 | 35% | 3% | 2422 | 6% | 1% | 2479 | 63% | 2% | 2536 | 41% | 2% |
| 2082 | 31% | 3% | 2285 | 8% | 1% | 2480 | 28% | 1% | 2537 | 26% | 2% |
| 2083 | 31% | 3% | 2424 | 8% | 1% | 2481 | 60% | 2% | | | |
| 2088 | 30% | 3% | 2294 | 5% | 1% | 2482 | 46% | 2% | | | |
| 2089 | 30% | 3% | 2303 | 6% | 1% | 2483 | 57% | 2% | | | |
| 2090 | 40% | 4% | 2312 | 8% | 1% | 2484 | 26% | 2% | | | |

TABLE 24f
Correction of L348fs using PE3 systems, experiment 1.

| PEG# | ng # | edit % | indel % | PEG# | ng # | edit % | indel % | PEG# | ng # | edit % | indel % |
|---|---|---|---|---|---|---|---|---|---|---|---|
| 2088 | 543 | 39 | 10 | 2097 | 543 | 52 | 15 | 1729 | none | 33 | 4 |
| 2097 | 543 | 34 | 7 | 2088 | 542 | 46 | 6 | 1989 | none | 45 | 3 |
| 2088 | 537 | 57 | 8 | 2097 | 542 | 45 | 9 | 2045 | none | 50 | 4 |
| 2097 | 537 | 49 | 11 | 2088 | 522 | 50 | 8 | 2021 | none | 52 | 4 |
| 2088 | 531 | 75 | 2 | 2097 | 522 | 42 | 10 | 1984 | 533 | 42 | 2 |
| 2097 | 531 | 51 | 1 | 1727 | 522 | 40 | 4 | 2040 | 533 | 54 | 6 |
| 2088 | 532 | 36 | 2 | 1984 | 522 | 49 | 2 | 2016 | 533 | 38 | 3 |
| 2097 | 532 | 27 | 2 | 2040 | 522 | 34 | 1 | 1729 | 533 | 50 | 13 |
| 2088 | 3211 | 35 | 2 | 2016 | 522 | 39 | 1 | 1989 | 533 | 57 | 4 |
| 2097 | 3211 | 38 | 5 | 1729 | 522 | 37 | 3 | 2045 | 533 | 53 | 3 |
| 1984 | 3209 | 51 | 3 | 1989 | 522 | 57 | 2 | 2021 | 533 | 71 | 5 |
| 2040 | 3209 | 33 | 2 | 2045 | 522 | 53 | 2 | 2088 | 549 | 29 | 5 |
| 2016 | 3209 | 52 | 3 | 2021 | 522 | 42 | 1 | 2097 | 549 | 30 | 5 |
| 1729 | 3209 | 63 | 8 | 2088 | 540 | 49 | 7 | 2088 | 546 | 71 | 4 |
| 1989 | 3209 | 66 | 3 | 2097 | 540 | 46 | 9 | 2097 | 546 | 38 | 2 |
| 2045 | 3209 | 56 | 2 | 2088 | 539 | 45 | 5 | 1727 | 526 | 78 | 1 |
| 2021 | 3209 | 58 | 2 | 2097 | 539 | 37 | 9 | 1984 | 526 | 69 | 1 |
| 1984 | 3210 | 54 | 1 | 2088 | 3215 | 42 | 3 | 2040 | 526 | 43 | 1 |
| 2040 | 3210 | 38 | 1 | 2097 | 3215 | 39 | 4 | 2016 | 526 | 64 | 1 |
| 2016 | 3210 | 44 | 1 | 1727 | 525 | 30 | 3 | 1729 | 526 | 67 | 2 |
| 1729 | 3210 | 56 | 4 | 1984 | 525 | 43 | 2 | 1989 | 526 | 60 | 1 |
| 1989 | 3210 | 57 | 2 | 2040 | 525 | 29 | 1 | 2045 | 526 | 63 | 1 |
| 2045 | 3210 | 67 | 0 | 2016 | 525 | 28 | 1 | 2021 | 526 | 59 | 1 |
| 2021 | 3210 | 53 | 2 | 1729 | 525 | 25 | 2 | 2088 | 548 | 72 | 6 |
| 1984 | 3208 | 55 | 2 | 1989 | 525 | 52 | 2 | 2097 | 548 | 65 | 3 |
| 2040 | 3208 | 60 | 3 | 2045 | 525 | 53 | 2 | 2088 | 550 | 24 | 9 |
| 2016 | 3208 | 43 | 2 | 2021 | 525 | 54 | 3 | 2097 | 550 | 18 | 8 |
| 1729 | 3208 | 67 | 7 | 2088 | 538 | 47 | 5 | 1727 | 527 | 41 | 8 |
| 1989 | 3208 | 57 | 2 | 2097 | 538 | 37 | 6 | 1984 | 527 | 61 | 3 |
| 2045 | 3208 | 58 | 3 | 2088 | 536 | 51 | 7 | 2040 | 527 | 45 | 3 |
| 2021 | 3208 | 67 | 2 | 2097 | 536 | 44 | 5 | 2016 | 527 | 65 | 4 |
| 1984 | 3207 | 61 | 2 | 2088 | 535 | 43 | 5 | 1729 | 527 | 46 | 7 |
| 2040 | 3207 | 59 | 3 | 2097 | 535 | 43 | 6 | 1989 | 527 | 54 | 3 |
| 2016 | 3207 | 51 | 3 | 1727 | 523 | 41 | 12 | 2045 | 527 | 53 | 3 |
| 1729 | 3207 | 65 | 8 | 1984 | 523 | 52 | 4 | 2021 | 527 | 69 | 4 |
| 1989 | 3207 | 71 | 3 | 2040 | 523 | 30 | 2 | 1727 | 3214 | 36 | 1 |
| 2045 | 3207 | 65 | 2 | 2016 | 523 | 45 | 3 | 1984 | 3214 | 51 | 1 |
| 2021 | 3207 | 79 | 3 | 1729 | 523 | 34 | 6 | 2040 | 3214 | 30 | 1 |
| 1984 | 3206 | 62 | 3 | 1989 | 523 | 45 | 3 | 2016 | 3214 | 50 | 1 |
| 2040 | 3206 | 62 | 1 | 2045 | 523 | 48 | 4 | 1729 | 3214 | 35 | 1 |
| 2016 | 3206 | 55 | 2 | 2021 | 523 | 43 | 2 | 1989 | 3214 | 49 | 1 |
| 1729 | 3206 | 64 | 4 | 1984 | 537 | 53 | 2 | 2045 | 3214 | 49 | 1 |
| 1989 | 3206 | 71 | 2 | 2040 | 3216 | 40 | 2 | 2021 | 3214 | 41 | 2 |
| 2045 | 3206 | 64 | 2 | 2016 | 3216 | 63 | 3 | 1727 | 3213 | 35 | 1 |
| 2021 | 3206 | 80 | 2 | 1729 | 3216 | 59 | 8 | 1984 | 3213 | 52 | 1 |
| 1984 | 3205 | 58 | 1 | 1989 | 3216 | 57 | 2 | 2040 | 3213 | 42 | 1 |
| 2040 | 3205 | 54 | 1 | 2045 | 3216 | 55 | 2 | 2016 | 3213 | 48 | 2 |
| 2016 | 3205 | 63 | 2 | 2021 | 3216 | 67 | 2 | 1729 | 3213 | 33 | 1 |
| 1729 | 3205 | 54 | 4 | 1727 | 529 | 40 | 14 | 1989 | 3213 | 52 | 1 |
| 1989 | 3205 | 62 | 1 | 1984 | 529 | 64 | 8 | 2045 | 3213 | 52 | 1 |
| 2045 | 3205 | 63 | 2 | 2040 | 529 | 44 | 8 | 2021 | 3213 | 51 | 1 |
| 2021 | 3205 | 75 | 2 | 2016 | 529 | 61 | 6 | 1727 | 3212 | 36 | 1 |
| 1984 | 3204 | 50 | 2 | 1729 | 529 | 38 | 12 | 1984 | 3212 | 55 | 1 |
| 2040 | 3204 | 50 | 2 | 1989 | 529 | 64 | 8 | 2040 | 3212 | 43 | 1 |
| 2016 | 3204 | 49 | 2 | 2045 | 529 | 62 | 7 | 2016 | 3212 | 61 | 2 |
| 1729 | 3204 | 52 | 4 | 2021 | 529 | 60 | 6 | 1729 | 3212 | 34 | 2 |
| 1989 | 3204 | 70 | 2 | 1727 | 531 | 57 | 14 | 1989 | 3212 | 50 | 1 |
| 2045 | 3204 | 53 | 2 | 1984 | 531 | 58 | 5 | 2045 | 3212 | 49 | 1 |
| 2021 | 3204 | 72 | 2 | 2040 | 531 | 54 | 5 | 2021 | 3212 | 62 | 1 |
| 2088 | 544 | 39 | 6 | 2016 | 531 | 64 | 4 | 2088 | 551 | 38 | 10 |
| 2097 | 544 | 45 | 8 | 1729 | 531 | 50 | 11 | 2097 | 551 | 34 | 8 |
| 1727 | 524 | 30 | 1 | 1989 | 531 | 65 | 3 | 2088 | 552 | 35 | 14 |
| 1984 | 524 | 46 | 1 | 2045 | 531 | 59 | 5 | 2097 | 552 | 31 | 14 |
| 2040 | 524 | 28 | 1 | 2021 | 531 | 69 | 4 | 2088 | 553 | 30 | 9 |
| 2016 | 524 | 40 | 1 | 1984 | 532 | 43 | 3 | 2097 | 553 | 33 | 12 |
| 1729 | 524 | 28 | 1 | 2040 | 532 | 43 | 3 | 2088 | 554 | 49 | 5 |
| 1989 | 524 | 57 | 1 | 2016 | 532 | 40 | 2 | 2097 | 554 | 45 | 5 |
| 2045 | 524 | 51 | 1 | 1729 | 532 | 46 | 7 | 1984 | 530 | 48 | 4 |
| 2021 | 524 | 50 | 1 | 1989 | 532 | 50 | 2 | 2040 | 530 | 44 | 4 |
| 1984 | 523 | 52 | 2 | 2045 | 532 | 53 | 3 | 2016 | 530 | 39 | 3 |
| 2040 | 523 | 40 | 2 | 2021 | 532 | 56 | 2 | 1729 | 530 | 41 | 8 |
| 2016 | 523 | 50 | 2 | 2088 | none | 37 | 2 | 1989 | 530 | 46 | 3 |
| 1729 | 523 | 59 | 8 | 2097 | none | 35 | 3 | 2045 | 530 | 47 | 4 |
| 1989 | 523 | 61 | 2 | 1727 | none | 30 | 4 | 2021 | 530 | 53 | 3 |
| 2045 | 523 | 57 | 2 | 1984 | none | 54 | 4 | | | | |
| 2021 | 523 | 66 | 2 | 2040 | none | 33 | 2 | | | | |

TABLE 24g
Correction of L348fs using PE3 systems, experiment 2.

| PEG# | ng # | edit % | indel % | PEG# | ng # | edit % | indel % | PEG# | ng # | edit % | indel % |
|---|---|---|---|---|---|---|---|---|---|---|---|
| 1984 | 3214 | 49.20% | 1.30% | 2045 | 3204 | 37.90% | 1.60% | 2016 | 531 | 46.50% | 3.90% |
| 1989 | 3214 | 42.40% | 1.50% | 2040 | 3204 | 34.00% | 0.90% | 1729 | 532 | 50.60% | 3.40% |
| 2021 | 3214 | 49.80% | 1.30% | 2021 | 3204 | 47.20% | 1.50% | 1727 | 532 | 49.40% | 4.90% |
| 2016 | 3214 | 42.10% | 1.20% | 2016 | 3204 | 32.70% | 1.10% | 1989 | 532 | 61.50% | 2.50% |
| 1984 | 3213 | 50.60% | 0.90% | 2097 | 544 | 41.50% | 8.00% | 1984 | 532 | 45.90% | 2.00% |
| 1989 | 3213 | 42.10% | 1.00% | 2088 | 544 | 41.20% | 6.70% | 2045 | 532 | 48.40% | 1.50% |
| 2021 | 3213 | 48.10% | 1.00% | 1729 | 524 | 45.30% | 12.80% | 2040 | 532 | 39.70% | 2.80% |
| 2016 | 3213 | 38.30% | 0.90% | 1727 | 524 | 36.40% | 13.20% | 2021 | 532 | 59.60% | 2.90% |
| 1984 | 3212 | 53.50% | 0.90% | 1984 | 524 | 48.20% | 8.80% | 2016 | 532 | 30.50% | 1.30% |
| 1989 | 3212 | 37.80% | 1.20% | 1989 | 524 | 45.00% | 5.10% | 1729 | 523 | 41.90% | 14.50% |
| 2021 | 3212 | 48.20% | 1.40% | 2040 | 524 | 37.70% | 6.80% | 1727 | 523 | 37.70% | 12.70% |
| 2016 | 3212 | 40.70% | 1.30% | 2045 | 524 | 32.90% | 4.10% | 1984 | 523 | 46.50% | 9.70% |
| 1729 | 3214 | 37.60% | 2.30% | 2021 | 524 | 45.10% | 6.10% | 1989 | 523 | 38.10% | 7.40% |
| 1727 | 3214 | 30.60% | 1.10% | 2016 | 524 | 42.60% | 4.80% | 2040 | 523 | 33.60% | 7.00% |
| 2040 | 3214 | 38.50% | 0.50% | 1729 | 523 | 54.70% | 3.70% | 2045 | 523 | 31.70% | 4.50% |
| 2045 | 3214 | 28.10% | 0.70% | 1727 | 523 | 43.80% | 3.30% | 2021 | 523 | 44.50% | 5.80% |
| 1729 | 3213 | 37.10% | 1.30% | 1989 | 523 | 56.40% | 2.00% | 2016 | 523 | 42.10% | 7.30% |
| 1727 | 3213 | 28.00% | 0.80% | 1984 | 523 | 55.70% | 1.00% | 1729 | 533 | 51.10% | 4.50% |
| 2040 | 3213 | 38.10% | 1.10% | 2045 | 523 | 48.20% | 1.40% | 1727 | 533 | 43.40% | 5.20% |
| 2045 | 3213 | 29.20% | 0.70% | 2040 | 523 | 45.10% | 2.40% | 1989 | 533 | 64.00% | 4.40% |
| 1729 | 3212 | 36.40% | 1.20% | 2021 | 523 | 57.00% | 2.40% | 1984 | 533 | 49.60% | 3.00% |
| 1727 | 3212 | 31.30% | 0.60% | 2016 | 523 | 37.60% | 1.90% | 2045 | 533 | 48.60% | 3.50% |
| 2040 | 3212 | 38.80% | 0.40% | 2097 | 543 | 52.20% | 10.90% | 2040 | 533 | 41.80% | 2.70% |
| 2045 | 3212 | 28.90% | 0.60% | 2088 | 543 | 44.70% | 6.80% | 2021 | 533 | 55.40% | 2.70% |
| 2097 | 543 | 41.00% | 6.40% | 2088 | 542 | 40.00% | 3.40% | 2016 | 533 | 35.50% | 1.90% |
| 2088 | 543 | 26.50% | 3.60% | 2097 | 522 | 42.70% | 6.80% | 2097 | 549 | 29.60% | 8.70% |
| 2088 | 537 | 49.40% | 8.30% | 1729 | 522 | 39.90% | 10.10% | 2088 | 549 | 24.70% | 6.40% |
| 2097 | 537 | 48.90% | 6.40% | 2088 | 522 | 39.80% | 5.20% | 2097 | 546 | 59.90% | 3.90% |
| 2097 | 526 | 63.40% | 2.10% | 1727 | 522 | 36.50% | 9.10% | 2088 | 546 | 56.30% | 2.50% |
| 2088 | 526 | 53.00% | 2.10% | 1984 | 522 | 49.30% | 7.30% | 1729 | 526 | 68.50% | 0.90% |
| 2097 | 545 | 34.20% | 2.10% | 1989 | 522 | 43.80% | 5.80% | 1727 | 526 | 64.50% | 0.80% |
| 2088 | 545 | 32.10% | 1.90% | 2040 | 522 | 29.40% | 5.80% | 1984 | 526 | 62.70% | 0.60% |
| 2097 | 3211 | 34.90% | 1.90% | 2045 | 522 | 29.40% | 5.10% | 1989 | 526 | 53.10% | 0.80% |
| 2088 | 3211 | 30.80% | 1.50% | 2021 | 522 | 39.80% | 6.80% | 2040 | 526 | 48.40% | 0.70% |
| 1729 | 3209 | 55.50% | 3.90% | 2016 | 522 | 35.60% | 5.70% | 2045 | 526 | 36.90% | 0.90% |
| 1727 | 3209 | 46.90% | 4.50% | 2097 | 540 | 47.70% | 10.30% | 2021 | 526 | 64.40% | 1.00% |
| 1989 | 3209 | 66.30% | 2.70% | 2088 | 540 | 42.40% | 8.60% | 2016 | 526 | 55.00% | 0.80% |
| 1984 | 3209 | 50.90% | 2.50% | 2097 | 539 | 39.80% | 5.70% | 2097 | 548 | 59.60% | 3.40% |
| 2045 | 3209 | 52.80% | 3.10% | 2088 | 539 | 36.50% | 4.10% | 2088 | 548 | 54.10% | 3.10% |
| 2040 | 3209 | 47.00% | 2.20% | 2097 | 521 | 45.20% | 3.00% | 2097 | 550 | 22.10% | 31.20% |
| 2021 | 3209 | 57.80% | 2.60% | 1729 | 521 | 34.30% | 9.00% | 2088 | 550 | 19.60% | 24.30% |
| 2016 | 3209 | 37.90% | 1.80% | 1727 | 521 | 31.10% | 7.10% | 1729 | 527 | 46.00% | 5.90% |
| 1729 | 3210 | 53.70% | 3.80% | 2088 | 521 | 30.70% | 2.20% | 1727 | 527 | 36.10% | 4.00% |
| 1727 | 3210 | 46.10% | 3.10% | 1984 | 521 | 47.50% | 6.60% | 1984 | 527 | 58.20% | 2.70% |
| 1989 | 3210 | 58.10% | 1.70% | 1989 | 521 | 36.20% | 5.80% | 1989 | 527 | 45.10% | 2.70% |
| 1984 | 3210 | 51.60% | 2.10% | 2040 | 521 | 24.90% | 4.10% | 2040 | 527 | 42.00% | 3.10% |
| 2045 | 3210 | 49.60% | 1.40% | 2045 | 521 | 23.60% | 3.90% | 2045 | 527 | 33.60% | 2.10% |
| 2040 | 3210 | 44.10% | 1.70% | 2016 | 521 | 35.00% | 5.40% | 2021 | 527 | 48.00% | 3.70% |
| 2021 | 3210 | 53.60% | 1.90% | 2021 | 521 | 32.60% | 6.90% | 2016 | 527 | 39.30% | 2.80% |
| 2016 | 3210 | 35.60% | 1.50% | 1729 | 525 | 37.10% | 19.10% | 2097 | 551 | 26.60% | 22.70% |
| 1729 | 3208 | 57.60% | 3.70% | 1727 | 525 | 27.70% | 16.50% | 2088 | 551 | 25.80% | 15.50% |
| 1727 | 3208 | 45.40% | 3.30% | 1984 | 525 | 48.00% | 13.50% | 2097 | 552 | 28.30% | 25.20% |
| 1989 | 3208 | 62.70% | 1.60% | 1989 | 525 | 43.30% | 9.40% | 2088 | 552 | 23.00% | 15.80% |
| 1984 | 3208 | 56.60% | 2.30% | 2040 | 525 | 30.60% | 9.80% | 2097 | 553 | 29.40% | 27.30% |
| 2045 | 3208 | 51.30% | 2.10% | 2045 | 525 | 24.80% | 4.10% | 2088 | 553 | 25.00% | 13.10% |
| 2040 | 3208 | 45.30% | 3.40% | 2016 | 525 | 39.70% | 7.80% | 2097 | 554 | 40.90% | 10.80% |
| 2021 | 3208 | 54.20% | 2.30% | 2021 | 525 | 39.10% | 9.00% | 2088 | 554 | 29.10% | 5.70% |
| 2016 | 3208 | 39.20% | 1.60% | 2097 | 538 | 37.70% | 5.40% | 1729 | 530 | 56.10% | 8.70% |
| 1729 | 3207 | 58.60% | 3.40% | 2088 | 538 | 34.70% | 5.40% | 1727 | 530 | 49.30% | 7.00% |
| 1727 | 3207 | 53.40% | 3.00% | 2088 | 536 | 47.00% | 6.70% | 1989 | 530 | 59.80% | 5.70% |
| 1989 | 3207 | 61.10% | 2.30% | 2097 | 536 | 46.80% | 5.50% | 1984 | 530 | 58.00% | 5.60% |
| 1984 | 3207 | 55.20% | 2.70% | 2097 | 535 | 45.40% | 4.80% | 2040 | 530 | 34.90% | 3.00% |
| 2045 | 3207 | 50.90% | 1.70% | 2088 | 535 | 45.30% | 4.90% | 2021 | 530 | 52.50% | 4.30% |
| 2040 | 3207 | 46.10% | 1.50% | 1729 | 3216 | 52.00% | 3.20% | 2016 | 530 | 34.20% | 2.30% |
| 2021 | 3207 | 57.80% | 2.20% | 1727 | 3216 | 45.00% | 2.70% | 2088 | none | 28.80% | 2.20% |
| 2016 | 3207 | 41.30% | 1.70% | 1989 | 3216 | 56.10% | 1.70% | 2088 | none | 27.10% | 1.50% |
| 1729 | 3206 | 55.90% | 1.70% | 1984 | 3216 | 52.80% | 2.10% | 2097 | none | 28.50% | 2.20% |
| 1727 | 3206 | 44.90% | 2.30% | 2040 | 3216 | 46.40% | 1.70% | 2097 | none | 27.50% | 2.30% |
| 1989 | 3206 | 59.70% | 1.50% | 2045 | 3216 | 43.80% | 1.00% | 1727 | none | 31.30% | 0.80% |
| 1984 | 3206 | 53.40% | 1.30% | 2021 | 3216 | 53.20% | 1.60% | 1727 | none | 29.10% | 0.80% |
| 2045 | 3206 | 52.20% | 1.30% | 2016 | 3216 | 31.30% | 1.50% | 1729 | none | 36.40% | 1.10% |
| 2040 | 3206 | 44.10% | 0.60% | 1729 | 529 | 45.00% | 8.00% | 1729 | none | 35.80% | 1.20% |
| 2021 | 3206 | 51.90% | 1.50% | 1727 | 529 | 40.80% | 7.70% | 2097 | none | 34.80% | 3.20% |
| 2016 | 3206 | 35.40% | 0.80% | 1984 | 529 | 52.00% | 5.60% | 2088 | none | 29.40% | 1.70% |
| 1729 | 3205 | 50.70% | 2.00% | 1989 | 529 | 48.20% | 3.90% | 1984 | none | 44.70% | 0.60% |
| 1727 | 3205 | 37.50% | 1.30% | 2045 | 529 | 36.40% | 2.60% | 1984 | none | 41.30% | 1.00% |
| 1989 | 3205 | 50.70% | 0.90% | 2040 | 529 | 33.90% | 4.40% | 1989 | none | 43.90% | 0.90% |
| 1984 | 3205 | 45.00% | 1.00% | 2016 | 529 | 43.20% | 4.10% | 1989 | none | 40.10% | 0.80% |
| 2045 | 3205 | 41.40% | 1.20% | 2021 | 529 | 37.50% | 2.30% | 2040 | none | 41.80% | 0.60% |
| 2040 | 3205 | 35.50% | 0.80% | 1729 | 531 | 54.20% | 5.40% | 2040 | none | 39.30% | 0.70% |
| 2021 | 3205 | 49.10% | 1.10% | 1727 | 531 | 46.70% | 5.70% | 2045 | none | 43.50% | 1.30% |
| 2016 | 3205 | 32.50% | 1.20% | 1984 | 531 | 62.30% | 5.10% | 2045 | none | 36.00% | 0.80% |
| 1729 | 3204 | 49.70% | 2.00% | 1989 | 531 | 49.10% | 2.70% | 2016 | none | 46.80% | 1.40% |
| 1727 | 3204 | 44.90% | 2.40% | 2040 | 531 | 44.10% | 3.80% | 2016 | none | 38.60% | 1.00% |
| 1984 | 3204 | 47.30% | 1.40% | 2045 | 531 | 38.20% | 2.40% | 2021 | none | 49.80% | 1.10% |
| 1989 | 3204 | 45.40% | 1.20% | 2021 | 531 | 52.30% | 2.80% | 2021 | none | 48.50% | 1.10% |

TABLE 24h
Correction of L348fs using PE3 systems, experiment 3.

| | | First trial | | | | | | Second trial | | | | | |
|---|---|---|---|---|---|---|---|---|---|---|---|---|---|
| | | Replicate A | | Replicate B | | Replicate C | | Replicate A | | Replicate B | | Replicate C | |
| PEG# | ng# | edit % | indel % | edit % | indel % | edit % | indel % | edit % | indel % | edit % | indel % | edit % | indel % |
| 1727 | 3206 | 53.4 | 2.7 | 54.0 | 2.6 | 55.9 | 3.1 | 54.2 | 1.3 | 53.9 | 1.7 | 52.5 | 2.4 |
| 1984 | 3206 | 74.3 | 1.8 | 74.8 | 2.0 | 79.4 | 1.9 | 71.1 | 1.9 | 76.0 | 1.8 | 77.7 | 2.0 |
| 2538 | 3206 | 0.1 | 0.0 | 0.1 | 0.0 | 0.1 | 0.0 | 0.0 | 0.0 | 0.2 | 0.0 | 1.4 | 0.1 |
| 2453 | 3206 | 60.6 | 1.2 | 67.1 | 1.7 | 71.4 | 1.7 | 60.9 | 1.6 | 55.3 | 0.9 | 60.9 | 1.2 |
| 2016 | 3206 | 66.1 | 2.1 | 74.9 | 3.0 | 77.4 | 2.5 | 71.8 | 2.5 | 77.3 | 2.8 | 72.9 | 2.2 |
| 2044 | 3206 | 63.7 | 2.6 | 66.7 | 3.3 | 68.7 | 3.3 | 62.5 | 2.7 | 56.7 | 2.5 | 66.2 | 3.0 |
| 1728 | 3206 | 56.4 | 3.5 | 63.6 | 4.2 | 67.5 | 5.1 | 59.6 | 3.1 | 62.8 | 3.1 | 61.9 | 3.0 |
| 2468 | 3206 | 62.4 | 2.2 | 72.3 | 3.1 | 69.4 | 2.4 | 69.6 | 2.4 | 75.0 | 2.1 | 76.5 | 2.5 |
| 2480 | 3206 | 58.7 | 2.5 | 55.9 | 2.4 | 49.7 | 2.2 | 52.3 | 2.1 | 58.5 | 2.3 | 64.7 | 2.3 |
| 2479 | 3206 | 48.9 | 1.6 | 66.9 | 2.0 | 80.6 | 2.6 | 66.6 | 2.0 | 70.8 | 2.1 | 76.2 | 2.0 |
| 2484 | 3206 | 41.5 | 2.2 | 49.5 | 2.7 | 58.1 | 3.2 | 52.6 | 2.5 | 54.9 | 3.3 | 53.7 | 3.4 |
| 2481 | 3206 | 66.9 | 2.4 | 68.9 | 2.2 | 72.9 | 2.6 | 68.6 | 2.3 | 74.3 | 2.3 | 73.2 | 2.4 |
| 1729 | 3206 | 64.4 | 4.2 | 64.6 | 4.2 | 68.1 | 4.0 | 57.0 | 2.5 | 65.9 | 3.0 | 68.2 | 3.0 |
| 2045 | 3206 | 59.6 | 2.2 | 64.2 | 2.4 | 72.3 | 3.2 | 69.5 | 2.6 | 66.3 | 2.5 | 75.4 | 2.4 |
| 1989 | 3206 | 58.3 | 1.3 | 63.7 | 1.7 | 77.1 | 2.6 | 62.2 | 1.3 | 57.0 | 1.7 | 69.4 | 1.7 |
| 2021 | 3206 | 61.3 | 5.2 | 61.5 | 4.7 | 65.3 | 4.8 | 60.1 | 4.6 | 63.7 | 5.7 | 65.3 | 4.5 |
| 1727 | 526 | 61.1 | 0.7 | 76.4 | 0.9 | 81.5 | 0.9 | 70.8 | 0.5 | 72.2 | 0.7 | 68.5 | 0.8 |
| 1984 | 526 | 77.0 | 1.4 | 80.9 | 1.3 | 83.4 | 1.0 | 79.3 | 1.1 | 79.4 | 0.8 | 82.3 | 1.5 |
| 2538 | 526 | 0.1 | 0.0 | 0.1 | 0.0 | 0.1 | 0.0 | 0.3 | 0.0 | 0.7 | 0.1 | 1.3 | 0.0 |
| 2453 | 526 | 71.3 | 1.0 | 70.6 | 1.1 | 72.3 | 1.0 | 63.1 | 0.7 | 55.7 | 0.6 | 67.0 | 0.8 |
| 2016 | 526 | 75.2 | 1.6 | 82.7 | 1.7 | 81.6 | 1.8 | 76.3 | 1.7 | 82.6 | 1.5 | 77.2 | 1.3 |
| 2044 | 526 | 72.8 | 2.1 | 73.7 | 2.5 | 73.8 | 2.3 | 67.6 | 2.7 | 59.8 | 1.7 | 69.8 | 1.6 |
| 1728 | 526 | 74.6 | 0.9 | 83.1 | 1.1 | 86.0 | 1.3 | 79.3 | 0.8 | 78.4 | 1.1 | 73.3 | 1.3 |
| 2468 | 526 | 73.8 | 1.5 | 76.9 | 1.5 | 79.3 | 1.9 | 80.3 | 1.8 | 78.0 | 1.7 | 80.8 | 1.3 |
| 2480 | 526 | 72.9 | 1.4 | 72.7 | 1.6 | 77.1 | 1.4 | 66.3 | 1.6 | 78.3 | 1.3 | 76.1 | 1.6 |
| 2479 | 526 | 64.8 | 1.4 | 71.7 | 1.4 | 81.2 | 1.9 | 67.4 | 1.7 | 65.7 | 1.6 | 78.5 | 1.7 |
| 2484 | 526 | 65.8 | 1.8 | 71.2 | 1.7 | 76.9 | 1.8 | 74.1 | 1.9 | 70.5 | 1.8 | 68.9 | 2.0 |
| 2481 | 526 | 68.0 | 1.3 | 75.1 | 1.6 | 82.4 | 1.7 | 74.4 | 1.5 | 71.2 | 1.5 | 72.7 | 1.8 |
| 1729 | 526 | 82.1 | 1.3 | 80.5 | 1.3 | 84.3 | 1.3 | 80.5 | 1.3 | 82.3 | 1.2 | 81.9 | 1.1 |
| 2045 | 526 | 68.5 | 1.5 | 76.5 | 1.8 | 83.0 | 2.2 | 75.2 | 2.2 | 76.4 | 1.9 | 78.7 | 2.1 |
| 1989 | 526 | 69.0 | 1.4 | 73.5 | 1.3 | 83.7 | 1.7 | 70.6 | 1.7 | 69.3 | 1.0 | 77.2 | 1.6 |
| 2021 | 526 | 67.8 | 3.7 | 71.3 | 3.6 | 74.8 | 3.8 | 69.9 | 4.0 | 66.2 | 3.3 | 69.0 | 3.6 |
| 2286 | 548 | 0.1 | 0.0 | 0.1 | 0.0 | 0.1 | 0.0 | 0.1 | 0.0 | 0.1 | 0.0 | 0.9 | 0.0 |
| 2295 | 548 | 13.3 | 1.5 | 19.2 | 1.6 | 29.3 | 3.1 | 16.9 | 1.4 | 17.2 | 1.9 | 21.0 | 2.1 |
| 2313 | 548 | 15.7 | 1.2 | 25.9 | 1.8 | 33.9 | 2.7 | 32.4 | 3.4 | 35.0 | 2.6 | 32.9 | 3.1 |
| 2322 | 548 | 22.0 | 1.6 | 25.4 | 1.8 | 28.1 | 2.1 | 25.1 | 2.1 | 22.9 | 2.2 | 26.0 | 1.9 |
| 2288 | 548 | 13.2 | 1.3 | 20.2 | 2.3 | 22.3 | 2.5 | 15.9 | 2.0 | 17.8 | 1.9 | 21.0 | 2.2 |
| 2289 | 548 | 14.9 | 1.3 | 15.9 | 1.3 | 15.6 | 1.0 | 13.4 | 1.0 | 13.1 | 1.2 | 15.6 | 1.4 |
| 2285 | 548 | 19.8 | 1.0 | 20.5 | 1.3 | 24.3 | 1.6 | 18.2 | 1.0 | 20.3 | 1.6 | 23.4 | 1.3 |
| 2312 | 548 | 15.1 | 1.0 | 25.3 | 2.0 | 19.5 | 1.2 | 17.9 | 0.9 | 20.3 | 1.8 | 22.5 | 1.1 |
| 2088 | 548 | 69.9 | 6.3 | 68.9 | 7.4 | 67.3 | 6.1 | 63.4 | 6.5 | 64.6 | 5.7 | 63.7 | 6.7 |
| 2097 | 548 | 71.6 | 9.3 | 66.7 | 7.6 | 63.1 | 6.5 | 59.9 | 6.6 | 63.5 | 6.3 | 61.4 | 7.0 |
| 2063 | 548 | 77.7 | 3.1 | 76.1 | 2.3 | 66.4 | 2.0 | 64.2 | 1.8 | 70.0 | 1.8 | 68.9 | 2.5 |
| 2072 | 548 | 77.5 | 4.2 | 74.2 | 3.1 | 69.3 | 3.0 | 60.6 | 2.3 | 63.1 | 2.7 | 64.6 | 2.6 |
| 2081 | 548 | 69.3 | 7.4 | 69.5 | 6.6 | 68.0 | 6.0 | 57.6 | 4.7 | 60.6 | 5.2 | 62.0 | 5.6 |
| 2090 | 548 | 72.3 | 10.4 | 70.8 | 10.6 | 65.7 | 9.1 | 57.8 | 6.2 | 59.1 | 7.3 | 67.3 | 8.4 |
| 2099 | 548 | 67.6 | 10.8 | 69.9 | 11.7 | 63.0 | 10.3 | 59.2 | 8.2 | 60.0 | 8.4 | 62.6 | 8.8 |
| 2411 | 548 | 73.0 | 5.9 | 86.7 | 3.6 | 63.4 | 5.2 | 64.3 | 5.5 | 62.4 | 5.9 | 60.7 | 5.7 |
| 2108 | 548 | 66.3 | 7.5 | 68.6 | 8.3 | 69.1 | 8.4 | 59.0 | 7.4 | 66.2 | 7.5 | 63.9 | 9.7 |
| 2073 | 548 | 73.6 | 2.9 | 73.1 | 2.6 | 68.7 | 2.2 | 66.4 | 2.2 | 69.5 | 1.8 | 70.3 | 3.8 |
| 2082 | 548 | 73.5 | 4.8 | 70.5 | 5.0 | 69.6 | 5.1 | 72.4 | 5.4 | 76.6 | 4.7 | 70.9 | 6.5 |
| 2091 | 548 | 61.8 | 6.8 | 71.2 | 9.4 | 78.2 | 10.0 | 70.2 | 7.7 | 69.0 | 8.5 | 68.9 | 9.0 |
| 2100 | 548 | 62.3 | 6.7 | 69.6 | 8.5 | 73.6 | 9.1 | 67.0 | 7.8 | 66.5 | 7.9 | 69.8 | 8.3 |
| 2412 | 548 | 76.6 | 6.4 | 77.9 | 6.1 | 69.7 | 5.8 | 70.8 | 4.7 | 68.0 | 6.1 | 67.9 | 6.2 |
| 2412 | 548 | 70.6 | 5.6 | 71.2 | 5.5 | 69.1 | 5.1 | 61.5 | 3.8 | 63.4 | 4.5 | 63.1 | 4.2 |
| 2092 | 548 | 75.7 | 6.6 | 71.7 | 6.1 | 71.9 | 5.7 | 69.9 | 6.8 | 66.5 | 6.5 | 61.6 | 6.6 |
| 2286 | 547 | 0.2 | 0.1 | 0.3 | 0.0 | 0.2 | 0.0 | 0.1 | 0.0 | 0.1 | 0.0 | 0.9 | 0.0 |
| 2295 | 547 | 14.8 | 1.2 | 19.1 | 1.5 | 29.5 | 2.7 | 23.2 | 1.7 | 20.7 | 1.7 | 22.2 | 1.8 |
| 2313 | 547 | 20.0 | 1.2 | 26.5 | 1.5 | 28.3 | 1.5 | 35.9 | 2.0 | 34.3 | 2.2 | 38.4 | 2.2 |
| 2322 | 547 | 18.0 | 1.0 | 29.0 | 1.4 | 28.0 | 1.6 | 27.0 | 1.3 | 23.5 | 0.9 | 29.2 | 1.2 |
| 2288 | 547 | 12.5 | 1.2 | 21.7 | 2.2 | 21.9 | 2.5 | 17.9 | 1.8 | 19.5 | 2.2 | 21.5 | 2.0 |
| 2289 | 547 | 15.2 | 1.0 | 14.9 | 1.0 | 15.0 | 0.9 | 15.7 | 1.4 | 17.7 | 1.3 | 17.5 | 1.2 |
| 2285 | 547 | 14.0 | 0.8 | 23.5 | 1.6 | 24.1 | 1.7 | 24.2 | 1.6 | 20.7 | 1.5 | 21.7 | 1.3 |
| 2312 | 547 | 15.3 | 0.7 | 25.0 | 1.1 | 23.3 | 1.3 | 20.2 | 1.0 | 22.8 | 1.0 | 25.0 | 1.3 |
| 2088 | 547 | 65.0 | 2.7 | 71.9 | 3.9 | 78.2 | 4.0 | 64.7 | 2.2 | 69.8 | 3.4 | 67.8 | 3.3 |
| 2097 | 547 | 68.2 | 3.8 | 74.3 | 3.8 | 77.0 | 4.0 | 70.6 | 3.1 | 75.7 | 3.3 | 74.7 | 4.7 |
| 2063 | 547 | 77.4 | 1.7 | 76.0 | 1.6 | 74.9 | 1.4 | 76.6 | 1.3 | 79.4 | 1.3 | 77.7 | 2.2 |
| 2072 | 547 | 67.5 | 1.3 | 73.5 | 1.7 | 85.1 | 1.7 | 76.6 | 1.5 | 79.4 | 1.7 | 74.1 | 2.1 |
| 2081 | 547 | 76.3 | 1.8 | 79.2 | 1.8 | 82.8 | 2.2 | 79.2 | 2.4 | 74.8 | 2.0 | 78.0 | 2.1 |
| 2090 | 547 | 75.6 | 5.6 | 78.9 | 5.7 | 81.9 | 6.4 | 75.4 | 5.9 | 76.8 | 5.7 | 72.6 | 5.2 |
| 2099 | 547 | 74.5 | 7.1 | 76.1 | 7.3 | 75.7 | 7.1 | 76.4 | 6.4 | 79.1 | 8.5 | 66.1 | 9.5 |
| 2411 | 547 | 71.9 | 4.0 | 70.8 | 4.0 | 72.6 | 3.7 | 64.8 | 3.0 | 59.8 | 3.4 | 57.2 | 4.0 |
| 2108 | 547 | 62.6 | 6.2 | 71.5 | 7.1 | 75.9 | 7.2 | 67.9 | 6.4 | 67.3 | 7.2 | 65.7 | 8.9 |
| 2073 | 547 | 71.2 | 1.4 | 73.6 | 1.6 | 74.6 | 1.2 | 74.9 | 1.0 | 74.7 | 1.2 | 73.7 | 1.8 |
| 2082 | 547 | 71.9 | 3.4 | 70.5 | 3.5 | 75.2 | 3.3 | 71.6 | 3.6 | 72.8 | 3.7 | 76.9 | 4.4 |
| 2091 | 547 | 73.3 | 4.4 | 74.2 | 4.8 | 79.9 | 6.3 | 74.8 | 3.8 | 77.6 | 4.9 | 73.2 | 5.0 |
| 2100 | 547 | 67.6 | 3.6 | 76.6 | 4.2 | 80.8 | 4.4 | 77.1 | 4.3 | 73.8 | 3.8 | 72.6 | 5.3 |
| 2412 | 547 | 74.2 | 4.4 | 77.0 | 4.6 | 77.7 | 4.5 | 74.4 | 4.0 | 82.1 | 4.7 | 73.8 | 5.8 |
| 2412 | 547 | 68.8 | 3.0 | 73.4 | 3.4 | 77.4 | 3.2 | 69.4 | 2.5 | 70.9 | 3.0 | 61.4 | 3.8 |
| 2092 | 547 | 71.5 | 4.1 | 77.0 | 4.5 | 76.7 | 4.5 | 67.0 | 4.3 | 60.1 | 3.8 | 65.1 | 4.8 |

TABLE 24i
Correction of L348fs using PE3 systems, experiment 4.

| PEG# | ng # | edit % | indel % |
|---|---|---|---|
| 1727 | 526 | 23.73 | 0.91 |
| 1984 | 526 | 55.18 | 1.11 |
| 1729 | 526 | 19.40 | 0.64 |
| 1989 | 526 | 10.73 | 0.85 |
| 2045 | 526 | 11.53 | 0.69 |
| 2021 | 526 | 9.73 | 0.77 |
| 2016 | 526 | 16.70 | 0.71 |
| 2044 | 526 | 21.74 | 1.01 |
| 1728 | 526 | 21.21 | 0.65 |
| 2468 | 526 | 42.29 | 1.26 |
| 2480 | 526 | 10.96 | 0.56 |
| 2479 | 526 | 6.08 | 0.35 |
| 2484 | 526 | 7.69 | 0.55 |
| 2481 | 526 | 7.51 | 0.71 |
| 2538 | 526 | 2.20 | 0.36 |
| 2453 | 526 | 19.93 | 0.59 |
| 1727 | 3201 | 4.65 | 0.78 |
| 1984 | 3201 | 8.70 | 0.53 |
| 1729 | 3201 | 4.49 | 0.62 |
| 1989 | 3201 | 3.79 | 0.54 |
| 2045 | 3201 | 5.06 | 0.82 |
| 2021 | 3201 | 4.07 | 0.64 |
| 2016 | 3201 | 4.96 | 0.81 |
| 2044 | 3201 | 4.95 | 0.67 |
| 1728 | 3201 | 3.40 | 0.41 |
| 2468 | 3201 | 9.48 | 0.68 |
| 2480 | 3201 | 35.93 | 6.01 |
| 2479 | 3201 | 3.74 | 0.65 |
| 2484 | 3201 | 4.63 | 0.63 |
| 2481 | 3201 | 3.85 | 0.61 |
| 2538 | 3201 | 3.39 | 0.57 |
| 2453 | 3201 | 3.62 | 0.72 |
| 1727 | 3202 | 12.02 | 0.52 |
| 1984 | 3202 | 35.96 | 0.86 |
| 1729 | 3202 | 12.66 | 0.52 |
| 1989 | 3202 | 3.61 | 0.43 |
| 2045 | 3202 | 5.09 | 0.70 |
| 2021 | 3202 | 5.09 | 0.50 |
| 2016 | 3202 | 7.23 | 0.45 |
| 2044 | 3202 | 7.23 | 0.64 |
| 1728 | 3202 | 10.58 | 0.39 |
| 2468 | 3202 | 32.88 | 1.22 |
| 2480 | 3202 | 6.50 | 0.47 |
| 2479 | 3202 | 4.14 | 0.43 |
| 2484 | 3202 | 3.91 | 0.50 |
| 2481 | 3202 | 5.79 | 0.47 |
| 2538 | 3202 | 2.05 | 0.27 |
| 2453 | 3202 | 6.91 | 0.47 |
| 1727 | 3203 | 12.76 | 0.50 |
| 1984 | 3203 | 41.90 | 0.98 |
| 1729 | 3203 | 10.89 | 0.54 |
| 1989 | 3203 | 8.17 | 0.55 |
| 2045 | 3203 | 4.83 | 0.58 |
| 2021 | 3203 | 6.67 | 0.67 |
| 2016 | 3203 | 6.07 | 0.44 |
| 2044 | 3203 | 10.28 | 0.46 |
| 1728 | 3203 | 32.98 | 0.83 |
| 2468 | 3203 | 33.78 | 1.17 |
| 2480 | 3203 | 9.65 | 0.51 |
| 2479 | 3203 | 9.31 | 0.66 |
| 2484 | 3203 | 6.61 | 0.51 |
| 2481 | 3203 | 8.29 | 0.47 |
| 2538 | 3203 | 2.14 | 0.30 |
| 2453 | 3203 | 10.32 | 0.51 |
| 1727 | none | 22.55 | 0.79 |
| 1984 | none | 44.21 | 1.14 |
| 1729 | none | 16.90 | 0.71 |
| 1989 | none | 8.92 | 0.49 |
| 2045 | none | 9.82 | 0.66 |
| 2021 | none | 6.54 | 0.87 |
| 2016 | none | 5.99 | 0.41 |
| 2044 | none | 10.87 | 0.88 |
| 1728 | none | 24.74 | 0.97 |
| 2468 | none | 43.72 | 1.58 |
| 2480 | none | 10.84 | 0.74 |
| 2479 | none | 10.43 | 0.63 |
| 2484 | none | 7.12 | 0.64 |
| 2481 | none | 11.19 | 0.78 |
| 2538 | none | 2.36 | 0.42 |
| 2453 | none | 16.81 | 0.70 |

TABLE 24j
Correction of G339C using PE3 systems in iPSC.

| | | | | Trial 1 | | | | Trial 2 | | | |
|---|---|---|---|---|---|---|---|---|---|---|---|
| | | | | Replcate A | | Replicate B | | Replcate A | | Replicate B | |
| PEG # | ng# | Dose | | Edit % | Indel % | Edit % | Indel % | Edit % | Indel % | Edit % | Indel % |
| 2021 | 527 | 486.7 | ng | 8.7 | 3.9 | | | | | | |
| 2021 | 527 | 324.5 | ng | 7.6 | 3.3 | 4.2 | 0.9 | 5.2 | 3.3 | 7.9 | 2 |
| 2021 | 527 | 216.4 | ng | 4.7 | 1.5 | 2.7 | 0.4 | 4 | 1.7 | 4.8 | 1 |
| 2021 | 527 | 144.3 | ng | 2.1 | 0.3 | 1.8 | 0.1 | 3.9 | 1 | | |
| 2021 | 527 | 96.1 | ng | 0.9 | 0.1 | 0.9 | 0.1 | 1.9 | 0.3 | 1.9 | 0.1 |
| 2021 | 527 | 64 | ng | 0.6 | 0. | 0.5 | 0.1 | 1 | 0.1 | 0.6 | 0.1 |
| 2021 | 527 | 42.7 | ng | 0.4 | 0 | 0.2 | 0 | 0.4 | 0 | 0.3 | 0 |
| 2021 | 527 | 28.5 | ng | 0.2 | 0 | 0.2 | 0 | 0.2 | 0.1 | 0.5 | 0 |
| 2021 | 527 | 19 | ng | 0.3 | 0 | 0.1 | 0 | 0.1 | 0 | 0.3 | 0 |
| 2021 | 527 | 12.7 | ng | 0.1 | 0.1 | 0.1 | 0 | 0.3 | 0 | 0.2 | 0 |
| 2021 | 527 | 8.5 | ng | 0.3 | 0 | 0.1 | 0 | 0.2 | 0 | 0.3 | 0 |
| 2021 | 527 | 5.6 | ng | 0.2 | 0 | 0.1 | 0 | 0.4 | 0 | 0.2 | 0 |
| 1727 | 526 | 486.7 | ng | 33.2 | 0.7 | | | | | 35.3 | 0.8 |
| 1727 | 526 | 324.5 | ng | 41.3 | 0.9 | 23.3 | 0.5 | 41.1 | 1.1 | 33.3 | 0.8 |
| 1727 | 526 | 216.4 | ng | 31.2 | 0.8 | 15 | 0.2 | 40.4 | 1 | 23.1 | 0.5 |
| 1727 | 526 | 144.3 | ng | 18 | 0.5 | 8.6 | 0.3 | 15.6 | 0.3 | 17.2 | 0.4 |
| 1727 | 526 | 96.1 | ng | 11.1 | 0.3 | 3.8 | 0 | 10.4 | 0.3 | 9.1 | 0.1 |
| 1727 | 526 | 64 | ng | 4.9 | 0.1 | 2.7 | 0.1 | 3.7 | 0.1 | 4.8 | 0.1 |
| 1727 | 526 | 42.7 | ng | 2.5 | 0.1 | 1.4 | 0.1 | 3.4 | 0 | 1.7 | 0 |
| 1727 | 526 | 28.5 | ng | 0.8 | 0 | 0.7 | 0 | 1.2 | 0 | 0.6 | 0 |
| 1727 | 526 | 19 | ng | 0.5 | 0 | 0.4 | 0 | 0.5 | 0 | 0.4 | 0 |
| 1727 | 526 | 12.7 | ng | 0.4 | 0 | 0.3 | 0 | 0.3 | 0 | 0.3 | 0 |
| 1727 | 526 | 8.5 | ng | 0.1 | 0 | 0.1 | 0 | 0.3 | 0 | 0.2 | 0 |
| 1727 | 526 | 5.6 | ng | 0.2 | 0 | 0.2 | 0 | 0.2 | 0 | 0.3 | 0 |
| 2088 | 547 | 486.7 | ng | 41.8 | 2.8 | 44.5 | | 47.8 | | 61 | 3.2 |
| 2088 | 547 | 324.5 | ng | 55.1 | 3.6 | 34.5 | 1.5 | 48.8 | 4.8 | 63.8 | 2.7 |
| 2088 | 547 | 216.4 | ng | 50.8 | 2.1 | 22.2 | 1.3 | 48.4 | 2.6 | 53.4 | 2 |
| 2088 | 547 | 144.3 | ng | 44 | 1.4 | 14.4 | 0.8 | 42.1 | 2 | 39.1 | 1.2 |
| 2088 | 547 | 96.1 | ng | 28.9 | 0.8 | 7.8 | 0.4 | 22 | 2 | 29.6 | 1 |
| 2088 | 547 | 64 | ng | 13.7 | 0.4 | 5.3 | 0.2 | 15.3 | 0.6 | 18.3 | 0.4 |
| 2088 | 547 | 42.7 | ng | 6.9 | 0.1 | 3.3 | 0.1 | 5.4 | 0.4 | 11.4 | 0.3 |
| 2088 | 547 | 28.5 | ng | 5.5 | 0.2 | 1.9 | 0.1 | 3.4 | 0.2 | 4.7 | 0.1 |
| 2088 | 547 | 19 | ng | 2.7 | 0.1 | 1.4 | 0 | 1.7 | 0.1 | 3.5 | 0.1 |
| 2088 | 547 | 12.7 | ng | 2 | 0 | 0.9 | 0 | 1 | 0.1 | 1.6 | 0.1 |
| 2088 | 547 | 8.5 | ng | 1.3 | 0.1 | 0.5 | 0 | 0.4 | 0 | 1.1 | 0 |
| 2088 | 547 | 5.6 | ng | 1.1 | 0 | | 0 | | 0 | 0.8 | 0 |
| 1989 | 3206 | 486.7 | ng | 12.1 | 1.8 | 6.9 | 0.4 | 4.2 | 0.2 | 10.8 | 1.4 |
| 1989 | 3206 | 324.5 | ng | 11.4 | 1.2 | 5.7 | 0.3 | 6.2 | 0.6 | 7.9 | 1 |
| 1989 | 3206 | 216.4 | ng | 11.2 | 1.2 | 2.4 | 0.1 | 3.8 | 0.3 | 5.6 | 0.7 |
| 1989 | 3206 | 144.3 | ng | 4.5 | 0.3 | 0.9 | 0.1 | 4.3 | 0.4 | 3.2 | 0.2 |
| 1989 | 3206 | 96.1 | ng | 1.6 | 0.1 | 0.5 | 0 | 2.2 | 0.2 | 1.7 | 0.2 |
| 1989 | 3206 | 64 | ng | 0.6 | 0 | 0.5 | 0.1 | 0.4 | 0.1 | 0.7 | 0.1 |
| 1989 | 3206 | 42.7 | ng | 0.3 | 0 | 0.3 | 0 | 0.3 | 0 | 0.3 | 0 |
| 1989 | 3206 | 28.5 | ng | 0.2 | 0 | 0.3 | 0 | 0.2 | 0.1 | 0.3 | 0 |
| 1989 | 3206 | 19 | ng | 0.2 | 0 | 0.3 | 0 | 0.1 | 0 | 0.3 | 0 |
| 1989 | 3206 | 12.7 | ng | 0.1 | 0 | 0.2 | 0 | 0.1 | 0 | 0.1 | 0 |
| 1989 | 3206 | 8.5 | ng | 0.2 | 0 | 0.3 | 0 | 0.2 | 0 | 0.1 | 0 |
| 1989 | 3206 | 5.6 | ng | 0.1 | 0 | 0.2 | 0 | 0.1 | 0 | 0.2 | 0 |
| 2021 | none | 486.7 | ng | 8.7 | 1.6 | | 0.6 | | 0.5 | | 0.8 |
| 2021 | none | 324.5 | ng | 7.6 | 0.7 | 4.2 | 0.3 | 5.2 | 1 | 7.9 | 1 |
| 2021 | none | 216.4 | ng | 4.7 | 0.9 | 2.7 | 0.2 | 4 | 0.5 | 4.8 | 0.5 |
| 2021 | none | 144.3 | ng | 2.1 | 0.1 | 1.8 | 0.1 | 3.9 | 0.8 | | 0.2 |
| 2021 | none | 96.1 | ng | 0.9 | 0.1 | 0.9 | 0 | 1.9 | 0.4 | 1.9 | 0.1 |
| 2021 | none | 64 | ng | 0.6 | 0 | 0.5 | 0 | 1 | 0 | 0.6 | 0 |
| 2021 | none | 42.7 | ng | 0.4 | 0 | 0.2 | 0 | 0.4 | 0.1 | 0.3 | 0.1 |
| 2021 | none | 28.5 | ng | 0.2 | 0 | 0.2 | 0 | 0.2 | 0.1 | 0.5 | 0 |
| 2021 | none | 19 | ng | 0.3 | 0 | 0.1 | 0 | 0.1 | 0 | 0.3 | 0 |
| 2021 | none | 12.7 | ng | 0.1 | 0 | 0.1 | 0 | 0.3 | 0 | 0.2 | 0 |
| 2021 | none | 8.5 | ng | 0.3 | 0 | 0.1 | 0 | 0.2 | 0 | 0.3 | 0 |
| 2021 | none | 5.6 | ng | 0.2 | 0 | 0.1 | 0 | 0.4 | 0 | 0.2 | 0 |
| 1727 | none | 486.7 | ng | 33.2 | 0.9 | | 0.3 | | 0.6 | 35.3 | 0.7 |
| 1727 | none | 324.5 | ng | 41.3 | 1 | 23.3 | 0.4 | 41.1 | 1 | 33.3 | 0.9 |
| 1727 | none | 216.4 | ng | 31.2 | 0.8 | 15 | 0.2 | 40.4 | 0.8 | 23.1 | 0.7 |
| 1727 | none | 144.3 | ng | 18 | 0.6 | 8.6 | 0.1 | 15.6 | 0.5 | 17.2 | 0.3 |
| 1727 | none | 96.1 | ng | 11.1 | 0.2 | 3.8 | 0.1 | 10.4 | 0.3 | 9.1 | 0.3 |
| 1727 | none | 64 | ng | 4.9 | 0.1 | 2.7 | 0.1 | 3.7 | 0.1 | 4.8 | 0.1 |
| 1727 | none | 42.7 | ng | 2.5 | 0 | 1.4 | 0 | 3.4 | 0 | 1.7 | 0.1 |
| 1727 | none | 28.5 | ng | 0.8 | 0 | 0.7 | 0 | 1.2 | 0 | 0.6 | 0 |
| 1727 | none | 19 | ng | 0.5 | 0 | 0.4 | 0 | 0.5 | 0 | 0.4 | 0 |
| 1727 | none | 12.7 | ng | 0.4 | 0 | 0.3 | 0 | 0.3 | 0 | 0.3 | 0 |
| 1727 | none | 8.5 | ng | 0.1 | 0 | 0.1 | 0 | 0.3 | 0 | 0.2 | 0 |
| 1727 | none | 5.6 | ng | 0.2 | 0 | 0.2 | 0 | 0.2 | 0 | 0.3 | 0 |
| 2088 | none | 486.7 | ng | 41.8 | 1.2 | 44.5 | 1.4 | 47.8 | 1.4 | 61 | 2 |
| 2088 | none | 324.5 | ng | 55.1 | 2.4 | 34.5 | 1.2 | 48.8 | 1.5 | 63.8 | 2 |
| 2088 | none | 216.4 | ng | 50.8 | 1.9 | 22.2 | 1.1 | 48.4 | 2.3 | 53.4 | 1.3 |
| 2088 | none | 144.3 | ng | 44 | 1.2 | 14.4 | 0.7 | 42.1 | 1.9 | 39.1 | 1.1 |
| 2088 | none | 96.1 | ng | 28.9 | 0.6 | 7.8 | 0.3 | 22 | 0.8 | 29.6 | 0.8 |
| 2088 | none | 64 | ng | 13.7 | 0.4 | 5.3 | 0.2 | 15.3 | 0.4 | 18.3 | 0.3 |
| 2088 | none | 42.7 | ng | 6.9 | 0.1 | 3.3 | 0.2 | 5.4 | 0.4 | 11.4 | 0.2 |
| 2088 | none | 28.5 | ng | 5.5 | 0.1 | 1.9 | 0.1 | 3.4 | 0.1 | 4.7 | 0.1 |
| 2088 | none | 19 | ng | 2.7 | 0.1 | 1.4 | 0.1 | 1.7 | 0.1 | 3.5 | 0.1 |
| 2088 | none | 12.7 | ng | 2 | 0.1 | 0.9 | 0.1 | 1 | 0.1 | 1.6 | 0.1 |
| 2088 | none | 8.5 | ng | 1.3 | 0 | 0.5 | 0.1 | 0.4 | 0 | 1.1 | 0 |
| 2088 | none | 5.6 | ng | 1.1 | 0 | | 0 | | 0 | 0.8 | 0 |
| 1989 | none | 486.7 | ng | 12.1 | 1.2 | 6.9 | 0.1 | 4.2 | 0.3 | 10.8 | 0.4 |
| 1989 | none | 324.5 | ng | 11.4 | 0.6 | 5.7 | 0.2 | 6.2 | 0.5 | 7.9 | 0.3 |
| 1989 | none | 216.4 | ng | 11.2 | 0.6 | 2.4 | 0.1 | 3.8 | 0.8 | 5.6 | 0.2 |
| 1989 | none | 144.3 | ng | 4.5 | 0.2 | 0.9 | 0.1 | 4.3 | 0.3 | 3.2 | 0 |
| 1989 | none | 96.1 | ng | 1.6 | 0.1 | 0.5 | 0 | 2.2 | 0.2 | 1.7 | 0.1 |
| 1989 | none | 64 | ng | 0.6 | 0 | 0.5 | 0 | 0.4 | 0 | 0.7 | 0 |
| 1989 | none | 42.7 | ng | 0.3 | 0 | 0.3 | 0 | 0.3 | 0 | 0.3 | 0 |
| 1989 | none | 28.5 | ng | 0.2 | 0 | 0.3 | 0 | 0.2 | 0 | 0.3 | 0 |
| 1989 | none | 19 | ng | 0.2 | 0 | 0.3 | 0 | 0.1 | 0 | 0.3 | 0 |
| 1989 | none | 12.7 | ng | 0.1 | 0 | 0.2 | 0 | 0.1 | 0 | 0.1 | 0 |
| 1989 | none | 8.5 | ng | 0.2 | 0.1 | 0.3 | 0 | 0.2 | 0 | 0.1 | 0 |
| 1989 | none | 5.6 | ng | 0.1 | 0 | 0.2 | 0 | 0.1 | 0 | 0.2 | 0 |

TABLE 24k
Partial list of sequences

| SEQ REF | |
|---|---|
| NO. | Sequence |
| 6043 | UCGUUGGCUAUGACUCCAAA |
| 6044 | GUUGGGAGGGGCACUCUCGU |
| 6045 | GGUGCCACACAAGUUGGGAG |
| 6046 | CGUAUGGCCCCAUUGCCGUU |
| 6047 | GUAUUUGGUUUCUCCUCGUA |
| 6048 | CUGGUAUUGGGAGCUGUAUU |
| 6049 | UGCCcuGUUU |
| 6050 | UGCacuGUUU |
| 6051 | UGCgcuGUUU |
| 6052 | UGCucuGUUU |
| 6053 | UUGCCcuGUUU |
| 6054 | UUGCacuGUUU |
| 6055 | UUGCgcuGUUU |
| 6056 | UUGCucuGUUU |
| 6057 | AUUGCCcuGUUU |
| 6058 | AUUGCacuGUUU |
| 6059 | AUUGCgcuGUUU |
| 6060 | AUUGCucuGUUU |
| 6061 | GCCCCAUUGCCcuGUUU |
| 6062 | GCCCCAUUGCacuGUUU |
| 6063 | GCCCCAUUGCgcuGUUU |
| 6064 | GCCCCAUUGCucuGUUU |
| 6065 | GGCCCCAUUGCCcuGUUU |
| 6066 | GGCCCCAUUGCacuGUUU |
| 6067 | GGCCCCAUUGCgcuGUUU |
| 6068 | GGCCCCAUUGCucuGUUU |
| 6069 | UGGCCCCAUUGCCcuGUUU |
| 6070 | UGGCCCCAUUGCacuGUUU |
| 6080 | UGGCCCCAUUGCgcuGUUU |
| 6072 | UGGCCCCAUUGCucuGUUU |
| 6081 | AUGGCCCCAUUGCCcuGUUU |
| 402 | AUGGCCCCAUUGCacuGUUU |
| 6082 | AUGGCCCCAUUGCgcuGUUU |
| 6076 | AUGGCCCCAUUGCucuGUUU |
| 6077 | UAUGGCCCCAUUGCCcuGUUU |
| 6078 | GUAUGGCCCCAUUGCCcuGUUU |
| 407 | UCGUAUGGCCCCAUUGCCcuGUUU |
| 408 | UCCUCGUAUGGCCCCAUUGCCcuGUUU |
| 409 | UCUCCUCGUAUGGCCCCAUUGCCcuGUUU |
| 410 | UUCUCCUCGUAUGGCCCCAUUGCCcuGUUU |
| 411 | UUUCUCCUCGUAUGGCCCCAUUGCCcuGUUU |
| 412 | GUUUCUCCUCGUAUGGCCCCAUUGCCcuGUUU |
| 413 | GGUUUCUCCUCGUAUGGCCCCAUUGCCcuGUUU |
| 414 | UGGUUUCUCCUCGUAUGGCCCCAUUGCCcuGUUU |
| 415 | UUGGUUUCUCCUCGUAUGGCCCCAUUGCCcuGUUU |
| 416 | UUUGGUUUCUCCUCGUAUGGCCCCAUUGCCcuGUUU |
| 417 | AUUUGGUUUCUCCUCGUAUGGCCCCAUUGCCcuGUUU |
| 418 | GCCcuGUUUGGAGUCAUAGCCAACG |
| 419 | UGCCcuGUUUGGAGUCAUAGCCAACG |
| 420 | UUGCCcuGUUUGGAGUCAUAGCCAACG |
| 421 | AUUGCCcuGUUUGGAGUCAUAGCCAACG |
| 422 | GCCCCAUUGCCcuGUUUGGAGUCAUAGCCAACG |
| 423 | GGCCCCAUUGCCcuGUUUGGAGUCAUAGCCAACG |
| 424 | UGGCCCCAUUGCCcuGUUUGGAGUCAUAGCCAACG |
| 425 | AUGGCCCCAUUGCCcuGUUUGGAGUCAUAGCCAACG |
| 426 | UAUGGCCCCAUUGCCcuGUUUGGAGUCAUAGCCAACG |
| 427 | GCCcuGUUUGGAGUCAUAGCCAACGAGAGUGCCCCUC |
| 428 | ACUCCAAACag |
| 429 | GACUCCAAACag |
| 430 | UGACUCCAAACag |
| 431 | AUGACUCCAAACag |
| 432 | UAUGACUCCAAACag |
| 433 | GCUAUGACUCCAAACag |
| 434 | GGCUAUGACUCCAAACag |
| 435 | UGGCUAUGACUCCAAACag |
| 436 | UUGGCUAUGACUCCAAACag |
| 437 | GUUGGCUAUGACUCCAAACag |
| 438 | UCGUUGGCUAUGACUCCAAACag |
| 439 | UCUCGUUGGCUAUGACUCCAAACag |
| 440 | ACUCUCGUUGGCUAUGACUCCAAACag |
| 441 | GCACUCUCGUUGGCUAUGACUCCAAACag |
| 442 | GGCACUCUCGUUGGCUAUGACUCCAAACag |
| 443 | GGGCACUCUCGUUGGCUAUGACUCCAAACag |
| 444 | GGGGCACUCUCGUUGGCUAUGACUCCAAACag |
| 445 | AGGGGCACUCUCGUUGGCUAUGACUCCAAACag |
| 446 | GAGGGGCACUCUCGUUGGCUAUGACUCCAAACag |
| 447 | GGAGGGGCACUCUCGUUGGCUAUGACUCCAAACag |
| 448 | GGGAGGGGCACUCUCGUUGGCUAUGACUCCAAACag |
| 449 | UGGGAGGGGCACUCUCGUUGGCUAUGACUCCAAACag |
| 450 | ACagGGCAAUGGGGCCAUAC |
| 451 | AACagGGCAAUGGGGCCAUAC |
| 452 | AAACagGGCAAUGGGGCCAUAC |
| 453 | UCCAAACagGGCAAUGGGGCCAUAC |
| 454 | ACUCCAAACagGGCAAUGGGGCCAUAC |
| 455 | GACUCCAAACagGGCAAUGGGGCCAUAC |
| 456 | UGACUCCAAACagGGCAAUGGGGCCAUAC |
| 457 | AUGACUCCAAACagGGCAAUGGGGCCAUAC |
| 458 | UAUGACUCCAAACagGGCAAUGGGGCCAUAC |
| 459 | GCUAUGACUCCAAACagGGCAAUGGGGCCAUAC |
| 460 | GGCUAUGACUCCAAACagGGCAAUGGGGCCAUAC |
| 461 | UGGCUAUGACUCCAAACagGGCAAUGGGGCCAUAC |
| 462 | UUGGCUAUGACUCCAAACagGGCAAUGGGGCCAUAC |
| 463 | GUUGGCUAUGACUCCAAACagGGCAAUGGGGCCAUAC |
| 464 | ACagGGCAAUGGGGCCAUACGAGGAGAAACCAAAU |
| 465 | AACagGGCAAUGGGGCCAUACGAGGAGAAACCAAAU |
| 466 | AAACagGGCAAUGGGGCCAUACGAGGAGAAACCAAAU |
| | GGAGUCA (SEQ REF NO: 467) |
| | GGAGUCAU (SEQ REF NO: 468) |
| | GGAGUCAUA (SEQ REF NO: 469) |
| 470 | GGAGUCAUAG |
| 471 | GGAGUCAUAGC |
| 472 | GGAGUCAUAGCC |
| 473 | GGAGUCAUAGCCA |
| 474 | GGAGUCAUAGCCAA |
| 475 | GGAGUCAUAGCCAAC |
| | AGAGUGC (SEQ REF NO: 476) |
| | AGAGUGCC (SEQ REF NO: 477) |
| | AGAGUGCCC (SEQ REF NO: 478) |
| 479 | AGAGUGCCCC |
| 480 | AGAGUGCCCCU |
| 481 | AGAGUGCCCCUC |
| 482 | AGAGUGCCCCUCC |
| 483 | AGAGUGCCCCUCCC |
| 484 | AGAGUGCCCCUCCCA |
| | CCAACUU (SEQ REF NO: 485) |
| | CCAACUUG (SEQ REF NO: 486) |
| | CCAACUUGU (SEQ REF NO: 487) |
| 488 | CCAACUUGUG |
| 489 | CCAACUUGUGU |
| 490 | CCAACUUGUGUG |
| 491 | CCAACUUGUGUGG |
| 492 | CCAACUUGUGUGGC |
| 493 | CCAACUUGUGUGGCA |
| | GGCAAUG (SEQ REF NO: 494) |
| | GGCAAUGG (SEQ REF NO: 495) |
| | GGCAAUGGG (SEQ REF NO: 496) |
| 497 | GGCAAUGGGG |
| 498 | GGCAAUGGGGC |
| 499 | GGCAAUGGGGCC |
| 500 | GGCAAUGGGGCCA |
| 501 | GGCAAUGGGGCCAU |
| 502 | GGCAAUGGGGCCAUA |
| | GAGGAGA (SEQ REF NO: 503) |
| | GAGGAGAA (SEQ REF NO: 504) |
| | GAGGAGAAA (SEQ REF NO: 505) |
| 506 | GAGGAGAAAC |
| 507 | GAGGAGAAACC |
| 508 | GAGGAGAAACCA |
| 509 | GAGGAGAAACCAA |
| 510 | GAGGAGAAACCAAA |
| 511 | GAGGAGAAACCAAAU |
| | ACAGCUC (SEQ REF NO: 512) |
| | ACAGCUCC (SEQ REF NO: 513) |
| | ACAGCUCCC (SEQ REF NO: 514) |
| 515 | ACAGCUCCCA |
| 516 | ACAGCUCCCAA |
| 517 | ACAGCUCCCAAU |
| 518 | ACAGCUCCCAAUA |
| 519 | ACAGCUCCCAAUAC |
| 520 | ACAGCUCCCAAUACC |
| 521 | AUUGUGGGACUCAUGGCCAA |
| 522 | CCACGCCAUUGUGGGACUCA |
| 523 | GGCACCUCCCACGCCAUUGU |
| 524 | UGGCACCUCCCACGCCAUUG |
| 525 | AGUGCCCCUCCCAACUUGUG |
| 526 | UAUGGCCCCAUUGCCcuGUU |
| 527 | GUAUUUGGUUUCUCCUCGUA |
| 528 | CUGGUAUUGGGAGCUGUAUU |
| 529 | CAGCUCUGGAUCCUGGUAUU |
| 530 | GCAGCUCUGGAUCCUGGUAU |
| 531 | CCCUUGGCAGCUCUGGAUCC |
| 532 | CACUCUGCCCUUGGCAGCUC |
| 533 | AUGCCACUCCACUCUGCCCU |
| 534 | UAGAAGUUAACACUUACCAU |
| 535 | AUUGGCCAUGAGUCCCACAA |
| 536 | CCAUGAGUCCCACAAUGGCG |
| 537 | CAUGAGUCCCACAAUGGCGU |
| 538 | GAGUCCCACAAUGGCGUGGG |
| 539 | GUGGGAGGUGCCACACAAGU |
| 540 | UGGGAGGUGCCACACAAGUU |
| 541 | GAGGUGCCACACAAGUUGGG |
| 542 | AGGUGCCACACAAGUUGGGA |
| 543 | GGUGCCACACAAGUUGGGAG |
| 544 | GUUGGGAGGGGCACUCUCGU |
| 545 | GUUGGCUAUGACUCCAAACa |
| 546 | UAUGACUCCAAACagGGCAA |
| 547 | AUGACUCCAAACagGGCAAU |
| 548 | UGACUCCAAACagGGCAAUG |
| 549 | CagGGCAAUGGGGCCAUACG |
| 550 | CAAAUACAGCUCCCAAUACC |
| 551 | ACCAGGAUCCAGAGCUGCCA |
| 552 | CCAGGAUCCAGAGCUGCCAA |
| 553 | CAGAGCUGCCAAGGGCAGAG |
| 554 | CUGCCAAGGGCAGAGUGGAG |
| 555 | AGUGGAGUGGCAUUCAGAGU |
| 3201 | UAUGGCCCCAUUGCUCUGUU |
| 3202 | UAUGGCCCCAUUGCACUGUU |
| 3203 | UAUGGCCCCAUUGCGCUGUU |
| 3204 | ACCAAGCCUUCUUCUUGCUU |
| 3205 | AGCCUUCUUCUUGCUUUGGC |
| 3206 | GCCUUCUUCUUGCUUUGGCA |
| 3207 | CCUUCUUCUUGCUUUGGCAG |
| 3208 | UCUUCUUGCUUUGGCAGGGG |
| 3209 | GCAGGGGUGGGUAGGCAGGU |
| 3210 | UUUGGCAGGGGUGGGUAGGC |
| 3211 | CGUUGGCUAUGACUCCAAAC |
| 3212 | GUAUUUGGUUUCUCAUCGUA |
| 3213 | GUAUUUGGUUUCUCGUCGUA |
| 3214 | GUAUUUGGUUUCUCUUCGUA |
| 3215 | CCCCUGCCAAAGCAAGAAGA |
| 3216 | UUGCUUUGGCAGGGGUGGGU |
| 6001 | CUAUGACUCCAAACag |
| 6003 | CCAAACagGGCAAUGGGGCCAUAC |
| 6004 | CUCCAAACagGGCAAUGGGGCCAUAC |
| 6005 | CAUUGCucuGUUU |
| 6006 | CAUUGCgcuGUUU |
| 6007 | CAUUGCacuGUUU |
| 6008 | CCAUUGCucuGUUU |
| 6009 | CCAUUGCgcuGUUU |
| 6010 | CCAUUGCacuGUUU |
| 6011 | CCCAUUGCucuGUUU |
| 6012 | CCCAUUGCgcuGUUU |
| 6013 | CCCAUUGCacuGUUU |
| 6014 | CCCCAUUGCucuGUUU |
| 6015 | CCCCAUUGCgcuGUUU |
| 6016 | CCCCAUUGCacuGUUU |
| 6017 | CCCCAUUGCCcuGUUU |
| 6018 | CAUUGCCcuGUUU |
| 6019 | CCAUUGCCcuGUUU |
| 6020 | CCCAUUGCCcuGUUU |

1. (canceled)

2. A prime editing guide RNA (PEgRNA) comprising:
a spacer that comprises a region of complementarity to a search target sequence on a target strand of an SLC37A4 gene,
an editing template that comprises an intended nucleotide edit compared to the SLC37A4 gene and a region of complementarity to an editing target sequence on a non-target strand of the SLC37A4 gene, a primer binding site (PBS) that comprises a region of complementarity to a region immediately upstream of a nick site in the non-target strand, and
a gRNA core that associates with a prime editor comprising a DNA binding domain and a DNA polymerase domain,
wherein the target strand and the non-target strand are complementary to each other, and wherein the editing target sequence comprises
i) a codon encoding cysteine corresponding to position 339 of a SLC37A4 wild-type peptide and the intended nucleotide edit comprises a T>G nucleotide substitution at position 1015 in the coding sequence of the SLC37A4 gene, or
ii) a 2-nucleotide deletion corresponding to positions 1042-1043 of a coding sequence of an SLC37A4 wild-type gene, and wherein the intended nucleotide edit comprises a CT insertion at the position corresponding to 1042 of the coding sequence of the SLC37A4 wild-type gene.

3.-11. (canceled)

12. The PEgRNA of claim 112, wherein the editing template is about 10 to 30 nucleotides in length.

13. (canceled)

14. The PEgRNA of claim 2, wherein the gRNA core is between the spacer and the editing template.

15.-16. (canceled)

17. The PEgRNA of claim 2, wherein the PBS and the editing template are directly adjacent to each other.

18.-19. (canceled)

20. The PEgRNA of claim 2, wherein the PBS is about 8 to 16 nucleotides in length.

21.-23. (canceled)

24. The PEgRNA of claim 2, wherein the search target sequence is complementary to a protospacer sequence in the SLC37A4 gene, and wherein the protospacer sequence is adjacent to a search target adjacent motif (PAM) in the SLC37A4 gene and wherein the PEgRNA guides the prime editor to incorporate a further intended nucleotide edit in the PAM when contacted with the SLC37A4 gene.

25.-35. (canceled)

36. The PEgRNA of claim 2, wherein the spacer comprises a sequence selected from the spacer sequences listed in Table 8.

37. The PEgRNA of claim 36, wherein the editing template comprises a sequence selected from the editing template sequences listed in Table 9a, Table 10a, Table 11a, Table 12a, Table 13a, Table 14a, Table 15a, Table 16a, Table 17a, Table 18a, Table 19a, Table 20a, Table 21a, Table 22a, Table 23a, and Table 24a.

38. The PEgRNA of any one of claim 37, wherein the PBS comprises a sequence selected from the PBS sequences listed in Table 9b, Table 10b, Table 11b, Table 12b, Table 13b, Table 14b, Table 15b, Table 16b, Table 17b, Table 18b, Table 19b, Table 20b, Table 21b, Table 22b, Table 23b, and Table 24b.

39. The PEgRNA of claim 38, wherein:
the spacer comprises the sequence of S01, the editing template comprises a sequence selected from the editing template sequences listed in Table 9a, and the PBS comprises a sequence selected from the PBS sequences listed in Table 9b; or
the spacer comprises the sequence of S02, the editing template comprises a sequence selected from the editing template sequences listed in Table 10a, and the PBS comprises a sequence selected from the PBS sequences listed in Table 10b; or
the spacer comprises the sequence of S03, the editing template comprises a sequence selected from the editing template sequences listed in Table 11a, and the PBS comprises a sequence selected from the PBS sequences listed in Table 11b; or
the spacer comprises the sequence of S04, the editing template comprises a sequence selected from the editing template sequences listed in Table 12a, and the PBS comprises a sequence selected from the PBS sequences listed in Table 12b; or
the spacer comprises the sequence of S05, the editing template comprises a sequence selected from the editing template sequences listed in Table 13a, and the PBS comprises a sequence selected from the PBS sequences listed in Table 13b; or
the spacer comprises the sequence of S06, the editing template comprises a sequence selected from the editing template sequences listed in Table 14a, and the PBS comprises a sequence selected from the PBS sequences listed in Table 14b; or
the spacer comprises the sequence of S07, the editing template comprises a sequence selected from the editing template sequences listed in Table 15a, and the PBS comprises a sequence selected from the PBS sequences listed in Table 15b; or
the spacer comprises the sequence of S08, the editing template comprises a sequence selected from the editing template sequences listed in Table 16a, and the PBS comprises a sequence selected from the PBS sequences listed in Table 16b; or
the spacer comprises the sequence of S09, the editing template comprises a sequence selected from the editing template sequences listed in Table 17a, and the PBS comprises a sequence selected from the PBS sequences listed in Table 17b; or
the spacer comprises the sequence of S10, the editing template comprises a sequence selected from the editing template sequences listed in Table 18a, and the PBS comprises a sequence selected from the PBS sequences listed in Table 18b; or
the spacer comprises the sequence of S11, the editing template comprises a sequence selected from the editing template sequences listed in Table 19a, and the PBS comprises a sequence selected from the PBS sequences listed in Table 19b; or
the spacer comprises the sequence of S12, the editing template comprises a sequence selected from the editing template sequences listed in Table 20a, and the PBS comprises a sequence selected from the PBS sequences listed in Table 20b; or
the spacer comprises the sequence of S13, the editing template comprises a sequence selected from the editing template sequences listed in Table 21a, and the PBS comprises a sequence selected from the PBS sequences listed in Table 21b; or
the spacer comprises the sequence of S14, the editing template comprises a sequence selected from the editing template sequences listed in Table 22a, and the PBS comprises a sequence selected from the PBS sequences listed in Table 22b; or
the spacer comprises the sequence of S15, the editing template comprises a sequence selected from the editing template sequences listed in Table 23a, and the PBS comprises a sequence selected from the PBS sequences listed in Table 23b; or
the spacer comprises the sequence of S16, the editing template comprises a sequence selected from the editing template sequences listed in Table 24a, and the PBS comprises a sequence selected from the PBS sequences listed in Table 24b.

40.-42. (canceled)

43. A PEgRNA system comprising the PEgRNA according to claim 2, further comprising a nick guide RNA (ngRNA), wherein the ngRNA comprises an ng spacer that comprises a region of complementarity to a second search target sequence in the SLC37A4 gene, and wherein the second search target sequence is on the non-target strand of the SLC37A4 gene.

44. (canceled)

45. The PEgRNA system of claim 43, wherein the ng spacer comprises a sequence listed in Table 8a, Table 8b, Table 8c, or Table 8d.

46. The PEgRNA system of claim 45, wherein: (a) the PEgRNA comprises RTT and PBS sequences listed together in Table 9c, Table 10c, Table 11c, Table 12c, Table 13c, Table 14c, Table 19c, Table 20c, or Table 21c, and the ngRNA comprises a sequence listed in Table 8a or Table 8c; or (b) the PEgRNA comprises RTT and PBS sequences listed in Table 15c, Table 16c, Table 17c, Table 18c, Table 22c, Table 23c, Table 24c and the ngRNA comprises a sequence listed in Table 8b or Table 8d.

47. (canceled)

48. A prime editing system comprising: (i) the PEgRNA of claim 2; and (ii) the prime editor comprising the DNA binding domain and the DNA polymerase domain, or one or more nucleotides encoding the prime editor.

49.-90. (canceled)

91. The prime editing system of claim 48, wherein the DNA binding domain is a Cas9 having nickase activity and wherein the DNA polymerase domain is a reverse transcriptase.

92. A prime editing system comprising: (i) the PEgRNA of claim 43; and (ii) the prime editor comprising the DNA binding domain and the DNA polymerase domain, or one or more nucleotides encoding the prime editor.

93. The prime editing system of claim 92, wherein the DNA binding domain is a Cas9 having nickase activity and wherein the DNA polymerase domain is a reverse transcriptase.

94. A lipid nanoparticle (LNP) or ribonucleoprotein (RNP) comprising the prime editing complex system of claim 43, or a component thereof.

95. A lipid nanoparticle (LNP) or ribonucleoprotein (RNP) comprising the prime editing complex system of claim 48, or a component thereof.

96. A method for editing an SLC37A4 gene, the method comprising contacting the SLC37A4 gene with:
(a) a Prime Editing guide RNA (PEgRNA), or one or more nucleic acids encoding the pegRNA, the pegRNA comprising:
(i) a spacer that comprises a region of complementarity to a search target sequence on a target strand of an SLC37A4 gene,
(ii) an editing template that comprises an intended nucleotide edit compared to the SLC37A4 gene and a region of complementarity to an editing target sequence on a non-target strand of the SLC37A4 gene,
(iii) a primer binding site (PBS) that comprises a region of complementarity to a region immediately upstream of a nick site in the non-target strand, and
(iv) a gRNA core that associates with a prime editor comprising a DNA binding domain and a DNA polymerase domain; and
(b) a prime editor, or one or more nucleic acids encoding the prime editor, the prime editor comprising a DNA binding domain and a DNA polymerase domain;
wherein the target strand and the non-target strand are complementary to each other, and
wherein the editing target sequence comprises:
(i) a codon encoding cysteine corresponding to position 339 of a SLC37A4 wild-type peptide and the intended nucleotide edit comprises a T>G nucleotide substitution at position 1015 in the coding sequence of the SLC37A4 gene, or
(ii) a 2-nucleotide deletion corresponding to positions 1042-1043 of a coding sequence of an SLC37A4 wild-type gene, and wherein the intended nucleotide edit comprises a CT insertion at the position corresponding to 1042 of the coding sequence of the SLC37A4 wild-type gene.
